(19) United States
(12) Patent Application Publication
(10) Pub. No.: US 2005/0266442 A1
(43) Pub. Date: Dec. 1, 2005

(54) Immortalized human Tuberous Sclerosis null angiomyolipoma cell and method of use thereof

(76) Inventors: Rachel Squillace, Guilford, CT (US); Michael P. Weiner, Guilford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/090,439

(22) Filed: Mar. 25, 2005

Related U.S. Application Data
(60) Provisional application No. 60/556,344, filed on Mar. 25, 2004.

(51) Int. Cl. C12Q 1/68; C12N 5/08

(52) U.S. Cl. 435/6; 435/366

(57) ABSTRACT

Disclosed are immortailized cells and cell lines that do not express the Tuberous Sclerosis Complex(TSC)-2 gene. Also sisclosed are methods of detecting TSC-related disorders using differentially expressed genes.

### RELATED APPLICATIONS

This application claims the benefit of U.S. Ser. No. 60/556,344, filed Mar. 25, 2004. he contents of this application is incorporated herein by reference in its entirety.

### FIELD OF THE INVENTION

The invention relates compositions and methods of treating and preventing Tuberous Sclerosis Complex (TSC) related disorders. More specifically, the invention provides a novel TSC^{-/-} cell line.

### BACKGROUND OF THE INVENTION

TSC is an autosomal dominant disorder characterized by widespread benign hamartomas, epilepsy, mental retardation, and autism. Occurring once in 6,000 live births, TSC is linked to mutations in the tumor suppressor genes, TSCI and TSC2. Mutation in either of these two genes leads to the clinical manifestations of TSC. Interestingly, loss of TSC gene function does not result in neoplastic transformation, but rather in increased cellular growth and benign tumor formation. While many of the features of TSC are neurological in nature, renal dysfunction is a common characteristic of the disease. Approximately 70-80% of TSC patients develop renal angiomyolipomas (AMLs). AMLs are heterogeneous, benign tumors composed of three distinct cell types including smooth muscle, blood vessel, and adipose cells.

TSC patients also present with evidence of a devastating form of lung disease called Lymphangioleiomyomatosis (LAM). LAM is a unique and rare cystic pulmonary disease that afflicts predominately premenopausal women. While its prevalence is not precisely known, up to one thousand women may be affected by LAM annually in the United States. The clinical symptoms are dysapnea, chronic cough, wheezing, pneumothorax, and chest pain. These symptoms occur and worsen as LAM cells migrate into the lung, causing cystic parenchymal destruction and progressive respiratory failure. LAM can occur as an independent condition (sporadic LAM) or as a secondary condition of TSC (TSC-LAM). The genetic connection between LAM and TSC is evident in work done by Henske et al., revealing inactivating mutations in the TSC2 gene in both TSC-LAM patients and sporadic LAM patients. TSC patients with clinically diagnosed LAM were thought to be quite rare (<4%), but recent studies using High Resolution Computed Tomography (HRCT) scans indicate evidence of LAM in 26-42% of women with TSC. Currently, the only treatment for LAM is lung transplantation.

AMLs are symptomatic of both LAM (50% of patients presenting) and TSC (70% of patients presenting), and there are no radiological, morphological, or genetic differences between AMLs from the two disorder. Designing therapies against AMLs has been slowed by the lack of reliable protein markers against which to design therapeutics. AMLs exhibit a characteristic expression of melanocyte differentiation markers such as silv/pMel17/gp 100 (silv) and melanA/MART1 (melan-A). However these markers have been shown to be upregulated in no more then 50% of AMLs from either TSC or LAM patients renewing the importance of identifying better candidate therapeutic targets. Because silv and melan-A are not expressed in many AMLs, the only reliable method for AML cell determination is TSC1^{-/-} or TSC2^{-/-} status determined by genomic sequencing. Thus, there is a need to identify other molecular markers to distinguish an AML cell, from a non-AML cell.

### SUMMARY OF THE INVENTION

The invention provides an immortalized cell that does not express a Tuberous Sclerosis-2 (TSC2) gene. The cell is refered to herein as TSC2^{-/-} cell or a TSC2 null cell. The cell is capable of phosporylating, e.g. constitutively, ribosomal S6 or S6 kinase. Additionally, the invention features a TSC2^{-/-} cell culture, e.g., an in-vitro culture. The culture is an adhesion culture. Alternatively, the cells in the culture are in suspension. The cell is from a mammal such as human, a primate, mouse, rat, dog, cat, cow, horse, pig. The cell contains a mutation in a TSC2 gene. The mutation is in exon 16 of the TSC2 gene. The mutation results in a single nucleotide transition. The transition is a guanine to adenine transition. The mutation is for example at nucleotide position 1832 of a TSC2 gene when numbered in accordance with a wild-type (i.e., non-mutated TSC2 gene). The cell contains a TSC2 gene that has a Pvu II restriction site. The Pvyu II restriction site is upstream of nucleotide position 1832 in exon 16, when numbered in accordance with a wild type TSC2 gene. Alternatively, the Pvu II restriction site is downstream of nucleotide position 1832 in exon 16, when numbered in accordance with a wild type TSC2 gene. For example, the Pvu II restriction site is at least 2, 4, 6, 8 10, 20, 40, 50, 75 or more nucleotides upstream or down stream of nucleotide position 1832 in exon 16 of a TSC2 gene.

Also included in the invention is the TSC^{-/-} cell line which was deposited at the American Type Tissue Collection and assigned ATCC designation ______, and ______.

The invention is further based the discovery of a pattern of gene expression correlated with angiomyolipomas. The genes that are differentially expressed in angiomyolipomas are collectively referred to herein as “TSC nucleic acids” or “TSC polynucleotides” and the corresponding encoded polypeptides are referred to as “TSCpolypeptides” or “TSC proteins.”

Accordingly, the invention features a method of diagnosing or determining a predisposition to a TSC-related disorder by providing a biological sample conataining genomic DNA, amplifying a region of the genomic DNA which contains position 1832 of Exon 16 of the TSC2 gene and digesting amplification product from with a Pvu II restriction endonucleases. Identifying a Pvu II restriction site upstream or downstream from position 1832 in the TSC2 gene indicates a TSC-related disorder or a predisposition to developing TSC related disorder in the subject.

TSC-related disorders or a predisposition to a TSC-related disorder is determined in a subject by determining a level of expression of TSC-associated gene in a patient derived tissue sample. By TSC-associated gene is meant a gene that is characterized by a level of expression which differs in a cell obtained from a cell from a patient with a TSC-related disorder compared to a normal cell. A normal cell is one obtained from a patiet without a TSC-related disorder. An TSC-associated gene includes for example TSC 1-26. An alteration, e.g., increase or decrease of the level of expression of the gene compared to a normal control level of the gene indicates that the subject suffers from or is at risk of developing a TSC-related disorder.

By normal control level is meant a level of gene expression detected in a normal, healthy individual or in a population of individuals known not to be suffering from a TSC-related disorder. A control level is a single expression pattern derived from a single reference population or from a plurality of expression patterns. For example, the control level can be a database of expression patterns from previously tested cells.

An increase in the level of TSC1-25 detected in a test sample compared to a normal control level indicates the subject (from which the sample was obtained) suffers from or is at risk of developing. In contrast, a decrease in the level of TSC 26 detected in a test sample compared to a normal control level indicates said subject suffers from or is at risk of developing A TSC-related disorder.

A TSC-related disorder includes for example seizures, mental retardation, autism, benign tumors, hamartomas, renal disease, angiomyolipomas, renal cell carcinoma, kidney disorders, polycystic kidney disease, Lymphangioleiomyomatosis, brain tumors such as cortical tubers, subependymal nodules, and giant-cell astrocytomas, fibromas of the finger and toenails, pitted teeth, dermatological lesions, hypomelanotic macules, confetti skin lesions, facial angiofibromas, ungual fibromas, Shagreen's patches, and forehead plaque.

Alternatively, expression of a panel of TSC-associated genes in the sample is compared to a TSC control level of the same panel of genes. By TSC control level is meant the expression profile of the TSC-associated genes found in a population suffering from a TSC related-disorder.

Gene expression is increased or decreased 10%, 25%, 50% compared to the control level. Alternately, gene expression is increased or decreased 1, 2, 5, 10, 20, 25 or more fold compared to the control level. Expression is determined by detecting hybridization, e.g., on a chip, of TSC gene probe to a gene transcript of the patient-derived tissue sample.

The alteration is statistically significant. By statistically significant is meant that the alteration is greater than what might be expected to happen by change alone. Statistical significance is determined by method known in the art. An alteration is statistically significant if the p-value is at least 0.05. Preferably, the p-value is 0.04, 0.03, 0.02, 0.01, 0.005, 0.001 or less.

The patient derived tissue sample is any tissue from a test subject, e.g., a patient known to or suspected of having a TSC related-disorder. For example, the tissue contains a primary angiomyolipmoma cancer cell.

The invention also provides TSC reference expression profile of a gene expression level of one or more of TSC 2, 4-26. Alternatively, the invention provides a TSC reference expression profile of the levels of expression two or more of TSC 1-26 The invention further provides methods of identifying an agent that inhibits or enhances the expression or activity of TSC-associated gene, e.g., TSC 1-26 by contacting a test cell expressing TSC associated gene with a test agent and determining the expression level of the TSC-associated gene. The test cell is a brain cell, a skin cell ,an eye cell, a heart cell, a kidney cell, a bone cell, a lung cell or an intestinal cell. A decrease of the level compared to a normal control level of the gene indicates that the test agent is an inhibitor of the TSC-associated gene and reduces a TSC-related disorder. Alternatively, an increase of the level or activity compared to a normal control level or activity of the gene indicates that said test agent is an enhancer of expression or function of the TSC-associated gene.

The invention also provides a kit with a detection reagent which binds to two or more TSC nucleic acid sequences or which binds to a gene product encoded by the nucleic acid sequences. Also provided is an array of nucleic acids that binds to two or more TSC nucleic acids.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A (left panel) is a photograph of a Southern Blot showing genomic analysis of TSC2 in AML primary sample and clones.

FIG. 1A (right panel) is an illustration showing that missense mutation in exon 16 of the TSC2 gene that results in a new PvuII restriction enzyme site and the elimination of an HpaII site.

FIG. 1B is a series of photomicrographs of AML TSC2−/− (AML-1, AML-2) and TSC2+/+ (wt1, wt2) clones. Images of each clone were taken at 100× magnification using a Zeiss Axiovert 25 microscope.

FIG. 2 is a series of photographs of Western Blots showing protein expression analysis of AML clones.

FIG. 3 is a series of line graphs showing that AML TSC2−/− cell lines are rapamycin sensitive.

FIG. 4 is a schematic showing hierarchical clustering of AMLs and normal tissue.

FIG. 5 is a series of bar graphs showing RTQ-PCR expression analysis of genes up-regulated in AMLs.

FIG. 6A is a photograph of a Western Blot showing GPNMB expression in melanoma and AML tissues. Expression of housekeeping genes varies between different tissues, but coomassie staining indicated equal protein loads.

FIG. 6B is a photograph of a Western Blot showingOA1 expression in melanoma and AML tissues. Expression of housekeeping genes varies between different tissues, but coomassie staining indicated equal protein loads.

FIG. 7 is a schematic representation of the TSC signaling pathway.

### DETAILED DESCRIPTION

The present invention is based in part upon the establishment and characterization of several continuous cell lines of immortalized human angiomyolypoma (AML) cell lines. Specifically, a human TSC-1-AML cell line and a set of matching TSC gene knock-in control cell lines have been developed. These cells lines provide an in vitro cellular model for Lymphangioleiomyomatosis (LAM) and Tuberous Sclerosis Complex (TSC) and are useful for differential gene expression profiling, the identification of therapeutically beneficial compounds for LAM and TSC, the elucidation the molecular mechanisms of aberrant LAM and TSC cell behavior and small molecule chemical screening and compound validation for compounds affecting the mTOR pathway, which is known to be involved in cancer and inflammation. The invention is further based on the discovery of changes in expression patterns of multiple nucleic acid sequences in cancer tissue from patients with sporadic LAM. The differences in gene expression were identified by using RTQ-PCR and a comprehensive cDNA microarray system. Microarray analysis of 4 primary AML tissues and a novel human AML TSC2^{-/-} cell lines compared with normal tissues has identified 289 transcripts over-expressed (t<0.05) in AMLs by >3-fold, 115>5-fold, and 25>10-fold. Of the up-regulated genes 26 have been identified as transmembrane or secreted proteins, including 7 Melanoma Associated Antigens (MAAs). These 26 genes and their encoded polypeptides (i.e., TSC1-26) are candidate targets for vaccine and antibody therapy development for TSC-related disorders. (See Table A)

The differntially expressed genes identified herein are used for diagnositic and prognostic purposes and to develop gene or protein targeted therapeutic approaches to TSC related disorders. The genes whose expression levels increased in patients with AML are summarized in Tables A-D and are collectively referred to herein as “TSC-associated genes”, “TSC nucleic acids” or “TSC polynucleotides” and the corresponding encoded polypeptides are referred to as “TSC polypeptides” or “TSC proteins.” Unless indicated otherwise, “TSC” is meant to refer to any of the sequences disclosed herein. The genes have been previously described and are presented along with a database accession number.

TABLE A
Transmembrane or Secreted Proteins Associated with TSC-Related Disorders

| | | | | | Gene | |
|---|---|---|---|---|---|---|
| TSC | ProbeSet ID | | | | Family | Cellular |
| No.t | Affymetrix | Gene Symbol | Gene Name | Descriptions | Name | localization |
| 1 | 206427_s_at | MLANA | melan-A | gb: U06654.1/DB_XREF = gi: 517022/ | — | integral to |
| | | | | FEA = FLmRNA/CNT = 66/ | | plasma |
| | | | | TID = Hs.154069.0/TIER = FL/STK = 5/ | | membrane |
| | | | | UG = Hs.154069/LL = 2315/ | | |
| | | | | UG_GENE = MLANA/DEF = Human | | |
| | | | | differentiation antigen melan-A protein | | |
| | | | | mRNA, complete cds./PROD = melan- | | |
| | | | | A protein/FL = gb: U06654.1 | | |
| | | | | gb: NM_005511.1 | | |
| 2 | 206696_at | OA1/GPR143 | Ocular albinism 1/G- | gb: NM_000273.1/ | GPCRs | membrane |
| | | | rotein-coupled | DB_XREF = gi: 4557806/GEN = OA1/ | | fraction /// |
| | | | receptor 143 | FEA = FLmRNA/CNT = 14/ | | cytoplasm /// |
| | | | | TID = Hs.74124.0/TIER = FL + Stack/ | | integral to |
| | | | | STK = 9/UG = Hs.74124/LL = 4935/ | | membrane |
| | | | | DEF = Homo sapiens ocular albinism 1 | | |
| | | | | (Nettleship-Falls) (OA1), mRNA./ | | |
| | | | | PROD = ocular albinism 1 (Nettleship- | | |
| | | | | Falls) protein/FL = gb: NM_00 | | |
| 3 | 209848_s_at | SILV | silver/gp100/pMel17 | gb: U01874.1/DB_XREF = gi: 494939/ | Pmel- | plasma |
| | | | | FEA = FLmRNA/CNT = 177/ | 17/NMB | membrane /// |
| | | | | TID = Hs.95972.0/TIER = FL/STK = 0/ | family | integral to |
| | | | | UG = Hs.95972/LL = 6490/ | | membrane |
| | | | | UG_GENE = SILV/DEF = Human | | |
| | | | | me20m mRNA, complete cds./ | | |
| | | | | PROD = me20m/FL = gb: NM_006928.1 | | |
| | | | | gb: BC001414.1 gb: U01874.1 | | |
| 4 | 218468_s_at | GREM1/DRM | gremlin 1 homolog, | gb: AF154054.1/ | — | extracellular |
| | | | cysteine knot | DB_XREF = gi: 10863087/GEN = DRM/ | | space |
| | | | superfamily | FEA = FLmRNA/CNT = 228/ | | |
| | | | (Xenopus laevis) | TID = Hs.40098.0/TIER = FL + Stack/ | | |
| | | | | STK = 20/UG = Hs.40098/LL = 26585/ | | |
| | | | | DEF = Homo sapiens DRM (DRM) | | |
| | | | | mRNA, complete cds./PROD = DRM/ | | |
| | | | | FL = gb: NM_013372.1 gb: AF110137.2 | | |
| | | | | gb: AF045800.1 gb: AF154054.1 | | |
| 5 | 243167_at | ABCB5 | ATP-binding cassette, | gb: AL040763/DB_XREF = gi: 5409709/ | — | — |
| | | | sub-family B (MDR/ | DB_XREF = DKFZp434C1815_s1/ | | |
| | | | TAP), member 5 | CLONE = DKFZp434C1815/FEA = EST/ | | |
| | | | | CNT = 6/TID = Hs.310735.0/ | | |
| | | | | TIER = ConsEnd/STK = 2/ | | |
| | | | | UG = Hs.310735/UG_TITLE = ESTs, | | |
| | | | | Moderately similar to ALU7_HUMAN | | |
| | | | | ALU SUBFAMILY SQ SEQUENCE | | |
| | | | | CONTAMINATION WARNING ENTRY | | |
| | | | | (H. sa | | |
| 6 | 206638_at | HTR2B | 5-hydroxytryptamine | gb: NM_000867.1/ | GPCRs | integral to |
| | | | (serotonin) receptor 2B | DB_XREF = gi: 4504538/GEN = HTR2B/ | | plasma |
| | | | | FEA = FLmRNA/CNT = 13/ | | membrane |
| | | | | TID = Hs.2507.0/TIER = FL + Stack/ | | |
| | | | | STK = 10/UG = Hs.2507/LL = 3357/ | | |
| | | | | DEF = Homo sapiens 5- | | |
| | | | | hydroxytryptamine (serotonin) receptor | | |
| | | | | 2B (HTR2B), mRNA./PROD = 5- | | |
| | | | | hydroxytryptamine (serotonin) receptor | | |
| | | | | 2B/FL | | |
| 7 | 220484_at | MCOLN3 | mucolipin 3 | gb: NM_018298.1/ | — | integral to |
| | | | | DB_XREF = gi: 8922819/ | | membrane |
| | | | | GEN = FLJ11006/FEA = FLmRNA/ | | |
| | | | | CNT = 6/TID = Hs.49344.0/TIER = FL/ | | |
| | | | | STK = 0/UG = Hs.49344/LL = 55283/ | | |
| | | | | DEF = Homo sapiens hypothetical | | |
| | | | | protein FLJ11006 (FLJ11006), mRNA. | | |
| | | | | PROD = hypothetical protein FLJ11006/ | | |
| | | | | FL = gb: NM_018298.1 | | |
| 8 | 213790_at | ADAM12 | a disintegrin and | gb: W46291/DB_XREF = gi: 1330989/ | peptidase | plasma |
| | | | metalloproteinase | DB_XREF = zc31b08.s1 | family | membrane /// |
| | | | domain 12 (meltrin | CLONE = IMAGE: 323895/FEA = EST/ | M12B | integral to |
| | | | alpha) | CNT = 27/TID = Hs.8850.2/TIER = Stack/ | | membrane |
| | | | | STK = 12/UG = Hs.8850/LL = 8038/ | | |
| | | | | UG_GENE = ADAM12/UG_TITLE = a/ | | |
| | | | | disintegrin and metalloproteinase | | |
| | | | | domain 12 (meltrin alpha) | | |
| 9 | 214156_at | MYRIP | myosin VIIA and Rab | gb: AL050090.1/ | — | — |
| | | | interacting protein | DB_XREF = gi: 4884109/ | | |
| | | | | GEN = DKFZp586F1018/FEA = mRNA/ | | |
| | | | | CNT = 28/TID = Hs.26970.0/ | | |
| | | | | TIER = Stack/STK = 19/UG = Hs.26970/ | | |
| | | | | LL = 25924/DEF = Homo sapiens | | |
| | | | | mRNA; cDNA DKFZp586F1018 (from | | |
| | | | | clone DKFZp586F1018)./ | | |
| | | | | PROD = hypothetical protein | | |
| 10 | 229150_at | MLPH | melanophilin | gb: AI810764/DB_XREF = gi: 5397330/ | — | mitochondrion |
| | | | | DB_XREF = tu04c11.x1/ | | |
| | | | | CLONE = IMAGE: 2250068/FEA = EST/ | | |
| | | | | CNT = 25/TID = Hs.102406.0/ | | |
| | | | | TIER = Stack/STK = 19/UG = Hs.102406/ | | |
| | | | | UG_TITLE = ESTs | | |
| 11 | 210246_s_at | ABCC8 | ATP-binding cassette, | gb: AF087138.1 | ABC | integral to |
| | | | sub-family C (CFTR/ | DB_XREF = gi: 3643189/GEN = SUR1/ | transporter | membrane |
| | | | MRP), member 8 | FEA = FLmRNA/CNT = 31/ | | |
| | | | | TID = Hs.54470.0/TIER = FL/STK = 0/ | | |
| | | | | UG = Hs.54470/LL = 6833/DEF = Homo | | |
| | | | | sapiens sulfonylurea receptor 1 | | |
| | | | | (SUR1) mRNA, complete cds./ | | |
| | | | | PROD = sulfonylurea receptor 1 | | |
| | | | | FL = gb: NM_000352.2 gb: L78207.1 | | |
| | | | | gb: AF08 | | |
| 12 | 205946_at | VIPR2 | vasoactive intestinal | gb: X95097.2/DB_XREF = gi: 4837717/ | GPCRs | integral to |
| | | | peptide receptor 2 | GEN = VIP2r/FEA = FLmRNA/CNT = 29/ | | plasma |
| | | | | TID = Hs.2126.0/TIER = ConsEnd/ | | membrane |
| | | | | STK = 0/UG = Hs.2126/LL = 7434/ | | |
| | | | | DEF = Homo sapiens mRNA for VIP | | |
| | | | | receptor 2./PROD = VIP2 receptor/ | | |
| | | | | FL = gb: NM_003382.1 gb: L36566.1 | | |
| 13 | 1558846_at | PNLIPRP3 | pancreatic lipase- | gb: AL833418.1/ | — | lysosome |
| | | | related protein 3 | DB_XREF = gi: 21734059/ | | (lumen) |
| | | | | TID = Hs2.376864.1/CNT = 7/ | | |
| | | | | FEA = mRNA/TIER = ConsEnd/STK = 0/ | | |
| | | | | UG = Hs.376864/UG_TITLE = Homo | | |
| | | | | sapiens mRNA; cDNA | | |
| | | | | DKFZp313P1022 (from clone | | |
| | | | | DKFZp313P1022)/DEF = Homo | | |
| | | | | sapiens mRNA; cDNA | | |
| | | | | DKFZp313P1022 (from clone | | |
| | | | | DKFZp313P1022). | | |
| 14 | 244444_at | PKD1L2 | polycystic kidney | gb: AW082870/DB_XREF = gi: 6038022/ | — | integral to |
| | | | disease 1-like 2 | DB_XREF = xb71f11.x1/ | | membrane |
| | | | | CLONE = IMAGE: 2581773/FEA = EST/ | | |
| | | | | CNT = 3/TID = Hs.210954.0/ | | |
| | | | | TIER = ConsEnd/STK = 3/ | | |
| | | | | UG = Hs.210954/UG_TITLE = ESTs | | |
| 15 | 213745_at | ATRNL1 | attractin-like 1 | gb: AW151108/DB_XREF = gi: 6199006/ | — | membrane |
| | | | | DB_XREF = xg33d03.x1/ | | |
| | | | | CLONE = IMAGE: 2629349/ | | |
| | | | | FEA = mRNA/CNT = 40/ | | |
| | | | | TID = Hs.196012.0/TIER = Stack/ | | |
| | | | | STK = 12/UG = Hs.196012/LL = 26033/ | | |
| | | | | UG_GENE = KIAA0534/ | | |
| | | | | UG_TITLE = KIAA0534 protein/ | | |
| 16 | 244353_s_at | SLC2A12 | solute carrier family 2 | gb: AI675682/DB_XREF = gi: 4876162/ | — | integral to |
| | | | (facilitated glucose | DB_XREF = wc45f07.x1/ | | membrane |
| | | | transporter), member | CLONE = IMAGE: 2321605/FEA = EST/ | | |
| | | | 12 | CNT = 8/TID = Hs.26691.1/ | | |
| | | | | TIER = ConsEnd/STK = 0/ | | |
| | | | | UG = Hs.26691/UG_TITLE = ESTs | | |
| 17 | 207938_at | PI15 | protease inhibitor 15 | gb: NM_015886.1/ | Allergen | extracellular |
| | | | | DB_XREF = gi: 7705675/GEN = R3HDM/ | V5/Tpx-1/ | |
| | | | | FEA = FLmRNA/CNT = 2 | related/ | |
| | | | | TID = Hs.129732.0/TIER = FL/STK = 0/ | | |
| | | | | UG = Hs.129732/LL = 51050/ | | |
| | | | | DEF = Homo sapiens R3H domain | | |
| | | | | (binds single-stranded nucleic acids) | | |
| | | | | containing (R3HDM), mRNA./ | | |
| | | | | PROD = R3H domain-containing | | |
| | | | | preproprotei | | |
| 18 | 210609_s_at | TP53I3 | tumor protein p53 | gb: BC000474.1/ | — | — |
| | | | inducible protein 3 | DB_XREF = gi: 12653408/ | | |
| | | | | FEA = FLmRNA/CNT = 7/ | | |
| | | | | TID = Hs.50649.1/TIER = FL/STK = 0/ | | |
| | | | | UG = Hs.50649/LL = 9540/ | | |
| | | | | UG_GENE = PIG3/DEF = Homo | | |
| | | | | sapiens, quinone oxidoreductase | | |
| | | | | homolog, clone MGC: 8642, mRNA, | | |
| | | | | complete cds./PROD = quinone | | |
| | | | | oxidoreductase homolog/FL = gb: BC | | |
| 19 | 213197_at | ASTN | astrotactin | gb: AB006627.1/ | — | integral to |
| | | | | DB_XREF = gi: 2564325/ | | membrane |
| | | | | GEN = KIAA0289/FEA = mRNA/ | | |
| | | | | CNT = 84/TID = Hs.6788.0/TIER = Stack/ | | |
| | | | | STK = 40/UG = Hs.6788/LL = 460/ | | |
| | | | | UG_TITLE = astrotactin/DEF = Homo | | |
| | | | | sapiens mRNA for KIAA0289 gene, | | |
| | | | | partial cds. | | |
| 20 | 1554018_at | GPNMB | glycoprotein | gb: BC011595.1/ | Polycystic | plasma |
| | | | (transmembrane) nmb | DB_XREF = gi: 15079529/ | kidney | membrane /// |
| | | | | TID = Hs2.82226.2/CNT = 21/ | disease/ | integral to |
| | | | | FEA = FLmRNA/TIER = FL/STK = 6/ | proteins | membrane |
| | | | | LL = 10457/UG_GENE = GPNMB/ | | |
| | | | | UG = Hs.82226/DEF = Homo sapiens,/ | | |
| | | | | Similar to glycoprotein | | |
| | | | | (transmembrane) nmb, clone | | |
| | | | | MGC: 1696 IMAGE: 3345861, mRNA, | | |
| | | | | complete cds./PROD = Similar t | | |
| 21 | 227202_at | CNTN1 | contactin 1 | gb: AW072790/DB_XREF = gi: 6027788/ | — | membrane |
| | | | | DB_XREF = xa42a10.x1/ | | fraction |
| | | | | CLONE = IMAGE: 2569434/FEA = EST/ | | |
| | | | | CNT = 43/TID = Hs.143434.2/ | | |
| | | | | TIER = Stack/STK = 9/UG = Hs.143434/ | | |
| | | | | LL = 1272/UG_GENE = CNTN1/ | | |
| | | | | UG_TITLE = contactin 1 | | |
| 22 | 203413_at | NELL2 | neural epidermal | gb: NM_006159.1/ | | Secreted |
| | | | growth factor like | DB_XREF = gi: 5453765/GEN = NELL2/ | | glycoprotein |
| | | | like-2 | FEA = FLmRNA/CNT = 141/ | | |
| | | | | TID = Hs.79389.0/TIER = FL + Stack/ | | |
| | | | | STK = 32/UG = Hs.79389/LL = 4753/ | | |
| | | | | DEF = Homo sapiens nel (chicken)-like/ | | |
| | | | | 2 (NELL2), mRNA./PROD = nel | | |
| | | | | (chicken)-like 2/FL = gb: D83018.1 | | |
| | | | | gb: NM_006159.1 | | |
| 23 | 205122_at | TMEFF1 | transmembrane protein | gb: BF439316/DB_XREF = gi: 11451833/ | | Integral to |
| | | | with EGF-like and two | DB_XREF = nab62g12.x1/ | | membrane |
| | | | follistatin-like domains | CLONE = IMAGE: 3272638/ | | |
| | | | 1 | FEA = FLmRNA/CNT = 65/ | | |
| | | | | TID = Hs.78531.0/TIER = Stack/ | | |
| | | | | STK = 27/UG = Hs.78531/LL = 8577/ | | |
| | | | | UG_GENE = TMEFF1/ | | |
| | | | | UG_TITLE = transmembrane protein | | |
| | | | | with EGF-like and two follistatin-like | | |
| | | | | domains 1/FL = gb: U19878.1 | | |
| | | | | gb: NM_003692.1 | | |
| 24 | 1569141_a_at | PPARGC1A | peroxisome | gb: BC029800.1/ | — | nucleus /// |
| | | | proliferative | DB_XREF = gi: 20987590/ | | DNA-directed |
| | | | activated receptor, | TID = Hs2.284627.1/CNT = 7/ | | RNA |
| | | | gamma, coactivator | FEA = mRNA/TIER = ConsEnd/STK = 0/ | | polymerase II, |
| | | | 1, alpha | UG = Hs.284627/UG_TITLE = Homo | | core complex |
| | | | | sapiens, Similar to peroxisome | | |
| | | | | proliferative activated receptor, | | |
| | | | | gamma, coactivator 1, clone | | |
| | | | | IMAGE: 5187727, mRNA/DEF = Homo | | |
| | | | | sapiens, Sim | | |
| 25 | 202828_s_at | MMP14 | matrix | gb: NM_004995.2/ | — | extracelluar |
| | | | metalloproteinase | DB_XREF = gi: 13027797/ | | matrix (sensu |
| | | | 14 (membrane- | GEN = MMP14/FEA = FLmRNA/ | | Metazoa) /// |
| | | | inserted) | CNT = 120/TID = Hs.2399.0/ | | integral to |
| | | | | TIER = FL + Stack/STK = 10/ | | plasma |
| | | | | UG = Hs.2399/LL = 4323/DEF = Homo | | membrane |
| | | | | sapiens matrix metalloproteinase 14 | | |
| | | | | (membrane-inserted) (MMP14), | | |
| | | | | mRNA./PROD = matrix | | |
| | | | | metalloproteinase 14 preproprotein | | |
| 26 | 206742_at | FIGF | vascular endothelial | gb: NM_004469.1/ | | secreted |
| | | | growth factor D | DB_XREF = gi: 4758377/GEN = FIGF/ | | glycoprotein |
| | | | | FEA = FLmRNA/CNT = 16/ | | |
| | | | | TID = Hs.11392.0/TIER = FL + Stack/ | | |
| | | | | STK = 11/UG = Hs.11392/LL = 2277/ | | |
| | | | | DEF = Homo sapiens c-fos induced | | |
| | | | | growth factor (vascular endothelial | | |
| | | | | growth factor D) (FIGF), mRNA./ | | |
| | | | | PROD = c-fos induced growth factor | | |
| | | | | (vascularendothelial growth factor D)/ | | |
| | | | | FL = gb: NM_004469.1 gb: D89630.1 | | |

TSC^{-/-} Cell Lines

The invention provides an immortalized cell that does not express the Tuberous Sclerosis Complex-2 gene(TSC2). By not expressing the TSC2 gene is meant that the gene is not functionally active in the cell. A TSC function includes for example, serum dependent S6 an S6K phosphorylation. The cell and cells lines are refered to herein as a TSC2^{-/-} cell or a TSC2 null cell. A TSC2^{-/-} cell is capable of self-maintenance, such that with each cell division, at least one daughter cell will also be a TSC^{-/-} cell. A TSC^{-/-} cell line is capable of being expanded (passaged) 10, 20, 50, 100, 250, 500, 1000, 2000, 3000, 4000, 5000 or more fold. The cells are adherent in culture.

By “normal cells”, “primary cells” or “non-immortalized cells” is meant to designate cells of which are collected from the a healthy adult not having crippling physiological or genetic deficiencies, and which can be cultured for a limited time without losing their original differentiation characteristics.

By “immortalized cells” is meant to designate cells which have undergone a genetic manipulation, by means of a DNA construct, which makes them capable of multiplying indefinitely.

By “passage” is meant the the process consisting in taking an aliquot of a confluent culture of a cell line, in inoculating into fresh medium, and in culturing the line until confluence or saturation is obtained. The cell lines are thus traditionally cultured by successive passages in fresh media.

Genomic sequencing determined that the cells possessed a missense mutation in one copy of the TSC2 gene and the other copy of the TSC2 was lossed due to a loss of heterozygosity (LOH) of the TSC2 gene locus. The missense mutation is a specific point mutation resulting is a guanine to adenine transition at position 1832 in exon 16 of the TSC2 gene. This mutation results in the loss of a HpaII restriction endonuclease site and the creation of a diagnostic PvuII restriction endonuclease site A TSC2^{-/-} cell line maintains in culture the elongated morphology of the primary AML cells.

The loss of TSC function is measured by phosphorylation of S6Kinase (S6K) and its substrate, ribosomal protein S6 (S6), in the absence of serum. In both TSC 1^{-/-} or TSC2^{-/-} cells, the absence of the inhibitory TSC complex mimics mitogenic stimulation and results in constitutively active S6K signaling.

General Methods for Measuring Gene Expression

By measuring expression of the various genes in a sample of cells, a TSC related disorder can be determined in a cell or population of cells. Similarly, by measuring the expression of these genes in response to various agents, and agents for treating TSC related disorders can be identified.

The invention involves determining (e.g., measuring) the expression of at least one, and up to all the TSC sequences listed in Table B. Using sequence information provided by the GeneBank database entries for the known sequences or the sequences provides herein the TSC-associated genes are detected and measured using techniques well known to one of ordinary skill in the art. For example, sequences within the sequence database entries corresponding to TSC sequences, can be used to construct probes for detecting TSC RNA sequences in, e.g., northern blot hybridization analyses. As another example, the sequences can be used to construct primers for specifically amplifying the TSC sequences in, e.g, amplification-based detection methods such as reverse-transcription based polymerase chain reaction. “Probes” refer to nucleic acid sequences of variable length, preferably between at least about 10 nucleotides (nt), 10 nt, 30 nt, 40 nt, 50nt, 75 nt, 100 nt, 250 nt, 500 nt or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

Hybridization is under stringent, moderate or low conditions. As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

Moderate stringency hybridization conditions are for example, hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

Low stringency hybridization conditions arefor example hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Expression level of one or more of the TSC sequences in the test cell population, e.g., a patient derived tissues sample is then compared to expression levels of the some sequences in a reference population. The reference cell population includes one or more cells for which the compared parameter is known, i.e., cancerous, non-cancerous, TSC or non-TSC.

Whether or not the gene expression levels in the test cell population compared to the reference cell population reveals the presence of the measured parameter depends upon on the composition of the reference cell population. For example, if the reference cell population is composed of non-cancer cells, a similar gene expression level in the test cell population and reference cell population indicates the test cell population is non-cancer. Conversely, if the reference cell population is made up of cancer cells, a similar gene expression profile between the test cell population and the reference cell population that the test cell population includes cancer cells.

An TSC sequence in a test cell population can be considered altered in levels of expression if its expression level varies from the reference cell population by more than 1.0, 1.5, 2.0, 5.0, 10.0 or more fold from the expression level of the corresponding TSC sequence in the reference cell population.

The alteration is statistically significant. By statistically significant is meant that the alteration is greater than what might be expected to happen by change alone. Statistical significance is determined by method known in the art. For example statistical significance is determined by p-value. The p-values is a measure of probability that a difference between groups during an experiment happened by chance. (P(z≧z_{observed})). For example, a p-value of 0.01 means that there is a 1 in 100 chance the result occurred by chance. The lower the p-value, the more likely it is that the difference between groups was caused by treatment. An alteration is statistically significant if the p-value is at least 0.05. Preferably, the p-value is 0.04, 0.03, 0.02, 0.01, 0.005, 0.001 or less.

If desired, comparison of differentially expressed sequences between a test cell population and a reference cell population can be done with respect to a control nucleic acid whose expression is independent of the parameter or condition being measured. For example, a control nucleic acid is one which is known not to differ depending on the cancerous or non-cancerous state of the cell. Expression levels of the control nucleic acid in the test and reference nucleic acid can be used to normalize signal levels in the compared populations. Control genes can be, e.g,. β-actin, glyceraldehyde 3-phosphate dehydrogenase or ribosomal protein P1 (36B4).

The test cell population is compared to multiple reference cell populations. Each of the multiple reference populations may differ in the known parameter. Thus, a test cell population may be compared to a second reference cell population known to contain, e.g., TSC-related disorder as well as a second reference population known to contain, e.g., non-TSC-related disorder (normal cells). The test cell is included in a tissue type or cell sample from a subject known to, or to be suspected of having a TSC-related disorder.

The test cell is obtained from a bodily tissue or a bodily fluid, e.g., biological fluid (such as blood, serum, or sputum). For example, the test cell is purified from a tissue. Preferably, the test cell population comprises a tumor cell. Alternatively, the test cell population is a lung cell, a kidney cell, an adipose cell , a smooth muscle cell, a blood vessel cell or a neuronal cell.

Cells in the reference cell population are derived from a tissue type as similar to test cell. Alternatively, the control cell population is derived from a database of molecular information derived from cells for which the assayed parameter or condition is known.

The subject is preferably a mammal. The mammal can be, e.g., a human, non-human primate, mouse, rat, dog, cat, horse, or cow.

The expression of 1, 2, 3, 4, 5, 25, 35, 50, or 100 or more of the sequences represented by TSC 1-26 is determined and if desired, expression of these sequences can be determined along with other sequences whose level of expression is known to be altered according to one of the herein described parameters or conditions, e.g., a TSC-related disorder.

Expression of the genes disclosed herein is determined at the RNA level using any method known in the art. For example, Northern hybridization analysis using probes which specifically recognize one or more of these sequences can be used to determine gene expression. Alternatively, expression is measured using reverse-transcription-based PCR assays, e.g., using primers specific for the differentially expressed sequences.

Expression is also determined at the protein level, i.e., by measuring the levels of polypeptides encoded by the gene products described herein. Such methods are well known in the art and include, e.g., immunoassays based on antibodies to proteins encoded by the genes.

When alterations in gene expression are associated with gene amplification or deletion, sequence comparisons in test and reference populations can be made by comparing relative amounts of the examined DNA sequences in the test and reference cell populations.

Diagnosing TSC Related Disorders

A TSC related disorder is diagnosed by examining the expression of one or more TSC nucleic acid sequences from a test population of cells, (i.e., a patient derived tissue sample). Preferably, the test cell population comprises a primary cancer cell. Alternatively, the test cell is a lung cell, a kidney cell, an adipose cell , a smooth muscle cell, a blood vessel cell or a neuronal cell. Gene expression is also measured from blood or other bodily fluids such as sputum.

Expression of one or more of TSC-associated gene, e.g., TSC 1-26 is determined in the test cell and compared to the expression of the normal control level. By normal control level is meant the expression profile of the TSC-associated genes typically found in a population not suffering from a TSC related disorder. An increase or a decrease of the level of expression in the patient derived tissue sample of the TSC-associated genes indicates that the subject is suffering from or is at risk of developing a TSC-related disorder.

When one or more of the TSC-associated genes are altered in the test population compared to the normal control level indicates that the subject suffers from or is at risk of developing a TSC-related disorder. 50%, 60%, 80%, 90% or more of the TSC -associated genes are altered.

Identifying Agents that Inhibit TSC-Associated Gene Expression

An agent that inhibits the expression or activity of TSC-associated gene is identified by contacting a test cell population expressing a TSC-associated upregulated gene with a test agent and determining the expression level of the TSC-associated gene. A decrease in expression compared to the normal control level indicates the agent is an inhibitor of a TSC-associated upregulated gene and useful to inhibit a TSC-related disorder.

The test cell population is any cell expressing the TSC-associated genes. For example, the test cell population contains a primary cancer cell or is derived from a primary cancer cell. For example, the test cell is immortalized cell line derived from a primary cancer cell such as a TSC2^{-/-} of the invention.

Assessing Efficacy of Treatment of a TSC-Related Disorder in a Subject

The differentially expressed TSC sequences identified herein also allow for the course of treatment of of a TSC-related disorder to be monitored. In this method, a test cell population is provided from a subject undergoing treatment for a TSC-related disorder. If desired, test cell populations are obtained from the subject at various time points before, during, or after treatment. Expression of one or more of the TSC sequences, in the cell population is then determined and compared to a reference cell population which includes cells whose TSC-related disorder state is known. The reference cells have not been exposed to the treatment.

If the reference cell population contains non-TSC related disorder cells, a similarity in expression between TSC sequences in the test cell population and the reference cell population indicates that the treatment is efficacious. However, a difference in expression between TSC sequences in the test population and this reference cell population indicates the a less favorable clinical outcome or prognosis.

By “efficacious” is meant that the treatment leads to a reduction in expression of a pathologically upregulated gene, increase in expression of a pathologically down-regulated gene or a decrease in size, prevalence, or metastatic potential of a TSC-related disorder in a subject. When treatment is applied prophylactically, “efficacious” means that the treatment retards or prevents a TSC-related disorder. Assesment of a TSC-related disorder is made using standard clinical protocols.

Efficaciousness is determined in association with any known method for diagnosing or treating a TSC-related disorder. TSC-realated disorders are diagnosed for example, by determing whether the subject has either two “Major Features” of TSC or one “Major Feature” and two “Minor Features”. The clinician should consider TSC probable when the patienthas one “Major Feature” and one “Minor Feature,” while a possible diagnosis results from the presence of either one “Major Feature” or two or more “Minor Features.” Major Features of TSC include: Facial angiofibromas or forehead plaque; Nontraumatic ungual or periungual fibroma; Hypomelanotic macules (three or more); Shagreen patch (connective tissue nevus); Multiple retinal nodular hamartomas; Cortical tuber; Subependymal nodule; Subependymal giant cell astrocytoma; Cardiac rhabdomyoma, single or multiple; Lymphangiomyomatosis; or Renal angiomyolipoma. Minor Features of TSC include: Multiple, randomly distributed pits in dental enamel; Hamartomatous rectal polypsc; Bone cystsd; Cerebral white matter radial migration linesa,d; Gingival fibromas; Nonrenal hamartomac; Retinal achromic patch; ‘Confetti’ skin lesions; or Multiple renal cysts.

Selecting a Therapeutic Agent for Treating a TSC-Related Disorder that is Appropriate for a Particular Individual

Differences in the genetic makeup of individuals can result in differences in their relative abilities to metabolize various drugs. An agent that is metabolized in a subject to act as an anti-colorectal cancer agent can manifest itself by inducing a change in gene expression pattern in the subject's cells from that characteristic of a TSC-rleated disorder state to a gene expression pattern characteristic of a non-TSC-related disorder state. Accordingly, the differentially expressed TSC sequences disclosed herein allow for a putative therapeutic or prophylactic anti-TSC-related disorder agent to be tested in a test cell population from a selected subject in order to determine if the agent is a suitable anti-TSC-related disorder agent in the subject.

To identify an anti-TSC-related disorder agent, that is appropriate for a specific subject, a test cell population from the subject is exposed to a therapeutic agent, and the expression of one or more of TSC 1-26 sequences is determined.

The test cell population contains a cell expressing TSC-associated gene. For example a test cell population is incubated in the presence of a candidate agent and the pattern of gene expression of the test sample is measured and compared to one or more reference profiles, e.g., TSC-related disorder reference expression profile or an non-TSC-related disorder reference expression profile.

A decrease in expression of one or more of the sequences TSC 1-26 in a test cell population relative to a reference cell population that has not been contacted with the candidate agent is indicative that the agent is therapeutic.

The test agent can be any compound or composition.

Screening Assays for Identifying Therapeutic Agents

The differentially expressed sequences disclosed herein can also be used to identify candidate therapeutic agents for treating a TSC-related disorder. The method is based on screening a candidate therapeutic agent to determine if it converts an expression profile of TSC 1-26 sequences characteristic of a TSC-related disorder state to a pattern indicative of a non-TSC-related disorder state.

In the method, a cell is exposed to a test agent or a combination of test agents (sequentially or consequentially) and the expression of one or more TSC 1-26 sequences in the cell is measured. The expression profile of the TSC sequences in the test population is compared to expression level of the TSC sequences in a reference cell population that is not exposed to the test agent.

An agent effective in stimulating expression of underexpressed genes, or in suppressing expression of overexpressed genes is deemed to lead to a clinical benefit such compounds are further tested for the ability to inhibit the progression of a TSC-related disorder.

Such screening of the present invention comprises, for example, the steps described below. Cells expressing a target gene include, for example, cell lines established from a subject having a TSC-related disorder; such cells can be used for this purpose.

(1) the step of contacting a candidate agent with cells expressing a target gene; and

(2) the step of selecting a candidate agent that alters the expression level of the target gene as compared with that in a control.

Alternatively, the screening of the present invention may comprise the steps described below. A protein required for the screening can be obtained as a recombinant protein by using the nucleotide sequence of the target gene. Based on the information on the target gene, one skilled in the art can select the biological activity of a protein as an index of screening and a measurement method for the activity.

(1) the step of contacting a candidate agent with the protein encoded by a target gene; and

(2) the step of selecting a candidate agent that alters the activity of the protein as compared with that in a control.

Alternatively, the screening of the present invention may comprise the steps described below. A reporter construct required for the screening can be prepared by using the transcriptional regulatory region of a target gene. When the transcriptional regulatory region of a target gene has been known to those skilled in the art, a reporter construct can be prepared by using the previous sequence information. When the transcriptional regulatory region of a target gene remains unidentified, a nucleotide segment containing the transcriptional regulatory region can be isolated from a genome library based on the nucleotide sequence information of the target gene.

(1) the step of preparing a reporter construct that ensures the expression of the reporter gene under control of the transcriptional regulatory region of the target gene;

(2) the step of contacting a candidate agent with host cells containing and capable of expressing the above-mentioned reporter construct; and

(3) the step of measuring the expression level of the reporter gene, and selecting a candidate agent that has an activity of altering the expression level when compared with that in a control.

In the screening method of the present invention, candidate agents to be selected have the activity of decreasing the expression levels as compared with those in a control. There is no limitation on the type of candidate agent in the screening of the present invention. The candidates of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection.

The biological library approach is limited to peptide libraries, while the other four approaches are applicable to peptide, non-peptide oligomer or small molecule libraries of compounds (Lam (1997)Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233. Libraries of compounds may be presented in solution (e.g., Houghten (1992) Bio Techniques 13:412), or on beads (Lam (1991) Nature 354:82), chips (Fodor (1993) Nature 364:555), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865) or phage (Scott and Smith (1990) Science 249:386; Devlin (1990) Science 249:404; Cwirla et al. (1990) Proc. Natl. Acad. Sci. USA 87:6378; and Felici (1991) J. Mol. Biol. 222:301).(U.S. Patent Application 20020103360)

Assessing the Prognosis of a Subject with a TSC-Related Disorder

Also provided is a method of assessing the prognosis of a subject with a TSC-related disorder by comparing the expression of one or more TSC sequences in a test cell population to the expression of the sequences in a reference cell population derived from patients over a spectrum of disease stages. By comparing gene expression of one or more TSC sequences in the test cell population and the reference cell population(s), or by comparing the pattern of gene expression over time in test cell populations derived from the subject, the prognosis of the subject can be assessed.

An increase of expression of one or more of the sequences TSC 1-26 compared to a normal control indicates less favorable prognosis.

Methods of Treating a TSC-Related Disorder

The invention provides a method for alleviating a symptom of a TSC-related disorder, inhibiting tumor growth or treating lesions of a TSC-related disorder in a subject. Therapeutic compounds are administered prophylactically or therapeutically to subject suffering from at risk of (or susceptible to) developing a TSC-related disorder. Such subjects are identified using standard clinical methods or by detecting an aberrant level of expression or activity of (e.g., TSC 1-26).

The method includes decreasing the expression, or function, or both, of one or more gene products of genes whose expression is aberrantly increased (“overexpressed gene”). Expression is inhibited in any of several ways known in the art. For example, expression is inhibited by administering to the subject a nucleic acid that inhibits, or antagonizes, the expression of the overexpressed gene or genes, e.g., an antisense oligonucleotide which disrupts expression of the overexpressed gene or genes.

Alternatively, function of one or more gene products of the overexpressed genes is inhibited by administering a compound that binds to or otherwise inhibits the function of the gene products. For example, the compound is an antibody which binds to the overexpressed gene product or gene products.

These modulatory methods are performed ex vivo or in vitro (e.g., by culturing the cell with the agent) or, alternatively, in vivo (e.g., by administering the agent to a subject). The method involves administering a protein or combination of proteins or a nucleic acid molecule or combination of nucleic acid, molecules as therapy to counteract aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the overexpressed gene or genes. Therapeutics that antagonize activity are administered therapeutically or prophylactically.

Therapeutics that may be utilized include, e.g., (i) a polypeptide, or analogs, derivatives, fragments or homologs thereof of the overexpressed or underexpressed sequence or sequences; (ii) antibodies to the overexpressed or underexpressed sequence or sequences; (iii) nucleic acids encoding the over or underexpressed sequence or sequences; (iv) antisense nucleic acids or nucleic acids that are “dysfunctional” (i.e., due to a heterologous insertion within the coding sequences of coding sequences of one or more overexpressed or underexpressed sequences); or (v) modulators (i.e., inhibitors, agonists and antagonists that alter the interaction between an over/underexpressed polypeptide and its binding partner. The dysfunctional antisense molecule are utilized to “knockout” endogenous function of a polypeptide by homologous recombination (see, e.g., Capecchi, Science 244: 1288-1292 1989)

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a polypeptide (or analogs, derivatives, fragments or homologs thereof) or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods includes contacting a cell with an agent that modulates one or more of the activities of the gene products of the differentially expressed genes. An agent that modulates protein activity includes a nucleic acid or a protein, a naturally-occurring cognate ligand of these proteins, a peptide, a peptidomimetic, or other small molecule. For example, the agent stimulates one or more protein activities of one or more of a differentially under-expressed gene.

Pharmaceutical Compositions for Treating a TSC-Related Disorder

Pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. The formulations are optionally packaged in discrete dosage units

Pharmaceutical formulations suitable for oral administration include capsules, cachets or tablets, each containing a predetermined amount of the active ingredient. Formulations also include powders, granules or solutions, suspensions or emulsions. The active ingredient os optionally administered as a bolus electuary or paste. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein. A package of tablets may contain one tablet to be taken on ech of the month. The formulation or does of medicament varies with respect to the phase (probe or sucretary) of the menstrual cycle.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration include suppositories with standard carriers such as cocoa butter or polyethylene glycol. Formulations for topical administration in the mouth, for example buccally or sublingually, include lozenges, which contain the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the compositions, e.g., polypeptides and organic compounds are administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity.

Kits

The invention also includes an TSC-detection reagent, e.g., a nucleic acid that specifically binds to or identifies one or more TSC nucleic acids such as oligonucleotide sequences, which are complementary to a portion of an TSC nucleic acid or antibodies which bind to proteins encoded by an TSC nucleic acid. An oligonucleotide is at least 5, 10, 15, 20, 25, 30, 40, 50, 75 or more nucleic acids in length. The reagents are packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay are included in the kit. The assay format of the kit is a Northern hybridization or a sandwich ELISA known in the art.

For example, TSC detection reagent, is immobilized on a solid matrix such as a porous strip to form at least one TSC detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of TSC present in the sample. The detection sites may be configured in any suitably detectable shape and are typically in the shape of a bar or dot spanning the width of a teststrip.

Alternatively, the kit contains a nucleic acid substrate array comprising one or more nucleic acid sequences. The nucleic acids on the array specifically identify one or more nucleic acid sequences represented by TSC 1-26. The expression of 2, 3, 4, 5, 6, 7, 8, 9, 10, 15, 20, 25, 40 or 50 or more of the sequences represented by TSC 1-26 are identified by virtue if the level of binding to an array test strip or chip. The substrate array can be on, e.g., a solid substrate, e.g., a “chip” as described in U.S. Pat. No. 5,744,305.

Arrays and Pluralities

The invention also includes a nucleic acid substrate array comprising one or more nucleic acid sequences. The nucleic acids on the array specifically corresponds to one or more nucleic acid sequences represented by TSC 1-26. The level expression of 2, 3, 4, 5, 6, 7, 8, 9, 10, 15, 20, 25, 40 or 50 or more of the sequences represented by TSC 1-26 are identified by detecting nucleic acid binding to the array.

The invention also includes an isolated plurality (i.e., a mixture if two or more nucleic acids) of nucleic acid sequences. The nucleic acid sequence are in a liquid phase or a solid phase, e.g., immobilized on a solid support such as a nitrocellulose membrane. The plurality includes one or more of the nucleic acid sequences represented by TSC 1-26. In various embodiments, the plurality includes 2, 3, 4, 5, 6, 7, 8, 9, 10, 15, 20, 25, 40 or 50 or more of the sequences represented by TSC 1-26.

Chips

The DNA chip is a device that is convenient to compare expression levels of a number of genes at the same time. DNA chip-based expression profiling can be carried out, for example, by the method as disclosed in “Microarray Biochip Technology” (Mark Schena, Eaton Publishing, 2000), etc.

A DNA chip comprises immobilized high-density probes to detect a number of genes. Thus, expression levels of many genes can be estimated at the same time by a single-round analysis. Namely, the expression profile of a specimen can be determined with a DNA chip. The DNA chip-based method of the present invention comprises the following steps of:

(1) synthesizing cRNAs or cDNAs corresponding to the marker genes;

(2) hybridizing the cRNAs or cDNAs with probes for marker genes; and

(3) detecting the cRNA or cDNA hybridizing with the probes and quantifying the amount of mRNA thereof.

The cRNA refers to RNA transcribed from a template cDNA with RNA polymerase. A cRNA transcription kit for DNA chip-based expression profiling is commercially available. With such a kit, cRNA can be synthesized from T7 promoter-attached cDNA as a template by using T7 RNA polymerase. On the other hand, by PCR using random primer, cDNA can be amplified using as a template a cDNA synthesized from mRNA.

On the other hand, the DNA chip comprises probes, which have been spotted thereon, to detect the marker genes of the present invention. There is no limitation on the number of marker genes spotted on the DNA chip. For example, it is allowed to select 5% or more, preferably 20% or more, more preferably 50% or more, still more preferably 70% or more of the marker genes of the present invention. Any other genes as well as the marker genes can be spotted on the DNA chip. For example, a probe for a gene whose expression level is hardly altered may be spotted on the DNA chip. Such a gene can be used to normalize assay results when assay results are intended to be compared between multiple chips or between different assays.

A probe is designed for each marker gene selected, and spotted on a DNA chip. Such a probe may be, for example, an oligonucleotide comprising 5-50 nucleotide residues. A method for synthesizing such oligonucleotides on a DNA chip is known to those skilled in the art. Longer DNAs can be synthesized by PCR or chemically. A method for spotting long DNA, which is synthesized by PCR or the like, onto a glass slide is also known to those skilled in the art. A DNA chip that is obtained by the method as described above can be used for diagnosing a disease X according to the present invention.

The prepared DNA chip is contacted with cRNA, followed by the detection of hybridization between the probe and cRNA. The cRNA can be previously labeled with a fluorescent dye. A fluorescent dye such as Cy3(red) and Cy5 (blue) can be used to label a cRNA. cRNAs from a subject and a control are labeled with different fluorescent dyes, respectively. The difference in the expression level between the two can be estimated based on a difference in the signal intensity. The signal of fluorescent dye on the DNA chip can be detected by a scanner and analyzed by using a special program. For example, the Suite from Affymetrix is a software package for DNA chip analysis.

Also the expression level of the marker gene(s) can be analyzed based on activity or quantity of protein(s) encoded by the marker gene(s). A method for determining the quantity of the protein(s) is known to those skilled in the art. For example, immunoasssay method is useful for determination of the protein in biological material. Any biological materials can be used for the determination of the protein or it's activity. For example, blood sample is analyzed for estimation of the protein encoded by serum markers. Another hand, a suitable method can be selected for the determination of the activity protein(s) encoded by the marker gene(s) according to the activity of each protein to be analyzed.

The invention will be further described in the following examples, which do not limit the scope of the invention described in the claims. The following examples illustrate the identification and characterization of genes differentially expressed in AML cells.

### EXAMPLE 1

### General Methods

Cell and Tissue Acquistion

A heterogeneous population of primary AML cells obtained from a sporadic LAM patient, designated #621 was acquired from Dr. E. P. Henske (Fox Chase Cancer Research Center, Philadelphia, Pa.). AML cells within the population were determined to be TSC2-1-by genomic sequencing (Yu, J., et al. 2003). Frozen AML tissue (AML548, AML564, AML576, AML823, AML1003) and normal donor tissue (kidney, liver, lung, heart, aorta, adipose donor 1 and 2) was obtained from the Maryland Brain and Tissue Bank (Baltimore, Mass.) via IRB approved protocols. Human melanoma cell lines; Malme3M, Sk-Mel2, Sk-Mel5, Sk-Mel28, UACC62, UACC257, and M14, were obtained from the Tumor Repository of the Division of Cancer Treatment and Diagnosis, National Cancer Institute, Frederick, Mass. Melanoma cell line A375 was obtained from American Type Culture Collection (ATCC, Manassus, Va.). Amphotropic retroviral producing cell line expressing the E6E7 genes of the human papilloma virus 16 (PA317 pLXSN 16E6E7) and the vector expressing control helper line (PA317 PLXSN) were obtained from ATCC.

Cell culture

Primary AML cells and AML cell lines were grown in DMEM/F12 basal media including 15% FBS, 0.2uM hydrocortisone, 10 uU/mL vasopressin, 1×FeSO4, 10 ng/mL EGF, 1×ITS, 0.01 nM triiolythryonine, 0.12% sodium bicarbonate, 1× cholesterol, 500ug/ml G418 (for clones only) and 1×penicillin/streptomycin/amphotericinB (PSA). Amphotropic retroviral helper cell lines from ATCC were grown in DMEM plus, 10% FBS, PSA in a BSL-2 level facility. Melanoma cell lines were grown according to ATCC and NCI instructions.

Cellular Immortalization

AML#621 heterogeneous cell suspension was infected with a replication deficient Moloney Murine Leukemia Virus (MoMLV) that carries the pLXSN vector encoding the E6, E7, and gentamicin (G418) resistance genes (ATCC). Retrovirus containing only the pLXSN vector with G418-resistance was used as a control. AML cells were plated the day before infection into 2, T-25 flasks at a density of 500,000 cells/flask, and incubated overnight at 37° C. Retroviral producing cell lines were grown to confluency in T-75 flasks. Medium was replaced with 10 mL of fresh growth media and incubated overnight at 32° C. Virus containing media was sterile filtered using a 0.45 micro syringe filter and polybrene added at a final concentration of 8 ug/mL. Medium from the AML cells was replaced with 5 mL viral sup and flasks were centrifuged at 2,500 rpm at 32° C. for 90 minutes. AMLs plus viral sup were then incubated overnight at 32° C. to continue the infection. 24 hours later cells were returned to 37° C. and virus containing medium replaced with fresh growth medium. 48 hours ost infection, successfully transduced clones were isolated via growth in G418-containing (800 ug/mL) medium. Once antibiotic-resistant cells were generated, individual clonal colonies were isolated by collaring, then expanded and frozen down.

PCR Restriction Digest Analysis of AML Clones

AML clones were assessed for the presence of a G183 1A mutation in exon 16 of the TSC2 gene by pcr-based restriction digest identification. This mutation results in a new PvuII restriction enzyme site and the elimination of a HpaII site. Genomic DNA was harvested and primary pcr was performed using primer pair 5′-gaagcacgcactctagagcag-3′; 5′-ccttcacagattgtgcagca-3′. One microliter of primary reaction was amplified in a nested reaction using primers 5′-gacca agctgtacac cctgcct-3′; 5′-cagaccgtcc ctcctctgca cccactgtgg ccgcagcctc cccagtcctg-3′. PCR products were digested with either hpaII or pvuII to assess the presence of the mutation. A wildtype clone obtained from a different AML sample that does not exhibit a mutation in exon 16 was used as a control.

Rapamycin Growth Assay.

1,000 cells/well were plated in triplicate of mouse embryonic fibroblasts (MEF's) TSC2^{+/+}; p53^{-/-} and TSC2^{-/-}; p53^{-/-}, and 3,000 cells/well in triplicate of 2 AML TSC2^{-/-} cell lines and 2 TSC2^{+/+} control lines generated from the same AML tumor. Rapamycin was added to cells at final concentrations of 0.01 nM, 0.1 nM, 1 nM, 10 nM, 100 nM, 1000 nM. Cells were grown for 72 hours and cell growth determined by MTS assay (Promega, Madison, Wis.).

Microarrays Analysis

Total RNA was harvested using the commercially available Trizol Reagent® [Life Technologies, GibcoBRL, (Gaithers-burg, Md.)]. Icoria (Research Triangle Park, N.C.) was provided with 100 ug total RNA from 2 TSC2−/− AML cell lines, and 4 primary AML tumors from different patients. Total RNA from 7 donor pooled normal tissues was purchased from Invitrogen (Carlsbad, Calif.) and provided to Icoria for gene expression profiling analysis. Hybridizations were performed with 1 ug of RNA converted to ssDNA of target on the GeneChip human genome U133 plus 2.0 oligonucleotide array containing over 54,000 probe sets representing more than 38,500 human genes (Affymetrix, Santa Clara, Calif.). Heirarchical clustering microarray data analysis was performed using the Spotfire DecisionSite for Functional Genomics™ software platform (Spotfire, Somerville, Mass.) and principal component anlysis was performed using Microsoft Excel. Genes that were up-regulated in AML tissues by >5-fold and determined to be likely cell surface expressed, were assessed by rtq-pcr.

RTQ-PCR

Five nanograms of total RNA for housekeeping genes and 500 ng for experimental genes, from AML cell lines, AML primary tissue, and normal tissue was added to a first-strand cDNA synthesis reaction using the commercially available Taqman Multiscribe ® Reverse Transcriptase Kit from ABI. Using the ABI Prism 7700 Thermocycler, complementary DNA (cDNA) synthesis on these samples was performed under the following conditions: 10 min at 25° C., 30 min at 48° C., followed by inactivation of the enzyme at 95° C. for 5 min. Fifty μl of the first-strand cDNA synthesis was placed into a TaqMan PCR reaction in triplicate. PCR conditions will be performed as follows: stage 1, 2 min at 50° C.; stage 2, 10 min at 95° C.; stage 3, 40 cycles of 15 s of melting at 95° C. followed by DNA synthesis for 1 min at 60° C. This PCR protocol will be optimized based on primer melting points (Tm) and experimental observations. PCR primers were designed using the computer program Primer Express® by ABI and based upon published or Genbank sequences. To assess the quantity and quality of the RNA/DNA, 2 housekeeping genes, GAPDH and β-actin, and were amplified for all samples and expression evaluated.

Immunoblotting

New AML and control cell lines were assessed for TSC2 expression by immunoblotting (C-20; Santa Cruz Biotechnology, Inc., Santa Cruz, Calif.), and constituitive phosphorylation of S6 (Ser 235/236) and S6kinase (Thr389) (Cell Signaling Technologies, Inc., Beverly, Mass.). AML and melanoma cell lines, AML and normal primary tissues were immunoblotted with antibodies against gpnmb (CR011; CuraGen Corp., Branford, Conn.), MelanA (C-20; Santa Cruz, Calif.), Silv (ZMD.254; Zymed, South San Francisco, Calif.), OA1 (W7; a gift from Dr. Schiaffino, Italy), mmp14 (Santa Cruz Biotechnology, Inc., Santa Cruz, Calif.).

### EXAMPLE 2

### Generation of TSC2^{-/-} AML Cell Lines

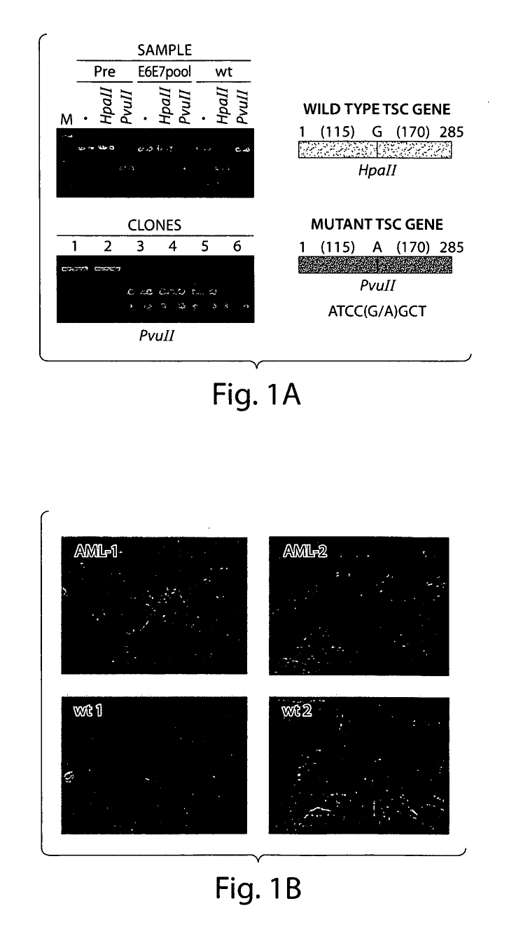

A heterogeneous AML tumor was obtained surgically from a sporadic LAM patient, designated patient #621. Genomic sequencing determined that the majority of cells present within the tumor possessed a missense mutation in one copy of the TSC2 gene. TSC2^{-/-} cells within the tumor resulted from a LOH of the TSC2 gene locus. Because patient #621 has sporadic LAM and not TSC-LAM, non-AML cells within the tumor mass are TSC2^{+/+}. The specific point mutation is a nucleotide G to A transition at position 1832 in exon 16 of the TSC2 gene. This mutation results in the loss of a HpaII restriction endonuclease site and the creation of a fortuitous diagnostic PvuII restriction endonuclease site (FIG. 1, right panel). The AML621 mixed cell population was infected with a retrovirus carrying the E6E7 genes of the human papilloma virus. Successfully infected cells were plated at a low enough density so as to be clonally isolated by collaring. Eighty individual clones were isolated, 70 TSC2^{-/-} and 10 TSC2^{+/+} as determined by genomic restriction digest analysis. Restriction digest confirmation of wildtype clones (wt-1, wt-2) and TSC-null clones (AML-1, AML-2, AML-3, AML-4) are shown (FIG. 1, lower panel).

Primary AML621 cells almost exclusively exhibit an elongated fiber-like morphology characteristic of the smooth muscle component of AMLs (FIG. 1, B). This is distinctly different from the epithelial shape of adjacent normal kidney cells. While most TSC2^{-/-} AML clones generated maintain the elongated morphology of the primary AML cells, wildtype clones generated from the same tumor mass possess either a fibroblast-like or epithelial morphology (FIG. 1, bottom panel).

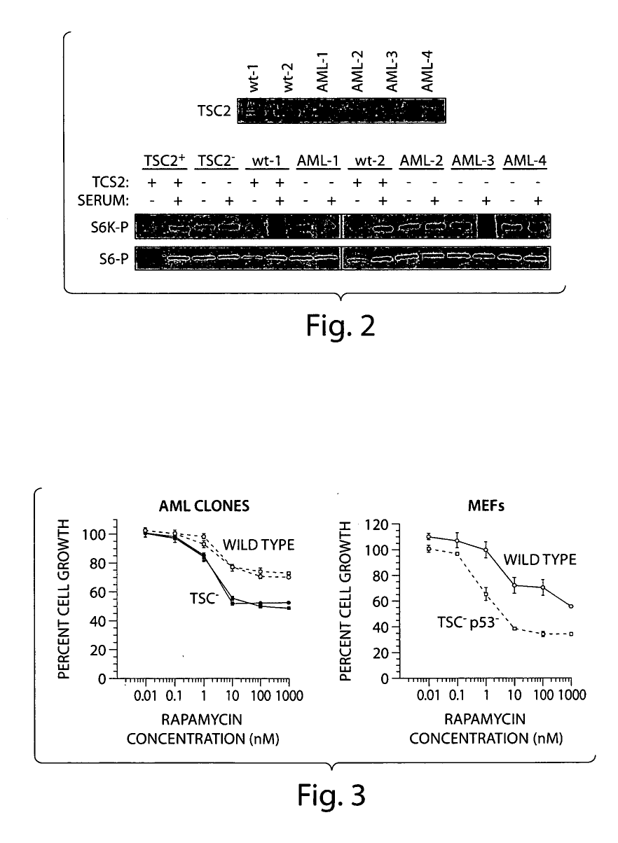

The loss of TSC function can be measured by phosphorylation of S6Kinase (S6K) and its substrate, ribosomal protein S6 (S6), in the absence of serum. In both TSC1^{-/-} or TSC2^{-/-} cells, the absence of the inhibitory TSC complex mimics mitogenic stimulation and results in constitutively active S6K signaling. To establish that AML621 clones are functionally TSC2-null, we isolated protein from wt-1, wt-2, AML-1, AML-2, AML-3, AML-4, and negative and positive TSC2 MEF control cells (TSC2−, TSC2+), and performed immunoblotting analysis for TSC2 expression and serum-independent S6 and S6K phosphorylation (FIG. 2). The wildtype clones express TSC2 while the AML clones do not. Wildtype-1 and 2 display serum-dependent S6 and S6K phosphorylation while AMLs1-4 express constitutively phosphorylated S6 and S6K, indicative of TSC2 loss.

The mTOR inhibitor, rapamycin, has been shown to inhibit growth of liver hemangiomas in TSC2 knockout mice, as well as of embryonic fibroblasts derived from knockout animals. We assessed rapamycin sensitivity of the AML clones (FIG. 3). Dose response growth assay demonstrates that while the growth of the AML clones is differentially inhibited as compared with wildtype lines generated from the 621 tumor mass, the human cell lines are less sensitive to rapamycin than the rodent cells (MEFs). Furthermore, p53−/− MEF cell lines and the Eker rat leiomyoma cell lines grow anchorage-independent colonies in soft agar, while the AML clones we developed do not (data not shown). This indicates that expression of E6E7 in AML cells does not result in transformation. Differences between the responses of human and rodent cells to rapamycin may reflect an inherent difference between the two species in how they will respond to therapeutics.

### EXAMPLE 3

### Microarray Analysis of Gene Expression in AMLS

In order to identify novel protein targets for the development of immunotherapeutics to treat TSC, microarray expression profiling was performed on 4 primary AML tumor tissues (AML548, AML564, AML576, AML1003) from different patients and TSC2^{-/-} AML cell lines (A-A2, A-C4) to identify genes up-regulated in AMLs. AML expression data was compared to 7 pooled normal tissues, including kidney, lung, trachea, aorta, left ventricle, uterus, and whole brain. Total RNA was converted to labeled cDNA and then hybridized to the Affymetrix GeneChip Human Genome U133 2.0 plus array containing more then 38,500 genes. The heirachical clustering analysis was performed using the Spotfire DecisionSite for Functional Genomics™ software platform (Spotfire, Somerville, Mass.).

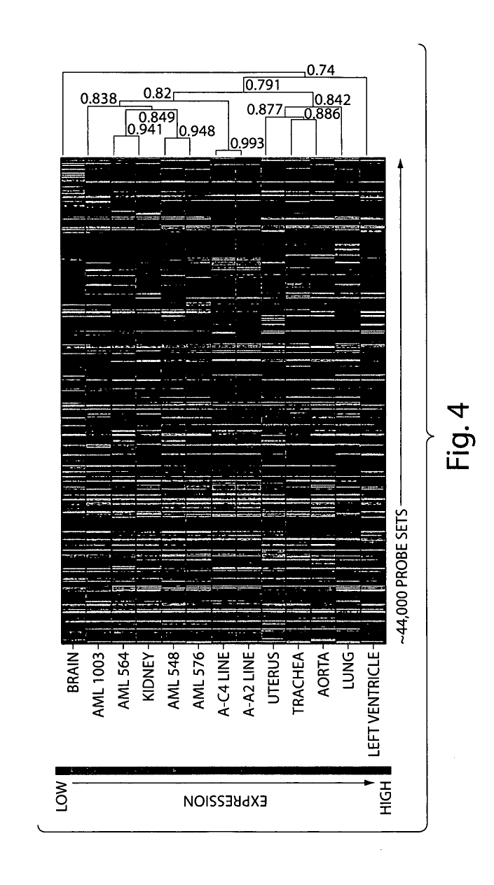

Heirarchical clustering algorithms are designed to assess how closely related multiple samples are to one another. In this case, how closely does the gene expression profile of one sample match the profile of every other sample, thereby generating a relative similarity percentage. As expected, the two AML clonal cell lines generated from the same AML are highly related (>99%) suggesting the immortalization process did not produce global changes in gene expression between clones (FIG. 4). Although there is diversity between primary AML samples ranging from 83.8% to 94.9% similarity, the AMLs are more like each other then almost all the normal samples, including the smooth muscle tissues of aorta, uterus, and trachea. The one exception is their high similarity to kidney (>83.8%). While AMLs are found almost exclusively on the kidney, the tumors themselves are composed of smooth muscle, adipose, and blood vessel. This apparent close relationship between AML and kidney might be explained by the accidental collection of adjacent kidney tissue during resection of the tumor and the heterogeneous nature of the AML. However, the AML cell lines are also much more similar to kidney then any other tissue, and these are clonally derived pure AML cell populations.

Principal component analysis of gene expression was performed as follows. Only genes that were expressed or ‘present’ in at least one of the 11 samples were selected for analysis. We performed a two-tailed T-Test for each gene to determine if the expression in group 1 (4 primary AMLs plus 1 AML cell line) and group 2 (all normal tissues except brain) are significantly different. For those genes significantly (T-value >0.05) expressed in AMLs compared with the normal tissues group, the fold change of median gene expression of group 1 compared with group 2 was determined. 115 genes were found to be up-regulated in AMLs by at least 5-fold with a T-value of <0.05 are shown (Table B). Silv, the antigen for the HNB45 antibody, known to be over-expressed in TSC-null cells, was expressed 50-fold greater in AMLs in this experiment. The membrane-type 1 matrix metalloproteinase (mmp14/MT1-MMP) shown to be highly expressed in LAM, is overexpressed 5-fold in AMLs as well (Matsui K., et al. 2000). In addition to silv, several genes associated with melanomas are also up-regulated in AMLs (Table D). MelanA, melanophillin, mmp14, OA1, ABCB5, gpnmb are all expressed significantly higher in TSC tissue. However not all genes associated with melanoma are overexpressed in AMLs as evident by nearly equal levels of expression between CD63, Dct, Tyrp1, and MAGE-1 and normal tissue. Transmembrane or secreted proteins that were identified as up-regulated in AMLs are listed in Table C.

Cytototoxic T lymphocytes (CTL) frequently recognize nonmutated endogenous proteins that are expressed both in normal tissues and in growing tumors. These Ags may be useful as vaccine targets, and CTLs targeted against them can cause tumor regression upon adoptive transfer. Tumor-associated antigens recognized by tumor-reactive T lymphocytes has led to the development of antigen-specific immunotherapy of cancer. Melanoma is particularly resistant to traditional chemotherapy and radiation treatments and has become an important target for the development of antibody therapies and peptide-based vaccines. Several proteins required for proper melanosomal function in melanocytes, are commonly over-expressed in various forms of melanoma. melan-A, silv, Tyrosinase, Trp2/DCT, Trp1/Tyrp1, OA1, and gpnmb/osteoactivin (gpnmb), are all transmembrane proteins normally expressed in melanosomes, but are upregulated in melanoma and have been dubbed, melanoma-associated antigens (MAAs). Several MAAs has shown promise as a target for vaccine development and CTL therapy for melanoma. Vaccine-induced circulating CD8+ T cells specific for melan-A, silv, and tyrosinase-derived peptides have already been tested successfully in clinical trials in patients with advanced melanoma. Thus, MAAs are potential targets for vaccine development in TSC-related disorders.

TABLE B

| Probe set ID | Accession No. | Gene | Clone | Protein | Fold Δ |
|---|---|---|---|---|---|
| 206696_at | NM_000273.1 | OA1/GPR143 | | Ocular albinism I | 96.7 |
| 209848_s_at | U01874.1 | SILV/PMEL17 | | Silver/pMEL17/gp100 | 50.6 |
| 229947_at | AI088609 | PI15 | | protease inhibitor 15 preproprotein | 35.6 |
| 229290_at | AI692575 | OCT6 | | transcription factor Oct-6 | 32.4 |
| 218468_s_at | AF154054.1 | DRM/GREM1 | | DRM/Gremlin-1 | 28.6 |
| 215767_at | AF052145.1 | EST | 24400 mRNA | | 25.5 |
| 218469_at | NM_013372.1 | DRM/GREM1 | | DRM/Gremlin-1 | 25.5 |
| 213482_at | BF593175 | DOCK3 | | dedicator of cyto-kinesis 3 | 24.8 |
| 214156_at | AL050090.1 | MYRIP | | myosin VIIA and Rab interacting protein | 23.0 |
| 232195_at | R41459 | EST | IMAGE: 29255 | KIAA1136 | 17.6 |
| 219279_at | NM_017718.1 | EST | FLJ20220 | hypothetical protein FLJ20220 | 13.7 |
| 214046_at | AA017721 | EST | DKFZp564N1662 | | 12.1 |
| 1558846_at | AL833418.1 | PNLIPRP3 | | Pancreatic lipase-related protein 3 | 12.0 |
| 203381_s_at | N33009 | APOE | | apolipoprotein E | 12.0 |
| 214586_at | T16257 | GPR37 | | protein-coupled receptor 37 (endothelin receptor type B-like) | 11.8 |
| 244444_at | AW082870 | PKD1L2 | | Polycystic kidney disease 1-like 2 | 11.7 |
| 244353_s_at | AI675682 | TBPL1 | | TBP-like 1 | 11.3 |
| 213790_at | W46291 | ADAM12 | | disintegrin and metalloproteinase-domain 12 | 11.0 |
| 203382_s_at | NM_000041.1 | APOE | | apolipoprotein E | 10.8 |
| 230401_at | BF197705 | NUPL2 | | Nucleoporin like 2 | 10.6 |
| 212806_at | AL138349 | EST | DKFZp762I1914 | KIAA0367 | 10.6 |
| 238969_at | BF512162 | EST | IMAGE: 3070060 | | 10.3 |
| 240423_at | R54953 | ABCB6 | | ATP-binding cassette, sub-family B, member 6 | 10.3 |
| 207938_at | NM_015886.1 | PI15 | | protease inhibitor 15 preproprotein | 10.2 |
| 226777_at | AA147933 | ADAM12 | | disintegrin and metalloproteinase domain 12 (meltrin alpha) | 10.1 |
| 219578_s_at | NM_030594.1 | CPEB1 | | cytoplasmic polyadenylation element binding protein 1 | 9.9 |
| 211207_s_at | AF129166.1 | LACS5 | | long-chain acyl-CoA synthetase 5 | 9.7 |
| 1558473_at | AK096402.1 | EST | FLJ39083 | | 9.6 |
| 218959_at | NM_017409.1 | HOXC10 | | homeo box C10 | 9.5 |
| 212805_at | AB002365.1 | EST | | KIAA0367 | 9.4 |
| 211162_x_at | AF116616.1 | SCD | | PRO0998 | 9.1 |
| 206030_at | NM_000049.1 | ASPA | | Aspartoacylase (aminoacylase 2, Canavan disease) | 9.0 |
| 240101_at | BF508153 | EST | IMAGE: 3089055 | | 8.9 |
| 226390_at | AA628398 | EST | IMAGE: 1032745 | | 8.8 |
| 212884_x_at | AI358867 | APOE | | apolipoprotein E | 8.7 |
| 1556346_at | AJ227860.1 | EST | | | 8.5 |
| 229725_at | AV705292 | ACSL6 | | Acyl-CoA synthetase long-chain family member 6 | 8.2 |
| 243885_x_at | AA526937 | EST | IMAGE: 969076 | | 8.2 |
| 202952_s_at | NM_003474.2 | ADAM12 | | disintegrin and metalloproteinase domain 12 (meltrin alpha), var. 1 | 8.1 |
| 211708_s_at | BC005807.1 | EST | MGC: 10264 | | 7.8 |
| 237265_at | BF062257 | EST | IMAGE: 3481213 | | 7.8 |
| 1562247_at | AL833160.1 | EST | DKFZp686J2011 | | 7.8 |
| 202450_s_at | NM_000396.1 | CTSK | | cathepsin K | 7.7 |
| 207400_at | NM_006174.1 | NPY5R | | neuropeptide Y receptor Y5 | 7.6 |
| 1554018_at | BC011595.1 | GPNMB | | glycoprotein nmb | 7.5 |
| 244684_at | AI432340 | EST | IMAGE: 2112610 | | 7.4 |
| 1560683_at | AL832227.1 | EST | DKFZp686P1536 | | 7.4 |
| 235737_at | AW118681 | EST | IMAGE: 2605355 | | 7.3 |
| 204044_at | NM_014298.2 | QPRT | | quinolinate phosphoribosyltransferase | 7.3 |
| 1557890_at | BC035182.1 | EST | IMAGE: 5266307 | | 7.2 |
| 1563787_a_at | AK097760.1 | CAGE1 | | cancertestis antigen gene 1 | 7.2 |
| 229715_at | AW006182 | EST | IMAGE: 2566376 | | 7.1 |
| 1564383_s_at | AK093253.1 | EST | IMAGE: 4869921 | | 7.1 |
| 203069_at | NM_014849.1 | EST | KIAA0736 | | 7.0 |
| 218211_s_at | NM_024101.1 | MLPH | | melanophilin/Slac2-c | 6.9 |
| 214147_at | AL046350 | EST | DKFZp434J097 | | 6.7 |
| 208510_s_at | NM_015869.1 | PPARG | | peroxisome proliferative activated receptor, gamma | 6.6 |
| 220484_at | NM_018298.1 | MCOLN3 | | Mucolipin3 | 6.6 |
| 201907_x_at | U49262.1 | DVL | | dishevelled | 6.6 |
| 239326_at | AA988134 | EST | IMAGE: 1604651 | | 6.6 |
| 214680_at | BF674712 | NTRK2 | | neurotrophic tyrosine kinase, receptor, type 2 | 6.5 |
| 237070_at | AI277662 | EST | IMAGE: 1878472 | | 6.5 |
| 227498_at | AI480314 | EST | IMAGE: 2157753 | | 6.5 |
| 205122_at | BF439316 | TMEFF1 | | transmembrane protein with EGF-like and two follistatin-like domains 1 | 6.5 |
| 228116_at | AW167298 | EST | IMAGE: 2634005 | | 6.4 |
| 224494_x_at | BC006283.1 | DHRS10 | | dehydrogenase/reductase (SDR family) member 10 | 6.4 |
| 200832_s_at | AB032261.1 | SCD | | stearoyl-CoA desaturase | 6.4 |
| 240236_at | N50117 | EST | IMAGE: 282792 | | 6.3 |
| 200831_s_at | AA678241 | SCD | | stearoyl-CoA desaturase (delta-9-desaturase) | 6.3 |
| 1565544_at | AI758773 | EST | IMAGE: 2279989 | | 6.3 |
| 228274_at | BE963955 | EST | IMAGE: 3875860 | | 6.2 |
| 1561513_at | BC043294.1 | EST | IMAGE: 5298087 | | 6.2 |
| 1562102_at | BC014579.1 | EST | IMAGE: 3681106 | | 6.2 |
| 219113_x_at | NM_016246.1 | DHRS10 | | retinal short-chain dehydrogenasereductase | 6.2 |
| 206617_s_at | NM_002910.4 | RENBP | | renin-binding protein | 6.1 |
| 224497_x_at | BC006294.1 | DHRS10 | | dehydrogenase/reductase (SDR family) member 10 | 6.1 |
| 242546_at | BE738279 | EST | IMAGE: 3839194 | | 6.1 |
| 229797_at | AI636080 | EST | IMAGE: 2296074 | | 6.0 |
| 229550_at | AB037830.1 | EST | KIAA1409 | | 6.0 |
| 1553768_a_at | NM_173674.1 | DCBLD1 | | discoidin, CUB and LCCL domain containing 1 | 6.0 |
| 210609_s_at | BC000474.1 | TP53I3 | | tumor protein p53 inducible protein 3, transcript, var.2 | 5.9 |
| 1563840_at | BC040569.1 | EFTUD1 | | elongation factor Tu GTP binding domain containing 1 | 5.9 |
| 231936_at | AK000445.1 | HOXC9 | | homeo box C9 | 5.8 |
| 235050_at | AI742872 | SLC2A12 | | Solute carrier family 2 (facilitated glucose transporter), member 12 | 5.8 |
| 207091_at | NM_002562.1 | P2RX7 | | Purinergic receptor P2X ligand-gated ionchannel 7 | 5.8 |
| 228415_at | AA205444 | AP1S2 | | adaptor-related protein complex 1, sigma 2 subunit | 5.8 |
| 215003_at | AA921844 | DGS-A | | DiGeorge Syndrome gene A | 5.8 |
| 204694_at | NM_001134.1 | AFP | | alpha-fetoprotein | 5.7 |
| 1562656_at | BC043591.1 | EST | IMAGE: 5248626 | | 5.7 |
| 205249_at | NM_000399.2 | EGR2 | | early growth response 2 | 5.7 |
| 205240_at | NM_013296.1 | LGN | | LGN protein | 5.7 |
| 213107_at | R59093 | EST | IMAGE: 41943 | KIAA0551 | 5.6 |
| 216222_s_at | AI561354 | MYO10 | | myosin X | 5.6 |
| 1570125_at | BC037977.1 | EST | IMAGE: 5229457 | | 5.6 |
| 238232_at | AI634355 | EST | IMAGE: 2232868 | | 5.5 |
| 211267_at | U82811.1 | HANF/HESX1 | | homeodomain-containing protein | 5.5 |
| 1557348_at | AI915861 | EST | IMAGE: 2378957 | | 5.5 |
| 204527_at | NM_000259.1 | MYO5A | | myosin VA (heavy polypeptide 12, myoxin) | 5.5 |
| 212664_at | AL567012 | TUBB5 | | tubulin, beta, 5 | 5.5 |
| 220324_at | NM_024882.1 | ORF | | Hypothetical protein FLJ13189 | 5.4 |
| 229526_at | AI886656 | AQP11 | | Aquaporin 11 | 5.4 |
| 1559789_a_at | AK097019.1 | ORF | | Hypothetical protein FLJ37549 | 5.3 |
| 1557292_a_at | AW665790 | MCOLN3 | | Mucolipin3 | 5.3 |
| 232606_at | AK021894.1 | ANK2 | | Ankyrin 2, neuronal | 5.3 |
| 237034_at | AW002876 | EST | IMAGE: 2480346 | | 5.2 |
| 221530_s_at | BE857425 | DEC2 | | bHLH protein DEC2 | 5.2 |
| 228262_at | AW237462 | EST | IMAGE: 2689732 | Hypothetical protein FLJ14503 | 5.2 |
| 227084_at | AW339310 | DTNA | | Dystrobrevin, alpha | 5.2 |
| 1554121_at | BC012536.1 | HSD17B12 | | Hydroxysteroid (17-beta) dehydrogenase 12 | 5.1 |
| 228807_at | AI078764 | EST | IMAGE: 1677212 | | 5.1 |
| 1553260_s_at | NM_152525.1 | EST | | hypothetical protein FLJ25351 | 5.1 |
| 237608_at | AW665177 | EST | IMAGE: 2979327 | | 5.1 |
| 217279_x_at | X83535.1 | MMP14 | | membrane-type matrix metalleoproteinase 14 | 5.0 |
| 1557745_at | BE551038 | EST | IMAGE: 3233325 | | 5.0 |
| 1554636_at | BC032569.1 | EST | IMAGE: 5242641 | | 5.0 |

TABLE C

| | | | | | | | | | | | | | | | | | | Fold |
|---|---|---|---|---|---|---|---|---|---|---|---|---|---|---|---|---|---|---|
| | | Gene | | | | | | | | | | | | | | | T-Test | Chang |
| TSC | | Family | Biological | | AML | AML | AML | AML | A-C4 | | | Left | | | | | (no | (no |
| No. | Gene Name | Name | Process | Molecular Function | 1003 | 564 | 548 | 576 | line | Brain | Lung | Ventricle | Uterus | Aorta | Trachea | Kidney | brain) | Brain) |
| 1 | melan-A | — | — | — | 10.5 | 2155.7 | 9168.7 | 7604.2 | 15.6 | 29.4 | 1.9 | 3.2 | 3.3 | 19.2 | 3.3 | 19.8 | 0.0583 | 653.2 |
| 2 | Ocular albinism | GPCRs | eye pigment | G-protein coupled | 7.3 | 283.8 | 1034.9 | 662.2 | 872 | 24.5 | 31.7 | 4.8 | 3 | 8.5 | 5.2 | 76.3 | 0.0105 | 127.3 |
| | 1/G-rotein-coupled | | biosynthesis /// | receptor activity | | | | | | | | | | | | | | |
| | receptor 143 | | signal | | | | | | | | | | | | | | | |
| | | | transduction /// | | | | | | | | | | | | | | | |
| | | | G-protein | | | | | | | | | | | | | | | |
| | | | coupled receptor | | | | | | | | | | | | | | | |
| | | | protein signaling | | | | | | | | | | | | | | | |
| | | | pathway /// | | | | | | | | | | | | | | | |
| | | | visual perception | | | | | | | | | | | | | | | |
| 3 | silver/gp100/pMel17 | Pmel- | melanin | — | 30.6 | 1821.2 | 5586.2 | 4340.8 | 7.2 | 13.8 | 72.4 | 13.6 | 87.5 | 53.9 | 18.1 | 11.3 | 0.0496 | 100.6 |
| | | 17/NMB | biosynthesis | | | | | | | | | | | | | | | |
| | | family | from tyrosine | | | | | | | | | | | | | | | |
| 4 | gremlin 1 homolog, | — | development /// | protein binding | 16.6 | 752.8 | 9928.2 | 6565.7 | 13048.5 | 417.2 | 127.3 | 11.9 | 332.4 | 341 | 37 | 355.4 | 0.0308 | 51.6 |
| | cysteine knot | | neurogenesis | | | | | | | | | | | | | | | |
| | superfamily | | | | | | | | | | | | | | | | | |
| | (Xenopus laevis) | | | | | | | | | | | | | | | | | |
| 5 | ATP-binding | — | — | — | 19.4 | 171.2 | 959 | 998.8 | 76.6 | 2.1 | 5.1 | 5.4 | 2.1 | 14.3 | 2.2 | 3.6 | 0.0536 | 47.6 |
| | Cassette, sub-family | | | | | | | | | | | | | | | | | |
| | B (MDR/TAP), | | | | | | | | | | | | | | | | | |
| | member 5 | | | | | | | | | | | | | | | | | |
| 6 | 5- | GPCRs | G-protein | rhodopsin-like | 121.3 | 1412.9 | 10599 | 80124 | 201.8 | 8.7 | 2.5 | 143.5 | 442.6 | 458.1 | 23.4 | 40.2 | 0.0811 | 35.1 |
| | hydroxytryptamine | | signaling, | receptor activity /// | | | | | | | | | | | | | | |
| | (serotonin) receptor | | coupled to IP3 | signal transducer | | | | | | | | | | | | | | |
| | 2B | | second | activity /// serotonin | | | | | | | | | | | | | | |
| | | | messenger | receptor activity | | | | | | | | | | | | | | |
| | | | (phospholipase | | | | | | | | | | | | | | | |
| | | | C activating) /// | | | | | | | | | | | | | | | |
| | | | circulation /// | | | | | | | | | | | | | | | |
| | | | positive | | | | | | | | | | | | | | | |
| | | | regulation of I- | | | | | | | | | | | | | | | |
| | | | kappaB | | | | | | | | | | | | | | | |
| | | | kinase/NF- | | | | | | | | | | | | | | | |
| | | | kappaB cascade | | | | | | | | | | | | | | | |
| 7 | mucolipin 3 | — | cation transport | cation channel activity | 2.2 | 320.9 | 575.1 | 595.4 | 487.8 | 13.6 | 11.2 | 2.8 | 114.6 | 15.3 | 118.9 | 121 | 0.0102 | 31.9 |
| 8 | a disintegrin and | peptidase | proteolysis and | metalloendopeptidase | 134.5 | 230.3 | 690.9 | 196 | 834.8 | 28.1 | 72.2 | 8.6 | 33.3 | 5.6 | 59.7 | 2.7 | 0.0158 | 26.8 |
| | metalloproteinase | family | peptidolysis /// | activity /// protein | | | | | | | | | | | | | | |
| | domain 12 (meltrin | M12B | cell adhesion /// | binding /// zinc ion | | | | | | | | | | | | | | |
| | alpha | | myoblast fusion | binding /// hydrolase | | | | | | | | | | | | | | |
| | | | | activity | | | | | | | | | | | | | | |
| 9 | myosin VIIA and | — | — | — | 3092.4 | 418.3 | 2741.3 | 1819.5 | 1.8 | 867.7 | 107 | 59.3 | 473.7 | 88 | 53.1 | 70.2 | 0.0271 | 25.9 |
| | Rab interacting | | | | | | | | | | | | | | | | | |
| | protein | | | | | | | | | | | | | | | | | |
| 10 | melanophilin | — | melanin | — | 1009.5 | 166.9 | 1929.9 | 1229.3 | 150.8 | 25.1 | 1037.9 | 57.7 | 41.2 | 11.1 | 256.8 | 5.1 | 0.0951 | 24.5 |
| | | | synthesis | | | | | | | | | | | | | | | |
| 11 | ATP-binding | ABC | carbohydrate | nucleotide binding /// | 3.5 | 298.8 | 1856.9 | 1211.7 | 3.8 | 524.3 | 19.2 | 19.4 | 12.6 | 14.1 | 12 | 8.5 | 0.0788 | 23.7 |
| | cassette, sub-family | transport | metabolism /// | receptor activity /// | | | | | | | | | | | | | | |
| | C (CFTR/MRP), | | transport /// | ATP binding /// | | | | | | | | | | | | | | |
| | member 8 | | potassium ion | sulfonylurea receptor | | | | | | | | | | | | | | |
| | | | transport | activity /// potassium | | | | | | | | | | | | | | |
| | | | | ion transporter activity | | | | | | | | | | | | | | |
| | | | | /// ATPase activity, | | | | | | | | | | | | | | |
| | | | | coupled to | | | | | | | | | | | | | | |
| | | | | transmembrane | | | | | | | | | | | | | | |
| | | | | movement of | | | | | | | | | | | | | | |
| | | | | substances | | | | | | | | | | | | | | |
| 12 | vasoactive intestinal | GPCRs | signal | G-protein coupled | 750.5 | 12.7 | 274.8 | 255.4 | 11.7 | 17.5 | 26.8 | 27.1 | 13.3 | 16.8 | 7 | 8.7 | 0.075 | 19.2 |
| | peptide receptor 2 | | transduction /// | receptor activity /// | | | | | | | | | | | | | | |
| | | | G-protein | vasoactive intestinal | | | | | | | | | | | | | | |
| | | | coupled receptor | polypeptide receptor | | | | | | | | | | | | | | |
| | | | protein signaling | activity | | | | | | | | | | | | | | |
| | | | pathway /// cell- | | | | | | | | | | | | | | | |
| | | | cell signaling | | | | | | | | | | | | | | | |
| 13 | pancreatic lipase- | — | — | — | 10.5 | 117.3 | 115.6 | 138.2 | 351.3 | 2.1 | 22.6 | 1.1 | 13.3 | 6.2 | 1.7 | 29.5 | 0.0265 | 18.9 |
| | related protein 3 | | | | | | | | | | | | | | | | | |
| 14 | polycystic kidney | — | cation transport | cation channel activity | 200.5 | 216.3 | 316.6 | 686.8 | 20 | 50.4 | 30 | 19.2 | 17.8 | 88.4 | 3.6 | 1.9 | 0.0292 | 12.2 |
| | disease 1-like 2 | | /// neuropeptide | /// sugar binding | | | | | | | | | | | | | | |
| | | | signaling | | | | | | | | | | | | | | | |
| | | | pathway | | | | | | | | | | | | | | | |
| 15 | attractin-like 1 | — | development | receptor activity /// | 1010.8 | 175.6 | 345.9 | 247 | 20.2 | 878.3 | 20.4 | 25 | 69.2 | 88.1 | 2.2 | 12.9 | 0.0668 | 12.1 |
| | | | | structural molecule | | | | | | | | | | | | | | |
| | | | | activity /// sugar | | | | | | | | | | | | | | |
| | | | | binding | | | | | | | | | | | | | | |
| 16 | solute carrier family | — | carbohydrate | transporter activity /// | 1023.2 | 499.7 | 1597.9 | 1481.4 | 26.1 | 194.5 | 45 | 370.5 | 54.7 | 118 | 93.2 | 87.7 | 0.0171 | 11.7 |
| | 2 (facilitated | | transport | sugar porter activity | | | | | | | | | | | | | | |
| | glucose | | | | | | | | | | | | | | | | | |
| | transporter), | | | | | | | | | | | | | | | | | |
| | member 12 | | | | | | | | | | | | | | | | | |
| 17 | protease inhibitor | Allergen | — | peptidase activity /// | 335.2 | 40.4 | 530.6 | 405.6 | 50.5 | 11.1 | 7.9 | 33.6 | 32 | 65.3 | 42.1 | 16 | 0.0244 | 10.5 |
| | 15 | V5/Tpx-1 | | trypsin inhibitor | | | | | | | | | | | | | | |
| | | related | | activity | | | | | | | | | | | | | | |
| 18 | tumor protein p53 | — | induction of | alcohol | 1283 | 751.1 | 2534 | 1900 | 510 | 82.4 | 515.7 | 88.3 | 127.5 | 106.3 | 308.8 | 584.9 | 0.0114 | 10.1 |
| | inducible protein 3 | | apoptosis by | dehydrogenase | | | | | | | | | | | | | | |
| | | | oxidative stress | activity, zinc- | | | | | | | | | | | | | | |
| | | | | dependent /// zinc ion | | | | | | | | | | | | | | |
| | | | | binding | | | | | | | | | | | | | | |
| 19 | astrotactin | — | cell adhesion /// | protein binding | 870.8 | 98.1 | 719.5 | 210.1 | 7.9 | 1806.4 | 84.5 | 53 | 31.3 | 24.7 | 13.6 | 9.1 | 0.0557 | 8.5 |
| | | | neuronal cell | | | | | | | | | | | | | | | |
| | | | adhesion /// cell | | | | | | | | | | | | | | | |
| | | | migration | | | | | | | | | | | | | | | |
| 20 | glycoprotein | Polycystic | negative | — | 3079.7 | 228.5 | 2072.3 | 1922 | 2027.1 | 80.8 | 58.1 | 894.8 | 373.4 | 252.6 | 299.3 | 71.5 | 0.0065 | 8.0 |
| | (transmembrane) | kidney | regulation of cell | | | | | | | | | | | | | | | |
| | nmb | disease | proliferation | | | | | | | | | | | | | | | |
| | | proteins | | | | | | | | | | | | | | | | |
| 21 | contactin 1 | — | cell adhesion | protein binding | 17.4 | 245.1 | 1110.5 | 1551.8 | 1.2 | 1424.8 | 31 | 14 | 65.8 | 94.7 | 28.1 | 63.1 | 0.0928 | 7.9 |
| 22 | neural epidermal | | | | 82.8 | 798.7 | 6293.4 | 9085.7 | 43.9 | 8064.2 | 436.9 | 119 | 124.3 | 125 | 420.5 | 181.9 | 0.1062 | 6.4 |
| | growth factor like | | | | | | | | | | | | | | | | | |
| | like-2 | | | | | | | | | | | | | | | | | |
| 23 | transmembrane | | signalling | | 256 | 183 | 2119.7 | 1123.2 | 507.2 | 2308.3 | 21.1 | 120.2 | 64.3 | 201.2 | 102.7 | 79.5 | 0.0498 | 6.4 |
| | protein with EGF- | | | | | | | | | | | | | | | | | |
| | like and two | | | | | | | | | | | | | | | | | |
| | follistatin-like | | | | | | | | | | | | | | | | | |
| | domains 1 | | | | | | | | | | | | | | | | | |
| 24 | peroxisome | — | thermoregulation | RNA binding /// | 7.3 | 61 | 563.9 | 393.9 | 6.6 | 22.3 | 8.6 | 20.5 | 19.9 | 9.8 | 4 | 34.8 | 0.099 | 6.2 |
| | proliferative | | /// cell glucose | transcription factor | | | | | | | | | | | | | | |
| | activated receptor, | | homeostasis /// | binding /// ligand- | | | | | | | | | | | | | | |
| | gamma, coactivator | | gluconeogenesis | dependent nuclear | | | | | | | | | | | | | | |
| | 1, alpha | | /// regulation of | receptor transcription | | | | | | | | | | | | | | |
| | | | transcription, | coactivator activity | | | | | | | | | | | | | | |
| | | | DNA-dependent | | | | | | | | | | | | | | | |
| | | | /// mRNA | | | | | | | | | | | | | | | |
| | | | processing /// | | | | | | | | | | | | | | | |
| | | | mitochondrion | | | | | | | | | | | | | | | |
| | | | organization and | | | | | | | | | | | | | | | |
| | | | biogenesis /// | | | | | | | | | | | | | | | |
| | | | RNA splicing /// | | | | | | | | | | | | | | | |
| | | | response to cold | | | | | | | | | | | | | | | |
| | | | /// fatty acid | | | | | | | | | | | | | | | |
| | | | oxidation /// | | | | | | | | | | | | | | | |
| | | | response | | | | | | | | | | | | | | | |
| 25 | matrix | — | proteolysis and | metalloendopeptidase | 575 | 111.7 | 572.4 | 351.8 | 494.9 | 16.6 | 98.7 | 27.9 | 240.4 | 164.8 | 164.7 | 39.3 | 0.0076 | 5.0 |
| | metalloproteinase | | peptidolysis | activity /// zinc ion | | | | | | | | | | | | | | |
| | 14 (membrane- | | | binding /// hydrolase | | | | | | | | | | | | | | |
| | inserted) | | | activity | | | | | | | | | | | | | | |
| 26 | vascular endothelial | | PDGE signalling | | 29.8 | 409.8 | 6947.4 | 1953.3 | 27.3 | 51 | 55.6 | 472.7 | 477.9 | 438.3 | 2541 | 26.6 | 0.3648 | −1.1 |
| | growth factor D | | | | | | | | | | | | | | | | | |

TABLE D

| ProbeSet ID | Gene | AML 1003 | AML 564 | AML 548 | AML 576 | A-C4 line |
|---|---|---|---|---|---|---|
| 206427_s_at | Melan-A/MART1¹ | 10.5 | 2155.7 | 9168.7 | 7604.2 | 15.6 |
| 243167_at | ABC85/p-glycoprotein¹ | 19.4 | 171.2 | 959 | 996.5 | 76.6 |
| 209848_s_at | Sllv/pMel17/gp 100¹ | 30.6 | 1821.2 | 5586.2 | 4340.8 | 7.2 |
| 206426_at | Melan-A/MART1¹ | 15.1 | 678.1 | 4319.2 | 3405.1 | 1.3 |
| 206696_at | Ocular Albhism 1 (OA1)¹ | 7.3 | 283.8 | 1034.9 | 662.2 | 872 |
| 1569072_s_at | ABC85/p-glycoprotein¹ | 32.4 | 213.1 | 1606 | 1192 | 35.2 |
| 214156_at | MYRIP | 3092.4 | 418.3 | 2741.3 | 1819.5 | 1.8 |
| 213790_at | ADAM12 variant 1² | 134.5 | 230.3 | 690.9 | 196 | 834.8 |
| 218211_s_at | Melanophllin (MLPH) | 4732.1 | 1217 | 8663.4 | 6886.4 | 2834.5 |
| 223795_at | Oculospanin (OCSP) | 13.1 | 72.1 | 785.2 | 1557.1 | 517 |
| 202952_s_at | ADAM12 variant 1² | 189.7 | 327.3 | 894.2 | 282.2 | 1837.2 |
| 1554018_at | gprmb/osto activin | 3079.7 | 228.5 | 2072.3 | 1922 | 2027.1 |
| 201141_at | gprmb/osto activin | 19753.8 | 7226.6 | 19413.1 | 21795.3 | 15081.2 |
| 202828_s_at | MMP14/MT1-MMP¹ | 575 | 111.7 | 572.4 | 351.8 | 494.9 |
| 202827_s_at | MMP14/MT1-MMP¹ | 764.9 | 43.9 | 998.5 | 404.4 | 866.2 |
| 221261_x_at | MAGE1¹ | 124.4 | 67.9 | 281.2 | 196 | 133.9 |
| 211602_s_at | TRYP1/TRP-1¹ | 76.8 | 110.9 | 407.8 | 467.6 | 146.5 |
| 205338_s_at | DCT/TRP-2¹ | 38.9 | 39.8 | 41.9 | 50.2 | 10.5 |
| 200663_at | melanoma 1 antigen (CD63)¹ | 12593 | 5328.4 | 14208.5 | 14690.7 | 14782.9 |
| 206630_at | Tyrosinase¹ | A | A | A | A | A |

| ProbeSet ID | Lung | L. Ventricle | Uterus | Aorte | Trachea | Kidney | T-test | Fold |
|---|---|---|---|---|---|---|---|---|
| 206427_s_at | 1.9 | 3.2 | 3.3 | 19.2 | 3.3 | 19.8 | 0.0583 | 653.2 |
| 243167_at | 5.1 | 5.4 | 2.1 | 14.3 | 2.2 | 3.6 | 0.0536 | 39.4 |
| 209848_s_at | 72.4 | 13.6 | 87.5 | 53.9 | 18.1 | 11.3 | 0.0496 | 50.6 |
| 206426_at | 34.3 | 8.1 | 18.3 | 34.5 | 21.2 | 29.1 | 0.0739 | 27.0 |
| 206696_at | 31.7 | 4.8 | 3 | 8.5 | 5.2 | 76.3 | 0.0105 | 96.7 |
| 1569072_s_at | 1.2 | 12.9 | 50.7 | 11.6 | 11.9 | 31.7 | 0.0752 | 17.2 |
| 214156_at | 107 | 59.3 | 473.7 | 88 | 53.1 | 70.2 | 0.0271 | 23.0 |
| 213790_at | 72.2 | 8.6 | 33.3 | 5.6 | 59.7 | 2.7 | 0.0158 | 11.0 |
| 218211_s_at | 2777 | 1092.4 | 204.9 | 274.5 | 1470.2 | 110.3 | 0.0152 | 6.9 |
| 223795_at | 118.1 | 42.7 | 22.5 | 81.5 | 42.4 | 70.9 | 0.0508 | 9.1 |
| 202952_s_at | 55.5 | 39.5 | 44.9 | 9.6 | 41.4 | 36 | 0.0400 | 8.1 |
| 1554018_at | 58.1 | 894.8 | 373.4 | 252.6 | 299.3 | 71.5 | 0.0065 | 7.3 |
| 201141_at | 2735.4 | 7953.5 | 5021.4 | 8042.4 | 4753.2 | 1306.3 | 0.0017 | 4.0 |
| 202828_s_at | 98.7 | 27.9 | 240.4 | 164.8 | 164.7 | 39.3 | 0.0076 | 3.8 |
| 202827_s_at | 252.5 | 68.1 | 233.5 | 151.7 | 219.1 | 153.3 | 0.0276 | 3.6 |
| 221261_x_at | 220.2 | 8.3 | 190.4 | 120.7 | 40.7 | 59.3 | 0.31277 | 1.5 |
| 211602_s_at | 27.6 | 111.1 | 437.1 | 116.7 | 130.2 | 67.8 | 0.3682 | 1.3 |
| 205338_s_at | 64.9 | 37.2 | 15.1 | 42.2 | 24 | 20.4 | 0.8281 | 1.3 |
| 200663_at | 17485.9 | 9608.9 | 11460.8 | 13589.8 | 10212.6 | 8787.5 | 0.5739 | 1.3 |
| 206630_at | A | A | A | A | A | A | N/A | N/A |

¹= Associated with melanome
²= Associated with carcinoma
A = Expression absent

### EXAMPLE 4

### RTQ-PCR Validation of Gene Expression in AMLs

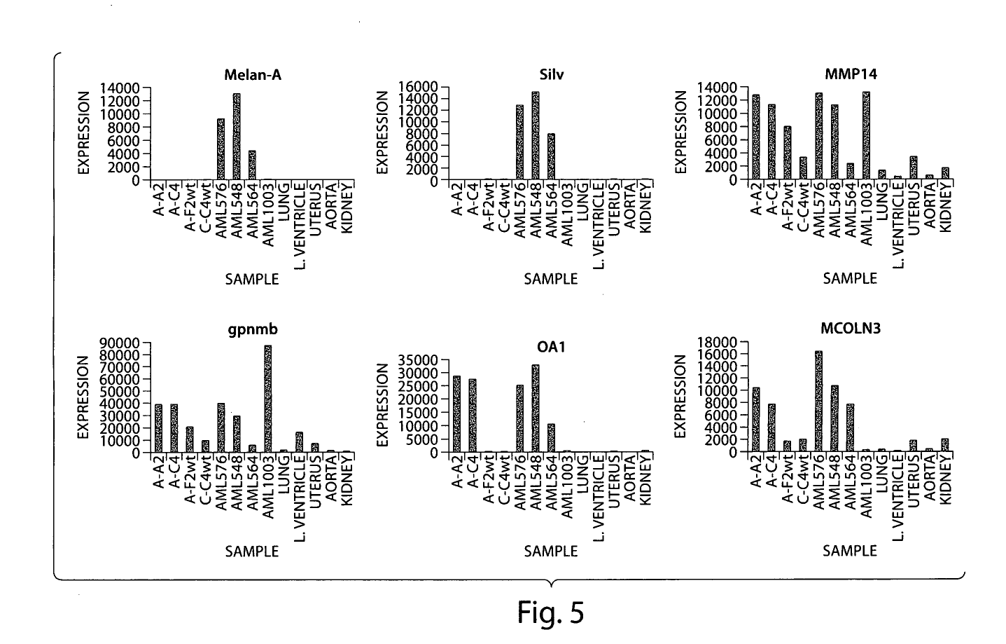

RTQ-PCR validation was performed on 32 genes identified by microarray analysis as expressed higher in AML tissue samples than normal control tissues by >5-fold, and are likely to be expressed on the cell surface. Of these genes, 22 were verified as up-regulated in at least 3 of 4 AML tissue samples. High expression of the melanoma associated genes, melanA, silv, OA1, gpnmb, and mmpl14 as determined by microarray, was supported by the RTQ-PCR results (FIG. 5). Interestingly, some genes appear to have nearly identical tissue expression patterns. Expression of silv and melanA are quite similar with a notably lack of expression in the AML cell lines, little to no expression in AML1003, and the highest expression in AML548. While this phenomena could be artifactual, it is possible that both genes may be regulated by the same signaling mechanism in the absence of TSC2.

Variation of gene expression between different AML tissue samples is evident by Most genes identified as up-regulated in AMLs, are not expressed in all 4 primary AMLs or cell lines. OA1 and mcoln3 are almost absent in AML1003, while mmp14 and gpnmb are only found at very low levels in AML564. This reflects the normal variation in gene expression found in AMLs between patients. The absence of expression some genes in the AML cell lines could be due to the inherent difference between gene expression in a 2-dimensional (cell line) and a 3-dimensional (primary tissue) environment, or the normal variation of gene expression between patients.

### EXAMPLE 5

### GPNMB and OA1 Expression in Melanoma and AMLs

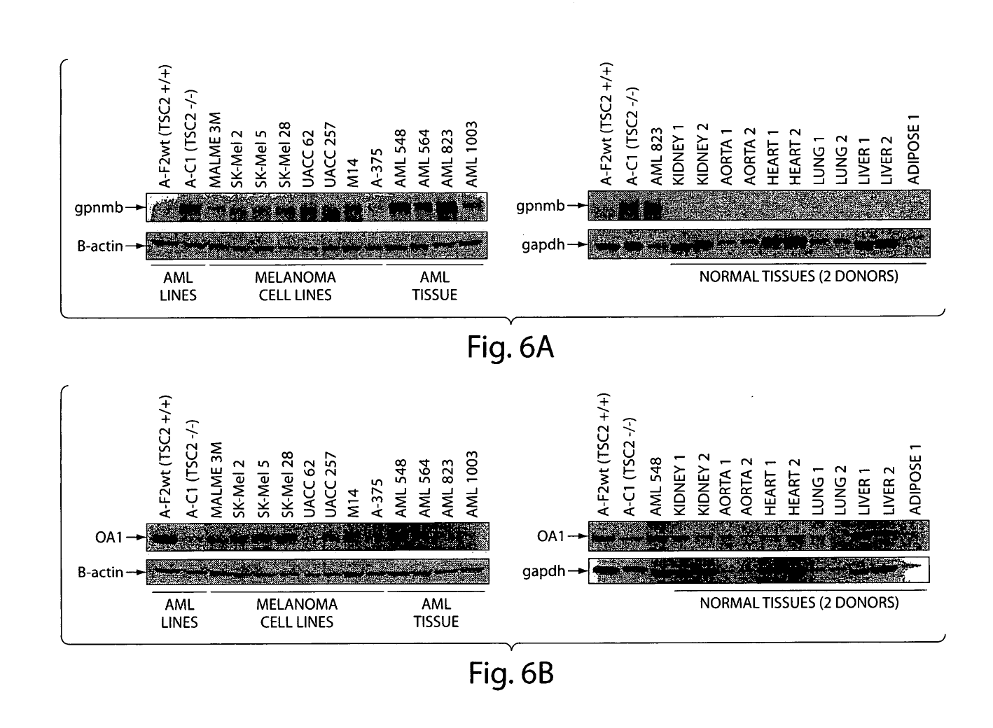

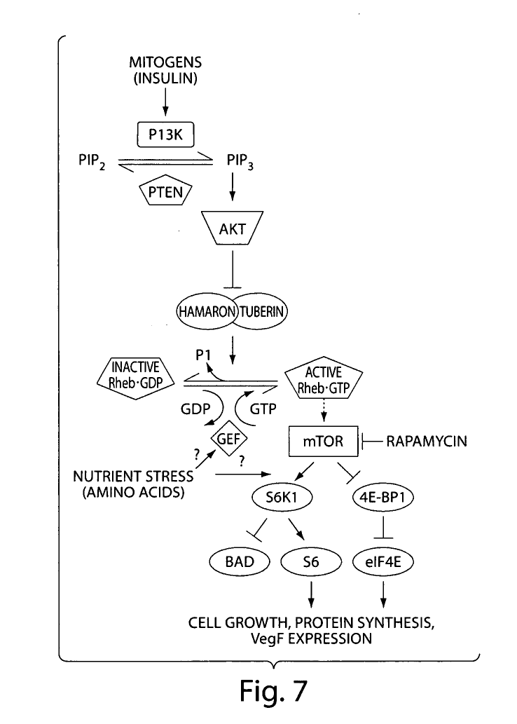

To assess the correlation between RNA levels and protein expression of gpnmb and OA1, we performed immunoblotting on 4 primary AMLs, 8 melanoma cell lines, 1 AML cell line and 1 control line, and 6 normal tissues from 2 donors (FIG. 6). Expression of gpnmb is very robust in primary AMLs and the TSC2^{-/-} AML cell line, with expression varying in the melanoma lines, and the lowest level observed the TSC2^{+/+} AML control line. Interestingly, expression of this MAA is actually higher in AML samples then melanoma, and appears to be TSC2 status dependent as indicated by the near absence of expression in the wildtype control line. There was no appreciable expression in any normal tissue tested. Housekeeping genes are traditionally used as load controls between samples, however expression varies between different tissues. GAPDH was used to compare loading of normal tissues. Despite the disparity of signal, similarity of GAPDH expression within a tissue type from different donors indicates tissue-dependent expression, not inequity of protein load. Coomassie staining verified that equal protein was loaded in all lanes.

OA1 expression also was strongest in AML primary tissue, although only in 2 of the 4 samples, and was not prevalent in the AML cell line. Expression was present in most melanoma lines as expected. OA1 was found to be significantly expressed in liver and to a lower extent, in heart.

### EXAMPLE 6

### TSC Nucleotide and Protein Sequences

Exemplary TSC nucleic acid and TSC polypeptide sequences are described below:

TSC1 Melan-A.

Both U06654. 1 and NM-0055 11 encode the protein sequence shown in Table 1C.

TABLE 1A

melan-A (U06654.1) nucleotide sequence
(SEQ ID NO:1)

CCGTCAGAAATCTAAACCCGTGACTATCATGGGACTCAAAACCAGCCCAA
AAAATAAGTCAAAACGATTAAGAGCCAGAGAAGCAGTCTTCATACACGCG
GCCAGCCAGCAGACAGAGGACTCTCATTAAGGAAGGTGTCCTGTGCCCTG
ACCCTACAAGATGCCAAGAGAAGATGCTCACTTCATCTATGGTTACCCCA
AGAAGGGGCACGGCCACTCTTACACCACGGCTGAAGAGGCCGCTGGGATC
GGCATCCTGACAGTGATCCTGGGAGTCTTACTGCTCATCGGCTGTTGGTA
TTGTAGAAGACGAAATGGATACAGAGCCTTGATGGATAAAAGTCTTCATG
TTGGCACTCAATGTGCCTTAACAAGAAGATGCCCACAAGAAGGGTTTGAT
CATCGGGACAGCAAAGTGTCTCTTCAAGAGAAAAACTGTGAACCTGTGGT
TCCCAATGCTCCACCTGCTTATGAGAAACTCTCTGCAGAACAGTCACCAC
CACCTTATTCACCTTAAGAGCCAGCGAGACACCTGAGACATGCTGAAATT
ATTTCTCTCACACTTTTGCTTGAATTTAATACAGACATCTAATGTTCTCC
TTTGGAATGGTGTAGGAAAAATGCAAGCCATCTCTAATAATAAGTCAGTG
TTAAAATTTTAGTAGGTCCGCTAGCAGTACTAATCATGTGAGGAAATGAT
GAGAAATATTAAATTGGGAAAACTCCATCAATAAATGTTGCAATGCATGA
TA.

TABLE 1B

melan-A (NM_005511) nucleotide sequence
(SEQ ID NO:2)

AGCAGACAGAGGACTCTCATTAAGGAAGGTGTCCTGTGCCCTGACCCTAC
AAGATGCCAAGAGAAGATGCTCACTTCATCTATGGTTACCCCAAGAAGGG
GCACGGCCACTCTTACACCACGGCTGAAGAGGCCGCTGGGATCGGCATCC
TGACAGTGATCCTGGGAGTCTTACTGCTCATCGGCTGTTGGTATTGTAGA
AGACGAAATGGATACAGAGCCTTGATGGATAAAAGTCTTCATGTTGGCAC
TCAATGTGCCTTAACAAGAAGATGCCCACAAGAAGGGTTTGATCATCGGG
ACAGCAAAGTGTCTCTTCAAGAGAAAAACTGTGAACCTGTGGTTCCCAAT
GCTCCACCTGCTTATGAGAAACTCTCTGCAGAACAGTCACCACCACCTTA
TTCACCTTAAGAGCCAGCGAGACACCTGAGACATGCTGAAATTATTTCTC
TCACACTTTTGCTTGAATTTAATACAGACATCTAATGTTCTCCTTTGGAA
TGGTGTAGGAAAAATGCAAGCCATCTCTAATAATAAGTCAGTGTTAAAAT
TTTAGTAGGTCCGCTAGCAGTACTAATCATGTGAGGAAATGATGAGAAAT
ATTAAATTGGGAAAACTCCATCAATAAATGTTGCAATGCATGATACTATC
TGTGCCAGAGGTAATGTTAGTAAATCCATGGTGTTATTTTCTGAGAGACA
GAATTCAAGTGGGTATTCTGGGGCCATCCAATTTCTCTTTACTTGAAATT
TGGCTAATAACAAACTAGTCAGGTTTTCGAACCTTGACCGACATGAACTG
TACACAGAATTGTTCCAGTACTATGGAGTGCTCACAAAGGATACTTTTAC
AGGTTAAGACAAAGGGTTGACTGGCCTATTTATCTGATCAAGAACATGTC
AGCAATGTCTCTTTGTGCTCTAAAATTCTATTATACTACAATAATATATT
GTAAAGATCCTATAGCTCTTTTTTTTTGAGATGGAGTTTCGCTTTTGTTG
CCCAGGCTGGAGTGCAATGGCGCGATCTTGGCTCACCATAACCTCCGCCT
CCCAGGTTCAAGCAATTCTCCTGCCTTAGCCTCCTGAGTAGCTGGGATTA
CAGGCGTGCGCCACTATGCCTGACTAATTTTGTAGTTTTAGTAGAGACGG
GGTTTCTCCATGTTGGTCAGGCTGGTCTCAAACTCCTGACCTCAGGTGAT
CTGCCCGCCTCAGCCTCCCAAAGTGCTGGAATTACAGGCGTGAGCCACCA
CGCCTGGCTGGATCCTATATCTTAGGTAAGACATATAACGCAGTCTAATT
ACATTTCACTTCAAGGCTCAATGCTATTCTAACTAATGACAAGTATTTTC
TACTAAACCAGAAATTGGTAGAAGGATTTAAATAAGTAAAAGCTACTATG
TACTGCCTTAGTGCTGATGCCTGTGTACTGCCTTAAATGTACCTATGGCA
ATTTAGCTCTCTTGGGTTCCCAAATCCCTCTCACAAGAATGTGCAGAAGA
AATCATAAAGGATCAGAGATTCTG.

TABLE 1C
Encoded melan-A protein sequence (SEQ ID NO:3)

MPREDAHFIYGYPKKGHGHSYTTAEEAAGIGILTVILGVLLLIGCWYCRR
RNGYRALMDKSLHVGTQCALTRRCPQEGFDHRDSKVSLQEKNCEPVVPNA
PPAYEKLSAEQSPPPYSP.

TSC2: Ocular Albinism 1/G-Protein-Coupled Receptor 143.

TABLE 2A

ocular albinism 1/G-protein-coupled receptor 143
(NM_000273.1) nucleotide sequence (SEQ ID NO:4)

ATGACCCAGGCAGGCCGGCGGGGTCCTGGCACACCCGAGCCGCGTCCGCG
AACACAGCCCATGGCCTCCCCGCGCCTAGGGACCTTCTGCTGCCCCACGC
GGGACGCAGCCACGCAGCTCGTGCTGAGCTTCCAGCCGCGGGCCTTCCAC
GCGCTCTGCCTGGGCAGCGGCGGGCTCCGCTTGGCGCTGGGCCTTCTGCA
GCTGCTGCCCGGCCGCCGGCCCGCGGGCCCCGGGTCCCCCGCGACGTCCC
CGCCGGCCTCGGTCCGCATCCTGCGCGCTGCCGCTGCCTGCGACCTTCTC
GGCTGCCTGGGTATGGTGATCCGGTCCACCGTGTGGTTAGGATTCCCAAA
TTTTGTTGACAGCGTCTCGGATATGAACCACACGGAAATTTGGCCTGCTG
CTTTCTGCGTGGGGAGTGCGATGTGGATCCAGCTGTTGTACAGTGCCTGC
TTCTGGTGGCTGTTTTGCTATGCAGTGGATGCTTATCTGGTGATCCGGAG
ATCGGCAGGACTGAGCACCATCCTGCTGTATCACATCATGGCGTGGGGCC
TGGCCACCCTGCTCTGTGTGGAGGGAGCCGCCATGCTCTACTACCCTTCC
GTGTCCAGGTGTGAGCGGGGCCTGGACCACGCCATCCCCCACTATGTCAC
CATGTACCTGCCCCTGCTGCTGGTTCTCGTGGCGAACCCCATCCTGTTCC
AAAAGACAGTGACTGCAGTGGCCTCTTTACTTAAAGGAAGACAAGGCATT
TACACGGAGAACGAGAGGAGGATGGGAGCCGTGATCAAGATCCGATTTTT
CAAATCATGCTGGTTTTAATTATTTGTTGGTTGTCGAATATCATCAATGA
AAGCCTTTTATTCTATCTTGAGATGCAAACAGATATCAATGGAGGTTCTT
TGAAACCTGTCAGAACTGCAGCCAAGACCACATGGTTTATTATGGGAATC
CTGAATCCAGCCCAGGGATTTCTCTTGTCTTTGGCCTTCTACGGCTGGAC
AGGATGCAGCCTGGGTTTTCAGTCTCCCAGGAAGGAGATCCAGTGGGAAT
CACTGACCACCTCGGCTGCTGAGGGGGCTCACCCATCCCCACTGATGCCC
CATGAAAACCCTGCTTCCGGGAAGGTGTCTCAAGTGGGTGGGCAGACTTC
TGACGAAGCCCTGAGCATGCTGTCTGAAGGTTCTGATGCCAGCACAATTG
AAATTCACACTGCAAGTGAATCCTGCAACAAAAATGAGGGTGACCCTGCT
CTCCCAACCCATGGAGACCTATGAAGGGGATGTGCTGGGGGTCCAGACCC
CATATTCCTCAGACTCAACAATTCTTGTTCTTTAGAACTGTGTTCTCACC
TTCCCAACACTGCACTGCCGAAGTGTAGCGGCCCCCAAACCTTGCTCTCA
TCACCAGCTAGAGCTTCTTCCCGAAGGGCCTTTAGGATAGGAGAAAGGGT
TCATGCACACACGTGTGAGAATGGAAGAGCCCCCTCCAGACCACTCTACA
GCTGCTCTAGCCTTAGTTGCCACTAGGAAGTTTTCTGAGGCTGGCTGTAA
AGTAAGTGTAAGGTCCACATCCTTGGGGAAGTAGTTAAATAAAATAGTTA
TGACTG.

TABLE 2B

Encoded ocular albinism 1/G-protein-coupled
receptor 143 protein sequence (SEQ ID NO:5)

MTQAGRRGPGTPEPRPRTQPMASPRLGTFCCPTRDAATQLVLSFQPRAFH
ALCLGSGGLRLALGLLQLLPGRRPAGPGSPATSPPASVRILRAAAACDLL
GCLGMVIRSTVWLGFPNFVDSVSDMNHTEIWPAAFCVGSAMWIQLLYSAC
FWWLFCYAVDAYLVIRRSAGLSTILLYHIMAWGLATLLCVEGAAMLYYPS
VSRCERGLDHAIPHYVTMYLPLLLVLVANPILFQKTVTAVASLLKGRQGI
YTENERRMGAVIKIRFFKIMLVLIICWLSNIINESLLFYLEMQTDINGGS
LKPVRTAAKTTWFIMGILNPAQGFLLSLAFYGWTGCSLGFQSPRKEIQWE
SLTTSAAEGAHPSPLMPHENPASGKVSQVGGQTSDEALSMLSEGSDASTI
EIHTASESCNKNEGDPALPTHGDL.

TSC3: Silver/gp100/pMell7.

U01874.1 and NM_{—}006928.1/2 (1/2are identical nucleic acid sequences) and 3 encode the protein sequence shown in Table 3D.

TABLE 3A

silver/gp100/pMel17 (U01874.1) nucleotide sequence
(SEQ ID NO:6)

CTCGAGATGGATCTGGTGCTAAAAAGATGCCTTCTTCATTTGGCTGTGAT
AGGTGCTTTGCTGGCTGTGGGGGCTACAAAAGTACCCAGAAACCAGGACT
GGCTTGGTGTCTCAAGGCAACTCAGAACCAAAGCCTGGAACAGGCAGCTG
TATCCAGAGTGGACAGAAGCCCAGAGACTTGACTGCTGGAGAGGTGGTCA
AGTGTCCCTCAAGGTCAGTAATGATGGGCCTACACTGATTGGTGCAAATG
CCTCCTTCTCTATTGCCTTGAACTTCCCTGGAAGCCAAAAGGTATTGCCA
GATGGGCAGGTTATCTGGGTCAACAATACCATCATCAATGGGAGCCAGGT
GTGGGGAGGACAGCCAGTGTATCCCCAGGAAACTGACGATGCCTGCATCT
TCCCTGATGGTGGACCTTGCCCATCTGGCTCTTGGTCTCAGAAGAGAAGC
TTTGTTTATGTCTGGAAGACCTGGGGCCAATACTGGCAAGTTCTAGGGGG
CCCAGTGTCTGGGCTGAGCATTGGGACAGGCAGGGCAATGCTGGGCACAC
ACACCATGGAAGTGACTGTCTACCATCGCCGGGGATCCCGGAGCTATGTG
CCTCTTGCTCATTCCAGCTCAGCCTTCACCATTACTGACCAGGTGCCTTT
CTCCGTGAGCGTGTCCCAGTTGCGGGCCTTGGATGGAGGGAACAAGCACT
TCCTGAGAAATCAGCCTCTGACCTTTGCCCTCCAGCTCCATGACCCCAGT
GGCTATCTGGCTGAAGCTGACCTCTCCTACACCTGGGACTTTGGAGACAG
TAGTGGAACCCTGATCTCTCGGGCACTTGTGGTCACTCATACTTACCTGG
AGCCTGGCCCAGTCACTGCCCAGGTGGTCCTGCAGGCTGCCATTCCTCTC
ACCTCCTGTGGCTCCTCCCCAGTTCCAGGCACCACAGATGGGCACAGGCC
AACTGCAGAGGCCCCTAACACCACAGCTGGCCAAGTGCCTACTACAGAAG
TTGTGGGTACTACACCTGGTCAGGCGCCAACTGCAGAGCCCTCTGGAACC
ACATCTGTGCAGGTGCCAACCACTGAAGTCATAAGCACTGCACCTGTGCA
GATGCCAACTGCAGAGAGCACAGGTATGACACCTGAGAAGGTGCCAGTTT
CAGAGGTCATGGGTACCACACTGGCAGAGATGTCAACTCCAGAGGCTACA
GGTATGACACCTGCAGAGGTATCAATTGTGGTGCTTTCTGGAACCACAGC
TGCACAGGTAACAACTACAGAGTGGGTGGAGACCACAGCTAGAGAGCTAC
CTATCCCTGAGCCTGAAGGTCCAGATGCCAGCTCAATCATGTCTACGGAA
AGTATTACAGGTTCCCTGGGCCCCCTGCTGGATGGTACAGCCACCTTAAG
GCTGGTGAAGAGACAAGTCCCCCTGGATTGTGTTCTGTATCGATATGGTT
CCTTTTCCGTCACCCTGGACATTGTCCAGGGTATTGAAAGTGCCGAGATC
CTGCAGGCTGTGCCGTCCGGTGAGGGGGATGCATTTGAGCTGACTGTGTC
CTGCCAAGGCGGGCTGCCCAAGGAAGCCTGCATGGAGATCTCATCGCCAG
GGTGCCAGCCCCCTGCCCAGCGGCTGTGCCAGCCTGTGCTACCCAGCCCA
GCCTGCCAGCTGGTTCTGCACCAGATACTGAAGGGTGGCTCGGGGACATA
CTGCCTCAATGTGTCTCTGGCTGATACCAACAGCCTGGCAGTGGTCAGCA
CCCAGCTTATCATGCCTGGTCAAGAAGCAGGGGGCCTTGGGCAGGTTCCG
CTGATCGTGGGCATCTTGCTGGTGTTGATGGCTGTGGTCCTTGCATCTCT
GATATATAGGCGCAGACTTATGAAGCAAGACTTCTCCGTACCCCAGTTGC
CACATAGCAGCAGTCACTGGCTGCGTCTACCCCGCATCTTCTGCTCTTGT
CCCATTGGTGAGAATAGCCCCCTCCTCAGTGGGCAGCAGGTCTGAGTACT
CTCATATGATGCTGTGATTGCGGCCG.

TABLE 3B

silver/gp100/pMel17 (NM_006928.1 and .2)
nucleotide sequence (SEQ ID NO:7)

ATGGATCTGGTGCTAAAAAGATGCCTTCTTCATTTGGCTGTGATAGGTGC
TTTGCTGGCTGTGGGGGCTACAAAAGTACCCAGAAACCAGGACTGGCTTG
GTGTCTCAAGGCAACTCAGAACCAAAGCCTGGAACAGGCAGCTGTATCCA
GAGTGGACAGAAGCCCAGAGACTTGACTGCTGGAGAGGTGGTCAAGTGTC
CCTCAAGGTCAGTAATGATGGGCCTACACTGATTGGTGGTGCAAATGCCT
CCTTCTCTATTGCCTTGAACTTCCCTGGAAGCCAAAAGGTATTGCCAGAT
GGGCAGGTTATCTGGGTCAACAATACCATCATCAATGGTAGCCAGGTGTG
+00
GGGAGGACAGCCAGTGTATCCCCAGGAAACTGACGATGCCTGCATCTTCC
CTGATGGTGGACCTTGCCCATCTGGCTCTTGGTCTCAGAAGAGAAGCTTT
GTTTATGTCTGGAAGACCTGGGGTCAATACTGGCAAGTTCTAGGGGGCCC
AGTGTCTGGGCTGAGCATTGGGACAGGCAGGGCAATGCTGGGCACACACA
CCATGGAAGTGACTGTCTACCATCGCCGGGGATCCCGGAGCTATGTGCCT
CTTGCTCATTCCAGCTCAGCCTTCACCATTACTGACCAGGTGCCTTTCTC
CGTGAGCGTGTCCCAGTTGCGGGCCTTGGATGGAGGGAACAAGCACTTCC
TGAGAAATCAGCCTCTGACCTTTGCCCTCCAGCTCCATGACCCCAGTGGC
TATCTGGCTGAAGCTGACCTCTCCTACACCTGGGACTTTGGAGACAGTAG
TGGAACCCTGATCTCTCGGGCACTTGTGGTCACTCATACTTACCTGGAGC
CTGGCCCAGTCACTGCCCAGGTGGTCCTGCAGGCTGCCATTCCTCTCACC
TCCTGTGGCTCCTCCCCAGTTCCAGGCACCACAGATGGGCACAGGCCAAC
TGCAGAGGCCCCTAACACCACAGCTGGCCAAGTGCCTACTACAGAAGTTG
TGGGTACTACACCTGGTCAGGCGCCAACTGCAGAGCCCTCTGGAACCACA
TCTGTGCAGGTGCCAACCACTGAAGTCATAAGCACTGCACCTGTGCAGAT
GCCAACTGCAGAGAGCACAGGTATGACACCTGAGAAGGTGCCAGTTTCAG
AGGTCATGGGTACCACACTGGCAGAGATGTCAACTCCAGAGGCTACAGGT
ATGACACCTGCAGAGGTATCAATTGTGGTGCTTTCTGGAACCACAGCTGC
ACAGGTAACAACTACAGAGTGGGTGGAGACCACAGCTAGAGAGCTACCTA
TCCCTGAGCCTGAAGGTCCAGATGCCAGCTCAATCATGTCTACGGAAAGT
ATTACAGGTTCCCTGGGCCCCCTGCTGGATGGTACAGCCACCTTAGGCTG
GTGAAGAGACAAGTCCCCCTGGATTGTGTTCTGTATCGATATGGTTCCTT
TTCCGTCACCCTGGACATTGTCCAGGGTATTGAAAGTGCCGAGATCCTGC
AGGCTGTGCCGTCCGGTGAGGGGGATGCATTTGAGCTGACTGTGTCCTGC
CAAGGCGGGCTGCCCAAGGAAGCCTGCATGGAGATCTCATCGCCAGGGTG
CCAGCCCCCTGCCCAGCGGCTGTGCCAGCCTGTGCTACCCAGCCCAGCCT
GCCAGCTGGTTCTGCACCAGATACTGAAGGGTGGCTCGGGGACATACTGC
CTCAATGTGTCTCTGGCTGATACCAACAGCCTGGCAGTGGTCAGCACCCA
GCTTATCATGCCTGGTCAAGAAGCAGGCCTTGGGCAGGTTCCGCTGATCG
TGGGCATCTTGCTGGTGTTGATGGCTGTGGTCCTTGCATCTCTGATATAT
AGGCGCAGACTTATGAAGCAAGACTTCTCCGTACCCCAGTTGCCACATAG
CAGCAGTCACTGGCTGCGTCTACCCCGCATCTTCTGCTCTTGTCCCATTG
GTGAGAATAGCCCCCTCCTCAGTGGGCAGCAGGTCTGA.

TABLE 3C

silver/gp100/pMel17 (NM_006928.3) nucleotide
sequence (SEQ ID NO:8)

AGTGCCTTTGGTTGCTGGAGGGAAGAACACAATGGATCTGGTGCTAAAAA
GATGCCTTCTTCATTTGGCTGTGATAGGTGCTTTGCTGGCTGTGGGGGCT
ACAAAAGTACCCAGAAACCAGGACTGGCTTGGTGTCTCAAGGCAACTCAG
AACCAAAGCCTGGAACAGGCAGCTGTATCCAGAGTGGACAGAAGCCCAGA
GACTTGACTGCTGGAGAGGTGGTCAAGTGTCCCTCAAGGTCAGTAATGAT
GGGCCTACACTGATTGGTGCAAATGCCTCCTTCTCTATTGCCTTGAACTT
CCCTGGAAGCCAAAAGGTATTGCCAGATGGGCAGGTTATCTGGGTCAACA
ATACCATCATCAATGGGAGCCAGGTGTGGGGAGGACAGCCAGTGTATCCC
CAGGAAACTGACGATGCCTGCATCTTCCCTGATGGTGGACCTTGCCCATC
TGGCTCTTGGTCTCAGAAGAGAAGCTTTGTTTATGTCTGGAAGACCTGGG
GCCAATACTGGCAAGTTCTAGGGGGCCCAGTGTCTGGGCTGAGCATTGGG
ACAGGCAGGGCAATGCTGGGCACACACACCATGGAAGTGACTGTCTACCA
TCGCCGGGGATCCCGGAGCTATGTGCCTCTTGCTCATTCCAGCTCAGCCT
TCACCATTACTGACCAGGTGCCTTTCTCCGTGAGCGTGTCCCAGTTGCGG
GCCTTGGATGGAGGGAACAAGCACTTCCTGAGAAATCAGCCTCTGACCTT
TGCCCTCCAGCTCCATGACCCCAGTGGCTATCTGGCTGAAGCTGACCTCT
CCTACACCTGGGACTTTGGAGACAGTAGTGGAACCCTGATCTCTCGGGCA
CTTGTGGTCACTCATACTTACCTGGAGCCTGGCCCAGTCACTGCCCAGGT
GGTCCTGCAGGCTGCCATTCCTCTCACCTCCTGTGGCTCCTCCCCAGTTC
CAGGCACCACAGATGGGCACAGGCCAACTGCAGAGGCCCCTAACACCACA
GCTGGCCAAGTGCCTACTACAGAAGTTGTGGGTACTACACCTGGTCAGGC
GCCAACTGCAGAGCCCTCTGGAACCACATCTGTGCAGGTGCCAACCACTG
AAGTCATAAGCACTGCACCTGTGCAGATGCCAACTGCAGAGAGCACAGGT
ATGACACCTGAGAAGGTGCCAGTTTCAGAGGTCATGGGTACCACACTGGC
AGAGATGTCAACTCCAGAGGCTACAGGTATGACACCTGCAGAGGTATCAA
TTGTGGTGCTTTCTGGAACCACAGCTGCACAGGTAACAACTACAGAGTGG
GTGGAGACCACAGCTAGAGAGCTACCTATCCCTGAGCCTGAAGGTCCAGA
TGCCAGCTCAATCATGTCTACGGAAAGTATTACAGGTTCCCTGGGCCCCC
TGCTGGATGGTACAGCCACCTTAAGGCTGGTGAAGAGACAAGTCCCCCTG
GATTGTGTTCTGTATCGATATGGTTCCTTTTCCGTCACCCTGGACATTGT
CCAGGGTATTGAAAGTGCCGAGATCCTGCAGGCTGTGCCGTCCGGTGAGG
GGGATGCATTTGAGCTGACTGTGTCCTGCCAAGGCGGGCTGCCCAAGGAA
GCCTGCATGGAGATCTCATCGCCAGGGTGCCAGCCCCCTGCCCAGCGGCT
GTGCCAGCCTGTGCTACCCAGCCCAGCCTGCCAGCTGGTTCTGCACCAGA
TACTGAAGGGTGGCTCGGGGACATACTGCCTCAATGTGTCTCTGGCTGAT
ACCAACAGCCTGGCAGTGGTCAGCACCCAGCTTATCATGCCTGGTCAAGA
AGCAGGCCTTGGGCAGGTTCCGCTGATCGTGGGCATCTTGCTGGTGTTGA
TGGCTGTGGTCCTTGCATCTCTGATATATAGGCGCAGACTTATGAAGCAA
GACTTCTCCGTACCCCAGTTGCCACATAGCAGCAGTCACTGGCTGCGTCT
ACCCCGCATCTTCTGCTCTTGTCCCATTGGTGAGAATAGCCCCCTCCTCA
GTGGGCAGCAGGTCTGAGTACTCTCATATGATGCTGTGATTTTCCTGGAG
TTGACAGAAACACCTATATTTCCCCCAGTCTTCCCTGGGAGACTACTATT
AACTGAATAAATACTCAGAGCCTGAAAAAAAAAAAAAAAAAA.

TABLE 3D

Encoded silver/gp100/pMel17 protein sequence
(SEQ ID NO:9)

MDLVLKRCLLHLAVIGALLAVGATKVPRNQDWLGVSRQLRTKAWNRQLYP
EWTEAQRLDCWRGGQVSLKVSNDGPTLIGANASFSIALNFPGSQKVLPDG
QVIWVNNTIINGSQVWGGQPVYPQETDDACIFPDGGPCPSGSWSQKRSFV
YVWKTWGQYWQVLGGPVSGLSIGTGRAMLGTHTMEVTVYHRRGSRSYVPL
AHSSSAFTITDQVPFSVSVSQLRALDGGNKHFLRNQPLTFALQLHDPSGY
LAEADLSYTWDFGDSSGTLISRALVVTHTYLEPGPVTAQVVLQAAIPLTS
CGSSPVPGTTDGHRPTAEAPNTTAGQVPTTEVVGTTPGQAPTAEPSGTTS
VQVPTTEVISTAPVQMPTAESTGMTPEKVPVSEVMGTTLAEMSTPEATGM
TPAEVSIVVLSGTTAAQVTTTEWVETTARELPIPEPEGPDASSIMSTESI
TGSLGPLLDGTATLRLVKRQVPLDCVLYRYGSFSVTLDIVQGIESAEILQ
AVPSGEGDAFELTVSCQGGLPKEACMEISSPGCQPPAQRLCQPVLPSPAC
QLVLHQILKGGSGTYCLNVSLADTNSLAVVSTQLIMPGQEAGGLGQVPLI
VGILLVLMAVVLASLIYRRRLMKQDFSVPQLPHSSSHWLRLPRIFCSCPI
GENSPLLSGQQV.

TSC4: Gremlin 1 Homolog, Cysteine Knot Superfamily (Xenopus laevis).

AF154054.1, AFI 10137.2 and AF045800.1 encode the protein sequence shown in Table 4D.

TABLE 4A

gremlin 1 homolog, cysteine knot superfamily
(Xenopus laevis) (AF154054.1) nucleotide sequence
(SEQ ID NO:10)

ATAATAATTAGGCCAAGCGTTGAATAGTACGGGGGGGGGGGGGGGGCGAG
CCCCGGCGGCTCTGGCCGCGGCCGCACTCAGCGCCACGCGTCGAAAGCGC
AGGCCCCGAGGACCCGCCGCACTGACAGTATGAGCCGCACAGCCTACACG
GTGGGAGCCCTGCTTCTCCTCTTGGGGACCCTGCTGCCGGCTGCTGAAGG
GAAAAAGAAAGGGTCCCAAGGTGCCATCCCCCCGCCAGACAAGGCCCAGC
ACAATGACTCAGAGCAGACTCAGTCGCCCCAGCAGCCTGGCTCCAGGAAC
CGGGGGCGGGGCCAAGGGCGGGGCACTGCCATGCCCGGGGAGGAGGTGCT
GGAGTCCAGCCAAGAGGCCCTGCATGTGACGGAGCGCAAATACCTGAAGC
GAGACTGGTGCAAAACCCAGCCGCTTAAGCAGACCATCCACGAGGAAGGC
TGCAACAGTCGCACCATCATCAACCGCTTCTGTTACGGCCAGTGCAACTC
TTTCTACATCCCCAGGCACATCCGGAAGGAGGAAGGTTCCTTTCAGTCCT
GCTCCTTCTGCAAGCCCAAGAAATTCACTACCATGATGGTCACACTCAAC
TGCCCTGAACTACAGCCACCTACCAAGAAGAAGAGAGTCACACGTGTGAA
GCAGTGTCGTTGCATATCCATCGATTTGGATTAAGCCAAATCCAGGTGCA
CCCAGCATGTCCTAGGAATGCAGACCCAGGAAGTCCCAGACCTAAAACAA
CCAGATTCTTACTTGGCTTAAACCTAGAGGCCAGAAGAACCCCCAGCTGC
CTCCTGGCAGGAGCCTGCTTGTGCGTAGTTCGTGTGCATGAGTGTGGATG
GGTGCCTGTGGGTGTTTTTAGACACCAGAGAAAACACAGTCTCTGCTAGA
GAGCACTTCCTATTTTGTAAACCTATCTGCTTTAATGGGGATGTACCAGA
AACCCACCTCACCCCGGCTCACATCTAAAGGGGCGGGGCCGTGGTCTGGT
TCTGACTTTGTGTTTTTGTGCCCTCCTGGGGACCAGAATCTCCTTTCGGA
ATGAATGTTCATGGAAGAGGCTCCTCTGAGGGCAAGAGACCTGTTTTAGT
GCTGCATTCGACATGGAAAAGTCCTTTTAACCTGTGCTTGCATCCTCCTT
TCCTCCTCCTCCTCACAATCCATCTCTTCTTAAGTTGACAGTGACTATGT
CAGTCTAATCTCTTGTTTGCCAGGGTTCCTAAATTAATTCACTTAACCAT
GATGCAAATGTTTTTCATTTGGTGAAGACCTCCAGACTCTGGGAGAGGCT
GGTGTGGGCAAGGACAAGCAGGATAGTGGAGTGAGAAAGGGAGGGTGGAG
GGTGAGGCCAAATCAGGTCCAGCAAAAGTCAGTAGGGACATTGCAGAAGC
TTGAAAGGCCAATACCAGAACACAGGCTGATGCTTCTGAGAAAGTCTTTT
CCTAGTATTTAACAAAACCCAAGTGAACAGAGGAGAAATGAGATTGCCAG
AAAGTGATTAACTTTGGCCGTTGCAATCTGCTCAAACCTAACACCAAACT
GAAAACATAAATACTGACCACTCCTATGTTCGGACCCAAGCAAGTTAGCT
AAACCAAACCAACTCCTCTGCTTTGTCCCTCAGGTGGAAAAGAGAGGTAG
TTTAGAACTCTCTGCATAGGGGTGGGAATTAATCAAAAACCTCAGAGGCT
GAAATTCCTAATACCTTTCCTTTATCGTGGTTATAGTCAGCTCATTTCCA
TTCCACTATTTCCCATAATGCTTCTGAGAGCCACTAACTTGATTGATAAA
GATCCTGCCTCTGCTGAGTGTACCTGACAGTAGTCTAAGATGAGAGAGTT
TAGGGACTACTCTGTTTTAACAAGAAATATTTTGGGGGTCTTTTTGTTTT
AACTATTGTCAGGAGATTGGGCTAAAGAGAAGACGACGAGAGTAAGGAAA
TAAAGGGAATTGCCTCTGGCTAGAGAGTAGTTAGGTGTTAATACCTGGTA
GAGATGTAAGGGATATGACCTCCCTTTCTTTATGTGCTCACTTGAGGATC
TGAGGGGACCCTGTTAGGAGAGCATAGCATCATGATGTATTAGCTGTTCA
TCTGCTACTGGTTGGATGGACATAACTATTGTAACTATTCAGTATTTACT
GGTAGGCACTGTCCTCTGATTAAACTTGGCCTACTGGCAATGGCTACTTA
GGATTGATCTAAGGGCCAAAGTGCAGGGTGGGTGAACTTTATTGTACTTT
GGATTTGGTTAACCTGTTTTCCTCAAGCCTGAGGTTTTATATACAAACTC
CCTGAATACTCTTTTTGCCTTGTTACTTCTCAGCCTCCTAGCCAAGTCCT
ATGTAATATGGAAAACAAACACTGCAGACTTGAGATTCAGTTGCCGATCA
AGGCTCTGGCATTCAGAGAACCCTTGCAACTCGAGAAGCTGTTTTTGATT
TCGTTTTTGTTTTGAACCGGTGCTCTCCCATCTAACAACTAACAAGGACC
ATTTCCAGGCGGGAGATATTTTAAACACCCAAAATGTTGGGTCTGATTTC
CAAACTTTTAAACTCACTACTGATGATTCTCACGCTAGGCGAATTTGTCC
AAACACATAGTGTGTGTGTTTTGTATACACTGTATGACCCCACCCCAAAT
CTTTGTATTGTCCACATTCTCCAACAATAAAGCACAGAGTGGATTTAATT
AAGCACACAAATGCTAAGGCAGAATTTTGAGGGTGGGAGAGAAGAAAAGG
GAAAGAAGCTGAAAATGTAAAACCACACCAGGGAGGAAAAATGACATTCA
GAACCACCAAACACTGAATTTCTCTTGTTGTTTTAACTCTCCCACAAGAA
TGCAATTTCGTTAATGGAGATGACTTAAGTTGGCAGCAGTAATCTTCTTT
TAGGAGCTTGTACCACAGTCTTGCACATAAGTGCAGATTTGCCCCAAGTA
AAGAGAATTTCCTCAACACTAACTTCACGGGGATAATCACCACGTAACTA
CCCTTAAAGCATATCACTAGCCAAAGAGGGGAATATCTGTTCTTCTTACT
GTGCCTATATTAAGACTAGTACAAATGTGGTGTGTCTTCCAACTTTCATT
GAAAATGCCATATCTATACCATATTTTATTCGAGTCACTGATGATGTAAT
GATATATTTTTTCATTATTATAGTAGAATATTTTTATGGCAAGAGATTTG
TGGTCTTGATCATACCTATTAAAATAATGCCAAACACCAAATATGAATTT
TATGATGTACACTTTGTGCTTGGCATTAAAAGAAAAAAACACACACGCC.

TABLE 4B

gremlin 1 homolog, cysteine knot superfamily
(Xenopus laevis) (AF110137.2) nucleotide sequence
(SEQ ID NO:11)

GCGGCCGCACTCAGCGCCACGCGTCGAAAGCGCAGGCCCCGAGGACCCGC
CGCACTGACAGTATGAGCCGCACAGCCTACACGGTGGGAGCCCTGCTTCT
CCTCTTGGGGACCCTGCTGCCGGCTGCTGAAGGGAAAAAGAAAGGGTCCC
AAGGTGCCATCCCCCCGCCAGACAAGGCCCAGCACAATGACTCAGAGCAG
ACTCAGTCGCCCCAGCAGCCTGGCTCCAGGAACCGGGGGCGGGGCCAAGG
GCGGGGCACTGCCATGCCCGGGGAGGAGGTGCTGGAGTCCAGCCAAGAGG
CCCTGCATGTGACGGAGCGCAAATACCTGAAGCGAGACTGGTGCAAAACC
CAGCCGCTTAAGCAGACCATCCACGAGGAAGGCTGCAACAGTCGCACCAT
CATCAACCGCTTCTGTTACGGCCAGTGCAACTCTTTCTACATCCCCAGGC
ACATCCGGAAGGAGGAAGGTTCCTTTCAGTCCTGCTCCTTCTGCAAGCCC
AAGAAATTCACTACCATGATGGTCACACTCAACTGCCCTGAACTACAGCC
ACCTACCAAGAAGAAGAGAGTCACACGTGTGAAGCAGTGTCGTTGCATAT
CCATCGATTTGGATTAAGCCAAATCCAGGTGCACCCAGCATGTCCTAGGA
ATGCAGCCCCAGGAAGTCCCAGACCTAAAACAACCAGATTCTTACTTGGC
TTAAACCTAGAGGCCAGAAGAACCCCCAGCTGCCTCCTGGCAGGAGCCTG
CTTGTGCGTAGTTCGTGTGCATGAGTGTGGATGGGTGCCTGTGGGTGTTT
TTAGACACCAGAGAAAACACAGTCTCTGCTAGAGAGCACTCCCTATTTTG
TAAACATATCTGCTTTAATGGGGATGTACCAGAAACCCACCTCACCCCGG
CTCACATCTAAAGGGGCGGGGCCGTGGTCTGGTTCTGACTTTGTGTTTTT
GTGCCCTCCTGGGGACCAGAATCTCCTTTCGGAATGAATGTTCATGGAAG
AGGCTCCTCTGAGGGCAAGAGACCTGTTTTAGTGCTGCATTCGACATGGA
AAAGTCCTTTTAACCTGTGCTTGCATCCTCCTTTCCTCCTCCTCCTCACA
ATCCATCTCTTCTTAAGTTGATAGTGACTATGTCAGTCTAATCTCTTGTT
TGCCAAGGTTCCTAAATTAATTCACTTAACCATGATGCAAATGTTTTTCA
TTTTGTGAAGACCCTCCAGACTCTGGGAGAGGCTGGTGTGGGCAAGGACA
AGCAGGATAGTGGAGTGAGAAAGGGAGGGTGGAGGGTGAGGCCAAATCAG
GTCCAGCAAAAGTCAGTAGGGACATTGCAGAAGCTTGAAAGGCCAATACC
AGAACACAGGCTGATGCTTCTGAGAAAGTCTTTTCCTAGTATTTAACAGA
ACCCAAGTGAACAGAGGAGAAATGAGATTGCCAGAGTGATTAACTTTGGC
CGTTGCAATCTGCTCAAACCTAACACCAAACTGAAAACATAAATACTGAC
CACTCCTATGTTCGGACCCAAGCAAGTTAGCTAAACCAAACCAACTCCTC
TGCTTTGTCCCTCAGGTGGAAAAGAGAGGTAGTTTAGAACTCTCTGCATA
GGGGTGGGAATTAATCAAAAACCKCAGAGGCTGAAATTCCTAATACCTTT
CCTTTATCGTGGTTATAGTCAGCTCATTTCCATTCCACTATTTCCCATAA
TGCTTCTGAGAGCCACTAACTTGATTGATAAAGATCCTGCCTCTGCTGAG
TGTACCTGACAGTAAGTCTAAAGATGARAGAGTTTAGGGACTACTCTGTT
TTAGCAAGARATATTKTGGGGGTCTTTTTGTTTTAACTATTGTCAGGAGA
TTGGGCTARAGAGAAGACGACGAGAGTAAGGAAATAAAGGGRATTGCCTC
TGGCTAGAGAGTAAGTTAGGTGTTAATACCTGGTAGAAATGTAAGGGATA
TGACCTCCCTTTCTTTATGTGCTCACTGAGGATCTGAGGGGACCCTGTTA
GGAGAGCATAGCATCATGATGTATTAGCTGTTCATCTGCTACTGGTTGGA
TGGACATAACTATTGTAACTATTCAGTATTTACTGGTAGGCACTGTCCTC
TGATTAAACTTGGCCTACTGGCAATGGCTACTTAGGATTGATCTAAGGGC
CAAAGTGCAGGGTGGGTGAACTTTATTGTACTTTGGATTTGGTTAACCTG
TTTTCTTCAAGCCTGAGGTTTTATATACAAACTCCCTGAATACTCTTTTT
GCCTTGTATCTTCTCAGCCTCCTAGCCAAGTCCTATGTAATATGGAAAAC
AAACACTGCAGACTTGAGATTCAGTTGCCGATCAAGGCTCTGGCATTCAG
AGAACCCTTGCAACTCGAGAAGCTGTTTTTATTTCGTTTTTGTTTTGATC
CAGTGCTCTCCCATCTAACAACTAAACAGGAGCCATTTCAAGGCGGGAGA
TATTTTAAACACCCAAAATGTTGGGTCTGATTTTCAAACTTTTAAACTCA
CTACTGATGATTCTCACGCTAGGCGAATTTGTCCAAACACATAGTGTGTG
TGTTTTGTATACACTGTATGACCCCACCCCAAATCTTTGTATTGTCCACA
TTCTCCAACAATAAAGCACAGAGTGGATTTAATTAAGCACACAAATGCTA
AGGCAGAATTTTGAGGGTGGGAGAGAAGAAAAGGGAAAGAAGCTGAAAAT
GTAAAACCACACCAGGGAGGAAAAATGACATTCAGAACCAGCAAACACTG
AATTTCTCTTGTTGTTTTAACTCTGCCACAAGAATGCAATTTCGTTAATG
GAGATGACTTAAGTTGGCAGCAGTAATCTTCTTTTAGGAGCTTGTACCAC
AGTCTTGCACATAAGTGCAGATTTGGCTCAAGTAAAGAGAATTTCCTCAA
CACTAACTTCACTGGGATAATCAGCAGCGTAACTACCCTAAAAGCATATC
ACTAGCCAAAGAGGGAAATATCTGTTCTTCTTACTGTGCCTATATTAAGA
CTAGTACAAATGTGGTGTGTCTTCCAACTTTCATTGAAAATGCCATATCT
ATACCATATTTTATTCGAGTCACTGATGATGTAATGATATATTTTTTCAT
TATTATAGTAGAATATTTTTATGGCAAGATATTTGTGGTCTTGATCATAC
CTATTAAAAATAATGCCAAACACCAAATATGAATTTTATGATGTACACTT
TGTGCTTGGCATTAAAAGAAAAAAACACACATCCTGGAAGTCTGTAAGTT
GTTTTTTGTTACTGTAGGTCTTCAAAGTTAAGAGTGTAAGTGAAAAATCT
GGAGGAGAGGATAATTTCCACTGTGTGGAATGTGAATAGTTAAATGAAAA
GTTATGGTTATTTAATGTAATTATTACTTCAAATCCTTTGGTCACTGTGA
TTTCAAGCATGTTTTCTTTTTCTCCTTTATATGACTTTCTCTGAGTTGGG
CAAAGAAGAAGCTGACACACCGTATGTTGTTAGAGTCTTTTATCTGGTCA
GGGGAAACAAAATCTTGACCCAGCTGAACATGTCTTCCTGAGTCAGTGCC
TGAATCTTTATTTTTTAAATTGAATGTTCCTTAAAGGTTAACATTTCTAA
AGCAATATTAAGAAAGACTTTAAATGTTATTTTGGAAGACTTACGATGCA
TGTATACAAACGAATAGCAGATAATGATGACTAGTTCACACATAAAGTCC
TTTTAAGGAGAAAATCTAAAATGAAAAGTGGATAAACAGAACATTTATAA
GTGATCAGTTAATGCCTAAGAGTGAAAGTAGTTCTATTGACATTCCTCAA
GATATTTAATATCAACTGCATTATGTATTATGTCTGCTTAAATCATTTAA
AAACGGCAAAGAATTATATAGACTATGAGGTACCTTGCTGTGTAGGAGGA
TGAAAGGGGAGTTGATAGTCTCATAAAACTAATTTGGCTTCAAGTTTCAT
GAATCTGTAACTAGAATTTAATTTTCACCCCAATAATGTTCTATATAGCC
TTTGCTAAAGAGCAACTAATAAATTAAACCTATTCTTTCAAAAAAAAA.

TABLE 4C

gremlin 1 homolog, cysteine knot superfamily
(Xenopus laevis) (AF045800.1) nucleotide
sequence (SEQ ID NO:12)

ATGAGCCGCACAGCCTACACGGTGGGAGCCCTGCTTCTCCTCTTGGGGAC
CCTGCTGCCGGCTGCTGAAGGGAAAAAGAAAGGGTCCCAAGGTGCCATCC
CCCCGCCAGACAAGGCCCAGCACAATGACTCAGAGCAGACTCAGTCGCCC
CAGCAGCCTGGCTCCAGGAACCGGGGGCGGGGCCAAGGGCGGGGCACTGC
CATGCCCGGGGAGGAGGTGCTGGAGTCCAGCCAAGAGGCCCTGCATGTGA
CGGAGCGCAAATACCTGAAGCGAGACTGGTGCAAAACCCAGCCGCTTAAG
CAGACCATCCACGAGGAAGGCTGCAACAGTCGCACCATCATCAACCGCTT
CTGTTACGGCCAGTGCAACTCTTTCTACATCCCCAGGCACATCCGGAAGG
AGGAAGGTTCCTTTCAGTCCTGCTCCTTCTGCAAGCCCAAGAAATTCACT
ACCATGATGGTCACACTCAACTGCCCTGAACTACAGCCACCTACCAAGAA
GAAGAGAGTCACACGTGTGAAGCAGTGTCGTTGCATATCCATCGATTTGG
ATTAA.

TABLE 4D

Encoded gremlin 1 homolog, cysteine knot
superfamily (Xenopus laevis) protein
sequence (SEQ ID NO:13)

MSRTAYTVGALLLLLGTLLPAAEGKKKGSQGAIPPPDKAQHNDSEQTQSP
QQPGSRNRGRGQGRGTAMPGEEVLESSQEALHVTERKYLKRDWCKTQPLK
QTIHEEGCNSRTIINRFCYGQCNSFYIPRHIRKEEGSFQSCSFCKPKKFT
TMMVTLNCPELQPPTKKKRVTRVKQCRCISIDLD.

TSC5: ATP-Binding Cassette, Sub-Family B (MDR/TAP), Member 5.

AL040763 does not possess a reading frame beyond 50 amino acids.

TABLE 5A

ATP-binding cassette, sub-family B (MDR/TAP),
member 5 (AL040763) nucleotide sequence
(SEQ ID NO:14)

TCCCCCATAATTATGCCACATAGCTGTTATTATTTTCATATATTGCCTTC
ATTTTTTTCACAGTTGCTATTTTGTGTAATTTTGGAATCAGTTTACAAAC
ATTCTGCATTCTTCTTTTTTCACTTTTGATAGATGTTTCATATTAACCAA
TAAGAATAACATTTATTAGTTTATCATGTCACCAAGCAACTATTTATTTT
AAAAGTCTGAACTAGTTTTGATTACCTAAAGTGATTACCAGTGGATGAAA
ATACTGCGGGCACAACATGAAACCTTCTAAACAATCAGAGAGCCTATTAC
AATACATTTTTTAAAATCTTATGTAACTGGCCGGGCGCGGAGACGCACAC
CTGTAATCCCAGCACTTTGGGAGGCCGAGGTGGGCGGATCACCTGAGGTC
AGGAGTTTGAGACCAGCCTGGTCAACATGGCAAAACCCCGTCTCTACTAA
AAATACAAAAATTAGCCGGGTGTGGTGATGCACGCCTGTACTCCCAGCTA
CTCAGGAGGCTGAGGCAGGAGAATCGCCTGAACACAGTGGGCAGAGGTTG
CACTCAAGCATGGGTGACAGAGCGAGGCTTGAATATAGTCTAAATACAGA
TCCCTGTCCTAGTTACTAAGTATAAAAAATGAATAAAATATTAGTCCTGT
CTTTGTATTTTCTGTACCAAGATGAACCAAATTGCCGAAGTGTCCACAGT
AAACAAAGATTATTTATCACACAAGC.

TSC6: 5-hydroxytryptamine (Serotonin) Receptor 2B.

TABLE 6A

5-hydroxytryptamine (serotonin) receptor 2B
(NM_000867.2) nucleotide sequence (SEQ ID NO:15)

GGGGGTATTTGTTTCACTGCTTTCAACCGCCTGTGCTGGAGGCTCAGAAT
AAGTCAATGGGAGGAGGATTTCAGTCACAGCAGCAAGCAAGTCTAGTGAA
CAGATAAGATGACATGCTCAGCAAAATAACAACGAAACCAGAGGGGGAAC
TCTCTGGCATGCAAGTTCAAACACGACTCTACAACTACGGCAGAAAAAGA
GAGAGAGAGAAACTAAAAATATATATATATCCTATTTTTTTCACAGCTAT
CAGTTTCTTTCACTGAGCTTTCCTAAATTTAAGCCTCTAGAAAATAATAA
ATACTTGGATATCTTACCTACAAACATGGACAGATGTGTGTATGCGCTCA
TTTTAGAGAACTTGAATTTTTTTTTTTAAAGGAAGGTGTCAACTTTGGCT
TTTGAGTGTTTGGCATGGTTACAATGCCTTAAAAAAACAGATGAGCAGCT
TAGCTACTAACCATGCTGACCACTGTTCGGAACGGGATTGAATCACAGAA
AAACAGCAAATGGCTCTCTCTTACAGAGTGTCTGAACTTCAAAGCACAAT
TCCTGAGCACATTTTGCAGAGCACCTTTGTTCACGTTATCTCTTCTAACT
GGTCTGGATTACAGACAGAATCAATACCAGAGGAAATGAAACAGATTGTT
GAGGAACAGGGAAATAAACTGCACTGGGCAGCTCTTCTGATACTCATGGT
GATAATACCCACAATTGGTGGAAATACCCTTGTTATTCTGGCTGTTTCAC
TGGAGAAGAAGCTGCAGTATGCTACTAATTACTTTCTAATGTCCTTGGCG
GTGGCTGATTTGCTGGTTGGATTGTTTGTGATGCCAATTGCCCTCTTGAC
AATAATGTTTGAGGCTATGTGGCCCCTCCCACTTGTTCTATGTCCTGCCT
GGTTATTTCTTGACGTTCTCTTTTCAACCGCATCCATCATGCATCTCTGT
GCCATTTCAGTGGATCGTTACATAGCCATCAAAAAGCCAATCCAGGCCAA
TCAATATAACTCACGGGCTACAGCATTCATCAAGATTACAGTGGTGTGGT
TAATTTCAATAGGCATTGCCATTCCAGTCCCTATTAAAGGGATAGAGACT
GATGTGGACAACCCAAACAATATCACTTGTGTGCTGACAAAGGAACGTTT
TGGCGATTTCATGCTCTTTGGCTCACTGGCTGCCTTCTTCACACCTCTTG
CAATTATGATTGTCACCTACTTTCTCACTATCCATGCTTTACAGAAGAAG
GCTTACTTAGTCAAAAACAAGCCACCTCAACGCCTAACATGGTTGACTGT
GTCTACAGTTTTCCAAAGGGATGAAACACCTTGCTCGTCACCGGAAAAGG
TGGCAATGCTGGATGGTTCTCGAAAGGACAAGGCTCTGCCCAACTCAGGT
GATGAAACACTTATGCGAAGAACATCCACAATTGGGAAAAAGTCAGTGCA
GACCATTTCCAACGAACAGAGAGCCTCAAAGGTCCTAGGGATTGTGTTTT
TCCTCTTTTTGCTTATGTGGTGTCCCTTCTTTATTACAAATATAACTTTA
GTTTTATGTGATTCCTGTAACCAAACTACTCTCCAAATGCTCCTGGAGAT
ATTTGTGTGGATAGGCTATGTTTCCTCAGGAGTGAATCCTTTGGTCTACA
CCCTCTTCAATAAGACATTTCGGGATGCATTTGGCCGATATATCACCTGC
AATTACCGGGCCACAAAGTCAGTAAAAACTCTCAGAAAACGCTCCAGTAA
GATCTACTTCCGGAATCCAATGGCAGAGAACTCTAAGTTTTTCAAGAAAC
ATGGAATTCGAAATGGGATTAACCCTGCCATGTACCAGAGTCCAATGAGG
CTCCGAAGTTCAACCATTCAGTCTTCATCAATCATTCTACTAGATACGCT
TCTCCTCACTGAAAATGAAGGTGACAAAACTGAAGAGCGAGTTAGTTATG
TATAGCAGAACTGGCAGTTGTCATCAAACATAATGATGAGTAAGATGATG
AATGAGATGTAAATGTGCCAAGAATATATTATATAAAGAATTTTATGTCA
TATATCAAATCATCTCTTTAACCTAAGATGTAAGTATTAAGAATATCTAA
TTTTCCTAATTTGGACAAGATTATTCCATGAGGAAAATAATTTTATATAG
CTACAAATGAAAACAATCCAGCACTCTGGTTAAATTTTAAGGTATTCGAA
TGAAATAAAGTCAAATCAATAAATTTCAGGCCAAAAAAAAAAAAAAAAAA
AAAAAAAAAA.

TABLE 6B

Encoded 5-hydroxytryptamine (serotonin) receptor
2B protein sequence (SEQ ID NO:16)

MALSYRVSELQSTIPEHILQSTFVHVISSNWSGLQTESIPEEMKQIVEEQ
GNKLHWAALLILMVIIPTIGGNTLVILAVSLEKKLQYATNYFLMSLAVAD
LLVGLFVMPIALLTIMFEAMWPLPLVLCPAWLFLDVLFSTASIMHLCAIS
VDRYIAIKKPIQANQYNSRATAFIKITVVWLISIGIAIPVPIKGIETDVD
NPNNITCVLTKERFGDFMLFGSLAAFFTPLAIMIVTYFLTIHALQKKAYL
VKNKPPQRLTWLTVSTVFQRDETPCSSPEKVAMLDGSRKDKALPNSGDET
LMRRTSTIGKKSVQTISNEQRASKVLGIVFFLFLLMWCPFFITNITLVLC
DSCNQTTLQMLLEIFVWIGYVSSGVNPLVYTLFNKTFRDAFGRYITCNYR
ATKSVKTLRKRSSKIYFRNPMAENSKFFKKHGIRNGINPAMYQSPMRLRS
STIQSSSIILLDTLLLTENEGDKTEERVSYV.

TSC7: Mucolipin 3.

TABLE 7A

Mucolipin 3 (NM 018298.9) nucleotide sequence
(SEQ ID NO:17)
CGGGGCTCGAGGCTGCTGGAGTCGCTCGCTGACTCGCCCTGCGCCCTCGC
CGCGGACACCGGAGCTGCGGCCGCTCCCCGCTGTCCCCCAGAGATGGCAG
ATCCTGAGGTAGTTGTGAGTAGCTGCAGCTCTCATGAAGAGGAAAATCGC
TGCAATTTTAACCAGCAAACATCTCCATCTGAGGAGCTTCTATTAGAAGA
CCAGATGAGGCGAAAACTCAAATTTTTTTTCATGAATCCCTGTGAGAAGT
TCTGGGCTCGAGGTAGAAAACCATGGAAACTTGCCATACAAATTCTAAAA
ATTGCAATGGTGACTATCCAGCTGGTCTTATTTGGGCTAAGTAACCAGAT
GGTGGTAGCTTTCAAGGAAGAGAATACTATAGCATTCAAACACCTTTTCC
TAAAAGGATATATGGACCGAATGGATGACACATATGCAGTGTACACACAA
AGTGACGTGTATGATCAGTTAATCTTCGCAGTAAACCAGTACTTGCAGCT
ATACAATGTCTCCGTTGGGAATCATGCTTATGAGAACAAAGGTACCAAGC
AATCTGCTATGGCAATCTGTCAGCACTTCTACAAGCGAGGAAACATCTAC
CCTGGAAATGATACCTTTGACATCGATCCAGAAATTGAAACTGAGTGTTT
CTTTGTGGAGCCAGATGAACCTTTTCACATTGGGACACCAGCAGAAAATA
AACTGAACTTAACACTGGACTTCCACAGACTCCTAACAGTGGAGCTTCAG
TTTAAACTGAAAGCCATTAATCTGCAGACAGTTCGTCATCAAGAACTCCC
TGACTGTTATGACTTTACTCTGACTATAACATTTGACAACAAGGCCCATA
GTGGAAGAATTAAAATAAGTTTAGATAATGACATTTCCATCAGAGAATGT
AAAGACTGGCATGTATCTGGATCAATTCAGAAGAACACTCATTACATGAT
GATCTTTGATGCCTTTGTCATTCTGACTTGCTTGGTTTCATTAATCCTCT
GCATTAGATCTGTGATTAGAGGACTTCAGCTTCAGCAGGAGTTTGTCAAT
TTTTTCCTCCTCCATTATAAGAAGGAAGTTTCTGTTTCTGATCAAATGGA
ATTTGTCAATGGATGGTACATTATGATTATTATTAGTGACATATTGACAA
TCATTGGATCAATTCTAAAAATGGAAATCCAAGCTAAGAGTCTAACTAGT
TATGATGTCTGTAGCATACTTCTTGGGACTTCTACCATGCTCGTGTGGCT
TGGAGTCATCCGATACCTCGGTTTCTTTGCAAAGTACAACCTCCTCATTT
TGACCCTTCAGGCAGCGCTGCCCAATGTCATCAGGTTCTGCTGCTGTGCA
GCTATGATTTACTTAGGTTACTGCTTCTGTGGATGGATCGTGCTGGGGCC
TTACCATGACAAGTTTCGTTCTCTGAACATGGTCTCTGAGTGCCTTTTCT
CTCTGATAAATGGAGATGATATGTTTGCCACGTTTGCAAAAATGCAGCAA
AAAAGTTACTTAGTCTGGCTGTTTAGTAGAATTTACCTCTACTCATTCAT
CAGCCTCTTTATATATATGATTTTAAGTCTTTTCATTGCACTGATCACTG
ATACATACGAAACAATTAAGCAATACCAACAAGATGGCTTCCCAGAGACT
GAACTTCGTACATTTATATCAGAATGCAAAGATCTACCCAACTCTGGAAA
ATACAGATTAGAAGATGACCCTCCAGTATCTTTATTCTGCTGTTGTAAAA
AGTAGCTATCAGGTTTATCTGTACTTTAGAGGAAAATATAATGTGTAGCT
GAGTTGGAACACTGTGGATATTCTGAGATCAGATGTAGTATGTTTGAAGA
CTGTTATTTTGAGCTAATTGAGACCTATAATTCACCAATAACTGTTTATA
TTTTTAAAAGCAATATTTAATGTCTTTGCAACTTTATGCTGGGATTGTTT
TTAAAAAAACTTTAATGAGGAAAGCTATTGGATTATTATTATTTCTTGTT
TATTTTGCCATGGCTTTAGAATGTATTCTGTATGCCTCTCTTTTGCTCTG
ATACTGTTGCTCCTGCTATTCTGATTGTGCAGACTGTGTAATTAGTGGAA
AACAATCCTTGGTCTGACTGTGACTTTGGACAACTCAGTAACCCTGGCTT
GGACCACTCTCAGGAGTCCATCCTTGAGAGAGTGGGTGTAGTTATCATTT
ATACAGTAATCATTGCATTTTAAAATCTTCTCTTGAAAGGAAGAATAAGA
GTGCACCAGAATAAGAGCGCACCAGAATAAGAGCGCACCAGCTAACAATG
TGATACGGCCATATGTCACTTAAGGATGGAGATATGTTCTGAGAAATGTG
TCATTAGGCGATTTTGTCATTAAACATCATAGCATGTACTTCCACAAACC
TAGATGGTATAGCCTACTACACACCTAGGCTATTTGGTATAGCCTGTTGG
TCCTGGGGTACAAATCTGTACAACATGTTACTGTATTGAATACAGTAGGC
AATTGTAACACAATGGTAAGTATCTAAACATAGAAAAGGGACAGTAAAAA
TATGGTTTTATAATCTTCTGGGACCACCATTGTATATGCGGTACATCATT
GACCAAAACATCGTTATCCAGCATATGACTGTATTTGGTTATGAAAGCCA
ACTGTTACTTGATTCTGCTTTTAGTTCTTAAGAGGATCAGGCTTTTAAAT
ACTCATTTACAAGTTTTCTATCCTCCTTCAGTGTTAAAGTAGAAAGTAAA
AAGAGTATCTTATACATGCATGAAATTAAAGCATATACCAAATGCAAAAA
AAAAAAAAAAAAA.

TABLE 7B

Encoded mucolipin 3 protein sequence
(SEQ ID NO:18)

MADPEVVVSSCSSHEEENRCNFNQQTSPSEELLLEDQMRRKLKFFFMNPC
EKFWARGRKPWKLAIQILKIAMVTIQLVLFGLSNQMVVAFKEENTIAFKH
LFLKGYMDRMDDTYAVYTQSDVYDQLIFAVNQYLQLYNVSVGNHAYENKG
TKQSAMAICQHFYKRGNIYPGNDTFDIDPEIETECFFVEPDEPFHIGTPA
ENKLNLTLDFHRLLTVELQFKLKAINLQTVRHQELPDCYDFTLTITFDNK
AHSGRIKISLDNDISIRECKDWHVSGSIQKNTHYMMIFDAFVILTCLVSL
ILCIRSVIRGLQLQQEFVNFFLLHYKKEVSVSDQMEFVNGWYIMIIISDI
LTIIGSILKMEIQAKSLTSYDVCSILLGTSTMLVWLGVIRYLGFFAKYNL
LILTLQAALPNVIRFCCCAAMIYLGYCFCGWIVLGPYHDKFRSLNMVSEC
LFSLINGDDMFATFAKMQQKSYLVWLFSRIYLYSFISLFIYMILSLFIAL
ITDTYETIKQYQQDGFPETELRTFISECKDLPNSGKYRLEDDPPVSLFCC
CKK.

TSC 8: A Disintegrin and Metalloproteinase Domain 12 (Meltrin Alpha).

W46291 does not possess a reading frame beyond 50 amino acids.

TABLE 8A

A disintegrin and metalloproteinase domain 12
(meltrin alpha) (W46291) nucleotide sequence
(SEQ ID NO:19)

TTTTTTGAGGATGCATTGATGTATTGATTTGCCTGGGAACAATGGCCTAT
AGTTCAGCCTGAGAATTCTCATAAAGTTAAGAAGGCATAAAAATGCCCCC
CCCGAGACTCGTCAGGAGTATTGACTCTCCTACAGTTTAATTTGCTGCTT
TTCGTCGGTTTCTGTGATGTCATCCCACATGTGTAAGCTGGAAAAATCCA
CGCTGTGAAGTGTAACCTCCTGTGTGTATTTCCACAATGGAGAATGTTAG
GCTTCGTTTCCCTCGGTTGCTACACATCTGATTACATGTGTCAGGAAAAC
AAACTTAAAAAATTTCAGGAGACAAACCTTTCAGCGGAATTGCCTGGAAC
CCATGAAGTGAGGTCATAGAACCTACAACTATAATAAGCTGTAGGAAGAA
AAGTAGCCTCTGGGCTACTTTGTGTCTAGTCACATTGACTTTCCAGGTGA
TGGCCCTACAAAACTCAAACCACCTCTATTATTCATGCCTAAAT.

TSC 9: Myosin VIIA and Rab Interacting Protein.

TABLE 9A

Myosin VIIA and Rab interacting protein
(AL50090.1) nucleotide sequence (SEQ ID NO:20)

GAAAATGTATACCTGGCAGCAGGCACTGTGTATGGACTGGAGACCCAGCT
GACTGAGCTAGAAGATGCCGCCCGCTGCATCCACAGCGGCACTGATGAGA
CCCATCTGGCGGATCTGGAGGACCAGGTGGCCACGGCTGCAGCCCAAGTC
CACCATGCTGAACTCCAGATTTCAGATATTGAGAGCCGGATTTCAGCCCT
GACCATTGCAGGATTAAACATAGCACCATGTGTGCGCTTCACAAGAAGAC
GGGATCAGAAGCAAAGGACCCAGGTACAAACCATAGATACATCAAGGCAG
CAAAGGAGGAAACTGCCTGCTCCACCGGTGAAAGCTGAAAAAATTGAGAC
ATCTTCAGTGACTACCATTAAAACATTTAACCACAACTTCATTCTCCAAG
GCTCCTCAACAAACAGGACTAAGGAAAGGAAAGGCACCACCAAGGATTTG
ATGGAGCCTGCTCTGGAGTCAGCTGTGATGTACTGACACCATGGAATTCC
ACTGCCAGTGACCCACTGCCTCCGGCCGTACACGACAGTGCCTTGACCCA
ACAGCCATCGAGTACTGTATGTATTTCCACCTGAGGAGAAGGCCTGGGGA
GGCCACAGTGCACCATTGCACAGGGCTGTCCTGATACCTCATCCAGAAAG
CCGTCTCAGACTTCAGCACTGCGGTCTTGCCCACTCTCTGCCTTAGGCTC
CCAGGGGAATCCAAGACAGAAAATGAAGACACTGGCTTCCAACAGCAGCG
CTCCATGTTTAAGATACATATTTTCCCTGTTTGCTTTGCTACTGTATGTT
GACTTTAAGATCTTTTTTTAAATACATTTGATTCAGCTAGTATTCCATGT
CAACAATTTGTCCAAAGGAAAACTGCTGGAGGGAGGTGGAGGGAGGAAGG
TGGGAATTATTATTTAATACATCATTAATGCTTATTAATCTCTCACAAGC
ATCTTTGTCTTGCAAATCCTAAGGGAAAAGCAAGTCCCTGCAGTGAGCAC
TAGGGACAGTCTAATTTGGGGATTGCTCAACCATCAAGACTGCAGGTCTC
CCTTCAGCCACCTCCTTCCTGCTAAAAGCTTAGCCTACCACACTACCAGT
CATTCCCATCGCTTTGCAATCACAAGCCACAGGATGAGAAGTTCTGACTC
ACTCATGCCATGCCCAGGGCTATCTGAAACAATGTCTCATTAAGAATTTA
GGGTTCTTCCATGGGCTTACTGACAGTTGCCCAGATCTGAAGGGGAAAGG
GTCTTGAGAAAGACCATCACTGGCTCAACTTTAGGGCACTGTCCAGAGTC
AACATGATGTGGTTTAGCAGTGATCACATCTAAACAAAGTTTAGGTAAAT
GAATTATCGCAGAGAAAAACCACATGAGAAAATTTTTGTACTCCAAATTT
ACTTCCCAATAAATATTCAGCAAAGTAGTAAAATGACCTTAAAGATAAAA
ATGATTAGGGAATAGCCTTAGAAAATTTATAGGTATAAAAAATTCAAGGA
CAAACTGTGCATTTAATGGACACAAGAATTGACTCTAACTCCATGTCTGT
GGTTTCTTTGAACCCATATCAAATGTATGACTATTTAGAGTGTTTATAAG
AGATAATGGAACTGAACTTTCACTCAATTAATTGGGCATTAACAACCTTC
TTTTATGTTTGTTCCTGATATAGTCTGAATCTTAGGAAGAAGGTAAAAGA
AAGGAGGCAAGAGAATAGTTATGATGAATATGTGTTAAGTGCCTGCTCTG
AAGGAGGCAATGTTCTTCTCATTTGAATCCTTATGGCAACCTTATTCAAT
AGGTTTTCCCATATTTCAGATTTAATAACTGAAGGCCAGAGAGATTAATT
TGCCAAAGCCACACCTTTATGCTAATTATGATTGGAATGCATCACAAAAG
CCTAACTCTGTTGTTTTCAACCTCTACGTTATTTTGCTGCTATGTGCATT
TCCAGATCTGATTTTCTGCTAACTTGTGTGCTATGATCCACTCCTGATGG
GGGTCTACATTAATCTTCCAGTACTCCTTGCTGATGCTGTGTTATGTGTC
ATCTAACAGAAATGACTCCTTTGAAATAAGTAAATCTTTGGCTTTTTGTT
CTGTTGGTGTGATTCAAAGCAAAACAAACAAACAAAAACAAATTTTAAGA
ACACAACAAAAAAGATTTGACTTCCGAATAGAATGTTTTCTTTAAGAGGC
ATGAAAAGCAACTATTGTTGTGTTACAGTGTTAAAAATATTCAGTTTTCT
TTGACAAAAATGTGTACTGTGTAAGCCTTGCAAACAAAAACAACAAAAAA
GAAGCAGCAGCAGCAGCCTGCTGTGTGGCATCTGAACTTTTATAAAGGTT
TCCTTGTGCCAAATAAGTGCAAAGATTTAATTTACTATTAAAAACCATAA
GCATATGTTATAGTTCCAGAAGAATTATTTTGTCATCAAGTGATTTTGAT
CTTTAGTGTCAATATTTATATTTAGATTAATTTTTATAAATGAAAATATT
TTAATGGTTTAAGAAAATGAGGACAACAGGATAATATCTTTGATGACTTC
TGAAAGTTATGCTTCCCTTCATGTTATATGCACATTGCCAAGAATTACTG
TCAAGAGAAATGATAAGTAAAAGTCATTTATGAAATAAAAAAAAAAAAAA
AAA.

TABLE 9B

Encoded Myosin VIIA and Rab interacting protein
sequence (SEQ ID NO:21)

ENVYLAAGTVYGLETQLTELEDAARCIHSGTDETHLADLEDQVATAAAQV
HHAELQISDIESRISALTIAGLNIAPCVRFTRRRDQKQRTQVQTIDTSRQ
QRRKLPAPPVKAEKIETSSVTTIKTFNHNFILQGSSTNRTKERKGTTKDL
MEPALESAVMY.

TSC10: Melanophilin.

A18 10764 does not possess a reading frame beyond 50 amino acids.

TABLE 10A

Melanophilin (A1810764) nucleotide sequence
(SEQ ID NO:22)

AAAGGCACAGCTTTCCCAGTGTTTGTGTTCCTTGCTTGCGCCCTGTTTTA
ATGTTGTAGTTACAGGTGTCCAGCAGGGAGGAATGCAGCCCCTGTGGGCG
CTTGGGGGAGCTGCTGGGAATCCAAGTTCAAGGAGCAGCTGTTTTCTGTT
TTCTGTTGCCCCACAGCGCCACCTCCTGGCCCCTTGGTGGTGATGATTTT
GAAGTCAGCAGGTTCTGGTGGGCCGTGTGAACTCCAGCAGCTCTGGGCTG
AGCTGTGGAAACACTGCGTCCTTTGAAATAATACAGCTTTCCTGAGCCCA
CCCCAGTCCCTAAAGACTGCCTCTGGGGTTGAGATTCTGAGATGCTTGAC
AGCATGGCTTTTCCCGGTGTTATGTGTCGTTTCTATCCTTAAGCCTGTTA
GGGGTGGACTGGAGGCTGGACCAAGCTCCACTGGCTGCAGGAGGACCCTT
CTGTGGGCTCCAGGCTGGCCGTGTGCGTGTGGGGAGGTGGGATTTGCTGC
TAGGCTTCATGATCACTGTGAAGAAGCAGCCCCCAAGAATAGGGTGATAG
GCCCTCCCCATGTCACCG.

TSC11: ATP-Binding Cassette, Sub-Family C (CFTR/MRP), Member 8.

TABLE 11A

ATP-binding cassette, sub-family C (CFTR/MRP),
member 8 (AF087138.1) nucleotide sequence
(SEQ ID NO:23)

AGCTGAGCCCGAGCCCAGACCGCGCCCGCGCCGCCATGCCCCTGGCCTTC
TGCGGCAGCGAGAACCACTCGGCCGCCTACCGGGTGGACCAGGGGGTCCT
CAACAACGGCTGCTTTGTGGACGCGCTCAACGTGGTGCCGCACGTCTTCC
TACTCTTCATCACCTTCCCCATCCTCTTCATTGGATGGGGAAGTCAGAGC
TCCAAGGTGCACATCCACCACAGCACATGGCTTCATTTCCCTGGGCACAA
CCTGCGGTGGATCCTGACCTTCATGCTGCTCTTCGTCCTGGTGTGTGAGA
TTGCAGAGGGCATCCTGTCTGATGGGGTGACCGAATCCCACCATCTGCAC
CTGTACATGCCAGCCGGGATGGCGTTCATGGCTGCTGTCACCTCCGTGGT
CTACTATCACAACATCGAGACTTCCAACTTCCCCAAGCTGCTAATTGCCC
TGCTGGTGTATTGGACCCTGGCCTTCATCACCAAGACCATCAAGTTTGTC
AAGTTCTTGGACCACGCCATCGGCTTCTCGCAGCTACGCTTCTGCCTCAC
AGGGCTGCTGGTGATCCTCTATGGGATGCTGCTCCTCGTGGAGGTCAATG
TCATCAGGGTGAGGAGATACATCTTCTTCAAGACACCGAGGGAGGTGAAG
CCTCCCGAGGACCTGCAAGACCTGGGGGTACGCTTCCTGCAGCCCTTCGT
GAATCTGCTGTCCAAAGGCACCTACTGGTGGATGAACGCCTTCATCAAGA
CTGCCCACAAGAAGCCCATCGACTTGCGAGCCATCGGGAAGCTGCCCATC
GCCATGAGGGCCCTCACCAACTACCAACGGCTCTGCGAGGCCTTTGACGC
CCAGGTGCGGAAGGACATTCAGGGCACTCAAGGTGCCCGGGCCATCTGGC
AGGCACTCAGCCATGCCTTCGGGAGGCGCCTGGTCCTCAGCAGCACTTTC
CGCATCTTGGCCGACCTGCTGGGCTTCGCCGGGCCACTGTGCATCTTTGG
GATCGTGGACCACCTTGGGAAGGAGAACGACGTCTTCCAGCCCAAGACAC
AATTTCTCGGGGTTTACTTTGTCTCATCCCAAGAGTTCCTTGCCAATGCC
TACGTCTTAGCTGTGCTTCTGTTCCTTGCCCTCCTACTGCAAAGGACATT
TCTGCAAGCATCCTACTATGTGGCCATTGAAACTGGAATTAACTTGAGAG
GAGCAATACAGACCAAGATTTACAATAAAATTATGCACCTGTCCACCTCC
AACCTGTCCATGGGAGAAATGACTGCTGGACAGATCTGTAATCTGGTTGC
CATCGACACCAATCAGCTCATGTGGTTTTTCTTCTTGTGCCCAAACCTCT
GGGCTATGCCAGTACAGATCATTGTGGGTGTGATTCTCCTCTACTACATA
CTCGGAGTCAGTGCCTTAATTGGAGCAGCTGTCATCATTCTACTGGCTCC
TGTCCAGTACTTCGTGGCCACCAAGCTGTCTCAGGCCCAGCGGAGCACAC
TGGAGTATTCCAATGAGCGGCTGAAGCAGACCAACGAGATGCTCCGCGGC
ATCAAGCTGCTGAAGCTGTACGCCTGGGAGAACATCTTCCGCACGCGGGT
GGAGACGACCCGCAGGAAGGAGATGACCAGCCTCAGGGCCTTTGCCATCT
ATACCTCCATCTCCATTTTCATGAACACGGCCATCCCCATTGCAGCTGTC
CTCATAACTTTCGTGGGCCATGTCAGCTTCTTCAAAGAGGCCGACTTCTC
GCCCTCCGTGGCCTTTGCCTCCCTCTCCCTCTTCCATATCTTGGTCACAC
CGCTGTTCCTGCTGTCCAGTGTGGTCCGATCTACCGTCAAAGCTCTAGTG
AGCGTGCAAAAGCTAAGCGAGTTCCTGTCCAGTGCAGAGATCCGTGAGGA
GCAGTGTGCCCCCCATGAGCCCACACCTCAGGGCCCAGCCAGCAAGTACC
AGGCGGTGCCCCTCAGGGTTGTGAACCGCAAGCGTCCAGCCCGGGAGGAT
TGTCGGGGCCTCACCGGCCCACTGCAGAGCCTGGTCCCCAGTGCAGATGG
CGATGCTGACAACTGCTGTGTCCAGATCATGGGAGGCTACTTCACGTGGA
CCCCAGATGGAATCCCCACACTGTCCAACATCACCATTCGTATCCCCCGA
GGCCAGCTGACTATGATCGTGGGGCAGGTGGGCTGCGGCAAGTCCTCGCT
CCTTCTAGCCGCACTGGGGGAGATGCAGAAGGTCTCAGGGGCTGTCTTCT
GGAGCAGCCTTCCTGACAGCGAGATAGGAGAGGACCCCAGCCCAGAGCGG
GAGACAGCGACCGACTTGGATATCAGGAAGAGAGGCCCCGTGGCCTATGC
TTCGCAGAAACCATGGCTGCTAAATGCCACTGTGGAGGAGAACATCATCT
TTGAGAGTCCCTTCAACAAACAACGGTACAAGATGGTCATTGAAGCCTGC
TCTCTGCAGCCAGACATCGACATCCTGCCCCATGGAGACCAGACCCAGAT
TGGGGAACGGGGCATCAACCTGTCTGGTGGTCAACGCCAGCGAATCAGTG
TGGCCCGAGCCCTCTACCAGCACGCCAACGTTGTCTTCTTGGATGACCCC
TTCTCAGCTCTGGATATCCATCTGAGTGACCACTTAATGCAGGCCGGCAT
CCTTGAGCTGCTCCGGGACGACAAGAGGACAGTGGTCTTAGTGACCCACA
AGCTACAGTACCTGCCCCATGCAGACTGGATCATTGCCATGAAGGATGGC
ACCATCCAGAGGGAGGGTACCCTCAAGGACTTCCAGAGGTCTGAATGCCA
GCTCTTTGAGCACTGGAAGACCCTCATGAACCGACAGGACCAAGAGCTGG
AGAAGGAGACTGTCACAGAGAGAAAAGCCACAGAGCCACCCCAGGGCCTA
TCTCGTGCCATGTCCTCGAGGGATGGCCTTCTGCAGGATGAGGAAGAGGA
GGAAGAGGAGGCAGCTGAGAGCGAGGAGGATGACAACCTGTCGTCCATGC
TGCACCAGCGTGCTGAGATCCCATGGCGAGCCTGCGCCAAGTACCTGTCC
TCCGCCGGCATCCTGCTCCTGTCGTTGCTGGTCTTCTCACAGCTGCTCAA
GCACATGGTCCTGGTGGCCATCGACTACTGGCTGGCCAAGTGGACCGACA
GCGCCCTGACCCTGACCCCTGCAGCCAGGAACTGCTCCCTCAGCCAGGAG
TGCACCCTCGACCAGACTGTCTATGCCATGGTGTTCACGGTGCTCTGCAG
CCTGGGCATTGTGCTGTGCCTCGTCACGTCTGTCACTGTGGAGTGGACAG
GGCTGAAGGTGGCCAAGAGACTGCACCGCAGCCTGCTAAACCGGATCATC
CTAGCCCCCATGAGGTTTTTTGAGACCACGCCCCTTGGGAGCATCCTGAA
CAGATTTTCATCTGACTGTAACACCATCGACCAGCACATCCCATCCACGC
TGGAGTGCCTGAGCCGCTCCACCCTGCTCTGTGTCTCAGCCCTGGCCGTC
ATCTCCTATGTCACACCTGTGTTCCTCGTGGCCCTCTTGCCCCTGGCCAT
CGTGTGCTACTTCATCCAGAAGTACTTCCGGGTGGCGTCCAGGGACCTGC
AGCAGCTGGATGACACCACCCAGCTTCCACTTCTCTCACACTTTGCCGAA
ACCGTAGAAGGACTCACCACCATCCGGGCCTTCAGGTATGAGGCCCGGTT
CCAGCAGAAGCTTCTCGAATACACAGACTCCAACAACATTGCTTCCCTCT
TCCTCACAGCTGCCAACAGATGGCTGGAAGTCCGAATGGAGTACATCGGT
GCATGTGTGGTGCTCATCGCAGCGGTGACCTCCATCTCCAACTCCCTGCA
CAGGGAGCTCTCTGCTGGCCTGGTGGGCCTGGGCCTTACCTACGCCCTAA
TGGTCTCCAACTACCTCAACTGGATGGTGAGGAACCTGGCAGACATGGAG
CTCCAGCTGGGGGCTGTGAAGCGCATCCATGGGCTCCTGAAAACCGAGGC
AGAGAGCTACGAGGGGCTCCTGGCACCATCGCTGATCCCAAAGAACTGGC
CAGACCAAGGGAAGATCCAGATCCAGAACCTGAGCGTGCGCTACGACAGC
TCCCTGAAGCCGGTGCTGAAGCACGTCAATGCCCTCATCTCCCCTGGACA
GAAGATCGGGATCTGCGGCCGCACCGGCAGTGGGAAGTCCTCCTTCTCTC
TTGCCTTCTTCCGCATGGTGGACACGTTCGAAGGGCACATCATCATTGAT
GGCATTGACATCGCCAAACTGCCGCTGCACACCCTGCGCTCACGCCTCTC
CATCATCCTGCAGGACCCCGTCCTCTTCAGCGGCACCATCCGATTTAACC
TGGACCCTGAGAGGAAGTGCTCAGATAGCACACTGTGGGAGGCCCTGGAA
ATCGCCCAGCTGAAGCTGGTGGTGAAGGCACTGCCAGGAGGCCTCGATGC
CATCATCACAGAAGGCGGGGAGAATTTCAGCCAGGGACAGAGGCAGCTGT
TCTGCCTGGCCCGGGCCTTCGTGAGGAAGACCAGCATCTTCATCATGGAC
GAGGCCACGGCTTCCATTGACATGGCCACGGAAAACATCCTCCAAAAGGT
GGTGATGACAGCCTTCGCAGACCGCACTGTGGTCACCATCGCGCATCGAG
TGCACACCATCCTGAGTGCAGACCTGGTGATCGTCCTGAAGCGGGGTGCC
ATCCTTGAGTTCGATAAGCCAGAGAAGCTGCTCAGCCGGAAGGACAGCGT
CTTCGCCTCCTTCGTCCGTGCAGACAAGTGACCTGCCAGAGCCCAAGTGC
CATCCCACATTCGGACCCTGCCCATA.

TABLE 11B

ATP-binding cassette, sub-family C (CFTR/MRP),
member 8 (AF087138.1) protein sequence
(SEQ ID NO:24)

MPLAFCGSENHSAAYRVDQGVLNNGCFVDALNVVPHVFLLFITFPILFIG
WGSQSSKVHIHHSTWLHFPGHNLRWILTFMLLFVLVCEIAEGILSDGVTE
SHHLHLYMPAGMAFMAAVTSVVYYHNIETSNFPKLLIALLVYWTLAFITK
TIKFVKFLDHAIGFSQLRFCLTGLLVILYGMLLLVEVNVIRVRRYIFFKT
PREVKPPEDLQDLGVRFLQPFVNLLSKGTYWWMNAFIKTAHKKPIDLRAI
GKLPIAMRALTNYQRLCEAFDAQVRKDIQGTQGARAIWQALSHAFGRRLV
LSSTFRILADLLGFAGPLCIFGIVDHLGKENDVFQPKTQFLGVYFVSSQE
FLANAYVLAVLLFLALLLQRTFLQASYYVAIETGINLRGAIQTKIYNKIM
HLSTSNLSMGEMTAGQICNLVAIDTNQLMWFFFLCPNLWAMPVQIIVGVI
LLYYILGVSALIGAAVIILLAPVQYFVATKLSQAQRSTLEYSNERLKQTN
EMLRGIKLLKLYAWENIFRTRVETTRRKEMTSLRAFAIYTSISIFMNTAI
PIAAVLITFVGHVSFFKEADFSPSVAFASLSLFHILVTPLFLLSSVVRST
VKALVSVQKLSEFLSSAEIREEQCAPHEPTPQGPASKYQAVPLRVVNRKR
PAREDCRGLTGPLQSLVPSADGDADNCCVQIMGGYFTWTPDGIPTLSNIT
IRIPRGQLTMIVGQVGCGKSSLLLAALGEMQKVSGAVFWSSLPDSEIGED
PSPERETATDLDIRKRGPVAYASQKPWLLNATVEENIIFESPFNKQRYKM
VIEACSLQPDIDILPHGDQTQIGERGINLSGGQRQRISVARALYQHANVV
FLDDPFSALDIHLSDHLMQAGILELLRDDKRTVVLVTHKLQYLPHADWII
AMKDGTIQREGTLKDFQRSECQLFEHWKTLMNRQDQELEKETVTERKATE
PPQGLSRAMSSRDGLLQDEEEEEEEAAESEEDDNLSSMLHQRAEIPWRAC
AKYLSSAGILLLSLLVFSQLLKHMVLVAIDYWLAKWTDSALTLTPAARNC
SLSQECTLDQTVYAMVFTVLCSLGIVLCLVTSVTVEWTGLKVAKRLHRSL
LNRIILAPMRFFETTPLGSILNRFSSDCNTIDQHIPSTLECLSRSTLLCV
SALAVISYVTPVFLVALLPLAIVCYFIQKYFRVASRDLQQLDDTTQLPLL
SHFAETVEGLTTIRAFRYEARFQQKLLEYTDSNNIASLFLTAANRWLEVR
MEYIGACVVLIAAVTSISNSLHRELSAGLVGLGLTYALMVSNYLNWMVRN
LADMELQLGAVKRIHGLLKTEAESYEGLLAPSLIPKNWPDQGKIQIQNLS
VRYDSSLKPVLKHVNALISPGQKIGICGRTGSGKSSFSLAFFRMVDTFEG
HIIIDGIDIAKLPLHTLRSRLSIILQDPVLFSGTIRFNLDPERKCSDSTL
WEALEIAQLKLVVKALPGGLDAIITEGGENFSQGQRQLFCLARAFVRKTS
IFIMDEATASIDMATENILQKVVMTAFADRTVVTIAHRVHTILSADLVIV
LKRGAILEFDKPEKLLSRKDSVFASFVRADK.

TABLE 11C

ATP-binding cassette, sub-family C (CFTR/MRP),
member 8 (NM_000352.2) nucleotide sequence
(SEQ ID NO:25)

CGGGGCCCGGGGGGCGGGGGCCTGACGGCCGGGCCGGGCGGCGGAGCTGC
AAGGGACAGAGGCGCGGCAGGCGCGCGGAGCCAGCGGAGCCAGCTGAGCC
CGAGCCCAGCCCGCGCCCGCGCCGCCATGCCCCTGGCCTTCTGCGGCAGC
GAGAACCACTCGGCCGCCTACCGGGTGGACCAGGGGGTCCTCAACAACGG
CTGCTTTGTGGACGCGCTCAACGTGGTGCCGCACGTCTTCCTACTCTTCA
TCACCTTCCCCATCCTCTTCATTGGATGGGGAAGTCAGAGCTCCAAGGTG
CACATCCACCACAGCACATGGCTTCATTTCCCCGGGCACAACCTGCGGTG
GATCCTGACCTTCATGCTGCTCTTCGTCCTGGTGTGTGAGATTGCAGAGG
GCATCCTGTCTGATGGGGTGACCGAATCCCACCATCTGCACCTGTACATG
CCAGCCGGGATGGCGTTCATGGCTGCTGTCACCTCCGTGGTCTACTATCA
CAACATCGAGACTTCCAACTTCCCCAAGCTGCTAATTGCCCTGCTGGTGT
ATTGGACCCTGGCCTTCATCACCAAGACCATCAAGTTTGTCAAGCTCTTG
GACCACGCCATCGGCTTCTCGCAGCTACGCTTCTGCCTCACAGGGCTGCT
GGTGATCCTCTATGGGATGCTGCTCCTCGTGGAGGTCAATGTCATCAGGG
TGAGGAGATACATCTTCTTCAAGACACCGAGGGAGGTGAAGCCTCCCGAG
GACCTGCAAGACCTGGGGGTACGCTTCCTGCAGCCCTTCGTGAATCTGCC
GTCCAAAGGCACCTACTGGTGGATGAACGCCTTCATCAAGACTGCCCACA
AGAAGCCCATCGACTTGCGAGCCATCGGGAAGCTGCCCATCGTTATGAGG
GCCCTCACCAACTACCAACGGCTCTGCGAGGCCTTTGACGCCCAGGTGCG
GAAGGACATTCAGGGCACTCAAGGTGCCCGGGCCATCTGGCAGGCACTCA
GCCATGCCTTCGGGAGGCGCCTGGTCCTCAGCAGCACTTTCCGCATCTTG
GCCGACCTGCTGGGCTTCGCCGGGCCACTGTGCATCTTTGGGATCGTGGA
CCACCTTGGGAAGGAGAACGACGTCTTCCAGCCCAAGACACAATTTCTCG
GGGTTTACTTTGTCTCATCCCAAGAGTTCCTTGCCAATGCCTACGTCTTA
GCTGTGCTTCTGTTCCTTGCCCTCCTACTGCAAAGGACATTTCTGCAAGC
ATCCTACTATGTGGCCATTGAAACTGGAATTAACTTGAGAGGAGCAATAC
AGACCAAGATTTACAATAAAATTATGCACCTGTCCACCTCCAACCTGTCC
ATGGGAGAAATGACTGCTGGACAGATCTGTAATCTGGTTGCCATCGACAC
CAATCAGCTCATGTGGTTTTTCTTCTTGTGCCCAAACCTCTGGGCTATGC
CAGTACAGATCATTGTGGGTGTGATTCTCCTCTACTACATACTCGGAGTC
AGTGCCTTAATTGGAGCAGCTGTCATCATTCTACTGGCTCCTGTCCAGTA
CTTCGTGGCCACCAAGCTGTCTCAGGCCCAGCGGAGCACACTGGAGTATT
CCAATGAGCGGCTGAAGCAGACCAACGAGATGCTCCGCGGCATCAAGCTG
CTGAAGCTGTACGCCTGGGAGAACATCTTCCGCACGCGGGTGGAGACGAC
CCGCAGGAAGGAGATGACCAGCCTCAGGGCCTTTGCCATCTATACCTCCA
TCTCCATTTTCATGAACACGGCCATCCCCATTGCAGCTGTCCTCATAACT
TTCGTGGGCCATGTCAGCTTCTTCAAAGAGGCCGACTTCTCGCCCTCCGT
GGCCTTTGCCTCCCTCTCCCTCTTCCATATCTTGGTCACACCGCTGTTCC
TGCTGTCCAGTGTGGTCCGATCTACCGTCAAAGCTCTAGTGAGCGTGCAA
AAGCTAAGCGAGTTCCTGTCCAGTGCAGAGATCCGTGAGGAGCAGTGTGC
CCCCCATGAGCCCACACCTCAGGGCCCAGCCAGCAAGTACCAGGCGGTGC
CCCTCAGGGTTGTGAACCGCAAGCGTCCAGCCCGGGAGGATTGTCGGGGC
CTCACCGGCCCACTGCAGAGCCTGGTCCCCAGTGCAGATGGCGATGCTGA
CAACTGCTGTGTCCAGATCATGGGAGGCTACTTCACGTGGACCCCAGATG
GAATCCCCACACTGTCCAACATCACCATTCGTATCCCCCGAGGCCAGCTG
ACTATGATCGTGGGGCAGGTGGGCTGCGGCAAGTCCTCGCTCCTTCTAGC
CGCACTGGGGGAGATGCAGAAGGTCTCAGGGGCTGTCTTCTGGAGCAGCC
TTCCTGACAGCGAGATAGGAGAGGACCCCAGCCCAGAGCGGGAGACAGCG
ACCGACTTGGATATCAGGAAGAGAGGCCCCGTGGCCTATGCTTCGCAGAA
ACCATGGCTGCTAAATGCCACTGTGGAGGAGAACATCATCTTTGAGAGTC
CCTTCAACAAACAACGGTACAAGATGGTCATTGAAGCCTGCTCTCTGCAG
CCAGACATCGACATCCTGCCCCATGGAGACCAGACCCAGATTGGGGAACG
GGGCATCAACCTGTCTGGTGGTCAACGCCAGCGAATCAGTGTGGCCCGAG
CCCTCTACCAGCACGCCAACGTTGTCTTCTTGGATGACCCCTTCTCAGCT
CTGGATATCCATCTGAGTGACCACTTAATGCAGGCCGGCATCCTTGAGCT
GCTCCGGGACGACAAGAGGACAGTGGTCTTAGTGACCCACAAGCTACAGT
ACCTGCCCCATGCAGACTGGATCATTGCCATGAAGGATGGGACCATCCAG
AGGGAGGGTACCCTCAAGGACTTCCAGAGGTCTGAATGCCAGCTCTTTGA
GCACTGGAAGACCCTCATGAACCGACAGGACCAAGAGCTGGAGAAGGAGA
CTGTCACAGAGAGAAAAGCCACAGAGCCACCCCAGGGCCTATCTCGTGCC
ATGTCCTCGAGGGATGGCCTTCTGCAGGATGAGGAAGAGGAGGAAGAGGA
GGCAGCTGAGAGCGAGGAGGATGACAACCTGTCGTCCATGCTGCACCAGC
GTGCTGAGATCCCATGGCGAGCCTGCGCCAAGTACCTGTCCTCCGCCGGC
ATCCTGCTCCTGTCGTTGCTGGTCTTCTCACAGCTGCTCAAGCACATGGT
CCTGGTGGCCATCGACTACTGGCTGGCCAAGTGGACCGACAGCGCCCTGA
CCCTGACCCCTGCAGCCAGGAACTGCTCCCTCAGCCAGGAGTGCACCCTC
GACCAGACTGTCTATGCCATGGTGTTCACGGTGCTCTGCAGCCTGGGCAT
TGTGCTGTGCCTCGTCACGTCTGTCACTGTGGAGTGGACAGGGCTGAAGG
TGGCCAAGAGACTGCACCGCAGCCTGCTAAACCGGATCATCCTAGCCCCC
ATGAGGTTTTTTGAGACCACGCCCCTTGGGAGCATCCTGAACAGATTTTC
ATCTGACTGTAACACCATCGACCAGCACATCCCATCCACGCTGGAGTGCC
TGAGCCGCTCCACCCTGCTCTGTGTCTCAGCCCTGGCCGTCATCTCCTAT
GTCACACCTGTGTTCCTCGTGGCCCTCTTGCCCCTCGCAGTCGTGTGCTA
CTTCATCCAGAAGTACTTCCGGGTGGCGTCCAGGGACCTGCAGCAGCTGG
ATGACACCACCCAGCTTCCACTTCTCTCACACTTTGCCGAAACCGTAGAA
GGACTCACCACCATCCGGGCCTTCAGGTATGAGGCCCGGTTCCAGCAGAA
GCTTCTCGAATACACAGACTCCAACAACATTGCTTCCCTCTTCCTCACAG
CTGCCAACAGATGGCTGGAAGTCCGAATGGAGTACATCGGTGCATGTGTG
GTGCTCATCGCAGCGGTGACCTCCATCTCCAACTCCCTGCACAGGGAGCT
CTCTGCTGGCCTGGTGGGCCTGGGCCTTACCTACGCCCTAATGGTCTCCA
ACTACCTCAACTGGATGGTGAGGAACCTGGCAGACATGGAGCTCCAGCTG
GGGGCTGTGAAGCGCATCCATGGGCTCCTGAAAACCGAGGCAGAGAGCTA
CGAGGGGCTCCTGGCACCATCGCTGATCCCAAAGAACTGGCCAGACCAAG
GGAAGATCCAGATCCAGAACCTGAGCGTGCGCTACGACAGCTCCCTGAAG
CCGGTGCTGAAGCACGTCAATGCCCTCATCTCCCCTGGACAGAAGATCGG
GATCTGCGGCCGCACCGGCAGTGGGAAGTCCTCCTTCTCTCTTGCCTTCT
TCCGCATGGTGGACACGTTCGAAGGGCACATCATCATTGATGGCATTGAC
ATCCGCAAACTGCCGCTGCACACCCTGCCGTCACGCCTCTCCATCATCCT
GCAGGACCCCGTCCTCTTCAGCGGCACCATCCGATTTAACCTGGACCCTG
AGAGGAAGTGCTCAGATAGCACACTGTGGGAGGCCCTGGAAATCGCCCAG
CTGAAGCTGGTGGTGAAGGCACTGCCAGGAGGCCTCGATGCCATCATCAC
AGAAGGCGGGGAGAATTTCAGCCAGGGACAGAGGCAGCTGTTCTGCCTGG
CCCGGGCCTTCGTGAGGAAGACCAGCATCTTCATCATGGACGAGGCCACG
GCTTCCATTGACATGGCCACGGAAAACATCCTCCAAAAGGTGGTGATGAC
AGCCTTCGCAGACCGCACTGTGGTCACCATCGCGCATCGAGTGCACACCA
TCCTGAGTGCAGACCTGGTGATCGTCCTGAAGCGGGGTGCCATCCTTGAG
TTCGATAAGCCAGAGAAGCTGCTCAGCCGGAAGGACAGCGTCTTCGCCTC
CTTCGTCCGTGCAGACAAGTGACCTGCCAGAGCCCAAGTGCCATCCCACA
TTCGGACCCTGCCCATACCCCTGCCTGGGTTTTCTAACTGTAAATCACTT
GTAAATAAATAGATTTGATTATTTCCT.

TABLE 11D

ATP-binding cassette, sub-family C (CFTR/MRP),
member 8 (NM_000352.2) protein sequence
(SEQ ID NO:26)

MPLAFCGSENHSAAYRVDQGVLNNGCFVDALNVVPHVFLLFITFPILFIG
WGSQSSKVHIHHSTWLHFPGHNLRWILTFMLLFVLVCEIAEGILSDGVTE
SHHLHLYMPAGMAFMAAVTSVVYYHNIETSNFPKLLIALLVYWTLAFITK
TIKFVKLLDHAIGFSQLRFCLTGLLVILYGMLLLVEVNVIRVRRYIFFKT
PREVKPPEDLQDLGVRFLQPFVNLPSKGTYWWMNAFIKTAHKKPIDLRAI
GKLPIVMRALTNYQRLCEAFDAQVRKDIQGTQGARAIWQALSHAFGRRLV
LSSTFRILADLLGFAGPLCIFGIVDHLGKENDVFQPKTQFLGVYFVSSQE
FLANAYVLAVLLFLALLLQRTFLQASYYVAIETGINLRGAIQTKIYNKIM
HLSTSNLSMGEMTAGQICNLVAIDTNQLMWFFFLCPNLWAMPVQIIVGVI
LLYYILGVSALIGAAVIILLAPVQYFVATKLSQAQRSTLEYSNERLKQTN
EMLRGIKLLKLYAWENIFRTRVETTRRKEMTSLRAFAIYTSISIFMNTAI
PIAAVLITFVGHVSFFKEADFSPSVAFASLSLFHILVTPLFLLSSVVRST
VKALVSVQKLSEFLSSAEIREEQCAPHEPTPQGPASKYQAVPLRVVNRKR
PAREDCRGLTGPLQSLVPSADGDADNCCVQIMGGYFTWTPDGIPTLSNIT
IRIPRGQLTMIVGQVGCGKSSLLLAALGEMQKVSGAVFWSSLPDSEIGED
PSPERETATDLDIRKRGPVAYASQKPWLLNATVEENIIFESPFNKQRYKM
VIEACSLQPDIDILPHGDQTQIGERGINLSGGQRQRISVARALYQHANVV
FLDDPFSALDIHLSDHLMQAGILELLRDDKRTVVLVTHKLQYLPHADWII
AMKDGTIQREGTLKDFQRSECQLFEHWKTLMNRQDQELEKETVTERKATE
PPQGLSRAMSSRDGLLQDEEEEEEEAAESEEDDNLSSMLHQRAEIPWRAC
AKYLSSAGILLLSLLVFSQLLKHMVLVAIDYWLAKWTDSALTLTPAARNC
SLSQECTLDQTVYAMVFTVLCSLGIVLCLVTSVTVEWTGLKVAKRLHRSL
LNRIILAPMRFFETTPLGSILNRFSSDCNTIDQHIPSTLECLSRSTLLCV
SALAVISYVTPVFLVALLPLAVVCYFIQKYFRVASRDLQQLDDTTQLPLL
SHFAETVEGLTTIRAFRYEARFQQKLLEYTDSNNIASLFLTAANRWLEVR
MEYIGACVVLIAAVTSISNSLHRELSAGLVGLGLTYALMVSNYLNWMVRN
LADMELQLGAVKRIHGLLKTEAESYEGLLAPSLIPKNWPDQGKIQIQNLS
VRYDSSLKPVLKHVNALISPGQKIGICGRTGSGKSSFSLAFFRMVDTFEG
HIIIDGIDIRKLPLHTLPSRLSIILQDPVLFSGTIRFNLDPERKCSDSTL
WEALEIAQLKLVVKALPGGLDAIITEGGENFSQGQRQLFCLARAFVRKTS
IFIMDEATASIDMATENILQKVVMTAFADRTVVTIAHRVHTILSADLVIV
LKRGAILEFDKPEKLLSRKDSVFASFVRADK.

TSC12: Vasoactive Intestinal Peptide Receptor 2.

X95097.2 and NM_{—}003382.2 Both Encode the Polypeptide Sequence Shown in Table 12C.

TABLE 12A

Vasoactive intestinal peptide receptor 2
(X95097.2) nucleotide sequence (SEQ ID NO:27)

GTGCATTGAGCGCGCTCCAGCTGCCGGGACGGAGGGGGCGGCCCCCGCGC
TCGGGGCGCTCGGCTACAGCTGCGGGGCCCGAGGTCTCCGCGCACTCGCT
CCCGGCCCATGCTGGAGGCGGCGGAACCGCGGGGACCTAGGACGGAGGCG
GCGGGCGCTGGGCGGCCCCCGGCACGCTGAGCTCGGGATGCGGACGCTGC
TGCCTCCCGCGCTGCTGACCTGCTGGCTGCTCGCCCCCGTGAACAGCATT
CACCCAGAATGCCGATTTCATCTGGAAATACAGGAGGAAGAAACAAAATG
TGCAGAGCTTCTGAGGTCTCAAACAGAAAAACACAAAGCCTGCAGTGGCG
TCTGGGACAACATCACGTGCTGGCGGCCTGCCAATGTGGGAGAGACCGTC
ACGGTGCCCTGCCCAAAAGTCTTCAGCAATTTTTACAGCAAAGCAGGAAA
CATAAGCAAAAACTGTACGAGTGACGGATGGTCAGAGACGTTCCCAGATT
TCGTCGATGCCTGTGGCTACAGCGACCCGGAGGATGAGAGCAAGATCACG
TTTTATATTCTGGTGAAGGCCATTTATACCCTGGGCTACAGTGTCTCTCT
GATGTCTCTTGCAACAGGAAGCATAATTCTGTGCCTCTTCAGGAAGCTGC
ACTGCACCAGGAATTACATCCACCTGAACCTGTTCCTGTCCTTCATCCTG
AGAGCCATCTCAGTGCTGGTCAAGGACGACGTTCTCTACTCCAGCTCTGG
CACGTTGCACTGCCCTGACCAGCCATCCTCCTGGGTGGGCTGCAAGCTGA
GCCTGGTCTTCCTGCAGTACTGCATCATGGCCAACTTCTTCTGGCTGCTG
GTGGAGGGGCTCTACCTCCACACCCTCCTGGTGGCCATGCTCCCCCCTAG
AAGGTGCTTCCTGGCCTACCTCCTGATCGGATGGGGCCTCCCCACCGTCT
GCATCGGTGCATGGACTGCGGCCAGGCTCTACTTAGAAGACACCGGTTGC
TGGGATACAAACGACCACAGTGTGCCCTGGTGGGTCATACGAATACCGAT
TTTAATTTCCATCATCGTCAATTTTGTCCTTTTCATTAGTATTATACGAA
TTTTGCTGCAGAAGTTAACATCCCCAGATGTCGGCGGCAACGACCAGTCT
CAGTACAAGAGGCTGGCCAAGTCCACGCTCCTGCTTATCCCGCTGTTCGG
CGTCCACTACATGGTGTTTGCCGTGTTTCCCATCAGCATCTCCTCCAAAT
ACCAGATACTGTTTGAGCTGTGCCTCGGGTCGTTCCAGGGCCTGGTGGTG
GCCGTCCTCTACTGTTTCCTGAACAGTGAGGTGCAGTGCGAGCTGAAGCG
AAAATGGCGAAGCCGGTGCCCGACCCCGTCCGCGAGCCGGGATTACAGGG
TCTGCGGTTCCTCCTTCTCCCGCAACGGCTCGGAGGGCGCCCTGCAGTTC
CACCGCGGCTCCCGCGCCCAGTCCTTCCTGCAAACGGAGACCTCGGTCAT
CTAGCCCCACCCCTGCCTGTCGGACGCGGCGGGAGGCCCACGGTTCGGGG
CTTCTGCGGGGCTGAGACGCCGGCTTCCTCCTTCCAGATGCCCGAGCACC
GTGTCGGGCAGGTCAGCGCGGTCCTGACTCCGTCAAGCTGGTTGTCCACT
AAACCCCATACCTGGAATTGGAGTCGTGTTGTCATTGACTCGATTTAAAC
TCCAGCATTTAGATAATCTTGTGCAAAATGTGTTTCAGCCGTATAGTGGA
TCCACTTTTTTTTTTTTTTTTTTTTGAGACGGAGTCTTGCTCTGTCGCCC
AGGCTGGAGTGCAGTGGCCTGATCTCTGCTCCCTGCAAGCTCCGCCTCCC
GGGTTCACGCCATTCTCCTGCCTCAGCCTCCCATAGCTGGGACTACAGGC
GCCCGCCAACACGCCTGGCTAATTTTTTGTATTTTTAGTAGAGACAGGGT
TTCACCATGTTAGCCAGGATGGTCTCGATCTCCTGACCTCGTGATGGGCC
CGCCTCGGCCTCCCAAAGTGCTGGGATTAAGGCGTGAGCCACTGCGCCCG
GCCCAAGAGATAGGGGAGCCAAGGAGGAAATGTGGAAACGCAGTTGTGTG
GCCCAGCACGAGCCTGGGCGACCACCGGGTGACATCCGTCCCACATCAGG
GCGGCCTCCCAGGTCCCATAAGGGTAGCCCCCTCATCTGCAGGACAGAGG
GAAGCCAGTCAGGGCCCCCCCGGACGTTAGGACCAGGAGAAATCAACAGG
AGGGCAGCCCGTCCTCTCTCTTGGGGCGCCCACCCGGCCCGGCTGAGCCC
TGCCCCACCCAACTCCACAGGGCTGTTTTGCCTCCCCACGGAAGGCGGGC
TGAGGAGACAACCAGATCAGGAGAGCAAGGTCATGAAGGAGGGGACCTCT
CCACACAGGTGTTCCGTGGGACCCTCAGCAGCTCTGGCTCTGCCTCAGGA
GGTCACCTGCCGCCCTGTGGGAGCCGCAGAGCCTGACGCTCAGCCCCAGG
CCAGCTGCGGCCAGGCCTGCGGGCCCCTGGTGATGGGGTTACGTGGGGTG
CGGGATACAGCTGAGTGGGAACCGGAAACCTATTCTCTTTTTAACAAAAA
TAATCTTAGGATAAGAATTATTTTAACAACATATAAAACTGTTTCAAGCC
CTCCTCCCCAGAGCTGGCGCTCAGCAGCCCTAGCGGCTGCTCCTTCAGGC
GAAGGGTGGTTTGCAGATGTGGGGAGGGTGTCTGGGGACGTTGCTGAGCT
GGCTGCAGAAGGGTGGGGATATCAGGGCACAGTCTCCATGTGTGTGCCAA
GCCCTGGCCCCCACAGCGCTCGATGGACCTCAGCAAGCTGCCCAGCCCTG
GCCCAGGTGCCCCGACTGTGGGACTCAGTTGTTCTGAGCACATTTGACTC
CACTTTTCCTTTAAAAATGAATGTCTTGTTCCTGTGCATTGGTGGCATCA
CAGACCCCAGCTGGGGCGCGATGTCAAAGGTCGGGACAGCTGTGCCGGGA
GGCAGCCACAGGGAAGCTCACACATCCTGTCAGTGTCACCTTGGTTTGCA
AAACCCATATCCCCGGTAAAATGAGGCCGGACAGAGGGGCTGTTAGGACA
GCAAAGCAGCAGTGTCCAGAGACCCCTCAATCCCCAAAGGTCCGCACCCT
GTCCTGCACACCCTGGGCCACGCCGGCCACACCCCTCTGCTGCAACAAGC
TCATCCCTGGACTTCTGGGAGAATGAACCCGAGGTTGGTTTGGGGAGACA
GGTGAGGCGGTTGGATCTACAGAACAACCCACCATTTCTGGGGGCCGCAG
AGGATCCATCACAGACGGATACTGGGGAGTAAACGGCCCAGGCCAGGTGC
CCAGGAAAGGACGGCTGAGCATGTGGAGCGAGAGGGAGGCAGGTGGACGC
TGCAGACCCCAGGTTCAGTGCGGCCCCTCGGCTGTTCCTCCCCTGTAGGG
TTTGGACAGACCCACCCCCAGCCTTGCCCAGCTTTCAAAGGACAAAAGGG
AGCATCCCCCACCTACTCTCAGGTTTTTGAGGAAACAAAGATTTGTGGTA
ACTGAAGGTGTTGGGTCAGTGGCCAGGTGCCGACACTGAGCTGTGACCCA
GAGGGGACGCTGAGGAAGTGGGCGTGAGTGGACATGTCAGGTGGTTACCA
GGCACTGGTTGTTGATGGTCGGTGGTTGGGTGTGGGCAGTCATCAGTCAT
CAGGTGTGCTCAGGGGACAATCTCCCCTCAACCGCACATGTGCCACTGTT
CAGCGGAGCTGACTGGTTTCTCCTGGTAGAGGGCCGGCTGTATCCTGACA
GATGCCTGGTGAGCAGGGGAAGCAGGACCCAGTGGTCAACAGGTGTCTTT
AACTGTCATTGTGTGTGGAATGTCGCAGACTCCTCCACGTGGCGGGAATG
AGCTGTGTAAATACTTCAATAAAGCCTGATCTCACATCTGCAAAAAAAAA
AAAAAAAAAAAAAAAAAAAAAAA.

TABLE 12B

Vasoactive intestinal peptide receptor 2
(NM_003382.2) nucleotide sequence (SEQ ID NO:28)

GTGCATTGAGCGCGCTCCAGCTGCCGGGACGGAGGGGGCGGCCCCCGCGC
TCGGGCGCTCGGCTACAGCTGCGGGGCCCGAGGTCTCCGCGCACTCGCTC
CCGGCCCATGCTGGAGGCGGCGGAACCGCGGGGACCTAGGACGGAGGCGG
CGGGCGCTGGGCGGCCCCCGGCACGCTGAGCTCGGGATGCGGACGCTGCT
GCCTCCCGCGCTGCTGACCTGCTGGCTGCTCGCCCCCGTGAACAGCATTC
ACCCAGAATGCCGATTTCATCTGGAAATACAGGAGGAAGAAACAAAATGT
GCAGAGCTTCTGAGGTCTCAAACAGAAAAACACAAAGCCTGCAGTGGCGT
CTGGGACAACATCACGTGCTGGCGGCCTGCCAATGTGGGAGAGACCGTCA
CGGTGCCCTGCCCAAAAGTCTTCAGCAATTTTTACAGCAAAGCAGGAAAC
ATAAGCAAAAACTGTACGAGTGACGGATGGTCAGAGACGTTCCCAGATTT
CGTCGATGCCTGTGGCTACAGCGACCCGGAGGATGAGAGCAAGATCACGT
TTTATATTCTGGTGAAGGCCATTTATACCCTGGGCTACAGTGTCTCTCTG
ATGTCTCTTGCAACAGGAAGCATAATTCTGTGCCTCTTCAGGAAGCTGCA
CTGCACCAGGAATTACATCCACCTGAACCTGTTCCTGTCCTTCATCCTGA
GAGCCATCTCAGTGCTGGTCAAGGACGACGTTCTCTACTCCAGCTCTGGC
ACGTTGCACTGCCCTGACCAGCCATCCTCCTGGGTGGGCTGCAAGCTGAG
CCTGGTCTTCCTGCAGTACTGCATCATGGCCAACTTCTTCTGGCTGCTGG
TGGAGGGGCTCTACCTCCACACCCTCCTGGTGGCCATGCTCCCCCCTAGA
AGGTGCTTCCTGGCCTACCTCCTGATCGGATGGGGCCTCCCCACCGTCTG
CATCGGTGCATGGACTGCGGCCAGGCTCTACTTAGAAGACACCGGTTGCT
GGGATACAAACGACCACAGTGTGCCCTGGTGGGTCATACGAATACCGATT
TTAATTTCCATCATCGTCAATTTTGTCCTTTTCATTAGTATTATACGAAT
TTTGCTGCAGAAGTTAACATCCCCAGATGTCGGCGGCAACGACCAGTCTC
AGTACAAGAGGCTGGCCAAGTCCACGCTCCTGCTTATCCCGCTGTTCGGC
GTCCACTACATGGTGTTTGCCGTGTTTCCCATCAGCATCTCCTCCAAATA
CCAGATACTGTTTGAGCTGTGCCTCGGGTCGTTCCAGGGCCTGGTGGTGG
CCGTCCTCTACTGTTTCCTGAACAGTGAGGTGCAGTGCGAGCTGAAGCGA
AAATGGCGAAGCCGGTGCCCGACCCCGTCCGCGAGCCGGGATTACAGGGT
CTGCGGTTCCTCCTTCTCCCGCAACGGCTCGGAGGGCGCCCTGCAGTTCC
ACCGCGGCTCCCGCGCCCAGTCCTTCCTGCAAACGGAGACCTCGGTCATC
TAGCCCCACCCCTGCCTGTCGGACGCGGCGGGAGGCCCACGGTTCGGGGC
TTCTGCGGGGCTGAGACGCCGGCTTCCTCCTTCCAGATGCCCGAGCACCG
TGTCGGGCAGGTCAGCGCGGTCCTGACTCCGTCAAGCTGGTTGTCCACTA
AACCCCATACCTGGAATTGGAGTCGTGTTGTCATTGACTCGATTTAAACT
CCAGCATTTAGATAATCTTGTGCAAAATGTGTTTCAGCCGTATAGTGGAT
CCACTTTTTTTTTTTTTTTTTTTTGAGACGGAGTCTTGCTCTGTCGCCCA
GGCTGGAGTGCAGTGGCCTGATCTCTGCTCCCTGCAAGCTCCGCCTCCCG
GGTTCACGCCATTCTCCTGCCTCAGCCTCCCATAGCTGGGACTACAGGCG
CCCGCCAACACGCCTGGCTAATTTTTTGTATTTTTAGTAGAGACAGGGTT
TCACCATGTTAGCCAGGATGGTCTCGATCTCCTGACCTCGTGATGGGCCC
GCCTCGGCCTCCCAAAGTGCTGGGATTAAGGCGTGAGCCACTGCGCCCGG
CCCAAGAGAATAGGGGAGCCAAGGAGGAAATGTGGAAACGCAGTTGTGTG
GCCCAGCACGAGCCTGGGCGACCACCGGGTGACATCCGTCCCACATCAGG
GCGGCCTCCCAGGTCCCATAAGGGTAGCCCCCTCATCTGCAGGACAGAGG
GAAGCCAGTCAGGGCCCCCCCGACGTTAGGACCAGGAGAAATCAACAGGA
GGGCAGCCCGTCCTCTCTCTTGGGGCGCCCACCCGGCCCGGCTGAGCCCT
GCCCCACCCAACTCCACAGGGCTGTTTTGCCTCCCCACGGAAGGCGGGCT
GAGGAGACAACCAGATCAGGAGAGCAAGGTCATGAAGGAGGGGACCTCTC
CACACAGGTGTTCCGTGGGACCCTCAGCAGCTCTGGCTCTGCCTCAGGAG
GTCACCTGCCGCCCTGTGGGAGCCGCAGAGCCTGACGCTCAGCCCCAGGC
CAGCTGCGGCCAGGCCTGCGGGCCCCTGGTGATGGGGTTACGTGGGGTGC
GGGATACAGCTGAGTGGGAACCGGAAACCTATTCTCTTTTTAACAAAAAT
AATCTTAGGATAAGAATTATTTTAACAACATATAAAACTGTTTCAAGCCC
TCCTCCCCAGAGCTGGCGCTCAGCAGCCCTAGCGGCTGCTCCTTCAGGCG
AAGGGTGGTTTGCAGATGTGGGGAGGGTGTCTGGGGACGTTGCTGAGCTG
GCTGCAGAAGGGTGGGGATATCAGGGCACAGTCTCCATGTGTGTGCCAAG
CCCTGGCCCCCACAGCGCTCGATGGACCTCAGCAAGCTGCCCAGCCCTGG
CCCAGGTGCCCCGACTGTGGGACTCAGTTGTTCTGAGCACATTTGACTCC
ACTTTTCCTTTAAAAATGAATGTCTTGTTCCTGTGCATTGGTGGCATCAC
AGACCCCAGCTGGGGCGCGATGTCAAAGGTCGGGACAGCTGTGCCGGGAG
GCAGCCACAGGGAAGCTCACACATCCTGTCAGTGTCACCTTGGTTTGCAA
AACCCATATCCCCGGTAAAATGAGGCCGGACAGAGGGGCTGTTAGGACAG
CAAAGCAGCAGTGTCCAGAGACCCCTCAATCCCCAAAGGTCCGCACCCTG
TCCTGCACACCCTGGGCCACGCCGGCCACACCCCTCTGCTGCAACAAGCT
CATCCCTGGACTTCTGGGAGAATGAACCCGAGGTTGGTTTGGGGAGACAG
GTGAGGCGGTTGGATCTACAGAACAACCCACCATTTCTGGGGGCCGCAGA
GGATCCATCACAGACGGATACTGGGGAGTAAACGGCCCAGGCCAGGTGCC
CAGGAAAGGACGGCTGAGCATGTGGAGCGAGAGGGAGGCAGGTGGACGCT
GCAGACCCCAGGTTCAGTGCGGCCCCTCGGCTGTTCCTCCCCTGTAGGGT
TTGGACAGACCCACCCCCAGCCTTGCCCAGCTTTCAAAGGACAAAAGGGA
GCATCCCCCACCTACTCTCAGGTTTTTGAGGAAACAAAGATTTGTGGTAA
CTGAAGGTGTTGGGTCAGTGGCCAGGTGCCGACACTGAGCTGTGACCCAG
AGGGGACGCTGAGGAAGTGGGCGTGAGTGGACATGTCAGGTGGTTACCAG
GCACTGGTTGTTGATGGTCGGTGGTTGGGTGTGGGCAGTCATCAGTCATC
AGGTGTGCTCAGGGGACAATCTCCCCTCAACCGCACATGTGCCACTGTTC
AGCGGAGCTGACTGGTTTCTCCTGGTAGAGGGCCGGCTGTATCCTGACAG
ATGCCTGGTGAGCAGGGGAAGCAGGACCCAGTGGTCAACAGGTGTCTTTA
ACTGTCATTGTGTGTGGAATGTCGCAGACTCCTCCACGTGGCGGGAATGA
GCTGTGTAAATACTTCAATAAAGCCTGACTTCACATCTGCAAAAAAAAAA
AAAAAAAA.

TABLE 12C

Vasoactive intestinal peptide receptor 2
(X95097/NM_003382.2) protein sequence
(SEQ ID NO:29)

MRTLLPPALLTCWLLLAPVNSIHPECRFHLEIQEEETKCAELLRSQTEKH
KACSGVWDNITCWRPANVGETVTVPCPKVFSNFYSKAGNISKNCTSDGWS
ETFPDFVDACGYSDPEDESKITFYILVKAIYTLGYSVSLMSLATGSIILC
LFRKLHCTRNYIHLNLFLSFILRAISVLVKDDVLYSSSGTLHCPDQPSSW
VGCKLSLVFLQYCIMANFFWLLVEGLYLHTLLVAMLPPRRCFLAYLLIGW
GLPTVCIGAWTAARLYLEDTGCWDTNDHSVPWWVIRIPILISIIVNFVLF
ISIIRILLQKLTSPDVGGNDQSQYKRLAKSTLLLIPLFGVHYMVFAVFPI
SISSKYQILFELCLGSFQGLVVAVLYCFLNSEVQCELKRKWRSRCPTPSA
SRDYRVCGSSFSRNGSEGALQFHRGSRAQSFLQTETSVI.

TABLE 12D

Vasoactive intestinal peptide receptor 2
(L36566.1) nucleotide sequence (SEQ ID NO:30)
CGGGACGAGGGGGCGGCCCCCGCGCTCGGGGCGCTCGGCTACAGCTGCGG
GGCCCGAGGTCTCCGCGCACTCGCTCCCGGCCCATGCTGGAGGCGGCGGA
ACCCGGGGGACCTAGGACGGAGGCGGCGGGCGCTGGGCGGCCCCCGGCAC
GCTGAGCTCGGGATGCGGACGCTGCTGCCTCCCGCGCTGCTGACCTGCTG
GCTGCTCGCCCCCGTGAACAGCATTCACCCAGAATGCCGATTTCATCTGG
AAATACAGGAGGAAGAAACAAAATGTACAGAGCTTCTGAGGTCTCAAACA
GAAAAACACAAAGCCTGCAGTGGCGTCTGGGACAACATCACGTGCTGGCG
GCCTGCCAATGTGGGAGAGACCGTCACGGTGCCCTGCCCAAAAGTCTTCA
GCAATTTTTACAGCAAAGCAGGAAACATAAGCAAAAACTGTACGAGTGAC
GGATGGTCAGAGACGTTCCCAGATTTCGTCGATGCCTGTGGCTACAGCGA
CCCGGAGGATGAGAGCAAGATCACGTTTTATATTCTGGTGAAGGCCATTT
ATACCCTGGGCTACAGTGTCTCTCTGATGTCTCTTGCAACAGGAAGCATA
ATTCTGTGCCTCTTCAGGAAGCTGCACTGCACCAGGAATTACATCCACCT
GAACCTGTTCCTGTCCTTCATCCTGAGAGCCATCTCAGTGCTGGTCAAGG
ACGACGTTCTCTACTCCAGCTCTGGCACGTTGCACTGCCCTGACCAGCCA
TCCTCCTGGGTGGGCTGCAAGCTGAGCCTGGTCTTCCTGCAGTACTGCAT
CATGGCCAACTTCTTCTGGCTGCTGGTGGAGGGGCTCTACCTCCACACCC
TCCTGGTGGCCATGCTCCCCCCTAGAAGGTGCTTCCTGGCCTACCTCCTG
ATCGGATGGGGCCTCCCCACCGTCTGCATCGGTGCATGGACTGCGGCCAG
GCTCTACTTAGAAGACACCGGTTGCTGGGATACAAACGACCACAGTGTGC
CCTGGTGGGTCATACGAATACCGATTTTAATTTCCATCATCGTCAATTTT
GTCCTTTTCATTAGTATTATACGAATTTTGCTGCAGAAGTTAACATCCCC
AGATGTCGGCGGCAACGACCAGTCTCAGTACAAGAGGCTGGCCAAGTCCA
CGCTCCTGCTTATCCCGCTGTTCGGCGTCCACTACATGGTGTTTGCCGTG
TTTCCCATCAGCATCTCCTCCAAATACCAGATACTGTTTGAGCTGTGCCT
CGGGTCGTTCCAGGGCCTGGTGGTGGCCGTCCTCTACTGTTTCCTGAACA
GTGAGGTGCAGTGCGAGCTGAAGCGAAAATGGCGAAGCCGGTGCCCGACC
CCGTCCGCGAGCCGGGATTACAGGGTCTGCGGTTCCTCCTTCTCCCACAA
CGGCTCGGAGGGCGCCCTGCAGTTCCACCGCGCGTCCCGAGCCCAGTCCT
TCCTGCAAACGGAGACCTCGGTCATCTAGCCCCACCCCTGCCTGTCGGAC
GCGGCGGGAGGCCCACGGTTCGGGGCTTCTGCGGGGCTGAGACGCCGGCT
TCCTCCTTCCAGATGCCCGAGCACCGTGTCGGGCAGGTCAGCGCGGTCCT
GACTCCGTCAAGCTGGTTGTCCACTAAACCCCATACCTGG.

TABLE 12E

Vasoactive intestinal peptide receptor 2
(L36566.1) protein sequence (SEQ ID NO:31)
MRTLLPPALLTCWLLAPVNSIHPECRFHLEIQEEETKCTELLRSQTEKHK
ACSGVWDNITCWRPANVGETVTVPCPKVFSNFYSKAGNISKNCTSDGWSE
TFPDFVDACGYSDPEDESKITFYILVKAIYTLGYSVSLMSLATGSIILCL
FRKLHCTRNYIHLNLFLSFILRAISVLVKDDVLYSSSGTLHCPDQPSSWV
GCKLSLVFLQYCIMANFFWLLVEGLYLHTLLVAMLPPRRCFLAYLLIGWG
LPTVCIGAWTAARLYLEDTGCWDTNDHSVPWWVIRIPILISIIVNFVLFI
SIIRILLQKLTSPDVGGNDQSQYKRLAKSTLLLIPLFGVHYMVFAVFPIS
ISSKYQILFELCLGSFQGLVVAVLYCFLNSEVQCELKRKWRSRCPTPSAS
RDYRVCGSSFSHNGSEGALQFHRASRAQSFLQTETSVI.

TSC13: Pancreatic Lipase-Related Protein 3.

TABLE 13A

Pancreatic lipase-related protein 3 (AL833418.1)
nucleotide sequence (SEQ ID NO:32)
GGGTGGGGGGAATAACATGTTCTTTTAAACGCAGAGTTTAAACATTGAGT
TGCATCATTGTGAGGAAAACCACTTAGTATTTTTAGTGAGGTGACTTTAC
AAGTAAAGATCTTCAAGAAGATTTTTATGTGATTTAAAAAATCAGCTTAG
ATGCTTGGAATTTGGATTGTTGCATTCTTGTTCTTTGGCACATCAAGAGG
AAAAGAAGTTTGCTATGAAAGGTTAGGGTGTTTCAAAGATGGTTTACCAT
GGACCAGGACTTTCTCAACAGAGTTGGTAGGTTTACCCTGGTCTCCAGAG
AAGATAAACACTCGTTTCCTGCTCTACACTATACACAATCCCAATGCCTA
TCAGGAGATCAGTGCGGTTAATTCTTCAACTATCCAAGCCTCATATTTTG
GAACAGACAAGATCACCCGTATCAACATAGCTGGATGGAAAACAGATGGC
AAATGGCAGAGAGACATGTGCAATGTGTTGCTACAGCTGGAAGATATAAA
TTGCATTAATTTAGATTGGATCAACGGTTCACGGGAATACATCCATGCTG
TAAACAATCTCCGTGTTGTTGGTGCTGAGGTGGCTTATTTTATTGATGTT
CTCATGAAAAAATTTGAATATTCCCCTTCTAAAGTGCACTTGATTGGCCA
CAGCTTGGGAGCACACCTGGCTGGGGAAGCTGGGTCAAGGATACCAGGCC
TTGGAAGAATAACTGGGTTGGACCCAGCTGGGCCATTTTTCCACAACACT
CCAAAGGAAGTCAGGCTAGACCCCTCGGATGCCAACTTTGTTGACGTTAT
TCATACAAATGCAGCTCGCATCCTCTTTGAGCTTGGTGTTGGAACCATTG
ATGCTTGTGGTCATCTTGACTTTTACCCAAATGGAGGGAAGCACATGCCA
GGATGTGAAGACTTAATTACACCTTTACTGAAATTTAACTTCAATGCTTA
CAAAAAAGAAATGGCTTCCTTCTTTGACTGTAACCATGCCCGAAGTTATC
AATTTTATGCTGAAAGCATTCTTAATCCTGATGCATTTATTGCTTATCCT
TGTAGATCCTACACATCTTTTAAAGCAGGAAATTGCTTCTTTTGTTCCAA
AGAAGGTTGCCCAACAATGGGTCATTTTGCTGATAGATTTCACTTCAAAA
ATATGAAGACTAATGGATCACATTATTTTTTAAACACAGGGTCCCTTTCC
CCATTTGCCCGTTGGAGGCACAAATTGTCTGTTAAACTCAGTGGAAGCGA
AGTCACTCAAGGAACTGTCTTTCTTCGTGTAGGCGGGGCAATTGGGAAAA
CTGGGGAGTTTGCCATTGTCAGTGGAAAACTTGAGCCAGGCATGACTTAC
ACAAAATTAATCGATGCAGATGTTAACGTTGGAAACATTACAAGTGTTCA
GTTCATCTGGAAAAAACATTTGTTTGAAGATTCTCAGAATAAGTTGGGAG
CAGAAATGGTGATAAATACATCTGGGAAATATGGATATAAATCTACCTAC
TGTAGCCAAGACATTATGGGACCTAATATTCTCCAGAACCTGAAACCATG
CTAATCTCAGATACAGTCTTGATGGATTTCTTTAGTAGGAGCAATGAAGA
AAAGTGTCTCCTTCCACCTGGCATCCAGACCAAATTTGACCCTTGTAAAT
GACTTAGTCATTTACAGGGGTCTTACTCAGAGTCAAGTACGGGTTTGCTT
TTTTTCTGTGTAGAATGTTCATCTAACTGCACCTTAAAAACACACTGAAC
CCTGGGACAAAAGATAATTACTATGATCTGTAGGAATCTGGATATCATTG
ACAAAATAGAGCTGTTTTGGAATTTTCCTGAATAAGAGGAGGTGATGCAA
ATGTATGTTGAGTGTATAAACTCACTGGACAAAAGTAAGCCTCTGGCTTG
CTGAGTTTTTGAAGTATATTTTCAGGTATAATAATCATTGTTCTAAAATT
ATATAAAACTATTTGTTATGTTGTTAAATCTTGCTGAGACAAATTATGAC
TATAGTGCATGATATATAGTAGATTATAACCTTGTGGGTTGATGTGTCTA
TCTAGTAATAATAAAAACTAATGAGATGGCACTAGTATTTCCAAGGTGTT
CCTTGGTGTTCAGGGTGTGCACAAGAGAGATTTTGGAGCTTATCTGTTAT
GTGTTCATCAGTTAGCAATGGGACCTGAAGTTCAACAACCCAGGGTATAG
CCCCCTTCCTCCAAAGTCCCTGCCACAGGAGAATTACTCCTCTCTCTGGG
TCTTGAATGCTCTATGGTGAATTTGTATTTAGCCTCAAGGCAGCATTTCA
TTTGTAAAGCACTTGGGTAACCCTTTGTTCTTGCAATAACAATATTATAA
TATTTAAAAAAAAAAAAAAAAAAA.

TABLE 13B

Pancreatic lipase-related protein 3 protein
sequence (SEQ ID NO:33)

MLGIWIVAFLFFGTSRGKEVCYERLGCFKDGLPWTRTFSTELVGLPWSPE
KINTRFLLYTIHNPNAYQEISAVNSSTIQASYFGTDKITRINIAGWKTDG
KWQRDMCNVLLQLEDINCINLDWINGSREYIHAVNNLRVVGAEVAYFIDV
LMKKFEYSPSKVHLIGHSLGAHLAGEAGSRIPGLGRITGLDPAGPFFHNT
PKEVRLDPSDANFVDVIHTNAARILFELGVGTIDACGHLDFYPNGGKHMP
GCEDLITPLLKFNFNAYKKEMASFFDCNHARSYQFYAESILNPDAFIAYP
CRSYTSFKAGNCFFCSKEGCPTMGHFADRFHFKNMKTNGSHYFLNTGSLS
PFARWRHKLSVKLSGSEVTQGTVFLRVGGAIGKTGEFAIVSGKLEPGMTY
TKLIDADVNVGNITSVQFIWKKHLFEDSQNKLGAEMVINTSGKYGYKSTY
CSQDIMGPNILQNLKPC.

TSC14: Polycystic Kidney Disease 1-like 2.

AW082870 does not possess a reading frame beyond 50 amino acids.

TABLE 14A

Polycystic kidney disease 1-like 2 (AW082870)
nucleotide sequence (SEQ ID NO:34)

TTTTTCCATGTAATATTTGTTTTATTTATAATAAGAGGAAATACATTTGA
ACAAAGAAGCTCTCATAGTATTGGCAATTTTACATATATCTCTGTTATTG
TAATTTTTTTTACTTGCTGGGCTTGGTAATTCTTCAATGGACATGAAAGC
TATGACCTAGAGAGACTATAGAGTCGCTGGTAAGCGTACGCCCGAGGCCC
TGGGCGTCCCCACTGGTAGATGGTGGCGTGTGGACGAACAGCTTAGTCCT
TGGGCAAAGCTTGTGCTGGTCGAGAGTGGCGAGTCTGGGACAGAGACCCA
GGCTGCTCCCTGCTGCTTCCAGGCTCCTCTCTCTTAGACTTAATGCCCAG
GAAACTGAGTATTTTCATCAGCAGCAAATCTACGATCTCCCCTTCCTCCG
ACAGCTGCAAGAGAAAGAACCAGGCAATGCCCATAGAACCATCTTCT.

TSC15: Attractin-Like 1.

AW151108 does not possess a reading frame beyond 50 amino acids.

TABLE 15A

Attractin-like 1 (AW151108) nucleotide sequence
(SEQ ID NO:35)

TTTTCTAAGAATTTGTCTTATTTTTAATGCATGGAAAATAGCAAAATTAT
CATGCCAACATGAGGAATATATACTATAATTCATAAATGCCTAATTATCA
AAATAATGACATAGTCATGGTTAGATGCAACCTAGAAATCTTATATAAGA
TGCAACTACATATTGTATGATCATTCCTCTTATATATGACATTCAATCCT
CATCAAATTCAGCTATGTATAAATGGCATTATGAAATAAACACTTAATAT
CACAATAGGGTCATAGTCTGCTACTGTACAACCATGGCATGCAAGTAACT
ATGCATTAGCTGTAAACAGTAAAGTGTCATAACCTTCCAGAAATCCAAAG
AATGTGAAAAGTACATATATAGTACTAAACATCAATTGTATTTAAAGGAC
CTTCATATTTAACAAAGCTATATCATATACAGCAGCTTTGGAGATTTCTG
TCACTGTTATACATATCTTGTCACCCTGAAGTGAGGAAACTGCAATTCCA
AACTATATCTGTTAATGCTACTG.

TSC16: Solute Carrier Family 2 (Facilitated Glucose Transporter), Member 12.

AI675682 does not possess a reading frame beyond 50 amino acids.

TABLE 16A

Solute carrier family 2 (facilitated glucose
transporter), member 12 (AI675682) nucleotide
sequence (SEQ ID NO:36)

TTTTTTTTTTTTTTTTTTTTCCTTTTTTTTTAAAAAAAGGGGTTTATTTC
CTTTTTTTTAAGATTCAGTAGGATAGCCAAATTCATAGAGAATAAAATTA
CATGAAAGAGTTACAAGCTCACTGTTTTAAAGACTTGACATTTTTCATTT
AGTTTTAATTAACAGTAATAAGACACCTCCTGTTTTTCAATGTTCACCAA
AAAAAGAAACATAGAATAGGGGGAAAACATGCTTATATAGCCAAGGTACA
GATCCAGATGATGTAACCTTTTTAGTATTCGCATGACTTGAAAACTGGGC
AGATCAATAGATAATCGAAGTGCTTTATCTGAAGGGAGAGGGTAAAGACA
GTGTGACCAGGTTTGTTTTCAGGGCTGCCGAATGAGCCTCACCTAACAGT
GTCCATGGGTAATTCGCTAACCTTAACAAAGATGGGAAGA.

TSC17: Protease Inhibitor 15.

TABLE 17A

Protease inhibitor 15 (NM_015886.1) nucleotide
sequence (SEQ ID NO:37)

CAAAGTAAACTCGGTGGCCTCTTCTTCTCCACCCCTCAAAATGATAGCAA
TCTCTGCCGTCAGCAGTGCACTCCTGTTCTCCCTTCTCTGTGAAGCAAGT
ACCGTCGTCCTACTCAATTCCACTGACTCATCCCCGCCAACCAATAATTT
CACTGATATTGAAGCAGCTCTGAAAGCACAATTAGATTCAGCGGATATCC
CCAAAGCCAGGCGGAAGCGCTACATTTCGCAGAATGACATGATCGCCATT
CTTGATTATCATAATCAAGTTCGGGGCAAAGTGTTCCCACCGGCAGCAAA
TATGGAATATATGGTTTGGGATGAAAATCTTGCAAAATCGGCAGAGGCTT
GGGCGGCTACTTGCATTTGGGACCATGGACCTTCTTACTTACTGAGATTT
TTGGGCCAAAATCTATCTGTACGCACTGGAAGATATCGCTCTATTCTCCA
GTTGGTCAAGCCATGGTATGATGAAGTGAAAGATTATGCTTTTCCATATC
CCCAGGATTGCAACCCCAGATGTCCTATGAGATGTTTTGGTCCCATGTGC
ACACATTATACGCAGATGGTTTGGGCCACTTCCAATCGGATAGGATGCGC
AATTCATACTTGCCAAAACATGAATGTTTGGGGATCTGTGTGGCGACGTG
CAGTTTACTTGGTATGCAACTATGCCCCAAAGGGCAATTGGATTGGAGAA
GCACCATATAAAGTAGGGGTACCATGTTCATCTTGTCCTCCAAGTTATGG
GGGATCTTGTACTGACAATCTGTGTTTTCCAGGAGTTACGTCAAACTACC
TGTACTGGTTTAAATAAGTTTACCTTTTCCTCCAGGAAATATAATGATTT
CTGGGAACATGGGCATGTATATATATATATGGAGAGAGAATTTTGCACAT
ATTATACATATTTTGTGCTAATCTTGTTTTCCTCTTAGTATTCCTTTGTA
TAAATTAGTGTTTGTCTAGCATGTTTGTTTAATCCTTTGAAATATTTGAA
ACATCAATTTCTATTTTCTGACCTCTAAGCCTAAATTAAGATATTGTATA
TGTAATGATGACATAGTTGATGCATCCAATCCTAAAACTTACATTCCAAA
GGAATTATATCATTATGTTCCTAAGGAGTAAATATATATTTGACCTGTAA
GTGTGTGTATGTATACATATACATATGTATGTGTATGGATTTATATATGC
ACACAAACATATAATATGTGATGTAACATGTAGATGATAATATGATTCAG
TAGTCAACTTGAGGGAAATTTTTAAAAAACTATTCTCAATTATATACGAG
GTGATGGGACTTCTTAACACACATTTCTATAATACCCATGAAATGATAAT
TTGTAAAATAACACTTAGTGATATCTGGAAATAATAATTCAATTAAGCAA
CCACGAATTTCACCCTGGAGATATTTTTTCTTATTTGAGTCCACCAAAGG
ATAATGCCAACTTATATAAGTTCTCAAATCATGCCTTCCGCTTAGTCTCA
TTTTATTCATTCAGTCGTCATGAGTTGAGTGCTTACTACATGCAAGGCAC
TCTGCTAGTTATATTCTAATAATGCAGAGATAATTAGACATGGTTCCCGC
CCTCAAGAAGCTCACAAAAGTATTCAGGAAATAATGCAGACTAGTGATTT
TGCTATAAAATTATTTTTGAAGGAAGCAGACACAGCAGTATTTACCTGTA
GGTGGAGCAAGTAATAAGCCATGCTGTGCAATATATACATAAAGCTTCTG
CTTCTCATGGGAATTTAGTTACAGTGCTTGGAATGAGAAGGGGAAGGAAA
GAATTAACAAATGCCAAGATTTCTGGAGCAGATTGTACAGCTGTGACTTT
GGAAAACAGAAAGTAAGACCCTCAGAAAACCAATGAAGTCTAAGAGAAAT
AAAATTTAGTGGACAGGTATGAAAAGTGTAATTGCGCCTAACTACCAGAT
GGAGAGCTTCAGAATGGGCTATCCTTAGAGTCTAGTACATCTTGAGGCCT
CTCAGCAGGAGACAAAGGATTCCAAAAAGAGATGTGGAGGTGCTGAGGGC
ACCTCTATCTCTTTGTTGTTTAGTCTGTCATTGAATCAATCTACTTATCA
TCTTGGGTCTTTGAGTATTGTATGAAAGATCCTTCGTGCACACCACACAC
AGTCATGATTTTGTTAAAGTAGCCTTCTTCAGATGCTTTTCTAAGGGCTA
GTTACCAACTTTATTTCTGTGTTTCTGTAGAAGAAACATTTTCAGTTCTT
CATTGAGTTTGATTATGGAAATCCATTCAAAGTCACTATGAAAATTTTAC
TCATGTAGTTTGGAAATGCAACATTTTCCTATCATGAAATCTCTTTCAGA
GAGGAGAATACAACATCTTAGTCCAGACATTTAACATACTGCATTTCAAG
TACATGTGTGTGTGTTTTATTCAGGTTGTGTAATGCTCCCGTAGAATTAT
AGAACAATTAAATATGGTTAGTTCCAGAGTGCAAATTACAGAAGGAAGCT
ACTTGTTTAAAATTCCATACACGTTTGCAGTTTCTTGTACACATTTGGAT
ACTTTGAAAGATGACAGATTGTTAAATCCATTCAATGGTAAAGAAACTCA
CCATCTGGAGATTGAGTCTACTTGTTAATGAATGACTAGCCCAATTATCC
TTATAAATTGAATATGGTGACCAAATGCTTTGATATCATACTACTCTGCC
TTTGTGGGCACATATGTAGACACTACTAAAAATAAATATTTTTGGAGATT
AAAATGGAGAATAGAAGTAATTACATTATTTAGGTCTTAATCCAACTTTT
TTCTAATATATCTAAACAATTGAAAGGGAAGCTTATTCATGGAATATTGG
CTTGATTTATCTAGAAAGTTTTTCCTTCTTCAATTTTACTATATTCATTC
TACAGGAACAGCAATAAGTACTATTAAACAGAAGATGGCTACACTAAGTT
CCAATTTTGTTGCTGAATTGCTTCTGTGAGTTCACTTTCAGTTCTAAGGA
AGAATAATATTTGCTACATATTTCACAGGGGTTCTTATGAAGGTAAATTT
ACCAGATTAATAAAAATTTATGAATATTAAAATTATCATTAATAATATAA
AACACTTATTTGAGATTAAATTAAATTTTTCATGAGCCCCTCTTTGGCAG
GAACTCTGTTTAATTCTTTGTATTTATCCCAGCTTCTTAAATGGTGGCTG
TAACATAATAAATGTTTAATAAATGCTTATATGAATGGATTTTTAGAATT
AACTAAGAAGCCAAAAATGGCAACAATTTACAGAAATCCCACCTTTCCAT
GCTTAAGACAAAAATGTCTTAAATATAAAGCTGTGATTATATCAAAAATC
CAGATAAATCATCAAATATATCAGATTAAGACCAGGGTTTACACACTTAG
GCAATAGTCCTTTCCAAACCATGACAAAAACTACAAGTTTATTTATAATT
TAACAACTCAGCTGAAAATATAACGGGTATATTTGTTATTCTAACTCTAT
TTTTTAAAGTTAATAATATAAAGTGGCCATGTAAAATATTTTATTTTCCA
GGCTAAAGCAAATGAAAGTTTGCTGGTATCAACACAGCCTGCCATATTTT
TCACAGCATGCAACAATGGTGCTAGGATAGCTATTTCTTACTGTAATTGC
CAGAGGCAGAAATGGTCTGGGTATAAGCTATTTCATAAAAGCAGCTTTAA
ATTGTCAGTATTAAGGTTTTCATGTGGAAAGGTGTCATTCAAAAAAAAAG
TAATTGGCATACATATTCCACACATCGATCCTCTCTGTGGTGTTAATTTT
TTTATATGACCAGTAGAAAAATTTTAATATTCTCACAATATAGGTTTTGG
GGCTTCCATATCATCAAAAGACTGAAAAATTATAATTTTAGAATTAAACT
GATGGATTTCATTATAGAATTATCTGTGAGTTGTGTAGACACAGTCTTAA
TGTTTCTGGTTATGACAGATAAGTTTGCTCAAAAAATGTGGATGAAGCCA
TTATTGTTATTATTGTTATTGCTTCTGTTCAGTTGTCTAAGTATCATCCC
TTCTGTGGCCCATCACGCAGCAGAGTTGCCCTACAAATTTCATTTGGCAG
CGCCATAACATTCATTTAAAAAGTTTATGAAAACATTCATTTGAAAGTTC
CATGCAGCTTTAGCACAGAGTTGACCAACACTGGCGTAAGTTCAATTTAC
ACAGAATATTTGAATTGAAACAATAGAAATTTTTCTCATAATATATACCT
ATGTGAACCAACTTATCTGCATAATTAAATCTAATACATATTTAAGCCAG
TTTAAGTGCTTTGTGTTGATGCCATGCTTATCAAATACATGCACAAGCTA
AACATAATTTGAATGGGTCTATGAAGGAAAAATAATGCTTAGACTTTGGT
GTAGGTTCTTCCTGTGTAGCCATATACCCAGGCTCTGCAGTATCGAAGGA
TGCAAATGTTGACATAGATGGAAGCTCTTACCTACCAAAGTGTTTAGGAA
GGATAGTTACATTTGTCTTAATTTCTAACATTATCTTTGCTTTTATGTTT
CATAAAAATTTGTCATTATTTATGCTGGTGAAACGTATAATCACATCCAA
TTATTTGAACACATGCAAAATAATTTTTTAAATTATGTTATTGTTTAAAT
TTGACTTATGGGAGATCAGTCAAAAACTTAGAAGGTTTAACACTTCACTG
ATTAATGGTGCTGAAAACACGTTACAATTACCACATATCCTTGCTATAAG
TTTTGAAGTTTCTTAGCAATTAAAGTTTTTTTATTCAGTGTGAACTGTCA
GTATCTATTCTGGTGCTAAATGTATGGTGCTAAATGAATTGTTAGTGTTG
ATGGCTTTAGTAATGCTCCTTTTATTCATTGCTAAATTTAGTGTTATCCA
TTTGATTCCTGATTCAGAAATATCAATAAAATCCTATGTTAAATTAATCT
TTACCAAAAACAGGCAAGTTAACTCTGTTGTTTTAATTCAACAGTCCAAC
ATTATTTAGGTGTTACAGAGTGTAAATATATTTCTTTGGGAGTTATTTTC
TTTTTAAAATCTTTTTATAGCTTGGCAATGTCCAAAGTCAAATATCACCT
AAACTGGTTAGATTACTTCTACAGCTAATAATATTGCAGGCACTGGCGCC
CTCTGGTGGTTATGAAGACAAATTCTTAATGGCTACTTGACCTACAGCAA
AAGCCATTTCTGTACCATAAAAATTTGTTGTGCAATATTAGAATTATCAT
ATGTTTCCTACATCTGACAGCACCTAAAATGTTTGATAATATTAACATGT
ATCTAAGAGGAAAAAAGAGTTAATATATTCTGGCACCCACTTTCCTAGTA
ATGTTTTCCATGATTTTCCAGTTCTGAGGCACTTATTAAAGTGCTTTTTT
TTTTCTGAATTAATTAGGTATTGGTAAAATATATTTTTAAATTTAGTTAG
CTTTATAAACACAATTAGAATTACAATTAATTAACAGAGGTATAATTGTC
TCACTTTCAGAAGTGATCATTTATTTTTATTTAGCACAGGTCATAAGAAA
AATATATAGAAAAATAATCAATTTCATATATAAAAGGATTATTTCTCCAC
CTTTAATTATTGGCCTATCATTTGTTAGTGTTATTTGGTCATATTATTGA
ACTAATGTATTATTCCATTCAAAGTCTTTCTAGATTTAAAAATGTATGCA
AAAGCTTAGGATTATATCATGTGTAACTATTATAGATAACATCCTAAACC
TTCAGTTTAGATATATAATTGACTGGGTGTAATCTCTTTTGTAATCTGTT
TTGACAGATTTCTTAAATTATGTTAGCATAATCAAGGAAGATTTACCTTG
AAGCACTTTCCAAATTGATACTTTCAAACTTATTTTAAAGCAGTAGAACC
TTTTCTATGAACTAAATCACATGCAAAACTCCAACCTGTAGTATACATAA
AATGGACTTACTTATTCCTCTCACCTTCTCCAGTGCCTAGGAATATTCTT
CTCTGAGCCCTAGGATTGATTCTATCACACAGAGCAACATTAATCTAAAT
GGTTTAGCTCCCTCTTTTTTCTCTAAAAACAATCAGCTAATAAAAAAAAA
ATTTGAGGGCCTAAATTATTTCAATGGTTGTTTGAAATATTCAGTTCAGT
TTGTACCTGTTAGCAGTCTTTCAGTTTGGGGGAGAATTAAATACTGTGCT
AAGCTGGTGCTTGGATACATATTACAGCATCTTGTGTTTTATTTGACAAA
CAGAATTTTGGTGCCATAATATTTTGAGAATTAGAGAAGATTGTGATGCA
TATATATAAACACTATTTTTAAAAAATATCTAAATATGTCTCACATATTT
ATATAATCCTCAAATATACTGTACCATTTTAGATATTTTTTAAACAGATT
AATTTGGAGAAGTTTTATTCATTACCTAATTCTGTGGCAAAAATGGTGCC
TCTGATGTTGTGATATAGTATTGTCAGTGTGTACATATATAAAACCTGTG
TAAACCTCTGTCCTTATGAACCATAACAAATGTAGCTTTTTAAAGTCCAT
TGTATTGTTTTTTCTTTCAATAAAAGAGTATAATTAA.

TABLE 17B

Protease inhibitor 15 protein sequence
(SEQ ID NO:38)

MIAISAVSSALLFSLLCEASTVVLLNSTDSSPPTNNFTDIEAALKAQLDS
ADIPKARRKRYISQNDMIAILDYHNQVRGKVFPPAANMEYMVWDENLAKS
AEAWAATCIWDHGPSYLLRFLGQNLSVRTGRYRSILQLVKPWYDEVKDYA
FPYPQDCNPRCPMRCFGPMCTHYTQMVWATSNRIGCAIHTCQNMVWGSVW
RRAVYLVCNYAPKGNWIGEAPYKVGVPCSSCPPSYGGSCTDNLCFPGVTS
NYLYWFK.

TSC18: Tumor Protein p53 Inducible Protein 3.

TABLE 18A

Tumor protein p53 inducible protein 3 (BC000474.1)
nucleotide sequence (SEQ ID NO:39)

AGGAGCCAGAACCACTCGGCGCCGCCTGGTGCATGGGAGGGGAGCCGGGC
CAGGACAATATGTTAGCCGTGCACTTTGACAAGCCGGGAGGACCGGAAAA
CCTCTACGTGAAGGAGGTGGCCAAGCCGAGCCCGGGGGAGGGTGAAGTCC
TCCTGAAGGTGGCGGCCAGCGCCCTGAACCGGGCGGACTTAATGCAGAGA
CAAGGCCAGTATGACCCACCTCCAGGAGCCAGCAACATTTTGGGACTTGA
GGCATCTGGACATGTGGCAGAGCTGGGGCCTGGCTGCCAGGGACACTGGA
AGATCGGGGACACAGCCATGGCTCTGCTCCCCGGTGGGGGCCAGGCTCAG
TACGTCACTGTCCCCGAAGGGCTCCTCATGCCTATCCCAGAGGGATTGAC
CCTGACCCAGGCTGCAGCCATCCCAGAGGCCTGGCTCACCGCCTTCCAGC
TGTTACATCTTGTGGGAAATGTTCAGGCTGGAGACTATGTGCTAATCCAT
GCAGGACTGAGTGGTGTGGGCACAGCTGCTATCCAACTCACCCGGATGGC
TGGAGCTATTCCTCTGGTCACAGCTGGCTCCCAGAAGAAGCTTCAAATGG
CAGAAAAGCTTGGAGCAGCTGCTGGATTCAATTACAAAAAAGAGGATTTC
TCTGAAGCAACGCTGAAATTCACCAAAGGTGCTGGAGTTAATCTTATTCT
AGACTGCATAGGCGGATCCTACTGGGAGAAGAACGTCAACTGCCTGGCTC
TTGATGGTCGATGGGTTCTCTATGGTCTGATGGGAGGAGGTGACATCAAT
GGGCCCCTGTTTTCAAAGCTACTTTTTAAGCGAGGAAGTCTGATCACCAG
TTTGCTGAGGTCTAGGGACAATAAGTACAAGCAAATGCTGGTGAATGCTT
TCACGGAGCAAATTCTGCCTCACTTCTCCACGGAGGGCCCCCAACGTCTG
CTGCCGGTTCTGGACAGAATCTACCCAGTGACCGAAATCCAGGAGGCCCA
TAAGTACATGGAGGCCAACAAGAACATAGGCAAGATCGTCCTGGAACTGC
CCCAGTGAAGGAGGATGGGGCAGGACAGGACGCGGCCACCCCAGGCCTTT
CCAGAGCAACCTGGAGAAGATTCACAATAGACAGGCCAAGAAACCCGGTG
CTTCCTCCAGAGCCGTTTAAAGCTGATATGAGGAAATAAAGAGTGAACTG
AAAAAAAAAAAAAAAAAAAAAAAAAAAAAAAAA.

TABLE 18B

Tumor protein p53 inducible protein 3 protein
sequence (SEQ ID NO:40)

MLAVHFDKPGGPENLYVKEVAKPSPGEGEVLLKVAASALNRADLMQRQGQ
YDPPPGASNILGLEASGHVAELGPGCQGHWKIGDTAMALLPGGGQAQYVT
VPEGLLMPIPEGLTLTQAAAIPEAWLTAFQLLHLVGNVQAGDYVLIHAGL
SGVGTAAIQLTRMAGAIPLVTAGSQKKLQMAEKLGAAAGFNYKKEDFSEA
TLKFTKGAGVNLILDCIGGSYWEKNVNCLALDGRWVLYGLMGGGDINGPL
FSKLLFKRGSLITSLLRSRDNKYKQMLVNAFTEQILPHFSTEGPQRLLPV
LDRIYPVTEIQEAHKYMEANKNIGKIVLELPQ.

TSC19: Astrotactin.

TABLE 19A

Astrotactin (AB006627.1) nucleotide sequence
(SEQ ID NO:41)

CCCACGCGTCCGGGCCGGGGCTCAAGATGGCTTTAGCCGGGCTCTGCGCC
CTGCTCGCCTGCTGCTGGGGGCCGGCGGCGGTGCTGGCCACGGCCGCCGG
CGACGTGGATCCATCCAAGGAGCTGGAGTGCAAGCTCAAAAGCATCACGG
TGTCGGCACTGCCCTTCCTGCGCGAGAACGACCTGAGCATCATGCACAGC
CCCTCGGCCTCGGAGCCCAAGCTCCTCTTCTCGGTGCGCAACGACTTCCC
GGGAGAAATGGTCGTGGTGGACGACCTGGAGAACACGGAGCTGCCCTACT
TCGTGCTGGAGATCTCAGGGAACACAGAGGATATCCCTTTGGTGCGCTGG
AGGCAGCAGTGGCTGGAGAATGGCACTTTGCTTTTTCACATTCATCACCA
AGATGGTGCCCCAAGCCTTCCTGGACAAGACCCCACTGAAGAACCCCAAC
ATGAGTCGGCAGAAGAGGAGCTGAGGATCCTCCACATCTCAGTCATGGGT
GGCATGATCGCTCTGCTGCTGTCCATCTTGTGCCTGGTGATGATCCTGTA
TACTCGCCGGCGCTGGTGCAAACGCCGCCGGGTCCCGCAGCCCCAGAAGA
GTGCCAGTGCTGAGGCAGCCAATGAGATTCACTACATTCCTTCTGTGCTG
ATCGGCGGGCACGGACGGGAGAGCCTGCGCAATGCCCGCGTGCAGGGCCA
CAACTCCAGTGGCACCCTGAGCATCCGGGAGACACCTATCCTGGACGGCT
ATGAGTATGACATCACTGATCTGCGCCACCATCTGCAGAGGGAGTGCATG
AACGGAGGGGAGGACTTTGCCAGCCAGGTCACGCGCACCCTCGACTCCCT
GCAGGGCTGCAATGAAAAGTCGGGGATGGACCTCACACCAGGAAGTGACA
ATGCCAAGCTGTCACTGATGAACAAGTATAAAGATAATATTATAGCCACT
AGCCCTGTGGACTCCAACCACCAGCAAGCCACCCTTCTCTCTCACACCTC
CAGCAGCCAGAGAAAGCGGATCAACAACAAAGCAAGAGCTGGTTCCGCCT
TCTTGAACCCTGAAGGGGATTCTGGCACAGAGGCAGAAAACGACCCCCAG
CTGACCTTTTACACGGATCCTTCAAGGAGCAGGAGGCGTAGTAGAGTGGG
TTCTCCCCGAAGTCCTGTGAATAAGACCACCTTGACCCTGATCAGCATCA
CCAGCTGTGTGATTGGCCTCGTGTGCTCCTCTCACGTCAACTGCCCTCTC
GTTGTCAAGATCACCCTGCATGTCCCTGAGCACCTGATTGCTGATGGGAG
CCGCTTCATCTTGCTGGAGGGGAGCCAGCTGGATGCCAGTGACTGGCTGA
ACCCTGCCCAAGTGGTTCTCTTCTCTCAGCAGAACTCCAGCGGACCCTGG
GCCATGGACCTCTGTGCCCGGCGGCTCCTGGACCCCTGTGAACACCAATG
TGACCCCGAAACTGGGGAATGCCTCTGCTATGAAGGCTACATGAAGGATC
CAGTACATAAGCACCTTTGCATTCGGAACGAATGGGGGACAAACCAGGGG
CCATGGCCTTACACAATATTTCAGCGAGGCTTTGACCTGGTTTTGGGAGA
GCAGCCCTCTGATAAAATATTTAGATTCACCTACACTCTTGGGGAGGGCA
TGTGGTTGCCCCTCAGCAAGCGCTTTGTGATTCCACCAGCCGAACTGGCC
ATCAATCCATCAGCAAAGTGCAAGACGGACATGACTGTGATGGAGGATGC
TGTGGAGGTCAGAGAGGAGCTGATGACTTCATCCTCCTTCGACAGCCTGG
AGGTTCTCTTAGATTCCTTTGGGCCGGTGCGCGACTGCAGCAAAGATAAC
GGGGGCTGCAGTAAGAATTTCCGCTGTATTTCAGATCGCAAGCTGGACTC
CACTGGTTGCGTGTGCCCATCTGGACTCAGTCCCATGAAGGACAGCTCTG
GCTGCTATGACCGCCACATCGGGGTGGACTGTTCCGACGGCTTCAACGGC
GGCTGTGAGCAGCTGTGCCTCCAGCAGATGGCGCCCTTCCCGGACGACCC
CACCTTGTATAACATCCTCATGTTCTGTGGGTGCATCGAGGACTACAAGC
TTGGTGTGGATGGACGCTCTTGCCAACTCATCACGGAGACCTGTCCAGAG
GGAAGTGACTGTGGGGAAAGCAGGGAGCTTCCCATGAACCAGACCCTCTT
TGGGGAGATGTTCTTTGGTTACAACAACCATTCCAAGGAAGTGGCTGCCG
GACAGGTGCTGAAAGGAACATTCAGGCAAAACAACTTTGCTCGTGGTTTA
GACCAGCAACTGCCAGATGGTCTTGTGGTGGCCACTGTGCCCCTGGAGAA
TCAATGCCTAGAGGAGATCTCGGAGCCCACCCCTGACCCTGACTTCCTGA
CTGGGATGGTGAACTTCAGTGAAGTGTCTGGGTACCCTGTGCTGCAGCAC
TGGAAGGTCCGGTCTGTGATGTACCACATCAAACTCAACCAAGTGGCCAT
CTCTCAGGCCCTCAGCAATGCTCTCCACTCGCTGGATGGGGCTACATCTC
GTGCAGATTTTGTGGCGCTGTTGGACCAGTTCGGCAACCATTACATCCAG
GAAGCTATCTACGGCTTTGAGGAGTCCTGTTCTATCTGGTACCCAAACAA
GCAGGTCCAGCGGCGACTCTGGCTGGAGTATGAAGACATCAGTAAAGGCA
ACTCCCCATCAGATGAGTCTGAGGAGCGGGAAAGAGACCCTAAGGTGCTG
ACATTCCCAGAATACATCACCAGCTTGTCAGACTCCGGCACCAAGCGCAT
GGCGGCTGGAGTCCGCATGGAGTGCCAGAGCAAGGGACGATGCCCCTCGT
CCTGCCCCCTGTGTCATGTGACATCCAGCCCTGACACCCCTGCTGAGCCG
GTTCTGCTGGAGGTGACCAAAGCAGCCCCCATCTATGAACTAGTGACCAA
CAACCAGACCCAGAGGCTCTTGCAGGAGGCTACCATGAGCTCTCTCTGGT
GCTCAGGGACTGGAGATGTCATCGAGGACTGGTGTCGATGTGACTCCACT
GCTTTTGGAGCTGATGGACTCCCCACCTGTGCGCCTCTCCCACAGCCTGT
GCTGAGACTCTCCACGGTTCACGAGCCCAGCAGCACTCTTGTGGTCCTGG
AGTGGGAACACTCAGAGCCACCAATCGGGGTGCAGATTGTAGATTACCTC
CTCCGTCAAGAGAAAGTCACTGACAGGATGGACCACTCCAAAGTGGAGAC
AGAAACAGTGCTGAGCTTTGTGGACGACATCATCTCTGGAGCAAAGTCTC
CTTGTGCAATGCCATCTCAGGTGCCGGACAAGCAGCTCACCACCATCTCT
CTGATCATACGATGCCTGGAACCTGACACCATTTACATGTTCACGCTGTG
GGGAGTGGACAACACAGGACGGCGCTCCAGGCCAAGCGACGTGATCGTGA
AGACCCCATGCCCCGTGGTGGATGATGTCAAGGCTCAAGAAATAGCAGAC
AAGATCTACAATCTCTTCAATGGCTACACTAGTGGGAAGGAGCAGCAGAC
CGCCTACAACACCCTCCTGGATCTGGGTTCCCCCACCTTACACCGGGTCC
TCTACCACTATAACCAGCACTATGAGAGTTTTGGGGAATTCACCTGGCGA
TGTGAGGATGAGTTAGGTCCCAGGAAAGCTGGTCTCATCCTTTCCCAGCT
TGGGGACCTCAGCAGTTGGTGCAATGGACTCCTTCAGGAACCCAAGATAA
GCTTGCGGCGCAGCTCACTCAAGTACCTGGGGTGCCGCTACAGCGAGATC
AAACCCTACGGACTTGACTGGGCGGAGCTCAGCCGGGACCTCAGGAAGAC
GTGTGAGGAGCAGACCCTGAGTATCCCCTACAACGACTATGGGGACAGCA
AAGAGATCTAGCACCATAAGGCCAGGGAGCTGCTGCCAGAATGAAGTAGG
AAAGAGGAGGGATCCATCTGGGTTGGTCTGTGGATTTTTAATATTTTTTA
ATGGAACATGAAAACCTCCACAGCAACATCGAAACCAGGGAGAAAGTGAT
CCTTGCTCCCTGCAGAACTTCTTCAGTATGATGTTCTCCATCTGCATGAT
TGGGAAATCTGCCAGCCAGTGGCTTCATGCAGTGCCATATTTCTTTAGAG
GATTACTTTGGGGTTTGCTTTGCCATTAATTTGTTCCATTCATTTTTTTT
TCCCTGAGAAGTTTACCAAAATGCTCAAGAGCTCTGCCGTGCTCCCCATG
AAAAGTCTATTAAGTAGGCACCCTGTGCTCACTCAGTTCCTAAATCCATT
GCAACTGGGAGCAGAGGTGAGGCCAGAAAGTTGTTAGGCCTGCCGCAGGC
CCCACCCTCAAGCATTCCTCAGGAAGCGTCTCACTCTGGGAGCCTTGGCC
CTGCTCACAGAGAGAGACAATAGAAAATTGAGGAAGGTGGCCCTTGTCTG
TGTCTCCTGGTTTTCTTCCTAGGCCTTGCTATCACTATTTCCATACCCGA
AAGGTGAAACCAGCTTTCATTATAGGCCCCAGTGGGCCACTTGGGTTTTG
AGATCCTTCCTTATTTTAAGCCAGGACTGGGATTAAATCTCCCTCTGTCA
GATCTCTGTCCCTTCCTCTGAACAACATGATCTTTGAGAGGGAACAAGAT
GCCATCTGTCAACTGCACCTTCAGAAAAGTCTACCTGGGAGACTAGTTAG
CAGTCCACATTCAAGAGAAGACTTGGAGTTTATTGTTTTTTAAAAAAACC
ATGCTTCCTTTGGATAGACTTCTCCAGCCTACCAATATATATCCATGTGC
CTGGATTATCTTTAACCCCCACACCTCTTACCTTGGACAGGTAAGGCTTG
GCCGATGTCTGATTGGGACCAGGAGGGGTCAACACTTTCATATCAGTGTT
ACAGTGAACTAAAGCTATTATTGATCACAAAAAACTTCTGTTCATCCCCA
CCTTGCTAAATTTGCTTGTGTTGCTAGTTTTGCAAATCGTTTCTCTGATG
ACCATAAGCAGGAGGATTCCACCATGGTCACTGCCCATCCAGTCACAGGG
ATTCTGTGTAGGGAAGCACCACTGATTGCAGTTAACATCTAGAGTGTTGT
TTCCATCCCACTGCCCAAGCATTGGCATGGTCATGAATGGTGGCCCAGCC
AACAGGAAGCCCAGCCTTTCAGAAAGAGCCTGGCACGGCCCTGTTGACTA
GCAATGGCCTTAGCTGTCCCACACAACTCAGTGGCCTGAACACACACCTT
CAGCCACCATGCCTTTGACCAGGGCTCCTCATCTGGAAACATATGAGAAA
GGTCAGCAAACAGATGCAAGACCTATAAGGCTAGTCATTGAGCTATATTG
GTTTTTTTTCTAAATAGTAGTAGTGACAGATAACATTTATTGAGTGCTTG
CTGTATGCCAGGCGCTGATGTAAGCACTTTAGGTATCATTGAATTCTCAC
AGCAACTCCTGAGGAAAGTGCTATTCTTGTCTCCATTTTAGTGTTGAGGA
AAACGAGGCAAAGAGAGGTTATACAACTTGCCTCAAATCCCTTGTGCACT
GTAACTCACACTGAGTTTCAGTGTGATTTCAGGCTGTTGGTCTCCAGGGC
ACAGGATCTTAGCTACTCTGGGATACCCAGTTCTGTTTTCAGTATCATCT
GGCACACAACTGTCCAAGCTCTCAGCCCCACAGGGAACCTGCCCAGAGAG
CTTTACCTTTCCCAAGCATCTGTGGCATGGACATGTCCTCTGTGCAGTGG
AAGGAGGAGGGGCGAAAGTACGCCCTTAGCCTTTGGAGCTAGAGCACCCT
TGGGACCCCTAGTTCCACTGCACATGGCCCTCCTCCCCACCCTCATGACT
GGGAAGGAAGCCTGTGATGAGGCTGAGATAAAGCACAGGGTGGTTTCACT
CTCCTCTCTCCTTCTTTCCAAACACTGAAGGATTTATTTCAAACTCTCTA
ATGCACCTGCCTCAGAGATTCCCCTACTTTCAAAGCAAAGATCAGCAGAA
AAATTGGCTGTCCCACCTGTGGCAAATGCTGGAGCCTCAGTTAAAGTGCC
TCAAGGGGCAAATATTTCACCATTGCCAGAGAAGATGTGACAGGCCAATC
AGACAGGGCCCAGAGCATCTCTTTGCTGCTACTGTTTTGCCATCCTTCTA
TTCATATCTGTGCAGAACACGGTGTTTTAAGCTTGAGTGAAAGGAGGGTG
AGGCTGCCGATGCCTTCCTGCCCAGAAGTGGATGATGTGGGAGTTGACAG
GCCAGGGAGAGGGTGAAGCAGGTATCAGAGTCACTCCTCTGTACCCTCTC
CTTCTGTTTTTATTTTAGGCACACTATCTTCCTCTCTCCTATCTTTCCCT
CAATCTCCCAAGTTCCTCTACCTTCTTTATCTTTGTCTTTTACTTCTTCT
TTCTGTGACCCTCCTTTTTTGGCCTCCTCTTTCCCCAAGACTTTCTTCCT
CCTGTTTCTCGTTGAGTTCTCCCCACTGAATGTGTGTATGTATGTACACA
CACACACACGTGTGCACACACAATGCACACAACTCCTATGACTGGCTCCT
ACTTACATTCAAGTTAAAAAGGCTGATATGAACAGGGCAGGGGAAAATCT
TAGGATGGTTGTACAATTGACTGGAGGATTTTTTCCCCTTGGAAGACACT
ATTGATCTCAACCTGCTGACTTTTCCTAATGCTTACCTGAAGGAACCCAT
CCTGGCTAGAAAGGGTGATGGTACTGGACCGGTATTCAACCTTGAGTTTT
CAAGCTGCCAAACAGGTCTTAAGGGAGGTGCTTATATCCCACCAACACTC
TCCCAGCTCCCATGTCCCCAAGACCTCTGGAGTTTCCTCTTGAATGTACA
TGAACCACTGTAATAGCATTAGACTTTTAATTGAGTGTGCAATCGTTTTC
CATGGAGTTTGGTCCGTTCATTATTTTTTAGTTAACTACACTTCTTGATA
TTCAAATGTTCTATTAAAAAAACTGAGTATGAAGAAAAACACTTTACTAC
TGCAGAAGGAAGAAAGAATATAATATGACCATCTTCAGGTATAACAGTGT
TGTTTAAAAGAGAATTATTGTATGATTATAAAAGATGAAATAATTAACTG
AATAATAAAACAAAGCTATTAGTAAGC.

TABLE 19B
Astrotactin protein sequence (SEQ ID NO:42)

HASGPGLKMALAGLCALLACCWGPAAVLATAAGDVDPSKELECKLKSITV
SALPFLRENDLSIMHSPSASEPKLLFSVRNDFPGEMVVVDDLENTELPYF
VLEISGNTEDIPLVRWRQQWLENGTLLFHIHHQDGAPSLPGQDPTEEPQH
ESAEEELRILHISVMGGMIALLLSILCLVMILYTRRRWCKRRRVPQPQKS
ASAEAANEIHYIPSVLIGGHGRESLRNARVQGHNSSGTLSIRETPILDGY
EYDITDLRHHLQRECMNGGEDFASQVTRTLDSLQGCNEKSGMDLTPGSDN
AKLSLMNKYKDNIIATSPVDSNHQQATLLSHTSSSQRKRINNKARAGSAF
LNPEGDSGTEAENDPQLTFYTDPSRSRRRSRVGSPRSPVNKTTLTLISIT
SCVIGLVCSSHVNCPLVVKITLHVPEHLIADGSRFILLEGSQLDASDWLN
PAQVVLFSQQNSSGPWAMDLCARRLLDPCEHQCDPETGECLCYEGYMKDP
VHKHLCIRNEWGTNQGPWPYTIFQRGFDLVLGEQPSDKIFRFTYTLGEGM
WLPLSKSFVIPPAELAINPSAKCKTDMTVMEDAVEVREELMTSSSFDSLE
VLLDSFGPVRDCSKDNGGCSKNFRCISDRKLDSTGCVCPSGLSPMKDSSG
CYDRHIGVDCSDGFNGGCEQLCLQQMAPFPDDPTLYNILMFCGCIEDYKL
GVDGRSCQLITETCPEGSDCGESRELPMNQTLFGEMFFGYNNHSKEVAAG
QVLKGTFRQNNFARGLDQQLPDGLVVATVPLENQCLEEISEPTPDPDFLT
GMVNFSEVSGYPVLQHWKVRSVMYHIKLNQVAISQALSNALHSLDGATSR
ADFVALLDQFGNHYIQEAIYGFEESCSIWYPNKQVQRRLWLEYEDISKGN
SPSDESEERERDPKVLTFPEYITSLSDSGTKRMAAGVRMECQSKGRCPSS
CPLCHVTSSPDTPAEPVLLEVTKAAPIYELVTNNQTQRLLQEATMSSLWC
SGTGDVIEDWCRCDSTAFGADGLPTCAPLPQPVLRLSTVHEPSSTLVVLE
WEHSEPPIGVQIVDYLLRQEKTDRMDHSKVETETVLSFVDDIISGAKSPC
AMPSQVPDKQLTTISLIIRCLEPDTIYMFTLWGVDNTGRRSRPSDVIVKT
PCPVVDDVKAQEIADKIYNLFNGYTSGKEQQTAYNTLLDLGSPTLHRVLY
HYNQHYESFGEFTWRCEDELGPRKAGLILSQLGDLSSWCNGLLQEPKISL
RRSSLKYLGCRYSEIKPYGLDWAELSRDLRKTCEEQTLSIPYNDYGDSKE
I.

TSC20: Glycoprotein (Transmembrane) nmb.

TABLE 20A

Glycoprotein (transmembrane) nmb (BC011595.1)
nucleotide sequence (SEQ ID NO:43)

GAGGAATTCAGAGTTAAACCTTGAGTGCCTGCGTCCGTGAGAATTCAGCA
TGGAATGTCTCTACTATTTCCTGGGATTTCTGCTCCTGGCTGCAAGATTG
CCACTTGATGCCGCCAAACGATTTCATGATGTGCTGGGCAATGAAAGACC
TTCTGCTTACATGAGGGAGCACAATCAATTAAATGGCTGGTCTTCTGATG
AAAATGACTGGAATGAAAAACTCTACCCAGTGTGGAAGCGGGGAGACATG
AGGTGGAAAAACTCCTGGAAGGGAGGCCGTGTGCAGGCGGTCCTGACCAG
TGACTCACCAGCCCTCGTGGGCTCAAATATAACATTTGCGGTGAACCTGA
TATTCCCTAGATGCCAAAAGGAAGATGCCAATGGCAACATAGTCTATGAG
AAGAACTGCAGAAATGAGGCTGGTTTATCTGCTGATCCATATGTTTACAA
CTGGACAGCATGGTCAGAGGACAGTGACGGGGAAAATGGCACCGGCCAAA
GCCATCATAACGTCTTCCCTGATGGGAAACCTTTTCCTCACCACCCCGGA
TGGAGAAGATGGAATTTCATCTACGTCTTCCACACACTTGGTTGGCTTTT
ACAAACCCCTAAGCTTCTTCTTTACCTTTCCTTAAAATTTCAACCTTCTC
TTTTCTTACTCTATAAATTGAGAATGATAACACAGAGAGTTAATAACAGT
CACCCTGCTAACTTTCCTTAGCATGAGTGAACAGTGAGAGATAAAAATGA
AATCTTGGTTAACCTTGCCAAATCTCCAGGACACCGAAGAGTTAAAAAGA
GAGAAAAACAAAAAGATTAAGCTCTTTTTCAAAAAAACAAAACCACTTAA
TTTTTTTCTACCTAAAACCATAACAAGAAAAAATGCTAACACTTATTTAT
TTGAATGGCACATGGAGACCGGGCATGTGGCTCACACTTGTAATCCCAGC
ACCTTGGAAGGCGGAGGCGGGTGGATCACCTGAAGTCAGGAGTTCAAGAC
CAGCCTGGCCAACATGGTGAAGTCCCGTCCCTACTAAAAATACAAAAATT
AGCCAGGTGTGGTGGTGCGCACCTGTAATCCCAGCTACTCAGGAGGCTGA
GGCAGGAGAATCACTTGAATCCGGGAGGTGGAGGTTGCAGTGAGAGGAGA
TTGAGCCATTGCACTCCAGCCTGGGCAACAGAGTGAGACTCCATCTCGAA
AACAAACAAACAAACAAAAAACAGAATGGCACATTGATGAGCATTCATTG
ATTGATTCTTTAGTTTTTTTATGTTCTCTAAAGAATTTTTAAGATTTAAA
GAAGCATGTGCTATTTATTTGTAGGAAATCCTCAGAAAAGGTACAAATAA
AAAATAAAAATTATCCATAATTAATACCAGAGATTATAATTGTTAATTAT
TATGGTGTTTTCTTTGCTAGTATTTAAGATCATTATTAAGATCACATACA
CATTTTTGCTTACTATCATTAGCATTTGATGATATGATTTTTTTAATTTT
TATACATTGTTTTAATGGCTGCACGATATTTCATTGTGTACAAATAAAAT
AACTACTGTTCCGCTTTGAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
.

TABLE 20B

Glycoprotein (transmembrane) nmb protein sequence
(SEQ ID NO:44)

MECLYYFLGFLLLAARLPLDAAKRFHDVLGNERPSAYMREHNQLNGWSSD
ENDWNEKLYPVWKRGDMRWKNSWKGGRVQAVLTSDSPALVGSNITFAVNL
IFPRCQKEDANGNIVYEKNCRNEAGLSADPYVYNWTAWSEDSDGENGTGQ
SHHNVFPDGKPFPHHPGWRRWNFIYVFHTLGWLLQTPKLLLYLSLKFQPS
LFLLYN.

TSC21: Contactin 1.

TABLE 21A

Contactin 1 (AW072790) nucleotide sequence
(SEQ ID NO:45)

TTTTTTTTGGGTAACATAAGACATTTATTACTTTATACTAATTTTTTCAT
TCATAAAAAGGACAAAGCACAGTCCTATACTACTCCATTGAAAAAATGAT
AAAAAATAACTAAAAAATCAATTCAATATTTATCAGTATCAAATAAAACT
ACTATCACCTTTCCTGAAATACAAAGAAACAACAGATGTATCTATACCTA
TATAAAGTTTAATTCAGAAATCTTGCGTCTTAAAGCAGATGATTATTAGT
TAGCTTGACAACAGTTTAAAACTGATGGTCCCAGTTAAATCTGTACAACT
GTATGAGAAAATGAAAAGCTTGAGTTATCAGTGTACGAGAGATTTTAAAC
TACTTTATCTCTGTCAGAAGTTCAAAACTAAACAACCTCCAAAGTCTGTT
TTCCTCTTACCTTTCAGAACCATTTCATGCAAAATCTAACCAGTTTTGCT
CGTTATTATCATATATTAGAAAATAAAAG.

TABLE 21B
Contactin 1 protein sequence (SEQ ID NO:46)

MVPVKSVQLYEKMKSLSYQCTRDFKLLYLCQKFKTKQPPKSVFLLPFRTI
SCKI.

TSC22: Neural Epidermal Growth Factor Like Like-2.

TABLE 22A

Neural epidermal growth factor like like-2
(NM_006159.1) nucleotide sequence (SEQ ID NO:47)

TTGGGAGGAGCAGTCTCTCCGCTCGTCTCCCGGAGCTTTCTCCATTGTCT
CTGCCTTTACAACAGAGGGAGACGATGGACTGAGCTGATCCGCACCATGG
AGTCTCGGGTCTTACTGAGAACATTCTGTTTGATCTTCGGTCTCGGAGCA
GTTTGGGGGCTTGGTGTGGACCCTTCCCTACAGATTGACGTCTTAACAGA
GTTAGAACTTGGGGAGTCCACGACCGGAGTGCGTCAGGTCCCGGGGCTGC
ATAATGGGACGAAAGCCTTTCTCTTTCAAGATACTCCCAGAAGCATAAAA
GCATCCACTGCTACAGCTGAACAGTTTTTTCAGAAGCTGAGAAATAAACA
TGAATTTACTATTTTGGTGACCCTAAAACAGACCCACTTAAATTCAGGAG
TTATTCTCTCAATTCACCACTTGGATCACAGGTACCTGGAACTGGAAAGT
AGTGGCCATCGGAATGAAGTCAGACTGCATTACCGCTCAGGCAGTCACCG
CCCTCACACAGAAGTGTTTCCTTACATTTTGGCTGATGACAAGTGGCACA
AGCTCTCCTTAGCCATCAGTGCTTCCCATTTGATTTTACACATTGACTGC
AATAAAATTTATGAAAGGGTAGTAGAAAAGCCCTCCACAGACTTGCCTCT
AGGCACAACATTTTGGCTAGGACAGAGAAATAATGCGCATGGATATTTTA
AGGGTATAATGCAAGATGTCCAATTACTTGTCATGCCCCAGGGATTTATT
GCTCAGTGCCCAGATCTTAATCGCACCTGTCCAACTTGCAATGACTTCCA
TGGACTTGTGCAGAAAATCATGGAGCTACAGGATATTTTAGCCAAAACAT
CAGCCAAGCTGTCTCGAGCTGAACAGCGAATGAATAGATTGGATCAGTGC
TATTGTGAAAGGACTTGCACCATGAAGGGAACCACCTACCGAGAATTTGA
GTCCTGGATAGACGGCTGTAAGAACTGCACATGCCTGAATGGAACCATCC
AGTGTGAAACTCTAATCTGCCCAAATCCTGACTGCCCACTTAAGTCGGCT
CTTGCGTATGTGGATGGCAAATGCTGTAAGGAATGCAAATCGATATGCCA
ATTTCAAGGACGAACCTACTTTGAAGGAGAAAGAAATACAGTCTATTCCT
CTTCTGGAGTATGTGTTCTCTATGAGTGCAAGGACCAGACCATGAAACTT
GTTGAGAGTTCAGGCTGTCCAGCTTTGGATTGTCCAGAGTCTCATCAGAT
AACCTTGTCTCACAGCTGTTGCAAAGTTTGTAAAGGTTATGACTTTTGTT
CTGAAAGGCATAACTGCATGGAGAATTCCATCTGCAGAAATCTGAATGAC
AGGGCTGTTTGTAGCTGTCGAGATGGTTTTAGGGCTCTTCGAGAGGATAA
TGCCTACTGTGAAGACATCGATGAGTGTGCTGAAGGGCGCCATTACTGTC
GTGAAAATACAATGTGTGTCAACACCCCGGGTTCTTTTATGTGCATCTGC
AAAACTGGATACATCAGAATTGATGATTATTCATGTACAGAACATGATGA
GTGTATCACAAATCAGCACAACTGTGATGAAAATGCTTTATGCTTCAACA
CTGTTGGAGGACACAACTGTGTTTGCAAGCCGGGCTATACAGGGAATGGA
ACGACATGCAAAGCATTTTGCAAAGATGGCTGTAGGAATGGAGGAGCCTG
TATTGCCGCTAATGTGTGTGCCTGCCCACAAGGCTTCACTGGACCCAGCT
GTGAAACGGACATTGATGAATGCTCTGATGGTTTTGTTCAATGTGACAGT
CGTGCTAATTGCATTAACCTGCCTGGATGGTACCACTGTGAGTGCAGAGA
TGGCTACCATGACAATGGGATGTTTTCACCAAGTGGAGAATCGTGTGAAG
ATATTGATGAGTGTGGGACCGGGAGGCACAGCTGTGCCAATGATACCATT
TGCTTCAATTTGGATGGCGGATATGATTGTCGATGTCCTCATGGAAAGAA
TTGCACAGGGGACTGCATCCATGATGGAAAAGTTAAGCACAATGGTCAGA
TTTGGGTGTTGGAAAATGACAGGTGCTCTGTGTGCTCATGTCAGAATGGA
TTCGTTATGTGTCGACGGATGGTCTGTGACTGTGAGAATCCCACAGTTGA
TCTTTTTTGCTGCCCTGAATGTGACCCAAGGCTTAGTAGTCAGTGCCTCC
ATCAAAATGGGGAAACTTTGTATAACAGTGGTGACACCTGGGTCCAGAAT
TGTCAACAGTGCCGCTGCTTGCAAGGGGAAGTTGATTGTTGGCCCCTGCC
TTGCCCAGATGTGGAGTGTGAATTCAGCATTCTCCCAGAGAATGAGTGCT
GCCCGCGCTGTGTCACAGACCCTTGCCAGGCTGACACCATCCGCAATGAC
ATCACCAAGACTTGCCTGGACGAAATGAATGTGGTTCGCTTCACCGGGTC
CTCTTGGATCAAACATGGCACTGAGTGTACTCTCTGCCAGTGCAAGAATG
GCCACATCTGTTGCTCAGTGGATCCACAGTGCCTTCAGGAACTGTGAAGT
TAACTGTCTCATGGGAGATTTCTGTTAAAAGAATGTTCTTTCATTAAAAG
ACCAAAAAGAAGTTAAAACTTAAATTGGGTGATTTGTGGGCAGCTAAATG
CAGCTTTGTTAATAGCTGAGTGAACTTTCAATTATGAAATTTGTGGAGCT
TGACAAAATCACAAAAGGAAAATTACTGGGGCAAAATTAGACCTCAAGTC
TGCCTCTACTGTGTCTCACATCACCATGTAGAAGAATGGGCGTACAGTAT
ATACCGTGACATCCTGAACCCTGGATAGAAAGCCTGAGCCCATTGGATCT
GTGAAAGCCTCTAGCTTCACTGGTGCAGAAAATTTTCCTCTAGATCAGAA
TCTTCAGAATCAGTTAGGTTCCTCACTGCAAGAAATAAAATGTCAGGCAG
TGAATGAATTATATTTTCAGAAGTAAAGCAAAGAAGCTATAACATGTTAT
GTACAGTACACTCTGAAAAGAAATCTGAAACAAGTTATTGTAATGATAAA
AATAATGCACAGGCATGGTTACTTAATATTTTCTAACAGGAAAAGTCATC
CCTATTTCCTTGTTTTACTGCACTTAATATTATTTGGTTGAATTTGTTCA
GTATAAGCTCGTTCTTGTGCAAAATTAAATAAATATTTCTCTTACCTT.

TABLE 22B

Neural epidermal growth factor like like-2
protein sequence (SEQ ID NO:48)

MESRVLLRTFCLIFGLGAVWGLGVDPSLQIDVLTELELGESTTGVRQVPG
LHNGTKAFLFQDTPRSIKASTATAEQFFQKLRNKHEFTILVTLKQTHLNS
GVILSIHHLDHRYLELESSGHRNEVRLHYRSGSHRPHTEVFPYILADDKW
HKLSLAISASHLILHIDCNKIYERVVEKPSTDLPLGTTFWLGQRNNAHGY
FKGIMQDVQLLVMPQGFIAQCPDLNRTCPTCNDFHGLVQKIMELQDILAK
TSAKLSRAEQRMNRLDQCYCERTCTMKGTTYREFESWIDGCKNCTCLNGT
IQCETLICPNPDCPLKSALAYVDGKCCKECKSICQFQGRTYFEGERNTVY
SSSGVCVLYECKDQTMKLVESSGCPALDCPESHQITLSHSCCKVCKGYDF
CSERHNCMENSICRNLNDRAVCSCRDGFRALREDNAYCEDIDECAEGRHY
CRENTMCVNTPGSFMCICKTGYIRIDDYSCTEHDECITNQHNCDENALCF
NTVGGHNCVCKPGYTGNGTTCKAFCKDGCRNGGACIAANVCACPQGFTGP
SCETDIDECSDGFVQCDSRANCINLPGWYHCECRDGYHDNGMFSPSGESC
EDIDECGTGRHSCANDTICFNLDGGYDCRCPHGKNCTGDCIHDGKVKHNG
QIWVLENDRCSVCSCQNGFVMCRRMVCDCENPTVDLFCCPECDPRLSSQC
LHQNGETLYNSGDTWVQNCQQCRCLQGEVDCWPLPCPDVECEFSILPENE
CCPRCVTDPCQADTIRNDITKTCLDEMNVVRFTGSSWIKHGTECTLCQCK
NGHICCSVDPQCLQEL.

TSC 23: Transmembrane Protein with EGF-like and Two Follistatin-Like Domains 1.

TABLE 23A

Transmembrane protein with EGF-like and two
follistatin-like domains 1 (BF439316) nucleotide
sequence (SEQ ID NO:49)

TTTATAGTGAAAACATTATATTATAACATGCTTTTGCAAACAAAATATTA
AAATTAATAATTTTTAACATATTCTTTAAATTCTACATGCATACTTTTGA
ATATCTAAACTACATGTTAAACAGCTGAATACATTCTACTCACACTTCAG
ATCTTTAAACACCAACAATCTATGAATATTAATCTATTACTACAGGACAA
ATTTGGATATACGTCTTGGATAAATTTTAAGCTCACTTTAAGAGCACCAA
TCATTAACAATCATTTGTGTATTTTATTCACAAACACTGATACGATTTGT
TTATTTATGTTAAAACAAACATTTTCTTTAAAAATGAATGTGTATTAAAG
TAGTTTAACTGGTAGAATAGGCTTTATTCCAATCTGTTTGTTAAACAGCC
TATTTTCACAATATCTATATCTACTTTTCATTGATCTGTTCCATCATTAC
TAACATATTTGTTCAAATTATTAGGACTATTTTTTCAAAGGGAGGAATAA
TCAAATTCCCCAGTCCATATATCTTATAAATATTTTACACCTAATACACA
CAGCTTTACAGT.

TABLE 23B

Transmembrane protein with EGF-like and two
follistatin-like domains 1 (BF439316) protein
sequence (SEQ ID NO:50)

MNINLLLQDKFGYTSWINFKLTLRAPIINNHLCILFTNTDTICLFMLKQT
FSLKMNVY.

TABLE 23C

Transmembrane protein with EGF-like and two
follistatin-like domains 1 (U19878.1) nucleotide
sequence (SEQ ID NO:51)

AAAAAAATTAAAAAAAAAAAAAAAAACAGAAAAAAAAACATAGTACATGC
CAAGATATTATTATGACAATTACAAATACAAATAAATTATGATCTTTGAC
CTCAGCATATTTATTAACTAAAAGGGAAGATAAAACAGGCACATAACTAT
AACAGGGGCACCAGTCATGGGCGCCGCAGCCGCTCAGGCGCCTCTCGGGC
TGCCTGCGGCCTCCGCTCGCCTTCTGCTGCTAGCGACGTCGGTGCTTCTG
CTCTTCGCCTTCTCTCTGCCCGGGAGCCGCGCGTCCAACCAGCCCCCGGG
TGGTGGCGGCGGCACGGGCGGGGACTGTCCCGGCGGCAAAGGCAAGAGCA
TCAACTGCTCAGAATTAAATGTGAGGGAGTCTGACGTAAGAGTTTGTGAT
GAGTCATCATGTAAATATGGAGGAGTCTGTAAAGAAGATGGAGATGGTTT
GAAATGTGCATGCCAATTTCAGTGCCATACAAATTATATTCCTGTCTGTG
GATCAAATGGGGACACTTATCAAAATGAATGCTTTCTCAGAAGGGCTGCT
TGTAAGCACCAGAAAGAGATAACAGTAATAGCAAGAGGACCATGCTACTC
TGATAATGGATCTGGATCTGGAGAAGGAGAAGAGGAAGGGTCAGGGGCAG
AAGTTCACAGAAAACACTCCAAGTGTGGACCCTGCAAATATAAAGCTGAG
TGTGATGAAGATGCAGAAAATGTTGGGTGTGTATGTAATATAGATTGCAG
TGGATACAGTTTTAATCCTGTGTGTGCTTCTGATGGGAGTTCCTATAACA
ATCCCTGTTTTGTTCGAGAAGCATCTTGTATAAAGCAAGAACAAATTGAT
ATAAGGCATCTTGGTCATTGCACAGATACAGATGACACTAGTTTGTTGGG
AAAGAAAGATGATGGACTACAATATCGACCAGATGTGAAAGATGCTAGTG
ATCAAAGAGAAGATGTTTATATTGGAAACCACATGCCTTGCCCTGAAAAC
CTCAATGGTTACTGCATCCATGGAAAATGTGAATTCATCTATCTACTCAG
AAGGGCTTCTTGTAGATGTGAATCTGGCTACACTGGACAGCACTGTGAAA
AGACAGACTTTAGTATTCTCTATGTAGTGCCAAGTAGGCAAAAGCTCACT
CATGTTCTTATTGCAGCAATTATTGGAGCTGTACAGATTGCCATCATAGT
AGCAATTGTAATGTGCATAACAAGAAAATGCCCCAAAAACAATAGAGGAC
GTCGACAGAAGCAAAACCTAGGTCATTTTACTTCAGATACGTCATCCAGA
ATGGTTTAAACTGATGACTTTTATATGTACACTGACCATGTGTATGTACA
TTTATTATGTCTTTTTTTAAAGAATGGAAATATTTATTTCAGAAGGCCTT
ATTTTTGGACATTTTATAGTGTAGTACTGTTGGCTCGATATTTGAATATT
CAGCTACGACAGTTTTGGACTGTTTAGTAGTCTTTGTTTTATGTTTTTAA
ATACAGAAATTGCTTCACAAATTTGTACCACATGGTAATTCTAAGACTTG
TTCTTTACCCATGGAATGTAATATTTTTGCAAAGATGGACTACTTCACAA
ATGGTTATAAAGTCATATCCACTTCTTCCACAATGACCACAGCAAATGAC
CCAAGCATGAACTAAAGAAGAG.

TABLE 23D

Transmembrane protein with EGF-like and two
follistatin-like domains 1 (U19878.1) protein
sequence (SEQ ID NO:52)

MGAAAAQAPLGLPAASARLLLLATSVLLLFAFSLPGSRASNQPPGGGGGT
GGDCPGGKGKSINCSELNVRESDVRVCDESSCKYGGVCKEDGDGLKCACQ
FQCHTNYIPVCGSNGDTYQNECFLRRAACKHQKEITVIARGPCYSDNGSG
SGEGEEEGSGAEVHRKHSKCGPCKYKAECDEDAENVGCVCNIDCSGYSFN
PVCASDGSSYNNPCFVREASCIKQEQIDIRHLGHCTDTDDTSLLGKKDDG
LQYRPDVKDASDQREDVYIGNHMPCPENLNGYCIHGKCEFIYLLRRASCR
CESGYTGQHCEKTDFSILYVVPSRQKLTHVLIAAIIGAVQIAIIVAIVMC
ITRKCPKNNRGRRQKQNLGHFTSDTSSRMV.

TABLE 23E

Transmembrane protein with EGF-like and two
follistatin-like domains 1 (NM_003692.1)
nucleotide sequence (SEQ ID NO:53)

AGCGGGCGGCTGCTAGGAGGCACCGAGGCAGCGGCGGGGCTCTGGGCGCG
CGGCTGGATGCCCCCGGCCTGCGGCTCCCTGCGCTTCCCGCCGTCCAGGG
GCACCAGTCATGGGCGCCGCAGCCGCTGAGGCGCCGCTCCGGCTGCCTGC
CGCGCCTCCGCTCGCCTTCTGCTGCTACACGTCGGTGCTTCTGCTCTTCG
CCTTCTCTCTGCCAGGGAGCCGCGCGTCCAACCAGCCCCCGGGTGGTGGC
GGCGGCAGCGGCGGGGACTGTCCCGGCGGCAAAGGCAAGAGCATCAACTG
CTCAGAATTAAATGTGAGGGAGTCTGACGTAAGAGTTTGTGATGAGTCAT
CATGTAAATATGGAGGAGTCTGTAAGAAGATGGAGATGGTTTGAAATGTG
CATGCCAATTTCAGTGCCATACAAATTATATTCCTGTCTGTGGATCAAAT
GGGGACACTTATCAAAATGAATGCTTTCTCAGAAGGGCTGCTTGTAAGCA
CCAGAAAGAGATAACAGTAATAGCAAGAGGACCATGCTACTCTGATAATG
GATCTGGATCTGGAGAAGGAGAAGAGGAAGGGTCAGGGGCAGAAGTTCAC
AGAAAACACTCCAAGTGTGGACCCTGCAAATATAAAGCTGAGTGTGATGA
AGATGCAGAAAATGTTGGGTGTGTATGTAATATAGATTGCAGTGGATACA
GTTTTAATCCTGTGTGTGCTTCTGATGGGAGTTCCTATAACAATCCCTGT
TTTGTTCGAGAAGCATCTTGTATAAAGCAAGAACAAATTGATATAAGGCA
TCTTGGTCATTGCACAGATACAGATGACACTAGTTTGTTGGGAAAGAAAG
ATGATGGACTACAATATCGACCAGATGTGAAAGATGCTAGTGATCAAAGA
GAAGATGTTTATATTGGAAACCACATGCCTTGCCCTGAAAACCTCAATGG
TTACTGCATCCATGGAAAATGTGAATTCATCTATTCTACTCAGAAGGCTT
CTTGTAGATGTGAATCTGGCTACACTGGACAGCACTGTGAAAAGACAGAC
TTTAGTATTCTCTATGTAGTGCCAAGTAGGCAAAAGCTCACTCATGTTCT
TATTGCAGCAATTATTGGAGCTGTACAGATTGCCATCATAGTAGCAATTG
TAATGTGCATAACAAGAAAATGCCCCAAAAACAATAGAGGACGTCGACAG
AAGCAAAACCTAGGTCATTTTACTTCAGATACGTCATCCAGAATGGTTTA
AACTGATGACTTTTATATGTACACTGACCATGTGATGTACATTTATTATG
TCTTTTTTTAAAGAATGGAAATATTTATTTCAGAGGCCTTATTTTTGGAC
ATTTTTAGTGTAGTACTGTTGGCTCGTATTTAGAATATTCAGCTACGACA
GTTTTGGACTGTTTAGTAGTCTTTGTTTTATGTTTTTAAATACAGAAATT
GCTTTCACAAATTTGTACCACATGGTAATTCTAAGACTTGTTCTTTACCC
ATGGAATGTAATATTTTTGCAAAGATGGACTACTTCACAAATGGTTATAA
AGTCATATCCACTTCTTCCACAATGACCACAGCAAATGACCAAGCATGAA
CTAAAGGTAAAGATGTTTACAGATTACTTTTCTTACAAAAAAATCTAGAA
GACACTGTGTTTAAATAGATATTTAAATGTTTTTGAGATTTAGTAACTGA
TTTTTTAGACACTGCCTATCGCATGAACTGTAAAGCTGTGTGTATTAGGT
GTAAAATATTTATAAGATATATGGACTGGGGAATTTGATTATTCCTCCCT
TTGAAAAAATAGTCCTAATAATTTGAACAAATATGTTAGTAATGATGGAA
CAGATCAATGAAAAGTAGATATAGATATTGTGAAAATAGGCTGTTTAACA
AACAGATTGGAATAAAGCCTATTCTACCAGTTAAACTACTTTAATACACA
TTCATTTTTAAAGAAAATGTTTGTTTTAACATAAATAAACAAATCGTATC
AGTGTTTGTGAATAAAATACAAAAATGATTGTTAATGATTGGTGCTCTTA
AAGTGAGCTTAAAATTTATCCAAGACGTATATCCAAATTTGTCCTGTAGT
AATAGATTAATATTCATAGATTGTTGGTGTTTAAAGATCTGAAGTGTGAG
TAGAATGTATTCAGCTGTTTAACATGTAGTTTAGATATTCAAAAGTATGC
ATGTAGAATTTAAAGAATATGTTAAAAATTATTAATCTTAATATTTTGTT
TGGAAAAGCATGTTATAATATAATGTTTTCACAAAAAAAAAAAAAAAA.

TABLE 23F

Transmembrane protein with EGF-like and two
follistatin-like domains 1 (NM_003692.1) protein
sequence (SEQ ID NO:54)

MGAAAAEAPLRLPAAPPLAFCCYTSVLLLFAFSLPGSRASNQPPGGGGGS
GGDCPGGKGKSINCSELNVRESDVRVCDESSCKYGGVCKEDGDGLKCACQ
FQCHTNYIPVCGSNGDTYQNECFLRRAACKHQKEITVIARGPCYSDNGSG
SGEGEEEGSGAEVHRKHSKCGPCKYKAECDEDAENVGCVCNIDCSGYSFN
PVCASDGSSYNNPCFVREASCIKQEQIDIRHLGHCTDTDDTSLLGKKDDG
LQYRPDVKDASDQREDVYIGNHMPCPENLNGYCIHGKCEFIYSTQKASCR
CESGYTGQHCEKTDFSILYVVPSRQKLTHVLIAAIIGAVQIAIIVAIVMC
ITRKCPKNNRGRRQKQNLGHFTSDTSSRMV.

TSC24: Peroxisome Proliferative Activated Receptor, Gamma, Coactivator 1, Alpha.

TABLE 24A

Peroxisome proliferative activated receptor,
gamma, coactivator 1, alpha (BC029800.1)
nucleotide sequence (SEQ ID NO:55)

GTTGCCTGCATGAGTGTGTGCTCTGTGTCACTGTGGATTGGAGTTGAAAA
AGCTTGACTGGCGTCATTCAGGAGCTGGATGGCGTGGGACATGTGCAACC
AGGACTCTGAGTCTGTATGGAGTGACATCGAGTGTGCTGCTCTGGTTGGT
GAAGACCAGCCTCTTTGCCCAGATCTTCCTGAACTTGATCTTTCTGAACT
AGATGTGAACGACTTGGATACAGACAGCTTTCTGGGTGGACTCAAGTGGT
GCAGTGACCAATCAGAAATAATATCCAATCAGTACAACAATGAGCCTTCA
AACATATTTGAGGTAAGGACATCCTTTGGAAACATTAATTTTTCATTGAG
TTTGGCTTGGGCCCGACTAACATGGTAATAGACCTGAATGCATAATGAGT
TCTTACTTTGCTATCATCAAAAGACTTTTCATCACAGTTACATACTTTCT
AATTTATGGAAAAACAGCATTTGGAAAACAAATGTTTTGTTTTTATTTTT
TTAAAGATTTAAAAAATAAATCAACTAGGGACTAGGAATCAACAACTGTG
AGTGAGTTAAACTGTGTTGAAATACTAAAGGGTTGTGAAAGATTAGTGAC
AAAGAAGAACAAAAGTCTAAACCTGTTTATTCCTGTCTATTTCCCACAGA
AAGAATGAGCAATAAATGGTACCTCATATAAATTAATAAATAAGAAGGCC
TTTCTTTTTAACCAAGGGGGTAGATGTCTACCTTTGTTTGCTTTACTAAT
TAGGTGAGCTCTTTTGATTATTATTATTAATTATATTTTTGGTTCATATC
TCTAATTTCTTTATATAATGGGAATTGCTAAACTTGACTAATCTACTGTA
TACTTATAAATCAGTCAAAATTCATTTACTTTTCAGTAGCAAGAATTACC
TCCGTGACTCCGGACTCTTATTATAAGCCTACCCTATAATAAAGAATGTT
AATCTATTCCTATTAAAGTGTTACTTTGAGAAAAGGAATTCTTTCCCACA
AGATCAGTACTCATTTACTTGAAATACATTATTTTTTATAGGAACAAACA
TTTAGTGAGTACTCTGGCAAGTGAATTAAACGAAGGATGGCATATCGGCT
AGTTTTCTTTATCACAACCGCCAGTGCCATCATCATCATCATCTAATGTT
TTCTGAGCACCTACTATGTGCTGGACTTTTCTTATTCATCAGCAAAGACA
TTTCTTATACTTCCATGTTATTTGGTTGAAATCTGAGTCTTAAGAAAGCC
AAGTTTAAAATATTAACTGAGTTTTGCTGGAACCCAGCATATAATACATG
CTGAATAAATGTTTGTTAAATCATGAGAGAATGATGAATATATTAATGTT
GATAACAATAATAGTAATGACAATAATGGCCAACATTTTGGTATACTCAT
GCTTAATATATGTCATCTCACTTATCCTTGAAAACAACCTATTGTTAGGT
CCTATTGTTATCATTCCTGTTTTACATATGCAGACACTGAAACTCAGAGA
GGTTATTTGTTTTCCCCAAATCACACAGTTGACAATAAAGACCTGGGATT
TGAACCCCAAATTTATCGGACTTCAAAGTTTATATCCTTGATAAAAATAG
AGAACCCATGAATTTGGGTGACAGCCATCCTTTACAAAGACATATCAAGC
TGCTTCTTTGCTCAATATATTTAAAAATAGAACAGAATCTTTCCTTTCAG
TGTTGGTTGGCACGGAGGTAGAGTTAGCTGGTGAGCCGAGACGGCTGTAG
GTTCTGCCTATGGCTGAGATTTTGTAATACCTCTGGAGATTAAGTGGGTT
TTAGAAGAATGCTTAAGGTAGTAGAGTTTGGACTGGGGAGACAGAACAAG
GTGGAGGGAACACTGAAGGTTGTGGTTAGTTCTACTTTCCCAGGCTTCTC
AGCCGGGTCTTAGAAAGTTCATGTGAGGACTTGGCCAGTTAGAACTAAAT
TGAACATTCCTCACTCAGTACACTTGTCAAGTTACAGGCCCATTCTAAGT
CATGCTGCATTAAATAAACAATGCTTTAAAAAATGTCCATCTCCATAGAC
CTCTTATCTAAAAATCACCTATTTCATATCAGGTGAATGCCATGCATTTT
GTAGAGGCTAGAGAATAACTTAGCGGTAGGGGAGATGGCATAGGAGTCAA
TGCCTCTCTGGTGGGCTACTTTAAAAAAAATACAACTTTCTCCATAACTT
TTATGTCCCTATATTTTGTGTTGTGGTATTTGTGAGAGGTACTTTGCATT
TATACCTTCAGGAGACTTGGTTCAGCATCTAGTTAATTATCTACATGTAG
GTGCTTAATATATGCCATGCCACCCTTTTTGTCTCCCTGTATCATATTCT
ATGCTAATAAAATTATTTTCAGCACTCTAAAAAAAAAAAAAAAAAAAAAA
AAAAAAAA.

TABLE 24B

Peroxisome proliferative activated receptor,
gamma, coactivator 1, alpha protein sequence
(SEQ ID NO:56)

MAWDMCNQDSESVWSDIECAALVGEDQPLCPDLPELDLSELDVNDLDTDS
FLGGLKWCSDQSEIISNQYNNEPSNIFEVRTSFGNINFSLSLAWARLTW.

TSC25: Matrix Metalloproteinase 14 (Membrane-Inserted).

TABLE 25A

Matrix metalloproteinase 14 (membrane-inserted)
(NM_004995.2) nucleotide sequence (SEQ ID NO:57)

CAGACCCCAGTTCGCCGACTAAGCAGAAGAAAGATCAAAAACCGGAAAAG
AGGAGAAGAGCAAACAGGCACTTTGAGGAACAATCCCCTTTAACTCCAAG
CCGACAGCGGTCTAGGAATTCAAGTTCAGTGCCTACCGAAGACAAAGGCG
CCCCGAGGGAGTGGCGGTGCGACCCCAGGGCGTGGGCCCGGCCGCGGAGC
CCACACTGCCCGGCTGACCCGGTGGTCTCGGACCATGTCTCCCGCCCCAA
GACCCCCCCGTTGTCTCCTGCTCCCCCTGCTCACGCTCGGCACCGCGCTC
GCCTCCCTCGGCTCGGCCCAAAGCAGCAGCTTCAGCCCCGAAGCCTGGCT
ACAGCAATATGGCTACCTGCCTCCCGGGGACCTACGTACCCACACACAGC
GCTCACCCCAGTCACTCTCAGCGGCCATCGCTGCCATGCAGAAGTTTTAC
GGCTTGCAAGTAACAGGCAAAGCTGATGCAGACACCATGAAGGCCATGAG
GCGCCCCCGATGTGGTGTTCCAGACAGTTTGGGGCTGAGATCAAGGCCAA
TGTTCGAAGGAAGCGCTACGCCATCCAGGGTCTCAAATGGCAACATAATG
AAATCACTTTCTGCATCCAGAATTACACCCCCAAGGTGGGCGAGTATGCC
ACATACGAGGCCATTCGCAAGGCGTTCCGCGTGTGGGAGAGTGCCACACC
ACTGCGCTTCCGCGAGGTGCCCTATGCCTACATCCGTGAGGGCCATGAGA
AGCAGGCCGACATCATGATCTTCTTTGCCGAGGGCTTCCATGGCGACAGC
ACGCCCTTCGATGGTGAGGGCGGCTTCCTGGCCCATGCCTACTTCCCAGG
CCCCAACATTGGAGGAGACACCCACTTTGACTCTGCCGAGCCTTGGACTG
TCAGGAATGAGGATCTGAATGGAAATGACATCTTCCTGGTGGCTGTGCAC
GAGCTGGGCCATGCCCTGGGGCTCGAGCATTCCAGTGACCCCTCGGCCAT
CATGGCACCCTTTTACCAGTGGATGGACACGGAGAATTTTGTGCTGCCCG
ATGATGACCGCCGGGGCATCCAGCAACTTTATGGGGGTGAGTCAGGGTTC
CCCACCAAGATGCCCCCTCAACCCAGGACTACCTCCCGGCCTTCTGTTCC
TGATAAACCCAAAAACCCCACCTATGGGCCCAACATCTGTGACGGGAACT
TTGACACCGTGGCCATGCTCCGAGGGGAGATGTTTGTCTTCAAGGAGCGC
TGGTTCTGGCGGGTGAGGAATAACCAAGTGATGGATGGATACCCAATGCC
CATTGGCCAGTTCTGGCGGGGCCTGCCTGCGTCCATCAACACTGCCTACG
AGAGGAAGGATGGCAAATTCGTCTTCTTCAAAGGAGACAAGCATTGGGTG
TTTGATGAGGCGTCCCTGGAACCTGGCTACCCCAAGCACATTAAGGAGCT
GGGCCGAGGGCTGCCTACCGACAAGATTGATGCTGCTCTCTTCTGGATGC
CCAATGGAAAGACCTACTTCTTCCGTGGAAACAAGTACTACCGTTTCAAC
GAAGAGCTCAGGGCAGTGGATAGCGAGTACCCCAAGAACATCAAAGTCTG
GGAAGGGATCCCTGAGTCTCCCAGAGGGTCATTCATGGGCAGCGATGAAG
TCTTCACTTACTTCTACAAGGGGAACAAATACTGGAAATTCAACAACCAG
AAGCTGAAGGTAGAACCGGGCTACCCCAAGTCAGCCCTGAGGGACTGGAT
GGGCTGCCCATCGGGAGGCCGGCCGGATGAGGGGACTGAGGAGGAGACGG
AGGTGATCATCATTGAGGTGGACGAGGAGGGCGGCGGGGCGGTGAGCGCG
GCTGCCGTGGTGCTGCCCGTGCTGCTGCTGCTCCTGGTGCTGGCGGTGGG
CCTTGCAGTCTTCTTCTTCAGACGCCATGGGACCCCCAGGCGACTGCTCT
ACTGCCAGCGTTCCCTGCTGGACAAGGTCTGACGCCCACCGCCGGCCCGC
CCACTCCTACCACAAGGACTTTGCCTCTGAAGGCCAGTGGCAGCAGGTGG
TGGTGGGTGGGCTGCTCCCATCGTCCCGAGCCCCCTCCCCGCAGCCTCCT
TGCTTCTCTCTGTCCCCTGGCTGGCCTCCTTCACCCTGACCGCCTCCCTC
CCTCCTGCCCCGGCATTGCATCTTCCCTAGATAGGTCCCCTGAGGGCTGA
GTGGGAGGGCGGCCCTTTCCAGCCTCTGCCCCTCAGGGGAACCCTGTAGC
TTTGTGTCTGTCCAGCCCCATCTGAATGTGTTGGGGGCTCTGCACTTGAA
GGCAGGACCCTCAGACCTCGCTGGTAAAGGTCAAATGGGGTCATCTGCTC
CTTTTCCATCCCCTGACATACCTTAACCTCTGAACTCTGACCTCAGGAGG
CTCTGGGCACTCCAGCCCTGAAAGCCCCAGGTGTACCCAATTGGCAGCCT
CTCACTACTCTTTCTGGCTAAAAGGAATCTAATCTTGTTGAGGGTAGAGA
CCCTGAGACAGTGTGAGGGGGTGGGGACTGCCAAGCCACCCTAAGACCTT
GGGAGGAAAACTCAGAGAGGGTCTTCGTTGCTCAGTCAGTCAAGTTCCTC
GGAGATCTGCCTCTGCCTCACCTACCCCAGGGAACTTCCAAGGAAGGAGC
CTGAGCCACTGGGGACTAAGTGGGCAGAAGAAACCCTTGGCAGCCCTGTG
CCTCTCGAATGTTAGCCTTGGATGGGGCTTTCACAGTTAGAAGAGCTGAA
ACCAGGGGTGCAGCTGTCAGGTAGGGTGGGGCCGGTGGGAGAGGCCCGGG
TCAGAGCCCTGGGGGTGAGCCTGAAGGCCACAGAGAAAGAACCTTGCCCA
AACTCAGGCAGCTGGGGCTGAGGCCCAAAGGCAGAACAGCCAGAGGGGGC
AGGAGGGGACCAAAAAGGAAAATGAGGACGTGCAGCAGCATTGGAAGGCT
GGGGCCGGGCAGGCCAGGCCAAGCCAAGCAGGGGGCCACAGGGTGGGCTG
TGGAGCTCTCAGGAAGGGCCCTGAGGAAGGCACACTTGCTCCTGTTGGTC
CCTGTCCTTGCTGCCCAGGCAGCGTGGAGGGGAAGGGTAGGGCAGCCAGA
GAAAGGAGCAGAGAAGGCACACAAACGAGGAATGAGGGGCTTCACGAGAG
GCCACAGGGCCTGGCTGGCCACGCTGTCCCGGCCTGCTCACCATCTCAGT
GAGGGGCAGGAGCTGGGGCTCGCTTAGGCTGGGTCCACGCTTCCCTGGTG
CCAGCACCCCTCAAGCCTGTCTCACCAGTGGCCTGCCCTCTCGCTCCCCC
ACCCAGCCCACCCATTGAAGTCTCCTTGGGCCACCAAAGGTGGTGGCCAT
GGTACCGGGGACTTGGGAGAGTGAGACCCAGTGGAGGGAGCAAGAGGAGA
GGGATGTCGGGGGGGTGGGGCACGGGGTAGGGGAAATGGGGTGAACGGTG
CTGGCAGTTCGGCTAGATTTCTGTCTTGTTTGTTTTTTTGTTTTGTTTAA
TGTATATTTTTATTATAATTATTATATATGAATTCCAAAAAAAAAAAAAA
AAAAAA.

TABLE 25B

Matrix metalloproteinase 14 (membrane-inserted)
protein sequence (SEQ ID NO:58)

MSPAPRPPRCLLLPLLTLGTALASLGSAQSSSFSPEAWLQQYGYLPPGDL
RTHTQRSPQSLSAAIAAMQKFYGLQVTGKADADTMKAMRRPRCGVPDKFG
AEIKANVRRKRYAIQGLKWQHNEITFCIQNYTPKVGEYATYEAIRKAFRV
WESATPLRFREVPYAYIREGHEKQADIMIFFAEGFHGDSTPFDGEGGFLA
HAYFPGPNIGGDTHFDSAEPWTVRNEDLNGNDIFLVAVHELGHALGLEHS
SDPSAIMAPFYQWMDTENFVLPDDDRRGIQQLYGGESGFPTKMPPQPRTT
SRPSVPDKPKNPTYGPNICDGNFDTVAMLRGEMFVFKERWFWRVRNNQVM
DGYPMPIGQFWRGLPASINTAYERKDGKFVFFKGDKHWVFDEASLEPGYP
KHIKELGRGLPTDKIDAALFWMPNGKTYFFRGNKYYRFNEELRAVDSEYP
KNIKVWEGIPESPRGSFMGSDEVFTYFYKGNKYWKFNNQKLKVEPGYPKS
ALRDWMGCPSGGRPDEGTEEETEVIIIEVDEEGGGAVSAAAVVLPVLLLL
LVLAVGLAVFFFRRHGTPRRLLYCQRSLLDKV.

TSC26: Vascular Endothelial Growth Factor D.

TABLE 26A

Vascular endothelial growth factor D
(NM_004469.2) nucleotide sequence (SEQ ID NO:59)

CAAGACTTCTCTGCATTTTCTGCCAAAATCTGTGTCAGATTTAAGACACA
TGCTTCTGCAAGCTTCCATGAAGGTTGTGCAAAAAAGTTTCAATCCAGAG
TTGGGTTCCAGCTTTCTGTAGCTGTAAGCATTGGTGGCCACACCACCTCC
TTACAAAGCAACTAGAACCTGCGGCATACATTGGAGAGATTTTTTTAATT
TTCTGGACATGAAGTAAATTTAGAGTGCTTTCTAATTTCAGGTAGAAGAC
ATGTCCACCTTCTGATTATTTTTGGAGAACATTTTGATTTTTTTCATCTC
TCTCTCCCCACCCCTAAGATTGTGCAAAAAAAGCGTACCTTGCCTAATTG
AAATAATTTCATTGGATTTTGATCAGAACTGATTATTTGGTTTTCTGTGT
GAAGTTTTGAGGTTTCAAACTTTCCTTCTGGAGAATGCCTTTTGAAACAA
TTTTCTCTAGCTGCCTGATGTCAACTGCTTAGTAATCAGTGGATATTGAA
ATATTCAAAATGTACAGAGAGTGGGTAGTGGTGAATGTTTTCATGATGTT
GTACGTCCAGCTGGTGCAGGGCTCCAGTAATGAACATGGACCAGTGAAGC
GATCATCTCAGTCCACATTGGAACGATCTGAACAGCAGATCAGGGCTGCT
TCTAGTTTGGAGGAACTACTTCGAATTACTCACTCTGAGGACTGGAAGCT
GTGGAGATGCAGGCTGAGGCTCAAAAGTTTTACCAGTATGGACTCTCGCT
CAGCATCCCATCGGTCCACTAGGTTTGCGGCAACTTTCTATGACATTGAA
ACACTAAAAGTTATAGATGAAGAATGGCAAAGAACTCAGTGCAGCCCTAG
AGAAACGTGCGTGGAGGTGGCCAGTGAGCTGGGGAAGAGTACCAACACAT
TCTTCAAGCCCCCTTGTGTGAACGTGTTCCGATGTGGTGGCTGTTGCAAT
GAAGAGAGCCTTATCTGTATGAACACCAGCACCTCGTACATTTCCAAACA
GCTCTTTGAGATATCAGTGCCTTTGACATCAGTACCTGAATTAGTGCCTG
TTAAAGTTGCCAATCATACAGGTTGTAAGTGCTTGCCAACAGCCCCCCGC
CATCCATACTCAATTATCAGAAGATCCATCCAGATCCCTGAAGAAGATCG
CTGTTCCCATTCCAAGAAACTCTGTCCTATTGACATGCTATGGGATAGCA
ACAAATGTAAATGTGTTTTGCAGGAGGAAAATCCACTTGCTGGAACAGAA
GACCACTCTCATCTCCAGGAACCAGCTCTCTGTGGGCCACACATGATGTT
TGACGAAGATCGTTGCGAGTGTGTCTGTAAAACACCATGTCCCAAAGATC
TAATCCAGCACCCCAAAAACTGCAGTTGCTTTGAGTGCAAAGAAAGTCTG
GAGACCTGCTGCCAGAAGCACAAGCTATTTCACCCAGACACCTGCAGCTG
TGAGGACAGATGCCCCTTTCATACCAGACCATGTGCAAGTGGCAAAACAG
CATGTGCAAAGCATTGCCGCTTTCCAAAGGAGAAAAGGGCTGCCCAGGGG
CCCCACAGCCGAAAGAATCCTTGATTCAGCGTTCCAAGTTCCCCATCCCT
GTCATTTTTAACAGCATGCTGCTTTGCCAAGTTGCTGTCACTGTTTTTTT
CCCAGGTGTTAAAAAAAAAATCCATTTTACACAGCACCACAGTGAATCCA
GACCAACCTTCCATTCACACCAGCTAAGGAGTCCCTGGTTCATTGATGGA
TGTCTTCTAGCTGCAGATGCCTCTGCGCACCAAGGAATGGAGAGGAGGGG
ACCCATGTAATCCTTTTGTTTAGTTTTGTTTTTGTTTTTTGGTGAATGAG
AAAGGTGTGCTGGTCATGGAATGGCAGGTGTCATATGACTGATTACTCAG
AGCAGATGAGGAAAACTGTAGTCTCTGAGTCCTTTGCTAATCGCAACTCT
TGTGAATTATTCTGATTCTTTTTTATGCAGAATTTGATTCGTATGATCAG
TACTGACTTTCTGATTACTGTCCAGCTTATAGTCTTCCAGTTTAATGAAC
TACCATCTGATGTTTCATATTTAAGTGTATTTAAAGAAAATAAACACCAT
TATTCAAGCCAAAAAAAAAAAAAAAAAA.

TABLE 26B

Vascular endothelial growth factor D
(NM_004469.2) protein sequence (SEQ ID NO:60)

MYREWVVVNVFMMLYVQLVQGSSNEHGPVKRSSQSTLERSEQQIRAASSL
EELLRITHSEDWKLWRCRLRLKSFTSMDSRSASHRSTRFAATFYDIETLK
VIDEEWQRTQCSPRETCVEVASELGKSTNTFFKPPCVNVFRCGGCCNEES
LICMNTSTSYISKQLFEISVPLTSVPELVPVKVANHTGCKCLPTAPRHPY
SIIRRSIQIPEEDRCSHSKKLCPIDMLWDSNKCKCVLQEENPLAGTEDHS
HLQEPALCGPHMMFDEDRCECVCKTPCPKDLIQHPKNCSCFECKESLETC
CQKHKLFHPDTCSCEDRCPFHTRPCASGKTACAKHCRFPKEKRAAQGPHS
RKNP.

TABLE 26C

Vascular endothelial growth factor D (D89630.1)
nucleotide sequence (SEQ ID NO:61)

CCAGCTTTCTGTAGCTGTAAGCATTGGTGGCCACACCACCTCCTTACAAA
GCAACTAGAACCTGCGGCATACATTGGAGAGATTTTTTTAATTTTCTGGA
CATGAAGTAAATTTAGAGTGCTTTCTAATTTCAGGTAGAAGACATGTCCA
CCTTCTGATTATTTTTGGAGAACATTTTGATTTTTTTCATCTCTCTCTCC
CCACCCCTAAGATTGTGCAAAAAAAGCGTACCTTGCCTAATTGAAATAAT
TTCATTGGATTTTGATCAGAACTGATCATTTGGTTTTCTGTGTGAAGTTT
TGAGGTTTCAAACTTTCCTTCTGGAGAATGCCTTTTGAAACAATTTTCTC
TAGCTGCCTGATGTCAACTGCTTAGTAATCAGTGGATATTGAAATATTCA
AAATGTACAGAGAGTGGGTAGTGGTGAATGTTTTCATGATGTTGTACGTC
CAGCTGGTGCAGGGCTCCAGTAATGAACATGGACCAGTGAAGCGATCATC
TCAGTCCACATTGGAACGATCTGAACAGCAGATCAGGGCTGCTTCTAGTT
TGGAGGAACTACTTCGAATTACTCACTCTGAGGACTGGAAGCTGTGGAGA
TGCAGGCTGAGGCTCAAAAGTTTTACCAGTATGGACTCTCGCTCAGCATC
CCATCGGTCCACTAGGTTTGCGGCAACTTTCTATGACATTGAAACACTAA
AAGTTATAGATGAAGAATGGCAAAGAACTCAGTGCAGCCCTAGAGAAACG
TGCGTGGAGGTGGCCAGTGAGCTGGGGAAGAGTACCAACACATTCTTCAA
GCCCCCTTGTGTGAACGTGTTCCGATGTGGTGGCTGTTGCAATGAAGAGA
GCCTTATCTGTATGAACACCAGCACCTCGTACATTTCCAAACAGCTCTTT
GAGATATCAGTGCCTTTGACATCAGTACCTGAATTAGTGCCTGTTAAAGT
TGCCAATCATACAGGTTGTAAGTGCTTGCCAACAGCCCCCCGCCATCCAT
ACTCAATTATCAGAAGATCCATCCAGATCCCTGAAGAAGATCGCTGTTCC
CATTCCAAGAAACTCTGTCCTATTGACATGCTATGGGATAGCAACAAATG
TAAATGTGTTTTGCAGGAGGAAAATCCACTTGCTGGAACAGAAGACCACT
CTCATCTCCAGGAACCAGCTCTCTGTGGGCCACACATGATGTTTGACGAA
GATCGTTGCGAGTGTGTCTGTAAAACACCATGTCCCAAAGATCTAATCCA
GCACCCCAAAAACTGCAGTTGCTTTGAGTGCAAAGAAAGTCTGGAGACCT
GCTGCCAGAAGCACAAGCTATTTCACCCAGACACCTGCAGCTGTGAGGAC
AGATGCCCCTTTCATACCAGACCATGTGCAAGTGGCAAAACAGCATGTGC
AAAGCATTGCCGCTTTCCAAAGGAGAAAAGGGCTGCCCAGGGGCCCCACA
GCCGAAAGAATCCTTGATTCAGCGTTCCAAGTTCCCCATCCCTGTCATTT
TTAACAGCATGCTGCTTTGCCAAGTTGCTGTCACTGTTTTTTTCCCAGGT
GTTAAAAAAAAAATCCATTTTACACAGCACCACAGTGAATCCAGACCAAC
CTTCCATTCACACCAGCTAAGGAGTCCCTGGTTCATTGATGGATGTCTTC
TAGCTGCAGATGCCTCTGCGCACCAAGGAATGGAGAGGAGGGGACCCATG
TAATCCTTTTGTTTAGTTTTGTTTTTGTTTTTTGGTGAATGAGAAAGGTG
TGCTGGTCATGGAATGGCAGGTGTCATATGACTGATTACTCAGAGCAGAT
GAGGAAAACTGTAGTCTCTGAGTCCTTTGCTAATCGCAACTCTTGTGAAT
TATTCTGATTCTTTTTTATGCAGAATTTGATTCGTATGATCAGTACTGAC
TTTCTGATTACTGTCCAGCTTATAGTCTTCCAGTTTAATGAACTACCATC
TGATGTTTCATATTTAAGTGTATTTAAGAAAATAAACACCATTATTCAAG
TCTAAAAAAAAAAAAAAAAAAAAAAAA.

TABLE 26D

Vascular endothelial growth factor D (D89630.1)
protein sequence (SEQ ID NO:62)

MYREWVVVNVFMMLYVQLVQGSSNEHGPVKRSSQSTLERSEQQIRAASSL
EELLRITHSEDWKLWRCRLRLKSFTSMDSRSASHRSTRFAATFYDIETLK
VIDEEWQRTQCSPRETCVEVASELGKSTNTFFKPPCVNVFRCGGCCNEES
LICMNTSTSYISKQLFEISVPLTSVPELVPVKVANHTGCKCLPTAPRHPY
SIIRRSIQIPEEDRCSHSKKLCPIDMLWDSNKCKCVLQEENPLAGTEDHS
HLQEPALCGPHMMFDEDRCECVCKTPCPKDLIQHPKNCSCFECKESLETC
CQKHKLFHPDTCSCEDRCPFHTRPCASGKTACAKHCRFPKEKRAAQGPHS
RKNP.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated immortalized cell, wherein said cell does not express a Tuberous Sclerosis Complex-2 (TSC2) gene.

2. The cell of claim 1, wherein said cell is human.

3. The cell of claim 1, wherein said cell comprises a mutation in said TSC2 gene.

4. The cell of claim 3, wherein said mhutation is in exon 16 of said TSC2 gene.

5. The cell of claim 4, wherein said mutation is a guanine to adenine transition at nucleotide position 1832 of exon 16 of said TSC2 gene.

6. The cell of claim 1, wherein said TSC2 gene comprises a Pvu II restriction site 6 or more nucleotides upstream or downstream from nucleotide position 1832 in Exon 16 of said TSC2 gene.

7. The cell of claiml, wherein said cell constitutively phosphorylates ribosomal protein S6 or S6 kinase.

8. A culture comprising the cell of claim 1.

9. A cell deposited under ATCC Accession No: [ ].

10. A method of diagnosing tuberous sclerosis complex (TSC) or a predisposition to developing TSC in a subject comprising:
a. providing a biological sample comprising genomic DNA;
b. amplifying a region of the genomic DNA which comprises position 1832 of Exon 16 of the TSC2 gene;
c. digesting amplification product from (b) with a Pvu II restriction endonucleases; and
d. identifying a Pvu II restriction site at least 6 bases upstream or downstream from position 1832, wherein the presence of said Pvu II restriction indicates TSC or a predisposition to developing TSC in said subject.

11. The method of claim 10, wherein the biological sample is a human biological sample.

12. The method of claim 10, where the biological sample is a human angiomyolipmoma tumor.

13. A method of diagnosing a TSC related disorder or a predisposition to developing TSC related disorder in a subject, comprising determining a level of expression of a TSC-associated gene in a patient derived tissue sample, wherein an increase of said level compared to a normal control level of said gene indicates that said subject suffers from or is at risk of developing a TSC related disorder.

14. The method of claim 13, wherein said TSC-associated gene is selected from the group consisting of TSC 2, and 4-26, wherein an increase in said level compared to a normal control level indicates said subject suffers from or is at risk of developing a TSC related disorder.

15. The method of claim 14, further comprising determining said level of expression of TSC1 or TSC3.

16. The method of claim 13, wherein said increase is at least 5-fold greater than said normal control level.

17. The method of claim 13, wherein said method further comprises determining said level of expression of a plurality of TSC-associated genes.

18. The method of claim 13, wherein said level of expression is determined by detecting a gene transcript of said TSC-associated gene.

19. The method of claim 13, wherein said TSC related disorder is angiomyolipmoma, lympangioleimyomatosis, cortical tubers, subependymal nodules, or giant-cell astrocytomas.

20. A TSC related disorder reference expression profile, comprising a pattern of gene expression of one or more genes selected from the group consisting of TSC 2 and 4-26.

21. The expression profile of claim 20, further comprising TS 1 or TSC3.

22. A method of assessing the prognosis of a subject with a TSC related disorder comprising:
a. measuring over time the expression one or more nucleic acid sequences selected from the group consisting of TSC 2 and 4-26 in a subject derived cell population to yield a subject profile; and
b. comparing said subject profile to a TSC reference profile, wherein an increase in similarity between said subject profile and said referenece profile over time indicates an adverse prognosis of said subject.

23. A method of assessing the prognosis of a subject with a TSC related disorder comprising:
a. measuring over time the expression one or more nucleic acid sequences selected from the group consisting of TSC 2 and 4-26 in a subject derived cell population to yield a subject profile; and
b. comparing said subject profile to a TSC reference profile, wherein an decrease in similarity between said subject profile and said reference profile over time indicates an favorable prognosis of said subject.

24. A method of assessing the efficacy of a treatment of a TSC related disorder in a subject, comprising:
a. measuring the expression one or more nucleic acid sequences selected from the group consisting of TSC 2 and 4-26 in a subject derived cell population to yield a subject profile; and
b. comparing said subject profile to a TSC reference profile, wherein an increase in similarity between said subject profile and said reference profile over time indicates the treatment is not efficacious.

25. A method of assessing the efficacy of a treatment of a TSC related disorder, comprising:
a. measuring the expression one or more nucleic acid sequences selected from the group consisting of TSC 2 and 4-26 in a subject derived cell population to yield a subject profile; and
b. comparing said subject profile to a TSC reference profile, wherein an decrease in similarity between said subject profile and said reference profile over time indicates the treatment is efficacious.

26. A method for identifying a therapeutic agent suitable for treating a TSC related disorder in a selected subject, comprising:
a. contacting a subject derived cell population with a test agent
b. measuring the expression one or more nucleic acid sequences selected from the group consisting of TSC 2 and 4-26 in said subject derived cell population; and
c. comparing the expression of said nucleic acid sequences to the expression of said nucleic acid sequences a reference profile, thereby identifying a therapeutic agent appropriate for said subject.

27. A method of identifying an agent that inhibits the expression or activity of a TSC-associated gene, comprising contacting a test cell expressing said TSC associated gene with a test agent and determining the expression level of said TSC associated gene, wherein a decrease of said level compared to a level of said gene in the absence of said test agent indicates that said test agent is an inhibitor of said TSC-associated gene.

28. A kit comprising a detection reagent which binds to two or more nucleic acid sequences selected from the group consisting of TSC 1-26

29. An array comprising a nucleic acid which binds to two or more nucleic acid sequences selected from the group consisting of TSC 1-26.


SEQUENCE LISTING (62 sequences)

SEQ ID NO: 1 - DNA, 752 bp, Homo sapiens
ccgtcagaaatctaaacccgtgactatcatgggactcaaaaccagcccaaaaaataagtcaaaacgattaagagccagagaagcagtcttcatacacgcggccagccagcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgata

SEQ ID NO: 2 - DNA, 1524 bp, Homo sapiens
agcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgatactatctgtgccagaggtaatgttagtaaatccatggtgttattttctgagagacagaattcaagtgggtattctggggccatccaatttctctttacttgaaatttggctaataacaaactagtcaggttttcgaaccttgaccgacatgaactgtacacagaattgttccagtactatggagtgctcacaaaggatacttttacaggttaagacaaagggttgactggcctatttatctgatcaagaacatgtcagcaatgtctctttgtgctctaaaattctattatactacaataatatattgtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgtgcagaagaaatcataaaggatcagagattctg

SEQ ID NO: 3 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 4 - DNA, 1607 bp, Homo sapiens
atgacccaggcaggccggcggggtcctggcacacccgagccgcgtccgcgaacacagcccatggcctccccgcgcctagggaccttctgctgccccacgcgggacgcagccacgcagctcgtgctgagcttccagccgcgggccttccacgcgctctgcctgggcagcggcgggctccgcttggcgctgggccttctgcagctgctgcccggccgccggcccgcgggccccgggtcccccgcgacgtccccgccggcctcggtccgcatcctgcgcgctgccgctgcctgcgaccttctcggctgcctgggtatggtgatccggtccaccgtgtggttaggattcccaaattttgttgacagcgtctcggatatgaaccacacggaaatttggcctgctgctttctgcgtggggagtgcgatgtggatccagctgttgtacagtgcctgcttctggtggctgttttgctatgcagtggatgcttatctggtgatccggagatcggcaggactgagcaccatcctgctgtatcacatcatggcgtggggcctggccaccctgctctgtgtggagggagccgccatgctctactacccttccgtgtccaggtgtgagcggggcctggaccacgccatcccccactatgtcaccatgtacctgcccctgctgctggttctcgtggcgaaccccatcctgttccaaaagacagtgactgcagtggcctctttacttaaaggaagacaaggcatttacacggagaacgagaggaggatgggagccgtgatcaagatccgatttttcaaaatcatgctggttttaattatttgttggttgtcgaatatcatcaatgaaagccttttattctatcttgagatgcaaacagatatcaatggaggttctttgaaacctgtcagaactgcagccaagaccacatggtttattatgggaatcctgaatccagcccagggatttctcttgtctttggccttctacggctggacaggatgcagcctgggttttcagtctcccaggaaggagatccagtgggaatcactgaccacctcggctgctgagggggctcacccatccccactgatgccccatgaaaaccctgcttccgggaaggtgtctcaagtgggtgggcagacttctgacgaagccctgagcatgctgtctgaaggttctgatgccagcacaattgaaattcacactgcaagtgaatcctgcaacaaaaatgagggtgaccctgctctcccaacccatggagacctatgaaggggatgtgctgggggtccagaccccatattcctcagactcaacaattcttgttctttagaactgtgttctcaccttcccaacactgcactgccgaagtgtagcggcccccaaaccttgctctcatcaccagctagagcttcttcccgaagggcctttaggataggagaaagggttcatgcacacacgtgtgagaatggaagagccccctccagaccactctacagctgctctagccttagttgccactaggaagttttctgaggctggctgtaaagtaagtgtaaggtccacatccttggggaagtagttaaataaaatagttatgactg

SEQ ID NO: 5 - protein, 424 aa, Homo sapiens
MTQAGRRGPGTPEPRPRTQPMASPRLGTFCCPTRDAATQLVLSFQPRAFHALCLGSGGLRLALGLLQLLPGRRPAGPGSPATSPPASVRILRAAAACDLLGCLGMVIRSTVWLGFPNFVDSVSDMNHTEIWPAAFCVGSAMWIQLLYSACFWWLFCYAVDAYLVIRRSAGLSTILLYHIMAWGLATLLCVEGAAMLYYPSVSRCERGLDHAIPHYVTMYLPLLLVLVANPILFQKTVTAVASLLKGRQGIYTENERRMGAVIKIRFFKIMLVLIICWLSNIINESLLFYLEMQTDINGGSLKPVRTAAKTTWFIMGILNPAQGFLLSLAFYGWTGCSLGFQSPRKEIQWESLTTSAAEGAHPSPLMPHENPASGKVSQVGGQTSDEALSMLSEGSDASTIEIHTASESCNKNEGDPALPTHGDL

SEQ ID NO: 6 - DNA, 2026 bp, Homo sapiens
ctcgagatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcagggggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattgcggccg

SEQ ID NO: 7 - DNA, 1986 bp, Homo sapiens
atggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatggtagccaggtgtggggaggacagccagtgtatccccaggaaactgacgatgcctgcatcttccctgatggtggaccttgcccatctggctcttggtctcagaagagaagctttgtttatgtctggaagacctggggtcaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctga

SEQ ID NO: 8 - DNA, 2143 bp, Homo sapiens
agtgcctttggttgctggagggaagaacacaatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaatactcagagcctgaaaaaaaaaaaaaaaaaa

SEQ ID NO: 9 - protein, 662 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGGLGQVPLIVGILLVLMAVVLASLIYRRRLMKQDFSVPQLPHSSSHWLRLPRIFCSCPIGENSPLLSGQQV

SEQ ID NO: 10 - DNA, 3299 bp, Homo sapiens
ataataattaggccaagcgttgaatagtacggggggggggggggggcgagccccggcggctctggccgcggccgcactcagcgccacgcgtcgaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagacccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcacttcctattttgtaaacctatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgacagtgactatgtcagtctaatctcttgtttgccagggttcctaaattaattcacttaaccatgatgcaaatgtttttcatttggtgaagacctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacaaaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttaacaagaaatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcacttgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcctcaagcctgaggttttatatacaaactccctgaatactctttttgccttgttacttctcagcctcctagccaagtcctatgtaatatggaaaacaaacactgcagacttgagattcagttgccgatcaaggctctggcattcagagaacccttgcaactcgagaagctgtttttgatttcgtttttgttttgaaccggtgctctcccatctaacaactaacaaggaccatttccaggcgggagatattttaaacacccaaaatgttgggtctgatttccaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccaccaaacactgaatttctcttgttgttttaactctcccacaagaatgcaatttcgttaatggagatgacttaagttggcagcagtaatcttcttttaggagcttgtaccacagtcttgcacataagtgcagatttgccccaagtaaagagaatttcctcaacactaacttcacggggataatcaccacgtaactacccttaaagcatatcactagccaaagaggggaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagagatttgtggtcttgatcatacctattaaaataatgccaaacaccaaatatgaattttatgatgtacactttgtgcttggcattaaaagaaaaaaacacacacgcc

SEQ ID NO: 11 - DNA, 4049 bp, Homo sapiens
gcggccgcactcagcgccacgcgtcgaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagccccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcactccctattttgtaaacatatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgatagtgactatgtcagtctaatctcttgtttgccaaggttcctaaattaattcacttaaccatgatgcaaatgtttttcattttgtgaagaccctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacagaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacckcagaggctgaaattcctaatacctttcctttatcgtggttatagtcagctcatttccattccactatttcccataatgcttctgagagccactaacttgattgataaagatcctgcctctgctgagtgtacctgacagtaagtctaaagatgaragagtttagggactactctgttttagcaagaratattktgggggtctttttgttttaactattgtcaggagattgggctaragagaagacgacgagagtaaggaaataaagggrattgcctctggctagagagtaagttaggtgttaatacctggtagaaatgtaagggatatgacctccctttctttatgtgctcactgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcttcaagcctgaggttttatatacaaactccctgaatactctttttgccttgtatcttctcagcctcctagccaagtcctatgtaatatggaaaacaaacactgcagacttgagattcagttgccgatcaaggctctggcattcagagaacccttgcaactcgagaagctgtttttatttcgtttttgttttgatccagtgctctcccatctaacaactaaacaggagccatttcaaggcgggagatattttaaacacccaaaatgttgggtctgattttcaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccagcaaacactgaatttctcttgttgttttaactctgccacaagaatgcaatttcgttaatggagatgacttaagttggcagcagtaatcttcttttaggagcttgtaccacagtcttgcacataagtgcagatttggctcaagtaaagagaatttcctcaacactaacttcactgggataatcagcagcgtaactaccctaaaagcatatcactagccaaagagggaaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagatatttgtggtcttgatcatacctattaaaataatgccaaacaccaaatatgaattttatgatgtacactttgtgcttggcattaaaagaaaaaaacacacatcctggaagtctgtaagttgttttttgttactgtaggtcttcaaagttaagagtgtaagtgaaaaatctggaggagaggataatttccactgtgtggaatgtgaatagttaaatgaaaagttatggttatttaatgtaattattacttcaaatcctttggtcactgtgatttcaagcatgttttctttttctcctttatatgactttctctgagttgggcaaagaagaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaaagagcaactaataaattaaacctattctttcaaaaaaaaa

SEQ ID NO: 12 - DNA, 555 bp, Homo sapiens
atgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaa

SEQ ID NO: 13 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 14 - DNA, 726 bp, Homo sapiens
tcccccataattatgccacatagctgttattattttcatatattgccttcatttttttcacagttgctattttgtgtaattttggaatcagtttacaaacattctgcattcttcttttttcacttttgatagatgtttcatattaaccaataagaataacatttattagtttatcatgtcaccaagcaactatttattttaaaagtctgaactagttttgattacctaaagtgattaccagtggatgaaaatactgcgggcacaacatgaaaccttctaaacaatcagagagcctattacaatacattttttaaaatcttatgtaactggccgggcgcggagacgcacacctgtaatcccagcactttgggaggccgaggtgggcggatcacctgaggtcaggagtttgagaccagcctggtcaacatggcaaaaccccgtctctactaaaaatacaaaaattagccgggtgtggtgatgcacgcctgtactcccagctactcaggaggctgaggcaggagaatcgcctgaacacagtgggcagaggttgcactcaagcatgggtgacagagcgaggcttgaatatagtctaaatacagatccctgtcctagttactaagtataaaaaatgaataaaatattagtcctgtctttgtattttctgtaccaagatgaaccaaattgccgaagtgtccacagtaaacaaagattatttatcacacaagc

SEQ ID NO: 15 - DNA, 2260 bp, Homo sapiens
gggggtatttgtttcactgctttcaaccgcctgtgctggaggctcagaataagtcaatgggaggaggatttcagtcacagcagcaagcaagtctagtgaacagataagatgacatgctcagcaaaataacaacgaaaccagagggggaactctctggcatgcaagttcaaacacgactctacaactacggcagaaaaagagagagagagaaactaaaaatatatatatatcctatttttttcacagctatcagtttctttcactgagctttcctaaatttaagcctctagaaaataataaatacttggatatcttacctacaaacatggacagatgtgtgtatgcgctcattttagagaacttgaatttttttttttaaaggaaggtgtcaactttggcttttgagtgtttggcatggttacaatgccttaaaaaaacagatgagcagcttagctactaaccatgctgaccactgttcggaacgggattgaatcacagaaaaacagcaaatggctctctcttacagagtgtctgaacttcaaagcacaattcctgagcacattttgcagagcacctttgttcacgttatctcttctaactggtctggattacagacagaatcaataccagaggaaatgaaacagattgttgaggaacagggaaataaactgcactgggcagctcttctgatactcatggtgataatacccacaattggtggaaatacccttgttattctggctgtttcactggagaagaagctgcagtatgctactaattactttctaatgtccttggcggtggctgatttgctggttggattgtttgtgatgccaattgccctcttgacaataatgtttgaggctatgtggcccctcccacttgttctatgtcctgcctggttatttcttgacgttctcttttcaaccgcatccatcatgcatctctgtgccatttcagtggatcgttacatagccatcaaaaagccaatccaggccaatcaatataactcacgggctacagcattcatcaagattacagtggtgtggttaatttcaataggcattgccattccagtccctattaaagggatagagactgatgtggacaacccaaacaatatcacttgtgtgctgacaaaggaacgttttggcgatttcatgctctttggctcactggctgccttcttcacacctcttgcaattatgattgtcacctactttctcactatccatgctttacagaagaaggcttacttagtcaaaaacaagccacctcaacgcctaacatggttgactgtgtctacagttttccaaagggatgaaacaccttgctcgtcaccggaaaaggtggcaatgctggatggttctcgaaaggacaaggctctgcccaactcaggtgatgaaacacttatgcgaagaacatccacaattgggaaaaagtcagtgcagaccatttccaacgaacagagagcctcaaaggtcctagggattgtgtttttcctctttttgcttatgtggtgtcccttctttattacaaatataactttagttttatgtgattcctgtaaccaaactactctccaaatgctcctggagatatttgtgtggataggctatgtttcctcaggagtgaatcctttggtctacaccctcttcaataagacatttcgggatgcatttggccgatatatcacctgcaattaccgggccacaaagtcagtaaaaactctcagaaaacgctccagtaagatctacttccggaatccaatggcagagaactctaagtttttcaagaaacatggaattcgaaatgggattaaccctgccatgtaccagagtccaatgaggctccgaagttcaaccattcagtcttcatcaatcattctactagatacgcttctcctcactgaaaatgaaggtgacaaaactgaagagcgagttagttatgtatagcagaactggcagttgtcatcaaacataatgatgagtaagatgatgaatgagatgtaaatgtgccaagaatatattatataaagaattttatgtcatatatcaaatcatctctttaacctaagatgtaagtattaagaatatctaattttcctaatttggacaagattattccatgaggaaaataattttatatagctacaaatgaaaacaatccagcactctggttaaattttaaggtattcgaatgaaataaagtcaaatcaataaatttcaggccaaaaaaaaaaaaaaaaaaaaaaaaaaaa

SEQ ID NO: 16 - protein, 481 aa, Homo sapiens
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEERVSYV

SEQ ID NO: 17 - DNA, 2813 bp, Homo sapiens
cggggctcgaggctgctggagtcgctcgctgactcgccctgcgccctcgccgcggacaccggagctgcggccgctccccgctgtcccccagagatggcagatcctgaggtagttgtgagtagctgcagctctcatgaagaggaaaatcgctgcaattttaaccagcaaacatctccatctgaggagcttctattagaagaccagatgaggcgaaaactcaaattttttttcatgaatccctgtgagaagttctgggctcgaggtagaaaaccatggaaacttgccatacaaattctaaaaattgcaatggtgactatccagctggtcttatttgggctaagtaaccagatggtggtagctttcaaggaagagaatactatagcattcaaacaccttttcctaaaaggatatatggaccgaatggatgacacatatgcagtgtacacacaaagtgacgtgtatgatcagttaatcttcgcagtaaaccagtacttgcagctatacaatgtctccgttgggaatcatgcttatgagaacaaaggtaccaagcaatctgctatggcaatctgtcagcacttctacaagcgaggaaacatctaccctggaaatgatacctttgacatcgatccagaaattgaaactgagtgtttctttgtggagccagatgaaccttttcacattgggacaccagcagaaaataaactgaacttaacactggacttccacagactcctaacagtggagcttcagtttaaactgaaagccattaatctgcagacagttcgtcatcaagaactccctgactgttatgactttactctgactataacatttgacaacaaggcccatagtggaagaattaaaataagtttagataatgacatttccatcagagaatgtaaagactggcatgtatctggatcaattcagaagaacactcattacatgatgatctttgatgcctttgtcattctgacttgcttggtttcattaatcctctgcattagatctgtgattagaggacttcagcttcagcaggagtttgtcaattttttcctcctccattataagaaggaagtttctgtttctgatcaaatggaatttgtcaatggatggtacattatgattattattagtgacatattgacaatcattggatcaattctaaaaatggaaatccaagctaagagtctaactagttatgatgtctgtagcatacttcttgggacttctaccatgctcgtgtggcttggagtcatccgatacctcggtttctttgcaaagtacaacctcctcattttgacccttcaggcagcgctgcccaatgtcatcaggttctgctgctgtgcagctatgatttacttaggttactgcttctgtggatggatcgtgctggggccttaccatgacaagtttcgttctctgaacatggtctctgagtgccttttctctctgataaatggagatgatatgtttgccacgtttgcaaaaatgcagcaaaaaagttacttagtctggctgtttagtagaatttacctctactcattcatcagcctctttatatatatgattttaagtcttttcattgcactgatcactgatacatacgaaacaattaagcaataccaacaagatggcttcccagagactgaacttcgtacatttatatcagaatgcaaagatctacccaactctggaaaatacagattagaagatgaccctccagtatctttattctgctgttgtaaaaagtagctatcaggtttatctgtactttagaggaaaatataatgtgtagctgagttggaacactgtggatattctgagatcagatgtagtatgtttgaagactgttattttgagctaattgagacctataattcaccaataactgtttatatttttaaaagcaatatttaatgtctttgcaactttatgctgggattgtttttaaaaaaactttaatgaggaaagctattggattattattatttcttgtttattttgccatggctttagaatgtattctgtatgcctctcttttgctctgatactgttgctcctgctattctgattgtgcagactgtgtaattagtggaaaacaatccttggtctgactgtgactttggacaactcagtaaccctggcttggaccactctcaggagtccatccttgagagagtgggtgtagttatcatttatacagtaatcattgcattttaaaatcttctcttgaaaggaagaataagagtgcaccagaataagagcgcaccagaataagagcgcaccagctaacaatgtgatacggccatatgtcacttaaggatggagatatgttctgagaaatgtgtcattaggcgattttgtcattaaacatcatagcatgtacttccacaaacctagatggtatagcctactacacacctaggctatttggtatagcctgttggtcctggggtacaaatctgtacaacatgttactgtattgaatacagtaggcaattgtaacacaatggtaagtatctaaacatagaaaagggacagtaaaaatatggttttataatcttctgggaccaccattgtatatgcggtacatcattgaccaaaacatcgttatccagcatatgactgtatttggttatgaaagccaactgttacttgattctgcttttagttcttaagaggatcaggcttttaaatactcatttacaagttttctatcctccttcagtgttaaagtagaaagtaaaaagagtatcttatacatgcatgaaattaaagcatataccaaatgcaaaaaaaaaaaaaaaaaa

SEQ ID NO: 18 - protein, 553 aa, Homo sapiens
MADPEVVVSSCSSHEEENRCNFNQQTSPSEELLLEDQMRRKLKFFFMNPCEKFWARGRKPWKLAIQILKIAMVTIQLVLFGLSNQMVVAFKEENTIAFKHLFLKGYMDRMDDTYAVYTQSDVYDQLIFAVNQYLQLYNVSVGNHAYENKGTKQSAMAICQHFYKRGNIYPGNDTFDIDPEIETECFFVEPDEPFHIGTPAENKLNLTLDFHRLLTVELQFKLKAINLQTVRHQELPDCYDFTLTITFDNKAHSGRIKISLDNDISIRECKDWHVSGSIQKNTHYMMIFDAFVILTCLVSLILCIRSVIRGLQLQQEFVNFFLLHYKKEVSVSDQMEFVNGWYIMIIISDILTIIGSILKMEIQAKSLTSYDVCSILLGTSTMLVWLGVIRYLGFFAKYNLLILTLQAALPNVIRFCCCAAMIYLGYCFCGWIVLGPYHDKFRSLNMVSECLFSLINGDDMFATFAKMQQKSYLVWLFSRIYLYSFISLFIYMILSLFIALITDTYETIKQYQQDGFPETELRTFISECKDLPNSGKYRLEDDPPVSLFCCCKK

SEQ ID NO: 19 - DNA, 494 bp, Homo sapiens
ttttttgaggatgcattgatgtattgatttgcctgggaacaatggcctatagttcagcctgagaattctcataaagttaagaaggcataaaaatgccccccccgagactcgtcaggagtattgactctcctacagtttaatttgctgcttttcgtcggtttctgtgatgtcatcccacatgtgtaagctggaaaaatccacgctgtgaagtgtaacctcctgtgtgtatttccacaatggagaatgttaggcttcgtttccctcggttgctacacatctgattacatgtgtcaggaaaacaaacttaaaaaatttcaggagacaaacctttcagcggaattgcctggaacccatgaagtgaggtcatagaacctacaactataataagctgtaggaagaaaagtagcctctgggctactttgtgtctagtcacattgactttccaggtgatggccctacaaaactcaaaccacctctattattcatgcctaaat

SEQ ID NO: 20 - DNA, 2655 bp, Homo sapiens
gaaaatgtatacctggcagcaggcactgtgtatggactggagacccagctgactgagctagaagatgccgcccgctgcatccacagcggcactgatgagacccatctggcggatctggaggaccaggtggccacggctgcagcccaagtccaccatgctgaactccagatttcagatattgagagccggatttcagccctgaccattgcaggattaaacatagcaccatgtgtgcgcttcacaagaagacgggatcagaagcaaaggacccaggtacaaaccatagatacatcaaggcagcaaaggaggaaactgcctgctccaccggtgaaagctgaaaaaattgagacatcttcagtgactaccattaaaacatttaaccacaacttcattctccaaggctcctcaacaaacaggactaaggaaaggaaaggcaccaccaaggatttgatggagcctgctctggagtcagctgtgatgtactgacaccatggaattccactgccagtgacccactgcctccggccgtacacgacagtgccttgacccaacagccatcgagtactgtatgtatttccacctgaggagaaggcctggggaggccacagtgcaccattgcacagggctgtcctgatacctcatccagaaagccgtctcagacttcagcactgcggtcttgcccactctctgccttaggctcccaggggaatccaagacagaaaatgaagacactggcttccaacagcagcgctccatgtttaagatacatattttccctgtttgctttgctactgtatgttgactttaagatctttttttaaatacatttgattcagctagtattccatgtcaacaatttgtccaaaggaaaactgctggagggaggtggagggaggaaggtgggaattattatttaatacatcattaatgcttattaatctctcacaagcatctttgtcttgcaaatcctaagggaaaagcaagtccctgcagtgagcactagggacagtctaatttggggattgctcaaccatcaagactgcaggtctcccttcagccacctccttcctgctaaaagcttagcctaccacactaccagtcattcccatcgctttgcaatcacaagccacaggatgagaagttctgactcactcatgccatgcccagggctatctgaaacaatgtctcattaagaatttagggttcttccatgggcttactgacagttgcccagatctgaaggggaaagggtcttgagaaagaccatcactggctcaactttagggcactgtccagagtcaacatgatgtggtttagcagtgatcacatctaaacaaagtttaggtaaatgaattatcgcagagaaaaaccacatgagaaaatttttgtactccaaatttacttcccaataaatattcagcaaagtagtaaaatgaccttaaagataaaaatgattagggaatagccttagaaaatttataggtataaaaaattcaaggacaaactgtgcatttaatggacacaagaattgactctaactccatgtctgtggtttctttgaacccatatcaaatgtatgactatttagagtgtttataagagataatggaactgaactttcactcaattaattgggcattaacaaccttcttttatgtttgttcctgatatagtctgaatcttaggaagaaggtaaaagaaaggaggcaagagaatagttatgatgaatatgtgttaagtgcctgctctgaaggaggcaatgttcttctcatttgaatccttatggcaaccttattcaataggttttcccatatttcagatttaataactgaaggccagagagattaatttgccaaagccacacctttatgctaattatgattggaatgcatcacaaaagcctaactctgttgttttcaacctctacgttattttgctgctatgtgcatttccagatctgattttctgctaacttgtgtgctatgatccactcctgatgggggtctacattaatcttccagtactccttgctgatgctgtgttatgtgtcatctaacagaaatgactcctttgaaataagtaaatctttggctttttgttctgttggtgtgattcaaagcaaaacaaacaaacaaaaacaaattttaagaacacaacaaaaaagatttgacttccgaatagaatgttttctttaagaggcatgaaaagcaactattgttgtgttacagtgttaaaaatattcagttttctttgacaaaaatgtgtactgtgtaagccttgcaaacaaaaaacaacaaaaaagaagcagcagcagcagcctgctgtgtggcatctgaacttttataaaggtttccttgtgccaaataagtgcaaagatttaatttactattaaaaaccataagcatatgttatagttccagaagaattattttgtcatcaagtgattttgatctttagtgtcaatatttatatttagattaatttttataaatgaaaatattttaatggtttaagaaaatgaggacaacaggataatatctttgatgacttctgaaagttatgcttcccttcatgttatatgcacattgccaagaattactgtcaagagaaatgataagtaaaagtcatttatgaaaataaaaaaaaaaaaaaaaa

SEQ ID NO: 21 - protein, 161 aa, Homo sapiens
ENVYLAAGTVYGLETQLTELEDAARCIHSGTDETHLADLEDQVATAAAQVHHAELQISDIESRISALTIAGLNIAPCVRFTRRRDQKQRTQVQTIDTSRQQRRKLPAPPVKAEKIETSSVTTIKTFNHNFILQGSSTNRTKERKGTTKDLMEPALESAVMY

SEQ ID NO: 22 - DNA, 568 bp, Homo sapiens
aaaggcacagctttcccagtgtttgtgttccttgcttgcgccctgttttaatgttgtagttacaggtgtccagcagggaggaatgcagcccctgtgggcgcttgggggagctgctgggaatccaagttcaaggagcagctgttttctgttttctgttgccccacagcgccacctcctggccccttggtggtgatgattttgaagtcagcaggttctggtgggccgtgtgaactccagcagctctgggctgagctgtggaaacactgcgtcctttgaaataatacagctttcctgagcccaccccagtccctaaagactgcctctggggttgagattctgagatgcttgacagcatggcttttcccggtgttatgtgtcgtttctatccttaagcctgttaggggtggactggaggctggaccaagctccactggctgcaggaggacccttctgtgggctccaggctggccgtgtgcgtgtggggaggtgggatttgctgctaggcttcatgatcactgtgaagaagcagcccccaagaatagggtgataggccctccccatgtcaccg

SEQ ID NO: 23 - DNA, 4826 bp, Homo sapiens
agctgagcccgagcccagaccgcgcccgcgccgccatgcccctggccttctgcggcagcgagaaccactcggccgcctaccgggtggaccagggggtcctcaacaacggctgctttgtggacgcgctcaacgtggtgccgcacgtcttcctactcttcatcaccttccccatcctcttcattggatggggaagtcagagctccaaggtgcacatccaccacagcacatggcttcatttccctgggcacaacctgcggtggatcctgaccttcatgctgctcttcgtcctggtgtgtgagattgcagagggcatcctgtctgatggggtgaccgaatcccaccatctgcacctgtacatgccagccgggatggcgttcatggctgctgtcacctccgtggtctactatcacaacatcgagacttccaacttccccaagctgctaattgccctgctggtgtattggaccctggccttcatcaccaagaccatcaagtttgtcaagttcttggaccacgccatcggcttctcgcagctacgcttctgcctcacagggctgctggtgatcctctatgggatgctgctcctcgtggaggtcaatgtcatcagggtgaggagatacatcttcttcaagacaccgagggaggtgaagcctcccgaggacctgcaagacctgggggtacgcttcctgcagcccttcgtgaatctgctgtccaaaggcacctactggtggatgaacgccttcatcaagactgcccacaagaagcccatcgacttgcgagccatcgggaagctgcccatcgccatgagggccctcaccaactaccaacggctctgcgaggcctttgacgcccaggtgcggaaggacattcagggcactcaaggtgcccgggccatctggcaggcactcagccatgccttcgggaggcgcctggtcctcagcagcactttccgcatcttggccgacctgctgggcttcgccgggccactgtgcatctttgggatcgtggaccaccttgggaaggagaacgacgtcttccagcccaagacacaatttctcggggtttactttgtctcatcccaagagttccttgccaatgcctacgtcttagctgtgcttctgttccttgccctcctactgcaaaggacatttctgcaagcatcctactatgtggccattgaaactggaattaacttgagaggagcaatacagaccaagatttacaataaaattatgcacctgtccacctccaacctgtccatgggagaaatgactgctggacagatctgtaatctggttgccatcgacaccaatcagctcatgtggtttttcttcttgtgcccaaacctctgggctatgccagtacagatcattgtgggtgtgattctcctctactacatactcggagtcagtgccttaattggagcagctgtcatcattctactggctcctgtccagtacttcgtggccaccaagctgtctcaggcccagcggagcacactggagtattccaatgagcggctgaagcagaccaacgagatgctccgcggcatcaagctgctgaagctgtacgcctgggagaacatcttccgcacgcgggtggagacgacccgcaggaaggagatgaccagcctcagggcctttgccatctatacctccatctccattttcatgaacacggccatccccattgcagctgtcctcataactttcgtgggccatgtcagcttcttcaaagaggccgacttctcgccctccgtggcctttgcctccctctccctcttccatatcttggtcacaccgctgttcctgctgtccagtgtggtccgatctaccgtcaaagctctagtgagcgtgcaaaagctaagcgagttcctgtccagtgcagagatccgtgaggagcagtgtgccccccatgagcccacacctcagggcccagccagcaagtaccaggcggtgcccctcagggttgtgaaccgcaagcgtccagcccgggaggattgtcggggcctcaccggcccactgcagagcctggtccccagtgcagatggcgatgctgacaactgctgtgtccagatcatgggaggctacttcacgtggaccccagatggaatccccacactgtccaacatcaccattcgtatcccccgaggccagctgactatgatcgtggggcaggtgggctgcggcaagtcctcgctccttctagccgcactgggggagatgcagaaggtctcaggggctgtcttctggagcagccttcctgacagcgagataggagaggaccccagcccagagcgggagacagcgaccgacttggatatcaggaagagaggccccgtggcctatgcttcgcagaaaccatggctgctaaatgccactgtggaggagaacatcatctttgagagtcccttcaacaaacaacggtacaagatggtcattgaagcctgctctctgcagccagacatcgacatcctgccccatggagaccagacccagattggggaacggggcatcaacctgtctggtggtcaacgccagcgaatcagtgtggcccgagccctctaccagcacgccaacgttgtcttcttggatgaccccttctcagctctggatatccatctgagtgaccacttaatgcaggccggcatccttgagctgctccgggacgacaagaggacagtggtcttagtgacccacaagctacagtacctgccccatgcagactggatcattgccatgaaggatggcaccatccagagggagggtaccctcaaggacttccagaggtctgaatgccagctctttgagcactggaagaccctcatgaaccgacaggaccaagagctggagaaggagactgtcacagagagaaaagccacagagccaccccagggcctatctcgtgccatgtcctcgagggatggccttctgcaggatgaggaagaggaggaagaggaggcagctgagagcgaggaggatgacaacctgtcgtccatgctgcaccagcgtgctgagatcccatggcgagcctgcgccaagtacctgtcctccgccggcatcctgctcctgtcgttgctggtcttctcacagctgctcaagcacatggtcctggtggccatcgactactggctggccaagtggaccgacagcgccctgaccctgacccctgcagccaggaactgctccctcagccaggagtgcaccctcgaccagactgtctatgccatggtgttcacggtgctctgcagcctgggcattgtgctgtgcctcgtcacgtctgtcactgtggagtggacagggctgaaggtggccaagagactgcaccgcagcctgctaaaccggatcatcctagcccccatgaggttttttgagaccacgccccttgggagcatcctgaacagattttcatctgactgtaacaccatcgaccagcacatcccatccacgctggagtgcctgagccgctccaccctgctctgtgtctcagccctggccgtcatctcctatgtcacacctgtgttcctcgtggccctcttgcccctggccatcgtgtgctacttcatccagaagtacttccgggtggcgtccagggacctgcagcagctggatgacaccacccagcttccacttctctcacactttgccgaaaccgtagaaggactcaccaccatccgggccttcaggtatgaggcccggttccagcagaagcttctcgaatacacagactccaacaacattgcttccctcttcctcacagctgccaacagatggctggaagtccgaatggagtacatcggtgcatgtgtggtgctcatcgcagcggtgacctccatctccaactccctgcacagggagctctctgctggcctggtgggcctgggccttacctacgccctaatggtctccaactacctcaactggatggtgaggaacctggcagacatggagctccagctgggggctgtgaagcgcatccatgggctcctgaaaaccgaggcagagagctacgaggggctcctggcaccatcgctgatcccaaagaactggccagaccaagggaagatccagatccagaacctgagcgtgcgctacgacagctccctgaagccggtgctgaagcacgtcaatgccctcatctcccctggacagaagatcgggatctgcggccgcaccggcagtgggaagtcctccttctctcttgccttcttccgcatggtggacacgttcgaagggcacatcatcattgatggcattgacatcgccaaactgccgctgcacaccctgcgctcacgcctctccatcatcctgcaggaccccgtcctcttcagcggcaccatccgatttaacctggaccctgagaggaagtgctcagatagcacactgtgggaggccctggaaatcgcccagctgaagctggtggtgaaggcactgccaggaggcctcgatgccatcatcacagaaggcggggagaatttcagccagggacagaggcagctgttctgcctggcccgggccttcgtgaggaagaccagcatcttcatcatggacgaggccacggcttccattgacatggccacggaaaacatcctccaaaaggtggtgatgacagccttcgcagaccgcactgtggtcaccatcgcgcatcgagtgcacaccatcctgagtgcagacctggtgatcgtcctgaagcggggtgccatccttgagttcgataagccagagaagctgctcagccggaaggacagcgtcttcgcctccttcgtccgtgcagacaagtgacctgccagagcccaagtgccatcccacattcggaccctgcccata

SEQ ID NO: 24 - protein, 1581 aa, Homo sapiens [sequence omitted]

SEQ ID NO: 25 - DNA, 4977 bp, Homo sapiens
cggggcccggggggcgggggcctgacggccgggccgggcggcggagctgcaagggacagaggcgcggcaggcgcgcggagccagcggagccagctgagcccgagcccagcccgcgcccgcgccgccatgcccctggccttctgcggcagcgagaaccactcggccgcctaccgggtggaccagggggtcctcaacaacggctgctttgtggacgcgctcaacgtggtgccgcacgtcttcctactcttcatcaccttccccatcctcttcattggatggggaagtcagagctccaaggtgcacatccaccacagcacatggcttcatttccccgggcacaacctgcggtggatcctgaccttcatgctgctcttcgtcctggtgtgtgagattgcagagggcatcctgtctgatggggtgaccgaatcccaccatctgcacctgtacatgccagccgggatggcgttcatggctgctgtcacctccgtggtctactatcacaacatcgagacttccaacttccccaagctgctaattgccctgctggtgtattggaccctggccttcatcaccaagaccatcaagtttgtcaagctcttggaccacgccatcggcttctcgcagctacgcttctgcctcacagggctgctggtgatcctctatgggatgctgctcctcgtggaggtcaatgtcatcagggtgaggagatacatcttcttcaagacaccgagggaggtgaagcctcccgaggacctgcaagacctgggggtacgcttcctgcagcccttcgtgaatctgccgtccaaaggcacctactggtggatgaacgccttcatcaagactgcccacaagaagcccatcgacttgcgagccatcgggaagctgcccatcgttatgagggccctcaccaactaccaacggctctgcgaggcctttgacgcccaggtgcggaaggacattcagggcactcaaggtgcccgggccatctggcaggcactcagccatgccttcgggaggcgcctggtcctcagcagcactttccgcatcttggccgacctgctgggcttcgccgggccactgtgcatctttgggatcgtggaccaccttgggaaggagaacgacgtcttccagcccaagacacaatttctcggggtttactttgtctcatcccaagagttccttgccaatgcctacgtcttagctgtgcttctgttccttgccctcctactgcaaaggacatttctgcaagcatcctactatgtggccattgaaactggaattaacttgagaggagcaatacagaccaagatttacaataaaattatgcacctgtccacctccaacctgtccatgggagaaatgactgctggacagatctgtaatctggttgccatcgacaccaatcagctcatgtggtttttcttcttgtgcccaaacctctgggctatgccagtacagatcattgtgggtgtgattctcctctactacatactcggagtcagtgccttaattggagcagctgtcatcattctactggctcctgtccagtacttcgtggccaccaagctgtctcaggcccagcggagcacactggagtattccaatgagcggctgaagcagaccaacgagatgctccgcggcatcaagctgctgaagctgtacgcctgggagaacatcttccgcacgcgggtggagacgacccgcaggaaggagatgaccagcctcagggcctttgccatctatacctccatctccattttcatgaacacggccatccccattgcagctgtcctcataactttcgtgggccatgtcagcttcttcaaagaggccgacttctcgccctccgtggcctttgcctccctctccctcttccatatcttggtcacaccgctgttcctgctgtccagtgtggtccgatctaccgtcaaagctctagtgagcgtgcaaaagctaagcgagttcctgtccagtgcagagatccgtgaggagcagtgtgccccccatgagcccacacctcagggcccagccagcaagtaccaggcggtgcccctcagggttgtgaaccgcaagcgtccagcccgggaggattgtcggggcctcaccggcccactgcagagcctggtccccagtgcagatggcgatgctgacaactgctgtgtccagatcatgggaggctacttcacgtggaccccagatggaatccccacactgtccaacatcaccattcgtatcccccgaggccagctgactatgatcgtggggcaggtgggctgcggcaagtcctcgctccttctagccgcactgggggagatgcagaaggtctcaggggctgtcttctggagcagccttcctgacagcgagataggagaggaccccagcccagagcgggagacagcgaccgacttggatatcaggaagagaggccccgtggcctatgcttcgcagaaaccatggctgctaaatgccactgtggaggagaacatcatctttgagagtcccttcaacaaacaacggtacaagatggtcattgaagcctgctctctgcagccagacatcgacatcctgccccatggagaccagacccagattggggaacggggcatcaacctgtctggtggtcaacgccagcgaatcagtgtggcccgagccctctaccagcacgccaacgttgtcttcttggatgaccccttctcagctctggatatccatctgagtgaccacttaatgcaggccggcatccttgagctgctccgggacgacaagaggacagtggtcttagtgacccacaagctacagtacctgccccatgcagactggatcattgccatgaaggatgggaccatccagagggagggtaccctcaaggacttccagaggtctgaatgccagctctttgagcactggaagaccctcatgaaccgacaggaccaagagctggagaaggagactgtcacagagagaaaagccacagagccaccccagggcctatctcgtgccatgtcctcgagggatggccttctgcaggatgaggaagaggaggaagaggaggcagctgagagcgaggaggatgacaacctgtcgtccatgctgcaccagcgtgctgagatcccatggcgagcctgcgccaagtacctgtcctccgccggcatcctgctcctgtcgttgctggtcttctcacagctgctcaagcacatggtcctggtggccatcgactactggctggccaagtggaccgacagcgccctgaccctgacccctgcagccaggaactgctccctcagccaggagtgcaccctcgaccagactgtctatgccatggtgttcacggtgctctgcagcctgggcattgtgctgtgcctcgtcacgtctgtcactgtggagtggacagggctgaaggtggccaagagactgcaccgcagcctgctaaaccggatcatcctagcccccatgaggttttttgagaccacgccccttgggagcatcctgaacagattttcatctgactgtaacaccatcgaccagcacatcccatccacgctggagtgcctgagccgctccaccctgctctgtgtctcagccctggccgtcatctcctatgtcacacctgtgttcctcgtggccctcttgcccctcgcagtcgtgtgctacttcatccagaagtacttccgggtggcgtccagggacctgcagcagctggatgacaccacccagcttccacttctctcacactttgccgaaaccgtagaaggactcaccaccatccgggccttcaggtatgaggcccggttccagcagaagcttctcgaatacacagactccaacaacattgcttccctcttcctcacagctgccaacagatggctggaagtccgaatggagtacatcggtgcatgtgtggtgctcatcgcagcggtgacctccatctccaactccctgcacagggagctctctgctggcctggtgggcctgggccttacctacgccctaatggtctccaactacctcaactggatggtgaggaacctggcagacatggagctccagctgggggctgtgaagcgcatccatgggctcctgaaaaccgaggcagagagctacgaggggctcctggcaccatcgctgatcccaaagaactggccagaccaagggaagatccagatccagaacctgagcgtgcgctacgacagctccctgaagccggtgctgaagcacgtcaatgccctcatctcccctggacagaagatcgggatctgcggccgcaccggcagtgggaagtcctccttctctcttgccttcttccgcatggtggacacgttcgaagggcacatcatcattgatggcattgacatccgcaaactgccgctgcacaccctgccgtcacgcctctccatcatcctgcaggaccccgtcctcttcagcggcaccatccgatttaacctggaccctgagaggaagtgctcagatagcacactgtgggaggccctggaaatcgcccagctgaagctggtggtgaaggcactgccaggaggcctcgatgccatcatcacagaaggcggggagaatttcagccagggacagaggcagctgttctgcctggcccgggccttcgtgaggaagaccagcatcttcatcatggacgaggccacggcttccattgacatggccacggaaaacatcctccaaaaggtggtgatgacagccttcgcagaccgcactgtggtcaccatcgcgcatcgagtgcacaccatcctgagtgcagacctggtgatcgtcctgaagcggggtgccatccttgagttcgataagccagagaagctgctcagccggaaggacagcgtcttcgcctccttcgtccgtgcagacaagtgacctgccagagcccaagtgccatcccacattcggaccctgcccatacccctgcctgggttttctaactgtaaatcacttgtaaataaatagatttgattatttcct

SEQ ID NO: 26 - protein, 1581 aa, Homo sapiens
MPLAFCGSENHSAAYRVDQGVLNNGCFVDALNVVPHVFLLFITFPILFIGWGSQSSKVHIHHSTWLHFPGHNLRWILTFMLLFVLVCEIAEGILSDGVTESHHLHLYMPAGMAFMAAVTSVVYYHNIETSNFPKLLIALLVYWTLAFITKTIKFVKLLDHAIGFSQLRFCLTGLLVILYGMLLLVEVNVIRVRRYIFFKTPREVKPPEDLQDLGVRFLQPFVNLPSKGTYWWMNAFIKTAHKKPIDLRAIGKLPIVMRALTNYQRLCEAFDAQVRKDIQGTQGARAIWQALSHAFGRRLVLSSTFRILADLLGFAGPLCIFGIVDHLGKENDVFQPKTQFLGVYFVSSQEFLANAYVLAVLLFLALLLQRTFLQASYYVAIETGINLRGAIQTKIYNKIMHLSTSNLSMGEMTAGQICNLVAIDTNQLMWFFFLCPNLWAMPVQIIVGVILLYYILGVSALIGAAVIILLAPVQYFVATKLSQAQRSTLEYSNERLKQTNEMLRGIKLLKLYAWENIFRTRVETTRRKEMTSLRAFAIYTSISIFMNTAIPIAAVLITFVGHVSFFKEADFSPSVAFASLSLFHILVTPLFLLSSVVRSTVKALVSVQKLSEFLSSAEIREEQCAPHEPTPQGPASKYQAVPLRVVNRKRPAREDCRGLTGPLQSLVPSADGDADNCCVQIMGGYFTWTPDGIPTLSNITIRIPRGQLTMIVGQVGCGKSSLLLAALGEMQKVSGAVFWSSLPDSEIGEDPSPERETATDLDIRKRGPVAYASQKPWLLNATVEENIIFESPFNKQRYKMVIEACSLQPDIDILPHGDQTQIGERGINLSGGQRQRISVARALYQHANVVFLDDPFSALDIHLSDHLMQAGILELLRDDKRTVVLVTHKLQYLPHADWIIAMKDGTIQREGTLKDFQRSECQLFEHWKTLMNRQDQELEKETVTERKATEPPQGLSRAMSSRDGLLQDEEEEEEEAAESEEDDNLSSMLHQRAEIPWRACAKYLSSAGILLLSLLVFSQLLKHMVLVAIDYWLAKWTDSALTLTPAARNCSLSQECTLDQTVYAMVFTVLCSLGIVLCLVTSVTVEWTGLKVAKRLHRSLLNRIILAPMRFFETTPLGSILNRFSSDCNTIDQHIPSTLECLSRSTLLCVSALAVISYVTPVFLVALLPLAVVCYFIQKYFRVASRDLQQLDDTTQLPLLSHFAETVEGLTTIRAFRYEARFQQKLLEYTDSNNIASLFLTAANRWLEVRMEYIGACVVLIAAVTSISNSLHRELSAGLVGLGLTYALMVSNYLNWMVRNLADMELQLGAVKRIHGLLKTEAESYEGLLAPSLIPKNWPDQGKIQIQNLSVRYDSSLKPVLKHVNALISPGQKIGICGRTGSGKSSFSLAFFRMVDTFEGHIIIDGIDIRKLPLHTLPSRLSIILQDPVLFSGTIRFNLDPERKCSDSTLWEALEIAQLKLVVKALPGGLDAIITEGGENFSQGQRQLFCLARAFVRKTSIFIMDEATASIDMATENILQKVVMTAFADRTVVTIAHRVHTILSADLVIVLKRGAILEFDKPEKLLSRKDSVFASFVRADK

SEQ ID NO: 27 - DNA, 3974 bp, Homo sapiens
gtgcattgagcgcgctccagctgccgggacggagggggcggcccccgcgctcggggcgctcggctacagctgcggggcccgaggtctccgcgcactcgctcccggcccatgctggaggcggcggaaccgcggggacctaggacggaggcggcgggcgctgggcggcccccggcacgctgagctcgggatgcggacgctgctgcctcccgcgctgctgacctgctggctgctcgcccccgtgaacagcattcacccagaatgccgatttcatctggaaatacaggaggaagaaacaaaatgtgcagagcttctgaggtctcaaacagaaaaacacaaagcctgcagtggcgtctgggacaacatcacgtgctggcggcctgccaatgtgggagagaccgtcacggtgccctgcccaaaagtcttcagcaatttttacagcaaagcaggaaacataagcaaaaactgtacgagtgacggatggtcagagacgttcccagatttcgtcgatgcctgtggctacagcgacccggaggatgagagcaagatcacgttttatattctggtgaaggccatttataccctgggctacagtgtctctctgatgtctcttgcaacaggaagcataattctgtgcctcttcaggaagctgcactgcaccaggaattacatccacctgaacctgttcctgtccttcatcctgagagccatctcagtgctggtcaaggacgacgttctctactccagctctggcacgttgcactgccctgaccagccatcctcctgggtgggctgcaagctgagcctggtcttcctgcagtactgcatcatggccaacttcttctggctgctggtggaggggctctacctccacaccctcctggtggccatgctcccccctagaaggtgcttcctggcctacctcctgatcggatggggcctccccaccgtctgcatcggtgcatggactgcggccaggctctacttagaagacaccggttgctgggatacaaacgaccacagtgtgccctggtgggtcatacgaataccgattttaatttccatcatcgtcaattttgtccttttcattagtattatacgaattttgctgcagaagttaacatccccagatgtcggcggcaacgaccagtctcagtacaagaggctggccaagtccacgctcctgcttatcccgctgttcggcgtccactacatggtgtttgccgtgtttcccatcagcatctcctccaaataccagatactgtttgagctgtgcctcgggtcgttccagggcctggtggtggccgtcctctactgtttcctgaacagtgaggtgcagtgcgagctgaagcgaaaatggcgaagccggtgcccgaccccgtccgcgagccgggattacagggtctgcggttcctccttctcccgcaacggctcggagggcgccctgcagttccaccgcggctcccgcgcccagtccttcctgcaaacggagacctcggtcatctagccccacccctgcctgtcggacgcggcgggaggcccacggttcggggcttctgcggggctgagacgccggcttcctccttccagatgcccgagcaccgtgtcgggcaggtcagcgcggtcctgactccgtcaagctggttgtccactaaaccccatacctggaattggagtcgtgttgtcattgactcgatttaaactccagcatttagataatcttgtgcaaaatgtgtttcagccgtatagtggatccacttttttttttttttttttttgagacggagtcttgctctgtcgcccaggctggagtgcagtggcctgatctctgctccctgcaagctccgcctcccgggttcacgccattctcctgcctcagcctcccatagctgggactacaggcgcccgccaacacgcctggctaattttttgtatttttagtagagacagggtttcaccatgttagccaggatggtctcgatctcctgacctcgtgatgggcccgcctcggcctcccaaagtgctgggattaaggcgtgagccactgcgcccggcccaagagaataggggagccaaggaggaaatgtggaaacgcagttgtgtggcccagcacgagcctgggcgaccaccgggtgacatccgtcccacatcagggcggcctcccaggtcccataagggtagccccctcatctgcaggacagagggaagccagtcagggcccccccggacgttaggaccaggagaaatcaacaggagggcagcccgtcctctctcttggggcgcccacccggcccggctgagccctgccccacccaactccacagggctgttttgcctccccacggaaggcgggctgaggagacaaccagatcaggagagcaaggtcatgaaggaggggacctctccacacaggtgttccgtgggaccctcagcagctctggctctgcctcaggaggtcacctgccgccctgtgggagccgcagagcctgacgctcagccccaggccagctgcggccaggcctgcgggcccctggtgatggggttacgtggggtgcgggatacagctgagtgggaaccggaaacctattctctttttaacaaaaataatcttaggataagaattattttaacaacatataaaactgtttcaagccctcctccccagagctggcgctcagcagccctagcggctgctccttcaggcgaagggtggtttgcagatgtggggagggtgtctggggacgttgctgagctggctgcagaagggtggggatatcagggcacagtctccatgtgtgtgccaagccctggcccccacagcgctcgatggacctcagcaagctgcccagccctggcccaggtgccccgactgtgggactcagttgttctgagcacatttgactccacttttcctttaaaaatgaatgtcttgttcctgtgcattggtggcatcacagaccccagctggggcgcgatgtcaaaggtcgggacagctgtgccgggaggcagccacagggaagctcacacatcctgtcagtgtcaccttggtttgcaaaacccatatccccggtaaaatgaggccggacagaggggctgttaggacagcaaagcagcagtgtccagagacccctcaatccccaaaggtccgcaccctgtcctgcacaccctgggccacgccggccacacccctctgctgcaacaagctcatccctggacttctgggagaatgaacccgaggttggtttggggagacaggtgaggcggttggatctacagaacaacccaccatttctgggggccgcagaggatccatcacagacggatactggggagtaaacggcccaggccaggtgcccaggaaaggacggctgagcatgtggagcgagagggaggcaggtggacgctgcagaccccaggttcagtgcggcccctcggctgttcctcccctgtagggtttggacagacccacccccagccttgcccagctttcaaaggacaaaagggagcatcccccacctactctcaggtttttgaggaaacaaagatttgtggtaactgaaggtgttgggtcagtggccaggtgccgacactgagctgtgacccagaggggacgctgaggaagtgggcgtgagtggacatgtcaggtggttaccaggcactggttgttgatggtcggtggttgggtgtgggcagtcatcagtcatcaggtgtgctcaggggacaatctcccctcaaccgcacatgtgccactgttcagcggagctgactggtttctcctggtagagggccggctgtatcctgacagatgcctggtgagcaggggaagcaggacccagtggtcaacaggtgtctttaactgtcattgtgtgtggaatgtcgcagactcctccacgtggcgggaatgagctgtgtaaatacttcaataaagcctgatctcacatctgcaaaaaaaaaaaaaaaaaaaaaaaaaaaaaaaa

SEQ ID NO: 28 - DNA, 3958 bp, Homo sapiens
gtgcattgagcgcgctccagctgccgggacggagggggcggcccccgcgctcgggcgctcggctacagctgcggggcccgaggtctccgcgcactcgctcccggcccatgctggaggcggcggaaccgcggggacctaggacggaggcggcgggcgctgggcggcccccggcacgctgagctcgggatgcggacgctgctgcctcccgcgctgctgacctgctggctgctcgcccccgtgaacagcattcacccagaatgccgatttcatctggaaatacaggaggaagaaacaaaatgtgcagagcttctgaggtctcaaacagaaaaacacaaagcctgcagtggcgtctgggacaacatcacgtgctggcggcctgccaatgtgggagagaccgtcacggtgccctgcccaaaagtcttcagcaatttttacagcaaagcaggaaacataagcaaaaactgtacgagtgacggatggtcagagacgttcccagatttcgtcgatgcctgtggctacagcgacccggaggatgagagcaagatcacgttttatattctggtgaaggccatttataccctgggctacagtgtctctctgatgtctcttgcaacaggaagcataattctgtgcctcttcaggaagctgcactgcaccaggaattacatccacctgaacctgttcctgtccttcatcctgagagccatctcagtgctggtcaaggacgacgttctctactccagctctggcacgttgcactgccctgaccagccatcctcctgggtgggctgcaagctgagcctggtcttcctgcagtactgcatcatggccaacttcttctggctgctggtggaggggctctacctccacaccctcctggtggccatgctcccccctagaaggtgcttcctggcctacctcctgatcggatggggcctccccaccgtctgcatcggtgcatggactgcggccaggctctacttagaagacaccggttgctgggatacaaacgaccacagtgtgccctggtgggtcatacgaataccgattttaatttccatcatcgtcaattttgtccttttcattagtattatacgaattttgctgcagaagttaacatccccagatgtcggcggcaacgaccagtctcagtacaagaggctggccaagtccacgctcctgcttatcccgctgttcggcgtccactacatggtgtttgccgtgtttcccatcagcatctcctccaaataccagatactgtttgagctgtgcctcgggtcgttccagggcctggtggtggccgtcctctactgtttcctgaacagtgaggtgcagtgcgagctgaagcgaaaatggcgaagccggtgcccgaccccgtccgcgagccgggattacagggtctgcggttcctccttctcccgcaacggctcggagggcgccctgcagttccaccgcggctcccgcgcccagtccttcctgcaaacggagacctcggtcatctagccccacccctgcctgtcggacgcggcgggaggcccacggttcggggcttctgcggggctgagacgccggcttcctccttccagatgcccgagcaccgtgtcgggcaggtcagcgcggtcctgactccgtcaagctggttgtccactaaaccccatacctggaattggagtcgtgttgtcattgactcgatttaaactccagcatttagataatcttgtgcaaaatgtgtttcagccgtatagtggatccacttttttttttttttttttttgagacggagtcttgctctgtcgcccaggctggagtgcagtggcctgatctctgctccctgcaagctccgcctcccgggttcacgccattctcctgcctcagcctcccatagctgggactacaggcgcccgccaacacgcctggctaattttttgtatttttagtagagacagggtttcaccatgttagccaggatggtctcgatctcctgacctcgtgatgggcccgcctcggcctcccaaagtgctgggattaaggcgtgagccactgcgcccggcccaagagaataggggagccaaggaggaaatgtggaaacgcagttgtgtggcccagcacgagcctgggcgaccaccgggtgacatccgtcccacatcagggcggcctcccaggtcccataagggtagccccctcatctgcaggacagagggaagccagtcagggcccccccgacgttaggaccaggagaaatcaacaggagggcagcccgtcctctctcttggggcgcccacccggcccggctgagccctgccccacccaactccacagggctgttttgcctccccacggaaggcgggctgaggagacaaccagatcaggagagcaaggtcatgaaggaggggacctctccacacaggtgttccgtgggaccctcagcagctctggctctgcctcaggaggtcacctgccgccctgtgggagccgcagagcctgacgctcagccccaggccagctgcggccaggcctgcgggcccctggtgatggggttacgtggggtgcgggatacagctgagtgggaaccggaaacctattctctttttaacaaaaataatcttaggataagaattattttaacaacatataaaactgtttcaagccctcctccccagagctggcgctcagcagccctagcggctgctccttcaggcgaagggtggtttgcagatgtggggagggtgtctggggacgttgctgagctggctgcagaagggtggggatatcagggcacagtctccatgtgtgtgccaagccctggcccccacagcgctcgatggacctcagcaagctgcccagccctggcccaggtgccccgactgtgggactcagttgttctgagcacatttgactccacttttcctttaaaaatgaatgtcttgttcctgtgcattggtggcatcacagaccccagctggggcgcgatgtcaaaggtcgggacagctgtgccgggaggcagccacagggaagctcacacatcctgtcagtgtcaccttggtttgcaaaacccatatccccggtaaaatgaggccggacagaggggctgttaggacagcaaagcagcagtgtccagagacccctcaatccccaaaggtccgcaccctgtcctgcacaccctgggccacgccggccacacccctctgctgcaacaagctcatccctggacttctgggagaatgaacccgaggttggtttggggagacaggtgaggcggttggatctacagaacaacccaccatttctgggggccgcagaggatccatcacagacggatactggggagtaaacggcccaggccaggtgcccaggaaaggacggctgagcatgtggagcgagagggaggcaggtggacgctgcagaccccaggttcagtgcggcccctcggctgttcctcccctgtagggtttggacagacccacccccagccttgcccagctttcaaaggacaaaagggagcatcccccacctactctcaggtttttgaggaaacaaagatttgtggtaactgaaggtgttgggtcagtggccaggtgccgacactgagctgtgacccagaggggacgctgaggaagtgggcgtgagtggacatgtcaggtggttaccaggcactggttgttgatggtcggtggttgggtgtgggcagtcatcagtcatcaggtgtgctcaggggacaatctcccctcaaccgcacatgtgccactgttcagcggagctgactggtttctcctggtagagggccggctgtatcctgacagatgcctggtgagcaggggaagcaggacccagtggtcaacaggtgtctttaactgtcattgtgtgtggaatgtcgcagactcctccacgtggcgggaatgagctgtgtaaatacttcaataaagcctgacttcacatctgcaaaaaaaaaaaaaaaaaa

SEQ ID NO: 29 - protein, 438 aa, Homo sapiens
MRTLLPPALLTCWLLAPVNSIHPECRFHLEIQEEETKCAELLRSQTEKHKACSGVWDNITCWRPANVGETVTVPCPKVFSNFYSKAGNISKNCTSDGWSETFPDFVDACGYSDPEDESKITFYILVKAIYTLGYSVSLMSLATGSIILCLFRKLHCTRNYIHLNLFLSFILRAISVLVKDDVLYSSSGTLHCPDQPSSWVGCKLSLVFLQYCIMANFFWLLVEGLYLHTLLVAMLPPRRCFLAYLLIGWGLPTVCIGAWTAARLYLEDTGCWDTNDHSVPWWVIRIPILISIIVNFVLFISIIRILLQKLTSPDVGGNDQSQYKRLAKSTLLLIPLFGVHYMVFAVFPISISSKYQILFELCLGSFQGLVVAVLYCFLNSEVQCELKRKWRSRCPTPSASRDYRVCGSSFSRNGSEGALQFHRGSRAQSFLQTETSVI

SEQ ID NO: 30 - DNA, 1640 bp, Homo sapiens
cgggacgagggggcggcccccgcgctcggggcgctcggctacagctgcggggcccgaggtctccgcgcactcgctcccggcccatgctggaggcggcggaacccgggggacctaggacggaggcggcgggcgctgggcggcccccggcacgctgagctcgggatgcggacgctgctgcctcccgcgctgctgacctgctggctgctcgcccccgtgaacagcattcacccagaatgccgatttcatctggaaatacaggaggaagaaacaaaatgtacagagcttctgaggtctcaaacagaaaaacacaaagcctgcagtggcgtctgggacaacatcacgtgctggcggcctgccaatgtgggagagaccgtcacggtgccctgcccaaaagtcttcagcaatttttacagcaaagcaggaaacataagcaaaaactgtacgagtgacggatggtcagagacgttcccagatttcgtcgatgcctgtggctacagcgacccggaggatgagagcaagatcacgttttatattctggtgaaggccatttataccctgggctacagtgtctctctgatgtctcttgcaacaggaagcataattctgtgcctcttcaggaagctgcactgcaccaggaattacatccacctgaacctgttcctgtccttcatcctgagagccatctcagtgctggtcaaggacgacgttctctactccagctctggcacgttgcactgccctgaccagccatcctcctgggtgggctgcaagctgagcctggtcttcctgcagtactgcatcatggccaacttcttctggctgctggtggaggggctctacctccacaccctcctggtggccatgctcccccctagaaggtgcttcctggcctacctcctgatcggatggggcctccccaccgtctgcatcggtgcatggactgcggccaggctctacttagaagacaccggttgctgggatacaaacgaccacagtgtgccctggtgggtcatacgaataccgattttaatttccatcatcgtcaattttgtccttttcattagtattatacgaattttgctgcagaagttaacatccccagatgtcggcggcaacgaccagtctcagtacaagaggctggccaagtccacgctcctgcttatcccgctgttcggcgtccactacatggtgtttgccgtgtttcccatcagcatctcctccaaataccagatactgtttgagctgtgcctcgggtcgttccagggcctggtggtggccgtcctctactgtttcctgaacagtgaggtgcagtgcgagctgaagcgaaaatggcgaagccggtgcccgaccccgtccgcgagccgggattacagggtctgcggttcctccttctcccacaacggctcggagggcgccctgcagttccaccgcgcgtcccgagcccagtccttcctgcaaacggagacctcggtcatctagccccacccctgcctgtcggacgcggcgggaggcccacggttcggggcttctgcggggctgagacgccggcttcctccttccagatgcccgagcaccgtgtcgggcaggtcagcgcggtcctgactccgtcaagctggttgtccactaaaccccatacctgg

SEQ ID NO: 31 - protein, 438 aa, Homo sapiens
MRTLLPPALLTCWLLAPVNSIHPECRFHLEIQEEETKCTELLRSQTEKHKACSGVWDNITCWRPANVGETVTVPCPKVFSNFYSKAGNISKNCTSDGWSETFPDFVDACGYSDPEDESKITFYILVKAIYTLGYSVSLMSLATGSIILCLFRKLHCTRNYIHLNLFLSFILRAISVLVKDDVLYSSSGTLHCPDQPSSWVGCKLSLVFLQYCIMANFFWLLVEGLYLHTLLVAMLPPRRCFLAYLLIGWGLPTVCIGAWTAARLYLEDTGCWDTNDHSVPWWVIRIPILISIIVNFVLFISIIRILLQKLTSPDVGGNDQSQYKRLAKSTLLLIPLFGVHYMVFAVFPISISSKYQILFELCLGSFQGLVVAVLYCFLNSEVQCELKRKWRSRCPTPSASRDYRVCGSSFSHNGSEGALQFHRASRAQSFLQTETSVI

SEQ ID NO: 32 - DNA, 2374 bp, Homo sapiens
gggtggggggaataacatgttcttttaaacgcagagtttaaacattgagttgcatcattgtgaggaaaaccacttagtatttttagtgaggtgactttacaagtaaagatcttcaagaagatttttatgtgatttaaaaaatcagcttagatgcttggaatttggattgttgcattcttgttctttggcacatcaagaggaaaagaagtttgctatgaaaggttagggtgtttcaaagatggtttaccatggaccaggactttctcaacagagttggtaggtttaccctggtctccagagaagataaacactcgtttcctgctctacactatacacaatcccaatgcctatcaggagatcagtgcggttaattcttcaactatccaagcctcatattttggaacagacaagatcacccgtatcaacatagctggatggaaaacagatggcaaatggcagagagacatgtgcaatgtgttgctacagctggaagatataaattgcattaatttagattggatcaacggttcacgggaatacatccatgctgtaaacaatctccgtgttgttggtgctgaggtggcttattttattgatgttctcatgaaaaaatttgaatattccccttctaaagtgcacttgattggccacagcttgggagcacacctggctggggaagctgggtcaaggataccaggccttggaagaataactgggttggacccagctgggccatttttccacaacactccaaaggaagtcaggctagacccctcggatgccaactttgttgacgttattcatacaaatgcagctcgcatcctctttgagcttggtgttggaaccattgatgcttgtggtcatcttgacttttacccaaatggagggaagcacatgccaggatgtgaagacttaattacacctttactgaaatttaacttcaatgcttacaaaaaagaaatggcttccttctttgactgtaaccatgcccgaagttatcaattttatgctgaaagcattcttaatcctgatgcatttattgcttatccttgtagatcctacacatcttttaaagcaggaaattgcttcttttgttccaaagaaggttgcccaacaatgggtcattttgctgatagatttcacttcaaaaatatgaagactaatggatcacattattttttaaacacagggtccctttccccatttgcccgttggaggcacaaattgtctgttaaactcagtggaagcgaagtcactcaaggaactgtctttcttcgtgtaggcggggcaattgggaaaactggggagtttgccattgtcagtggaaaacttgagccaggcatgacttacacaaaattaatcgatgcagatgttaacgttggaaacattacaagtgttcagttcatctggaaaaaacatttgtttgaagattctcagaataagttgggagcagaaatggtgataaatacatctgggaaatatggatataaatctacctactgtagccaagacattatgggacctaatattctccagaacctgaaaccatgctaatctcagatacagtcttgatggatttctttagtaggagcaatgaagaaaagtgtctccttccacctggcatccagaccaaatttgacccttgtaaatgacttagtcatttacaggggtcttactcagagtcaagtacgggtttgctttttttctgtgtagaatgttcatctaactgcaccttaaaaacacactgaaccctgggacaaaagataattactatgatctgtaggaatctggatatcattgacaaaatagagctgttttggaattttcctgaataagaggaggtgatgcaaatgtatgttgagtgtataaactcactggacaaaagtaagcctctggcttgctgagtttttgaagtatattttcaggtataataatcattgttctaaaattatataaaactatttgttatgttgttaaatcttgctgagacaaattatgactatagtgcatgatatatagtagattataaccttgtgggttgatgtgtctatctagtaataataaaaactaatgagatggcactagtatttccaaggtgttccttggtgttcagggtgtgcacaagagagattttggagcttatctgttatgtgttcatcagttagcaatgggacctgaagttcaacaacccagggtatagcccccttcctccaaagtccctgccacaggagaattactcctctctctgggtcttgaatgctctatggtgaatttgtatttagcctcaaggcagcatttcatttgtaaagcacttgggtaaccctttgttcttgcaataacaatattataatatttaaaaaaaaaaaaaaaaaaa

SEQ ID NO: 33 - protein, 467 aa, Homo sapiens
MLGIWIVAFLFFGTSRGKEVCYERLGCFKDGLPWTRTFSTELVGLPWSPEKINTRFLLYTIHNPNAYQEISAVNSSTIQASYFGTDKITRINIAGWKTDGKWQRDMCNVLLQLEDINCINLDWINGSREYIHAVNNLRVVGAEVAYFIDVLMKKFEYSPSKVHLIGHSLGAHLAGEAGSRIPGLGRITGLDPAGPFFHNTPKEVRLDPSDANFVDVIHTNAARILFELGVGTIDACGHLDFYPNGGKHMPGCEDLITPLLKFNFNAYKKEMASFFDCNHARSYQFYAESILNPDAFIAYPCRSYTSFKAGNCFFCSKEGCPTMGHFADRFHFKNMKTNGSHYFLNTGSLSPFARWRHKLSVKLSGSEVTQGTVFLRVGGAIGKTGEFAIVSGKLEPGMTYTKLIDADVNVGNITSVQFIWKKHLFEDSQNKLGAEMVINTSGKYGYKSTYCSQDIMGPNILQNLKPC

SEQ ID NO: 34 - DNA, 447 bp, Homo sapiens
tttttccatgtaatatttgttttatttataataagaggaaatacatttgaacaaagaagctctcatagtattggcaattttacatatatctctgttattgtaattttttttacttgctgggcttggtaattcttcaatggacatgaaagctatgacctagagagactatagagtcgctggtaagcgtacgcccgaggccctgggcgtccccactggtagatggtggcgtgtggacgaacagcttagtccttgggcaaagcttgtgctggtcgagagtggcgagtctgggacagagacccaggctgctccctgctgcttccaggctcctctctcttagacttaatgcccaggaaactgagtattttcatcagcagcaaatctacgatctccccttcctccgacagctgcaagagaaagaaccaggcaatgcccatagaaccatcttct

SEQ ID NO: 35 - DNA, 523 bp, Homo sapiens
ttttctaagaatttgtcttatttttaatgcatggaaaatagcaaaattatcatgccaacatgaggaatatatactataattcataaatgcctaattatcaaaataatgacatagtcatggttagatgcaacctagaaatcttatataagatgcaactacatattgtatgatcattcctcttatatatgacattcaatcctcatcaaattcagctatgtataaatggcattatgaaataaacacttaatatcacaatagggtcatagtctgctactgtacaaccatggcatgcaagtaactatgcattagctgtaaacagtaaagtgtcataaccttccagaaatccaaagaatgtgaaaagtacatatatagtactaaacatcaattgtatttaaaggaccttcatatttaacaaagctatatcatatacagcagctttggagatttctgtcactgttatacatatcttgtcaccctgaagtgaggaaactgcaattccaaactatatctgttaatgctactg

SEQ ID NO: 36 - DNA, 440 bp, Homo sapiens
ttttttttttttttttttttcctttttttttaaaaaaaggggtttatttccttttttttaagattcagtaggatagccaaattcatagagaataaaattacatgaaagagttacaagctcactgttttaaagacttgacatttttcatttagttttaattaacagtaataagacacctcctgtttttcaatgttcaccaaaaaaagaaacatagaatagggggaaaacatgcttatatagccaaggtacagatccagatgatgtaacctttttagtattcgcatgacttgaaaactgggcagatcaatagataatcgaagtgctttatctgaagggagagggtaaagacagtgtgaccaggtttgttttcagggctgccgaatgagcctcacctaacagtgtccatgggtaattcgctaaccttaacaaagatgggaaga

SEQ ID NO: 37 - DNA, 6593 bp, Homo sapiens
caaagtaaactcggtggcctcttcttctccacccctcaaaatgatagcaatctctgccgtcagcagtgcactcctgttctcccttctctgtgaagcaagtaccgtcgtcctactcaattccactgactcatccccgccaaccaataatttcactgatattgaagcagctctgaaagcacaattagattcagcggatatccccaaagccaggcggaagcgctacatttcgcagaatgacatgatcgccattcttgattatcataatcaagttcggggcaaagtgttcccaccggcagcaaatatggaatatatggtttgggatgaaaatcttgcaaaatcggcagaggcttgggcggctacttgcatttgggaccatggaccttcttacttactgagatttttgggccaaaatctatctgtacgcactggaagatatcgctctattctccagttggtcaagccatggtatgatgaagtgaaagattatgcttttccatatccccaggattgcaaccccagatgtcctatgagatgttttggtcccatgtgcacacattatacgcagatggtttgggccacttccaatcggataggatgcgcaattcatacttgccaaaacatgaatgtttggggatctgtgtggcgacgtgcagtttacttggtatgcaactatgccccaaagggcaattggattggagaagcaccatataaagtaggggtaccatgttcatcttgtcctccaagttatgggggatcttgtactgacaatctgtgttttccaggagttacgtcaaactacctgtactggtttaaataagtttaccttttcctccaggaaatataatgatttctgggaacatgggcatgtatatatatatatggagagagaattttgcacatattatacatattttgtgctaatcttgttttcctcttagtattcctttgtataaattagtgtttgtctagcatgtttgtttaatcctttgaaatatttgaaacatcaatttctattttctgacctctaagcctaaattaagatattgtatatgtaatgatgacatagttgatgcatccaatcctaaaacttacattccaaaggaattatatcattatgttcctaaggagtaaatatatatttgacctgtaagtgtgtgtatgtatacatatacatatgtatgtgtatggatttatatatgcacacaaacatataatatgtgatgtaacatgtagatgataatatgattcagtagtcaacttgagggaaatttttaaaaaactattctcaattatatacgaggtgatgggacttcttaacacacatttctataatacccatgaaatgataatttgtaaaataacacttagtgatatctggaaataataattcaattaagcaaccacgaatttcaccctggagatattttttcttatttgagtccaccaaaggataatgccaacttatataagttctcaaatcatgccttccgcttagtctcattttattcattcagtcgtcatgagttgagtgcttactacatgcaaggcactctgctagttatattctaataatgcagagataattagacatggttcccgccctcaagaagctcacaaaagtattcaggaaataatgcagactagtgattttgctataaaattatttttgaaggaagcagacacagcagtatttacctgtaggtggagcaagtaataagccatgctgtgcaatatatacataaagcttctgcttctcatgggaatttagttacagtgcttggaatgagaaggggaaggaaagaattaacaaatgccaagatttctggagcagattgtacagctgtgactttggaaaacagaaagtaagaccctcagaaaaccaatgaagtctaagagaaataaaatttagtggacaggtatgaaaagtgtaattgcgcctaactaccagatggagagcttcagaatgggctatccttagagtctagtacatcttgaggcctctcagcaggagacaaaggattccaaaaagagatgtggaggtgctgagggcacctctatctctttgttgtttagtctgtcattgaatcaatctacttatcatcttgggtctttgagtattgtatgaaagatccttcgtgcacaccacacacagtcatgattttgttaaagtagccttcttcagatgcttttctaagggctagttaccaactttatttctgtgtttctgtagaagaaacattttcagttcttcattgagtttgattatggaaatccattcaaagtcactatgaaaattttactcatgtagtttggaaatgcaacattttcctatcatgaaatctctttcagagaggagaatacaacatcttagtccagacatttaacatactgcatttcaagtacatgtgtgtgtgttttattcaggttgtgtaatgctcccgtagaattatagaacaattaaatatggttagttccagagtgcaaattacagaaggaagctacttgtttaaaattccatacacgtttgcagtttcttgtacacatttggatactttgaaagatgacagattgttaaatccattcaatggtaaagaaactcaccatctggagattgagtctacttgttaatgaatgactagcccaattatccttataaattgaatatggtgaccaaatgctttgatatcatactactctgcctttgtgggcacatatgtagacactactaaaaataaatatttttggagattaaaatggagaatagaagtaattacattatttaggtcttaatccaacttttttctaatatatctaaacaattgaaagggaagcttattcatggaatattggcttgatttatctagaaagtttttccttcttcaattttactatattcattctacaggaacagcaataagtactattaaacagaagatggctacactaagttccaattttgttgctgaattgcttctgtgagttcacttttcagttctaaggaagaataatatttgctacatatttcacaggggttcttatgaaggtaaatttaccagattaataaaaatttatgaatattaaaattatcattaataatataaaacacttatttgagattaaattaaatttttcatgagcccctctttggcaggaactctgtttaattctttgtatttatcccagcttcttaaatggtggctgtaacataataaatgtttaataaatgcttatatgaatggatttttagaattaactaagaagccaaaaatggcaacaatttacagaaatcccacctttccatgcttaagacaaaaatgtcttaaatataaagctgtgattatatcaaaaatccagataaatcatcaaatatatcagattaagaccagggtttacacacttaggcaatagtcctttccaaaccatgacaaaaactacaagtttatttataatttaacaactcagctgaaaatataacgggtatatttgttattctaactctattttttaaagttaataatataaagtggccatgtaaaatattttattttccaggctaaagcaaatgaaagtttgctggtatcaacacagcctgccatatttttcacagcatgcaacaatggtgctaggatagctatttcttactgtaattgccagaggcagaaatggtctgggtataagctatttcataaaagcagctttaaattgtcagtattaaggttttcatgtggaaaggtgtcattcaaaaaaaaagtaattggcatacatattccacatcatcgatcctctctgtggtgttaatttttttatatgaccagtagaaaaattttaatattctcacaatataggttttggggcttccatatcatcaaaagactgaaaaattataattttagaattaaactgatggatttcattatagaattatctgtgagttgtgtagacacagtcttaatgtttctggttatgacagataagtttgctcaaaaaatgtggatgaagccattattgttattattgttattgcttctgttcagttgtctaagtatcatcccttctgtggcccatcacgcagcagagttgccctacaaatttcatttggcagcgccataacattcatttaaaaagtttatgaaaacattcatttgaaagttccatgcagctttagcacagagttgaccaaacactggcgtaagttcaatttacacagaatatttgaattgaaacaatagaaatttttctcataatatatacctatgtgaaaccaacttatctgcataattaaatctaatacatatttaagccagtttaagtgctttgtgttgatgccatgcttatcaaatacatgcacaagctaaacataatttgaatgggtctatgaaggaaaaataatgcttagactttggtgtaggttcttcctgtgtagccatatacccaggctctgcagtatcgaaggatgcaaatgttgacatagatggaagctcttacctaccaaagtgtttaggaaggataaagttacatttgtcttaatttctaacattatctttgcttttatgtttcataaaaatttgtcattatttatgctggtgaaacgtataatcacatccaattatttgaacacatgcaaaataattttttaaattatgttattgtttaaatttgacttatgggagatcagtcaaaaacttagaaggtttaacacttcactgattaatggtgctgaaaacacgttacaattaccacatatccttgctataagttttgaagtttcttagcaattaaagtttttttattcagtgtgaactgtcagtatctattctggtgctaaatgtatggtgctaaatgaattgttagtgttgatggctttagtaatgctccttttattcattgctaaatttagtgttatccatttgattcctgattcagaaatatcaataaaatcctatgttaaattaatctttaccaaaaacaggcaagttaactctgttgttttaattcaacagtccaacattatttaggtgttacagagtgtaaatatatttctttgggagttattttctttttaaaatctttttatagcttggcaatgtccaaagtcaaatatcacctaaactggttagattacttctacagctaataatattgcaggcactggcgccctctggtggttatgaagacaaattcttaatggctacttgacctacagcaaaagccatttctgtaccataaaaatttgttgtgcaatattagaattatcatatgtttcctacatctgacagcacctaaaatgtttgataatattaacatgtatctaagaggaaaaaagagttaatatattctggcacccactttcctagtaatgttttccatgattttccagttctgaggcacttattaaagtgcttttttttttctgaattaattaggtattggtaaaatatatttttaaatttagttagctttataaacacaattagaattacaattaattaacagaggtataattgtctcactttcagaagtgatcatttatttttatttagcacaggtcataagaaaaatatatagaaaaataatcaatttcatatataaaaggattatttctccacctttaattattggcctatcatttgttagtgttatttggtcatattattgaactaatgtattattccattcaaagtctttctagatttaaaaatgtatgcaaaagcttaggattatatcatgtgtaactattatagataacatcctaaaccttcagtttagatatataattgactgggtgtaatctcttttgtaatctgttttgacagatttcttaaattatgttagcataatcaaggaagatttaccttgaagcactttccaaattgatactttcaaacttattttaaagcagtagaaccttttctatgaactaaatcacatgcaaaactccaacctgtagtatacataaaatggacttacttattcctctcaccttctccagtgcctaggaatattcttctctgagccctaggattgattctatcacacagagcaacattaatctaaatggtttagctccctcttttttctctaaaaacaatcagctaataaaaaaaaaatttgagggcctaaattatttcaatggttgtttgaaatattcagttcagtttgtacctgttagcagtctttcagtttgggggagaattaaatactgtgctaagctggtgcttggatacatattacagcatcttgtgttttatttgacaaacagaattttggtgccataatattttgagaattagagaagattgtgatgcatatatataaacactatttttaaaaaatatctaaatatgtctcacatatttatataatcctcaaatatactgtaccattttagatattttttaaacagattaatttggagaagttttattcattacctaattctgtggcaaaaatggtgcctctgatgttgtgatatagtattgtcagtgtgtacatatataaaacctgtgtaaacctctgtccttatgaaccataacaaatgtagctttttaaagtccattgtattgttttttctttcaataaaagagtataattaa

SEQ ID NO: 38 - protein, 258 aa, Homo sapiens
MIAISAVSSALLFSLLCEASTVVLLNSTDSSPPTNNFTDIEAALKAQLDSADIPKARRKRYISQNDMIAILDYHNQVRGKVFPPAANMEYMVWDENLAKSAEAWAATCIWDHGPSYLLRFLGQNLSVRTGRYRSILQLVKPWYDEVKDYAFPYPQDCNPRCPMRCFGPMCTHYTQMVWATSNRIGCAIHTCQNMNVWGSVWRRAVYLVCNYAPKGNWIGEAPYKVGVPCSSCPPSYGGSCTDNLCFPGVTSNYLYWFK

SEQ ID NO: 39 - DNA, 1235 bp, Homo sapiens
aggagccagaaccactcggcgccgcctggtgcatgggaggggagccgggccaggaacaatatgttagccgtgcactttgacaagccgggaggaccggaaaacctctacgtgaaggaggtggccaagccgagcccgggggagggtgaagtcctcctgaaggtggcggccagcgccctgaaccgggcggacttaatgcagagacaaggccagtatgacccacctccaggagccagcaacattttgggacttgaggcatctggacatgtggcagagctggggcctggctgccagggacactggaagatcggggacacagccatggctctgctccccggtgggggccaggctcagtacgtcactgtccccgaagggctcctcatgcctatcccagagggattgaccctgacccaggctgcagccatcccagaggcctggctcaccgccttccagctgttacatcttgtgggaaatgttcaggctggagactatgtgctaatccatgcaggactgagtggtgtgggcacagctgctatccaactcacccggatggctggagctattcctctggtcacagctggctcccagaagaagcttcaaatggcagaaaagcttggagcagctgctggattcaattacaaaaaagaggatttctctgaagcaacgctgaaattcaccaaaggtgctggagttaatcttattctagactgcataggcggatcctactgggagaagaacgtcaactgcctggctcttgatggtcgatgggttctctatggtctgatgggaggaggtgacatcaatgggcccctgttttcaaagctactttttaagcgaggaagtctgatcaccagtttgctgaggtctagggacaataagtacaagcaaatgctggtgaatgctttcacggagcaaattctgcctcacttctccacggagggcccccaacgtctgctgccggttctggacagaatctacccagtgaccgaaatccaggaggcccataagtacatggaggccaacaagaacataggcaagatcgtcctggaactgccccagtgaaggaggatggggcaggacaggacgcggccaccccaggcctttccagagcaaacctggagaagattcacaatagacaggccaagaaacccggtgcttcctccagagccgtttaaagctgatatgaggaaataaagagtgaactgaaaaaaaaaaaaaaaaaaaaaaaaaaaaaaaaa

SEQ ID NO: 40 - protein, 332 aa, Homo sapiens
MLAVHFDKPGGPENLYVKEVAKPSPGEGEVLLKVAASALNRADLMQRQGQYDPPPGASNILGLEASGHVAELGPGCQGHWKIGDTAMALLPGGGQAQYVTVPEGLLMPIPEGLTLTQAAAIPEAWLTAFQLLHLVGNVQAGDYVLIHAGLSGVGTAAIQLTRMAGAIPLVTAGSQKKLQMAEKLGAAAGFNYKKEDFSEATLKFTKGAGVNLILDCIGGSYWEKNVNCLALDGRWVLYGLMGGGDINGPLFSKLLFKRGSLITSLLRSRDNKYKQMLVNAFTEQILPHFSTEGPQRLLPVLDRIYPVTEIQEAHKYMEANKNIGKIVLELPQ

SEQ ID NO: 41 - DNA, 7127 bp, Homo sapiens
cccacgcgtccgggccggggctcaagatggctttagccgggctctgcgccctgctcgcctgctgctgggggccggcggcggtgctggccacggccgccggcgacgtggatccatccaaggagctggagtgcaagctcaaaagcatcacggtgtcggcactgcccttcctgcgcgagaacgacctgagcatcatgcacagcccctcggcctcggagcccaagctcctcttctcggtgcgcaacgacttcccgggagaaatggtcgtggtggacgacctggagaacacggagctgccctacttcgtgctggagatctcagggaacacagaggatatccctttggtgcgctggaggcagcagtggctggagaatggcactttgctttttcacattcatcaccaagatggtgccccaagccttcctggacaagaccccactgaagaaccccaacatgagtcggcagaagaggagctgaggatcctccacatctcagtcatgggtggcatgatcgctctgctgctgtccatcttgtgcctggtgatgatcctgtatactcgccggcgctggtgcaaacgccgccgggtcccgcagccccagaagagtgccagtgctgaggcagccaatgagattcactacattccttctgtgctgatcggcgggcacggacgggagagcctgcgcaatgcccgcgtgcagggccacaactccagtggcaccctgagcatccgggagacacctatcctggacggctatgagtatgacatcactgatctgcgccaccatctgcagagggagtgcatgaacggaggggaggactttgccagccaggtcacgcgcaccctcgactccctgcagggctgcaatgaaaagtcggggatggacctcacaccaggaagtgacaatgccaagctgtcactgatgaacaagtataaagataatattatagccactagccctgtggactccaaccaccagcaagccacccttctctctcacacctccagcagccagagaaagcggatcaacaacaaagcaagagctggttccgccttcttgaaccctgaaggggattctggcacagaggcagaaaacgacccccagctgaccttttacacggatccttcaaggagcaggaggcgtagtagagtgggttctccccgaagtcctgtgaataagaccaccttgaccctgatcagcatcaccagctgtgtgattggcctcgtgtgctcctctcacgtcaactgccctctcgttgtcaagatcaccctgcatgtccctgagcacctgattgctgatgggagccgcttcatcttgctggaggggagccagctggatgccagtgactggctgaaccctgcccaagtggttctcttctctcagcagaactccagcggaccctgggccatggacctctgtgcccggcggctcctggacccctgtgaacaccaatgtgaccccgaaactggggaatgcctctgctatgaaggctacatgaaggatccagtacataagcacctttgcattcggaacgaatgggggacaaaccaggggccatggccttacacaatatttcagcgaggctttgacctggttttgggagagcagccctctgataaaatatttagattcacctacactcttggggagggcatgtggttgcccctcagcaagagctttgtgattccaccagccgaactggccatcaatccatcagcaaagtgcaagacggacatgactgtgatggaggatgctgtggaggtcagagaggagctgatgacttcatcctccttcgacagcctggaggttctcttagattcctttgggccggtgcgcgactgcagcaaagataacgggggctgcagtaagaatttccgctgtatttcagatcgcaagctggactccactggttgcgtgtgcccatctggactcagtcccatgaaggacagctctggctgctatgaccgccacatcggggtggactgttccgacggcttcaacggcggctgtgagcagctgtgcctccagcagatggcgcccttcccggacgaccccaccttgtataacatcctcatgttctgtgggtgcatcgaggactacaagcttggtgtggatggacgctcttgccaactcatcacggagacctgtccagagggaagtgactgtggggaaagcagggagcttcccatgaaccagaccctctttggggagatgttctttggttacaacaaccattccaaggaagtggctgccggacaggtgctgaaaggaacattcaggcaaaacaactttgctcgtggtttagaccagcaactgccagatggtcttgtggtggccactgtgcccctggagaatcaatgcctagaggagatctcggagcccacccctgaccctgacttcctgactgggatggtgaacttcagtgaagtgtctgggtaccctgtgctgcagcactggaaggtccggtctgtgatgtaccacatcaaactcaaccaagtggccatctctcaggccctcagcaatgctctccactcgctggatggggctacatctcgtgcagattttgtggcgctgttggaccagttcggcaaccattacatccaggaagctatctacggctttgaggagtcctgttctatctggtacccaaacaagcaggtccagcggcgactctggctggagtatgaagacatcagtaaaggcaactccccatcagatgagtctgaggagcgggaaagagaccctaaggtgctgacattcccagaatacatcaccagcttgtcagactccggcaccaagcgcatggcggctggagtccgcatggagtgccagagcaagggacgatgcccctcgtcctgccccctgtgtcatgtgacatccagccctgacacccctgctgagccggttctgctggaggtgaccaaagcagcccccatctatgaactagtgaccaacaaccagacccagaggctcttgcaggaggctaccatgagctctctctggtgctcagggactggagatgtcatcgaggactggtgtcgatgtgactccactgcttttggagctgatggactccccacctgtgcgcctctcccacagcctgtgctgagactctccacggttcacgagcccagcagcactcttgtggtcctggagtgggaacactcagagccaccaatcggggtgcagattgtagattacctcctccgtcaagagaaagtcactgacaggatggaccactccaaagtggagacagaaacagtgctgagctttgtggacgacatcatctctggagcaaagtctccttgtgcaatgccatctcaggtgccggacaagcagctcaccaccatctctctgatcatacgatgcctggaacctgacaccatttacatgttcacgctgtggggagtggacaacacaggacggcgctccaggccaagcgacgtgatcgtgaagaccccatgccccgtggtggatgatgtcaaggctcaagaaatagcagacaagatctacaatctcttcaatggctacactagtgggaaggagcagcagaccgcctacaacaccctcctggatctgggttcccccaccttacaccgggtcctctaccactataaccagcactatgagagttttggggaattcacctggcgatgtgaggatgagttaggtcccaggaaagctggtctcatcctttcccagcttggggacctcagcagttggtgcaatggactccttcaggaacccaagataagcttgcggcgcagctcactcaagtacctggggtgccgctacagcgagatcaaaccctacggacttgactgggcggagctcagccgggacctcaggaagacgtgtgaggagcagaccctgagtatcccctacaacgactatggggacagcaaagagatctagcaccataaggccagggagctgctgccagaatgaagtaggaaagaggagggatccatctgggttggtctgtggatttttaatattttttaatggaacatgaaaacctccacagcaacatcgaaaccagggagaaagtgatccttgctccctgcagaacttcttcagtatgatgttctccatctgcatgattgggaaatctgccagccagtggcttcatgcagtgccatatttctttagaggattactttggggtttgctttgccattaatttgttccattcatttttttttccctgagaagtttaccaaaatgctcaagagctctgccgtgctccccatgaaaagtctattaagtaggcaccctgtgctcactcagttcctaaatccattgcaactgggagcagaggtgaggccagaaagttgttaggcctgccgcaggccccaccctcaagcattcctcaggaagcgtctcactctgggagccttggccctgctcacagagagagacaatagaaaattgaggaaggtggcccttgtctgtgtctcctggttttcttcctaggccttgctatcactatttccatacccgaaaggtgaaaccagctttcattataggccccagtgggccacttgggttttgagatccttccttattttaagccaggactgggattaaatctccctctgtcagatctctgtcccttcctctgaacaacatgatctttgagagggaacaagatgccatctgtcaactgcaccttcagaaaagtctacctgggagactagttagcagtccacattcaagagaagacttggagtttattgttttttaaaaaaaccatgcttcctttggatagacttctccagcctaccaatatatatccatgtgcctggattatctttaacccccacacctcttaccttggacaggtaaggcttggccgatgtctgattgggaccaggaggggtcaacactttcatatcagtgttacagtgaactaaagctattattgatcacaaaaaacttctgttcatccccaccttgctaaatttgcttgtgttgctagttttgcaaatcgtttctctgatgaccataagcaggaggattccaccatggtcactgcccatccagtcacagggattctgtgtagggaagcaccactgattgcagttaacatctagagtgttgtttccatcccactgcccaagcattggcatggtcatgaatggtggcccagccaacaggaagcccagcctttcagaaagagcctggcacggccctgttgactagcaatggccttagctgtcccacacaactcagtggcctgaacacacaccttcagccaccatgcctttgaccagggctcctcatctggaaacatatgagaaaggtcagcaaacagatgcaagacctataaggctagtcattgagctatattggttttttttctaaatagtagtagtgacagataacatttattgagtgcttgctgtatgccaggcgctgatgtaagcactttaggtatcattgaattctcacagcaactcctgaggaaagtgctattcttgtctccattttagtgttgaggaaaacgaggcaaagagaggttatacaacttgcctcaaatcccttgtgcactgtaactcacactgagtttcagtgtgatttcaggctgttggtctccagggcacaggatcttagctactctgggatacccagttctgttttcagtatcatctggcacacaactgtccaagctctcagccccacagggaacctgcccagagagctttacctttcccaagcatctgtggcatggacatgtcctctgtgcagtggaaggaggaggggcgaaagtacgcccttagcctttggagctagagcacccttgggacccctagttccactgcacatggccctcctccccaccctcatgactgggaaggaagcctgtgatgaggctgagataaagcacagggtggtttcactctcctctctccttctttccaaacactgaaggatttatttcaaactctctaatgcacctgcctcagagattcccctactttcaaagcaaagatcagcagaaaaattggctgtcccacctgtggcaaatgctggagcctcagttaaagtgcctcaaggggcaaatatttcaccattgccagagaagatgtgacaggccaatcagacagggcccagagcatctctttgctgctactgttttgccatccttctattcatatctgtgcagaacacggtgttttaagcttgagtgaaaggagggtgaggctgccgatgccttcctgcccagaagtggatgatgtgggagttgacaggccagggagagggtgaagcaggtatcagagtcactcctctgtaccctctccttctgtttttattttaggcacactatcttcctctctcctatctttccctcaatctcccaagttcctctaccttctttatctttgtcttttacttcttctttctgtgaccctccttttttggcctcctctttccccaagactttcttcctcctgtttctcgttgagttctccccactgaatgtgtgtatgtatgtacacacacacacacgtgtgcacacacaatgcacacaactcctatgactggctcctacttacattcaagttaaaaaggctgatatgaacagggcaggggaaaatcttaggatggttgtacaattgactggaggattttttccccttggaagacactattgatctcaacctgctgacttttcctaatgcttacctgaaggaacccatcctggctagaaagggtgatggtactggaccggtattcaaccttgagttttcaagctgccaaacaggtcttaagggaggtgcttatatcccaccaacactctcccagctcccatgtccccaagacctctggagtttcctcttgaatgtacatgaaccactgtaatagcattagacttttaattgagtgtgcaatcgttttccatggagtttggtccgttcattattttttagttaactacacttcttgatattcaaatgttctattaaaaaaactgagtatgaagaaaaacactttactactgcagaaggaagaaagaatataatatgaccatcttcaggtataacagtgttgtttaaaagagaattattgtatgattataaaagatgaaataattaactgaataataaaacaaagctattagtaagc

SEQ ID NO: 42 - protein, 1302 aa, Homo sapiens
HASGPGLKMALAGLCALLACCWGPAAVLATAAGDVDPSKELECKLKSITVSALPFLRENDLSIMHSPSASEPKLLFSVRNDFPGEMVVVDDLENTELPYFVLEISGNTEDIPLVRWRQQWLENGTLLFHIHHQDGAPSLPGQDPTEEPQHESAEEELRILHISVMGGMIALLLSILCLVMILYTRRRWCKRRRVPQPQKSASAEAANEIHYIPSVLIGGHGRESLRNARVQGHNSSGTLSIRETPILDGYEYDITDLRHHLQRECMNGGEDFASQVTRTLDSLQGCNEKSGMDLTPGSDNAKLSLMNKYKDNIIATSPVDSNHQQATLLSHTSSSQRKRINNKARAGSAFLNPEGDSGTEAENDPQLTFYTDPSRSRRRSRVGSPRSPVNKTTLTLISITSCVIGLVCSSHVNCPLVVKITLHVPEHLIADGSRFILLEGSQLDASDWLNPAQVVLFSQQNSSGPWAMDLCARRLLDPCEHQCDPETGECLCYEGYMKDPVHKHLCIRNEWGTNQGPWPYTIFQRGFDLVLGEQPSDKIFRFTYTLGEGMWLPLSKSFVIPPAELAINPSAKCKTDMTVMEDAVEVREELMTSSSFDSLEVLLDSFGPVRDCSKDNGGCSKNFRCISDRKLDSTGCVCPSGLSPMKDSSGCYDRHIGVDCSDGFNGGCEQLCLQQMAPFPDDPTLYNILMFCGCIEDYKLGVDGRSCQLITETCPEGSDCGESRELPMNQTLFGEMFFGYNNHSKEVAAGQVLKGTFRQNNFARGLDQQLPDGLVVATVPLENQCLEEISEPTPDPDFLTGMVNFSEVSGYPVLQHWKVRSVMYHIKLNQVAISQALSNALHSLDGATSRADFVALLDQFGNHYIQEAIYGFEESCSIWYPNKQVQRRLWLEYEDISKGNSPSDESEERERDPKVLTFPEYITSLSDSGTKRMAAGVRMECQSKGRCPSSCPLCHVTSSPDTPAEPVLLEVTKAAPIYELVTNNQTQRLLQEATMSSLWCSGTGDVIEDWCRCDSTAFGADGLPTCAPLPQPVLRLSTVHEPSSTLVVLEWEHSEPPIGVQIVDYLLRQEKVTDRMDHSKVETETVLSFVDDIISGAKSPCAMPSQVPDKQLTTISLIIRCLEPDTIYMFTLWGVDNTGRRSRPSDVIVKTPCPVVDDVKAQEIADKIYNLFNGYTSGKEQQTAYNTLLDLGSPTLHRVLYHYNQHYESFGEFTWRCEDELGPRKAGLILSQLGDLSSWCNGLLQEPKISLRRSSLKYLGCRYSEIKPYGLDWAELSRDLRKTCEEQTLSIPYNDYGDSKEI

SEQ ID NO: 43 - DNA, 1690 bp, Homo sapiens
gaggaattcagagttaaaccttgagtgcctgcgtccgtgagaattcagcatggaatgtctctactatttcctgggatttctgctcctggctgcaagattgccacttgatgccgccaaacgatttcatgatgtgctgggcaatgaaagaccttctgcttacatgagggagcacaatcaattaaatggctggtcttctgatgaaaatgactggaatgaaaaactctacccagtgtggaagcggggagacatgaggtggaaaaactcctggaagggaggccgtgtgcaggcggtcctgaccagtgactcaccagccctcgtgggctcaaatataacatttgcggtgaacctgatattccctagatgccaaaaggaagatgccaatggcaacatagtctatgagaagaactgcagaaatgaggctggtttatctgctgatccatatgtttacaactggacagcatggtcagaggacagtgacggggaaaatggcaccggccaaagccatcataacgtcttccctgatgggaaaccttttcctcaccaccccggatggagaagatggaatttcatctacgtcttccacacacttggttggcttttacaaacccctaagcttcttctttacctttccttaaaatttcaaccttctcttttcttactctataattgagaatgataacacagagagttaataacagtcaccctgctaactttccttagcatgagtgaacagtgagagataaaaatgaaatcttggttaaccttgccaaatctccaggacaccgaagagttaaaaagagagaaaaacaaaaagattaagctctttttcaaaaaaacaaaaccacttaatttttttctacctaaaaccataacaagaaaaaatgctaacacttatttatttgaatggcacatggagaccgggcatgtggctcacacttgtaatcccagcaccttggaaggcggaggcgggtggatcacctgaagtcaggagttcaagaccagcctggccaacatggtgaagtcccgtccctactaaaaatacaaaaattagccaggtgtggtggtgcgcacctgtaatcccagctactcaggaggctgaggcaggagaatcacttgaatccgggaggtggaggttgcagtgagaggagattgagccattgcactccagcctgggcaacagagtgagactccatctcgaaaaacaaacaaacaaacaaaaaacagaatggcacattgatgagcattcattgattgattctttagtttttttatgttctctaaagaatttttaagatttaaagaagcatgtgctatttatttgtaggaaatcctcagaaaaggtacaaataaaaaataaaaattatccataattaataccagagattataattgttaattattatggtgttttctttgctagtatttaagatcattattaagatcacatacacatttttgcttactatcattagcatttgatgatatgatttttttaatttttatacattgttttaatggctgcacgatatttcattgtgtacaaataaaataactactgttccgctttgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - protein, 206 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGWLLQTPKLLLYLSLKFQPSLFLLYN

SEQ ID NO: 45 - DNA, 479 bp, Homo sapiens
ttttttttgggtaacataagacatttattactttatactaattttttcattcataaaaaggacaaagcacagtcctatactactccattgaaaaaatgataaaaaataactaaaaaatcaattcaatatttatcagtatcaaataaaactactatcacctttcctgaaatacaaagaaacaacagatgtatctatacctatataaagtttaattcagaaatcttgcgtcttaaagcagatgattattagttagcttgacaacagtttaaaactgatggtcccagttaaatctgtacaactgtatgagaaaatgaaaagcttgagttatcagtgtacgagagattttaaactactttatctctgtcagaagttcaaaactaaacaacctccaaagtctgttttcctcttacctttcagaaccatttcatgcaaaatctaaccagttttgctcgttattatcatatattagaaaataaaag

SEQ ID NO: 46 - protein, 54 aa, Homo sapiens
MVPVKSVQLYEKMKSLSYQCTRDFKLLYLCQKFKTKQPPKSVFLLPFRTISCKI

SEQ ID NO: 47 - DNA, 3198 bp, Homo sapiens
ttgggaggagcagtctctccgctcgtctcccggagctttctccattgtctctgcctttacaacagagggagacgatggactgagctgatccgcaccatggagtctcgggtcttactgagaacattctgtttgatcttcggtctcggagcagtttgggggcttggtgtggacccttccctacagattgacgtcttaacagagttagaacttggggagtccacgaccggagtgcgtcaggtcccggggctgcataatgggacgaaagcctttctctttcaagatactcccagaagcataaaagcatccactgctacagctgaacagttttttcagaagctgagaaataaacatgaatttactattttggtgaccctaaaacagacccacttaaattcaggagttattctctcaattcaccacttggatcacaggtacctggaactggaaagtagtggccatcggaatgaagtcagactgcattaccgctcaggcagtcaccgccctcacacagaagtgtttccttacattttggctgatgacaagtggcacaagctctccttagccatcagtgcttcccatttgattttacacattgactgcaataaaatttatgaaagggtagtagaaaagccctccacagacttgcctctaggcacaacattttggctaggacagagaaataatgcgcatggatattttaagggtataatgcaagatgtccaattacttgtcatgccccagggatttattgctcagtgcccagatcttaatcgcacctgtccaacttgcaatgacttccatggacttgtgcagaaaatcatggagctacaggatattttagccaaaacatcagccaagctgtctcgagctgaacagcgaatgaatagattggatcagtgctattgtgaaaggacttgcaccatgaagggaaccacctaccgagaatttgagtcctggatagacggctgtaagaactgcacatgcctgaatggaaccatccagtgtgaaactctaatctgcccaaatcctgactgcccacttaagtcggctcttgcgtatgtggatggcaaatgctgtaaggaatgcaaatcgatatgccaatttcaaggacgaacctactttgaaggagaaagaaatacagtctattcctcttctggagtatgtgttctctatgagtgcaaggaccagaccatgaaacttgttgagagttcaggctgtccagctttggattgtccagagtctcatcagataaccttgtctcacagctgttgcaaagtttgtaaaggttatgacttttgttctgaaaggcataactgcatggagaattccatctgcagaaatctgaatgacagggctgtttgtagctgtcgagatggttttagggctcttcgagaggataatgcctactgtgaagacatcgatgagtgtgctgaagggcgccattactgtcgtgaaaatacaatgtgtgtcaacaccccgggttcttttatgtgcatctgcaaaactggatacatcagaattgatgattattcatgtacagaacatgatgagtgtatcacaaatcagcacaactgtgatgaaaatgctttatgcttcaacactgttggaggacacaactgtgtttgcaagccgggctatacagggaatggaacgacatgcaaagcattttgcaaagatggctgtaggaatggaggagcctgtattgccgctaatgtgtgtgcctgcccacaaggcttcactggacccagctgtgaaacggacattgatgaatgctctgatggttttgttcaatgtgacagtcgtgctaattgcattaacctgcctggatggtaccactgtgagtgcagagatggctaccatgacaatgggatgttttcaccaagtggagaatcgtgtgaagatattgatgagtgtgggaccgggaggcacagctgtgccaatgataccatttgcttcaatttggatggcggatatgattgtcgatgtcctcatggaaagaattgcacaggggactgcatccatgatggaaaagttaagcacaatggtcagatttgggtgttggaaaatgacaggtgctctgtgtgctcatgtcagaatggattcgttatgtgtcgacggatggtctgtgactgtgagaatcccacagttgatcttttttgctgccctgaatgtgacccaaggcttagtagtcagtgcctccatcaaaatggggaaactttgtataacagtggtgacacctgggtccagaattgtcaacagtgccgctgcttgcaaggggaagttgattgttggcccctgccttgcccagatgtggagtgtgaattcagcattctcccagagaatgagtgctgcccgcgctgtgtcacagacccttgccaggctgacaccatccgcaatgacatcaccaagacttgcctggacgaaatgaatgtggttcgcttcaccgggtcctcttggatcaaacatggcactgagtgtactctctgccagtgcaagaatggccacatctgttgctcagtggatccacagtgccttcaggaactgtgaagttaactgtctcatgggagatttctgttaaaagaatgttctttcattaaaagaccaaaaagaagttaaaacttaaattgggtgatttgtgggcagctaaatgcagctttgttaatagctgagtgaactttcaattatgaaatttgtggagcttgacaaaatcacaaaaggaaaattactggggcaaaattagacctcaagtctgcctctactgtgtctcacatcaccatgtagaagaatgggcgtacagtatataccgtgacatcctgaaccctggatagaaagcctgagcccattggatctgtgaaagcctctagcttcactggtgcagaaaattttcctctagatcagaatcttcagaatcagttaggttcctcactgcaagaaataaaatgtcaggcagtgaatgaattatattttcagaagtaaagcaaagaagctataacatgttatgtacagtacactctgaaaagaaatctgaaacaagttattgtaatgataaaaataatgcacaggcatggttacttaatattttctaacaggaaaagtcatccctatttccttgttttactgcacttaatattatttggttgaatttgttcagtataagctcgttcttgtgcaaaattaaataaatatttctcttacctt

SEQ ID NO: 48 - protein, 816 aa, Homo sapiens
MESRVLLRTFCLIFGLGAVWGLGVDPSLQIDVLTELELGESTTGVRQVPGLHNGTKAFLFQDTPRSIKASTATAEQFFQKLRNKHEFTILVTLKQTHLNSGVILSIHHLDHRYLELESSGHRNEVRLHYRSGSHRPHTEVFPYILADDKWHKLSLAISASHLILHIDCNKIYERVVEKPSTDLPLGTTFWLGQRNNAHGYFKGIMQDVQLLVMPQGFIAQCPDLNRTCPTCNDFHGLVQKIMELQDILAKTSAKLSRAEQRMNRLDQCYCERTCTMKGTTYREFESWIDGCKNCTCLNGTIQCETLICPNPDCPLKSALAYVDGKCCKECKSICQFQGRTYFEGERNTVYSSSGVCVLYECKDQTMKLVESSGCPALDCPESHQITLSHSCCKVCKGYDFCSERHNCMENSICRNLNDRAVCSCRDGFRALREDNAYCEDIDECAEGRHYCRENTMCVNTPGSFMCICKTGYIRIDDYSCTEHDECITNQHNCDENALCFNTVGGHNCVCKPGYTGNGTTCKAFCKDGCRNGGACIAANVCACPQGFTGPSCETDIDECSDGFVQCDSRANCINLPGWYHCECRDGYHDNGMFSPSGESCEDIDECGTGRHSCANDTICFNLDGGYDCRCPHGKNCTGDCIHDGKVKHNGQIWVLENDRCSVCSCQNGFVMCRRMVCDCENPTVDLFCCPECDPRLSSQCLHQNGETLYNSGDTWVQNCQQCRCLQGEVDCWPLPCPDVECEFSILPENECCPRCVTDPCQADTIRNDITKTCLDEMNVVRFTGSSWIKHGTECTLCQCKNGHICCSVDPQCLQEL

SEQ ID NO: 49 - DNA, 562 bp, Homo sapiens
tttatagtgaaaacattatattataacatgcttttgcaaacaaaatattaaaattaataatttttaacatattctttaaattctacatgcatacttttgaatatctaaactacatgttaaacagctgaatacattctactcacacttcagatctttaaacaccaacaatctatgaatattaatctattactacaggacaaatttggatatacgtcttggataaattttaagctcactttaagagcaccaatcattaacaatcatttgtgtattttattcacaaacactgatacgatttgtttatttatgttaaaacaaacattttctttaaaaatgaatgtgtattaaagtagtttaactggtagaataggctttattccaatctgtttgttaaacagcctattttcacaatatctatatctacttttcattgatctgttccatcattactaacatatttgttcaaattattaggactattttttcaaagggaggaataatcaaattccccagtccatatatcttataaatattttacacctaatacacacagctttacagt

SEQ ID NO: 50 - protein, 58 aa, Homo sapiens
MNINLLLQDKFGYTSWINFKLTLRAPIINNHLCILFTNTDTICLFMLKQTFSLKMNVY

SEQ ID NO: 51 - DNA, 1672 bp, Homo sapiens
aaaaaaattaaaaaaaaaaaaaaaaacagaaaaaaaaacatagtacatgccaagatattattatgacaattacaaatacaaataaattatgatctttgacctcagcatatttattaactaaaagggaagataaaacaggcacataactataacaggggcaccagtcatgggcgccgcagccgctcaggcgcctctcgggctgcctgcggcctccgctcgccttctgctgctagcgacgtcggtgcttctgctcttcgccttctctctgcccgggagccgcgcgtccaaccagcccccgggtggtggcggcggcacgggcggggactgtcccggcggcaaaggcaagagcatcaactgctcagaattaaatgtgagggagtctgacgtaagagtttgtgatgagtcatcatgtaaatatggaggagtctgtaaagaagatggagatggtttgaaatgtgcatgccaatttcagtgccatacaaattatattcctgtctgtggatcaaatggggacacttatcaaaatgaatgctttctcagaagggctgcttgtaagcaccagaaagagataacagtaatagcaagaggaccatgctactctgataatggatctggatctggagaaggagaagaggaagggtcaggggcagaagttcacagaaaacactccaagtgtggaccctgcaaatataaagctgagtgtgatgaagatgcagaaaatgttgggtgtgtatgtaatatagattgcagtggatacagttttaatcctgtgtgtgcttctgatgggagttcctataacaatccctgttttgttcgagaagcatcttgtataaagcaagaacaaattgatataaggcatcttggtcattgcacagatacagatgacactagtttgttgggaaagaaagatgatggactacaatatcgaccagatgtgaaagatgctagtgatcaaagagaagatgtttatattggaaaccacatgccttgccctgaaaacctcaatggttactgcatccatggaaaatgtgaattcatctatctactcagaagggcttcttgtagatgtgaatctggctacactggacagcactgtgaaaagacagactttagtattctctatgtagtgccaagtaggcaaaagctcactcatgttcttattgcagcaattattggagctgtacagattgccatcatagtagcaattgtaatgtgcataacaagaaaatgccccaaaaacaatagaggacgtcgacagaagcaaaacctaggtcattttacttcagatacgtcatccagaatggtttaaactgatgacttttatatgtacactgaccatgtgtatgtacatttattatgtctttttttaaagaatggaaatatttatttcagaaggccttatttttggacattttatagtgtagtactgttggctcgatatttgaatattcagctacgacagttttggactgtttagtagtctttgttttatgtttttaaatacagaaattgcttcacaaatttgtaccacatggtaattctaagacttgttctttacccatggaatgtaatatttttgcaaagatggactacttcacaaatggttataaagtcatatccacttcttccacaatgaccacagcaaatgacccaagcatgaactaaagaagag

SEQ ID NO: 52 - protein, 380 aa, Homo sapiens
MGAAAAQAPLGLPAASARLLLLATSVLLLFAFSLPGSRASNQPPGGGGGTGGDCPGGKGKSINCSELNVRESDVRVCDESSCKYGGVCKEDGDGLKCACQFQCHTNYIPVCGSNGDTYQNECFLRRAACKHQKEITVIARGPCYSDNGSGSGEGEEEGSGAEVHRKHSKCGPCKYKAECDEDAENVGCVCNIDCSGYSFNPVCASDGSSYNNPCFVREASCIKQEQIDIRHLGHCTDTDDTSLLGKKDDGLQYRPDVKDASDQREDVYIGNHMPCPENLNGYCIHGKCEFIYLLRRASCRCESGYTGQHCEKTDFSILYVVPSRQKLTHVLIAAIIGAVQIAIIVAIVMCITRKCPKNNRGRRQKQNLGHFTSDTSSRMV

SEQ ID NO: 53 - DNA, 2299 bp, Homo sapiens
agcgggcggctgctaggaggcaccgaggcagcggcggggctctgggcgcgcggctggatgcccccggcctgcggctccctgcgcttcccgccgtccaggggcaccagtcatgggcgccgcagccgctgaggcgccgctccggctgcctgccgcgcctccgctcgccttctgctgctacacgtcggtgcttctgctcttcgccttctctctgccagggagccgcgcgtccaaccagcccccgggtggtggcggcggcagcggcggggactgtcccggcggcaaaggcaagagcatcaactgctcagaattaaatgtgagggagtctgacgtaagagtttgtgatgagtcatcatgtaaatatggaggagtctgtaaagaagatggagatggtttgaaatgtgcatgccaatttcagtgccatacaaattatattcctgtctgtggatcaaatggggacacttatcaaaatgaatgctttctcagaagggctgcttgtaagcaccagaaagagataacagtaatagcaagaggaccatgctactctgataatggatctggatctggagaaggagaagaggaagggtcaggggcagaagttcacagaaaacactccaagtgtggaccctgcaaatataaagctgagtgtgatgaagatgcagaaaatgttgggtgtgtatgtaatatagattgcagtggatacagttttaatcctgtgtgtgcttctgatgggagttcctataacaatccctgttttgttcgagaagcatcttgtataaagcaagaacaaattgatataaggcatcttggtcattgcacagatacagatgacactagtttgttgggaaagaaagatgatggactacaatatcgaccagatgtgaaagatgctagtgatcaaagagaagatgtttatattggaaaccacatgccttgccctgaaaacctcaatggttactgcatccatggaaaatgtgaattcatctattctactcagaaggcttcttgtagatgtgaatctggctacactggacagcactgtgaaaagacagactttagtattctctatgtagtgccaagtaggcaaaagctcactcatgttcttattgcagcaattattggagctgtacagattgccatcatagtagcaattgtaatgtgcataacaagaaaatgccccaaaaacaatagaggacgtcgacagaagcaaaacctaggtcattttacttcagatacgtcatccagaatggtttaaactgatgacttttatatgtacactgaccatgtgatgtacatttattatgtctttttttaaagaatggaaatatttatttcagaggccttatttttggacatttttagtgtagtactgttggctcgtatttagaatattcagctacgacagttttggactgtttagtagtctttgttttatgtttttaaatacagaaattgctttcacaaatttgtaccacatggtaattctaagacttgttctttacccatggaatgtaatatttttgcaaagatggactacttcacaaatggttataaagtcatatccacttcttccacaatgaccacagcaaatgaccaagcatgaactaaaggtaaagatgtttacagattacttttcttacaaaaaaatctagaagacactgtgtttaaatagatatttaaatgtttttgagatttagtaactgattttttagacactgcctatcgcatgaactgtaaagctgtgtgtattaggtgtaaaatatttataagatatatggactggggaatttgattattcctccctttgaaaaaatagtcctaataatttgaacaaatatgttagtaatgatggaacagatcaatgaaaagtagatatagatattgtgaaaataggctgtttaacaaacagattggaataaagcctattctaccagttaaactactttaatacacattcatttttaaagaaaatgtttgttttaacataaataaacaaatcgtatcagtgtttgtgaataaaatacaaaaatgattgttaatgattggtgctcttaaagtgagcttaaaatttatccaagacgtatatccaaatttgtcctgtagtaatagattaatattcatagattgttggtgtttaaagatctgaagtgtgagtagaatgtattcagctgtttaacatgtagtttagatattcaaaagtatgcatgtagaatttaaagaatatgttaaaaattattaatcttaatattttgtttggaaaagcatgttataatataatgttttcacaaaaaaaaaaaaaaaa

SEQ ID NO: 54 - protein, 380 aa, Homo sapiens
MGAAAAEAPLRLPAAPPLAFCCYTSVLLLFAFSLPGSRASNQPPGGGGGSGGDCPGGKGKSINCSELNVRESDVRVCDESSCKYGGVCKEDGDGLKCACQFQCHTNYIPVCGSNGDTYQNECFLRRAACKHQKEITVIARGPCYSDNGSGSGEGEEEGSGAEVHRKHSKCGPCKYKAECDEDAENVGCVCNIDCSGYSFNPVCASDGSSYNNPCFVREASCIKQEQIDIRHLGHCTDTDDTSLLGKKDDGLQYRPDVKDASDQREDVYIGNHMPCPENLNGYCIHGKCEFIYSTQKASCRCESGYTGQHCEKTDFSILYVVPSRQKLTHVLIAAIIGAVQIAIIVAIVMCITRKCPKNNRGRRQKQNLGHFTSDTSSRMV

SEQ ID NO: 55 - DNA, 2408 bp, Homo sapiens
gttgcctgcatgagtgtgtgctctgtgtcactgtggattggagttgaaaaagcttgactggcgtcattcaggagctggatggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgaggtaaggacatcctttggaaacattaatttttcattgagtttggcttgggcccgactaacatggtaatagacctgaatgcataatgagttcttactttgctatcatcaaaagacttttcatcacagttacatactttctaatttatggaaaaacagcatttggaaaacaaatgttttgtttttatttttttaaagatttaaaaaataaatcaactagggactaggaatcaacaactgtgagtgagttaaactgtgttgaaatactaaagggttgtgaaagattagtgacaaagaagaacaaaagtctaaacctgtttattcctgtctatttcccacagaaagaatgagcaataaatggtacctcatataaattaataaataagaaggcctttctttttaaccaagggggtagatgtctacctttgtttgctttactaattaggtgagctcttttgattattattattaattatatttttggttcatatctctaatttctttatataatgggaattgctaaacttgactaatctactgtatacttataaatcagtcaaaattcatttacttttcagtagcaagaattacctccgtgactccggactcttattataagcctaccctataataaagaatgttaatctattcctattaaagtgttactttgagaaaaggaattctttcccacaagatcagtactcatttacttgaaatacattattttttataggaacaaacatttagtgagtactctggcaagtgaattaaacgaaggatggcatatcggctagttttctttatcacaaccgccagtgccatcatcatcatcatctaatgttttctgagcacctactatgtgctggacttttcttattcatcagcaaagacatttcttatacttccatgttatttggttgaaatctgagtcttaagaaagccaagtttaaaatattaactgagttttgctggaacccagcatataatacatgctgaataaatgtttgttaaatcatgagagaatgatgaatatattaatgttgataacaataatagtaatgacaataatggccaacattttggtatactcatgcttaatatatgtcatctcacttatccttgaaaacaacctattgttaggtcctattgttatcattcctgttttacatatgcagacactgaaactcagagaggttatttgttttccccaaatcacacagttgacaataaagacctgggatttgaaccccaaatttatcggacttcaaagtttatatccttgataaaaatagagaacccatgaatttgggtgacagccatcctttacaaagacatatcaagctgcttctttgctcaatatatttaaaaatagaacagaatctttcctttcagtgttggttggcacggaggtagagttagctggtgagccgagacggctgtaggttctgcctatggctgagattttgtaatacctctggagattaagtgggttttagaagaatgcttaaggtagtagagtttggactggggagacagaacaaggtggagggaacactgaaggttgtggttagttctactttcccaggcttctcagccgggtcttagaaagttcatgtgaggacttggccagttagaactaaattgaacattcctcactcagtacacttgtcaagttacaggcccattctaagtcatgctgcattaaataaacaatgctttaaaaaatgtccatctccatagacctcttatctaaaaatcacctatttcatatcaggtgaatgccatgcattttgtagaggctagagaataacttagcggtaggggagatggcataggagtcaatgcctctctggtgggctactttaaaaaaaatacaactttctccataacttttatgtccctatattttgtgttgtggtatttgtgagaggtactttgcatttataccttcaggagacttggttcagcatctagttaattatctacatgtaggtgcttaatatatgccatgccaccctttttgtctccctgtatcatattctatgctaataaaattattttcagcactctaaaaaaaaaaaaaaaaaaaaaaaaaaaaaa

SEQ ID NO: 56 - protein, 99 aa, Homo sapiens
MAWDMCNQDSESVWSDIECAALVGEDQPLCPDLPELDLSELDVNDLDTDSFLGGLKWCSDQSEIISNQYNNEPSNIFEVRTSFGNINFSLSLAWARLTW

SEQ ID NO: 57 - DNA, 3558 bp, Homo sapiens
cagaccccagttcgccgactaagcagaagaaagatcaaaaaccggaaaagaggagaagagcaaacaggcactttgaggaacaatcccctttaactccaagccgacagcggtctaggaattcaagttcagtgcctaccgaagacaaaggcgccccgagggagtggcggtgcgaccccagggcgtgggcccggccgcggagcccacactgcccggctgacccggtggtctcggaccatgtctcccgccccaagacccccccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccaccgccggcccgcccactcctaccacaaggactttgcctctgaaggccagtggcagcaggtggtggtgggtgggctgctcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctgggcactccagccctgaaagccccaggtgtacccaattggcagcctctcactactctttctggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatctgcctctgcctcacctaccccagggaacttccaaggaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagcctgaaggccacagagaaagaaccttgcccaaactcaggcagctggggctgaggcccaaaggcagaacagccagagggggcaggaggggaccaaaaaggaaaatgaggacgtgcagcagcattggaaggctggggccgggcaggccaggccaagccaagcagggggccacagggtgggctgtggagctctcaggaagggccctgaggaaggcacacttgctcctgttggtccctgtccttgctgcccaggcagcgtggaggggaagggtagggcagccagagaaaggagcagagaaggcacacaaacgaggaatgaggggcttcacgagaggccacagggcctggctggccacgctgtcccggcctgctcaccatctcagtgaggggcaggagctggggctcgcttaggctgggtccacgcttccctggtgccagcacccctcaagcctgtctcaccagtggcctgccctctcgctcccccacccagcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtcttgtttgtttttttgttttgtttaatgtatatttttattataattattatatatgaattccaaaaaaaaaaaaaaaaaaaaa

SEQ ID NO: 58 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 59 - DNA, 2128 bp, Homo sapiens
caagacttctctgcattttctgccaaaatctgtgtcagatttaagacacatgcttctgcaagcttccatgaaggttgtgcaaaaaagtttcaatccagagttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 60 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 61 - DNA, 2028 bp, Homo sapiens
ccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgatcatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagtctaaaaaaaaaaaaaaaaaaaaaaaa

SEQ ID NO: 62 - protein, 354 aa, Homo sapiens; identical to SEQ ID NO: 60